(19) United States
(12) Patent Application Publication
(10) Pub. No.: US 2024/0229143 A1
(43) Pub. Date: Jul. 11, 2024

(54) FORMULATION OF PEPTIDE IMMUNOTHERAPIES

(71) Applicant: IOGENETICS, LLC, Madison, WI (US)

(72) Inventors: Jane Homan, Marshall, WI (US); Robert D. Bremel, Marshall, WI (US); Hassan Benameur, Lyon (FR)

(21) Appl. No.: 18/256,243

(22) PCT Filed: Dec. 7, 2021
(86) PCT No.: PCT/US2021/062140

§ 371 Date: Jun. 7, 2023

Related U.S. Application Data
(60) Provisional application No. 63/122,195, filed on Dec. 7, 2020.
(60) Provisional application No. 63/122,196, filed on Dec. 7, 2020.

(51) Int. Cl. C12Q 1/6886; A61K 38/03; A61K 39/00

(52) U.S. Cl. CPC C12Q 1/6886; A61K 38/03; A61K 39/0011; C12Q 2600/156

(57) ABSTRACT

This invention provides a method for maximizing the immune response to mutated tumor specific proteins, either by means of stimulation of dendritic cells or T cells in vitro followed by administration of these cells to a patient, or by means of administration of a neoantigen vaccine in which de novo peptides, or their encoding nucleic acids, have been designed to ensure an appropriate level of binding affinity to a particular cancer patient's MHC alleles. This invention further provides for modulating the immune response in an immunopathology

### SEQUENCE LISTING STATEMENT

The contents of the electronic sequence listing titled IOGEN_39031_252_SequenceListing_Corrected.xml (Size: 1,700,585 bytes; and Date of Creation: Dec. 13, 2023) is herein incorporated by reference in its entirety.

### FIELD OF THE INVENTION

The present invention relates to methods to stimulate T cell responses to a particular target peptide in a protein, where the target peptide comprises an amino acid of interest. In some cases the target protein is a tumor protein that comprises a mutated amino acid. In other cases the target protein is a tumor protein that does not comprise a mutated amino acid but is upregulated or overexpressed. In yet other cases the target protein is one that comprises an epitope that elicits an immune response that is excessive or is deficient in an immunopathology. Stimulation of the T cell response may be intended to up-regulate the response or to dampen or modulate the T cell response. The methods further comprise delivery of a target peptide to a subject as a peptide or encoded in a nucleic acid.

To facilitate delivery of target peptides to a subject, the present invention provides methods for enhancing the manufacturing and formulation of peptides that are selected as antigens for application as a personalized vaccine to subjects affected by cancer or an immunopathology, in which peptides, or their encoding nucleic acids, have been designed to ensure an appropriate level of binding affinity to a particular subject's MHC alleles and to enhance or modulate the immune response. To enhance manufacturing and formulation peptides are further selected based on the amino acid composition that favors stability, solubility and reduced aggregation.

### BACKGROUND OF THE INVENTION

Immunotherapies which employ neoepitope vaccines have shown significant benefits to cancer patients, either as peptides or as their encoding nucleic acids, by stimulating T cell responses. In some instances, these have been peptides derived from unmutated tumor associated antigens proteins. In other instances, the peptides comprise neoantigens that are specific to tumor cells due to mutations, insertions, deletions, fusions or splicing they embody. In immunopathologies other than solid tumors, including but not limited to autoimmunity, allergies and inflammation, an excessive immune response by T cells may drive the pathology. In such a situation the provision of a very high affinity MHC binding peptide may allow dampening, or modulation, of the T cell response by causing specific clones to become exhausted and anergic. Alternatively, the immunopathology may arise from a deficient immune response that requires boosting. As these are clonal-specific interventions and focused on specific epitopes, the design of peptides which can bring about such modulation is, in most cases, specific to the individual subject.

The present invention addresses interventions to bring about a desired T cell mediated immune response which may be needed in particular situations. These include stimulating a T cell response to a tumor protein which contains a unique mutation, stimulating a T cell response to a protein in a tumor in which particular proteins are increased number, or modulating a T cell response that is contributing to an immunopathology or which is deficient in a subject affected by an immunopathology. T cell responses may be driven by either CD8+ cytotoxic T cells targeting peptides bound in MHC I molecules or CD4+ helper responses targeting peptides bound in MHC II molecules, or the combination of presentation of peptides on MHC I and peptides presented on MHC II molecules. In each case a combination of amino acids in the peptide is exposed to and engages with the T cell receptor, while other flanking amino acids in the peptide determine the binding to the MHC molecular groove. The binding affinity of the peptide to the MHC is determined by the allele of each MHC, a distinct combination for any individual subject.

A challenge in manufacturing and delivering peptide immunogens is that the composition is determined by the specific T cell epitope and this cannot be changed. This may result in a peptide which is more or less soluble or stable in a particular carrier. Furthermore, a cancer vaccine typically comprises multiple peptides, each with different characteristics.

By modifying the flanking amino acids, while maintaining the T cell exposed motif constant, two key objectives can be addressed. First, the binding affinity may be optimized to ensure presentation of the peptide to T cells by the MHC molecules of the particular individual and to modulate the binding affinity to achieve the desired duration and frequency of engagement, and hence modulate the T cell stimulation. Secondly, by modifying the flanking amino acids the characteristics of the peptide may be adjusted to facilitate manufacturing, formulation, and delivery of the peptide to the subject. The present invention therefore addresses several aspects needed by the art to improve the selection, design and delivery of immunotherapy to subjects affected by cancer or by immunopathologies, including the selection of suitable peptide targets to present epitopes for effective stimulation of T cells in a particular individual, and the design of such peptide targets to optimize the presentation of such epitopes and to facilitate their manufacturing, formulation, and delivery to a particular subject in need of T cell stimulation.

### SUMMARY OF THE INVENTION

The present invention provides methods for designing synthetic peptides intended to serve as personalized vaccines for individuals affected by cancer or immunopathologies, and the design of such peptides in order to facilitate their manufacture and formulation.

In some preferred embodiments, the present invention provides methods for treating cancer in a subject by targeting T cells to personal tumor-specific mutations. The methods comprise designing a group of one or more tumor-specific T-cell stimulating peptides, or nucleic acids encoding T cell stimulating peptides, which have a desired predicted binding affinity for the MHC alleles of the subject and which are further designed to have characteristics that facilitate formulation and delivery. Such methods comprise the following steps: obtaining a biopsy of the subject's tumor and a comparative sample of normal tissue; sequencing DNA in both biopsy and normal samples, and sequencing the RNA in the biopsy, obtaining sequences of the proteins in the biopsy and identifying the mutated amino acids in the proteins of the biopsy and the peptide comprising each the mutated amino acids; determining T cell exposed motifs which comprise mutated amino acids in each of the proteins; determining the predicted binding affinity to the subject's MHC alleles of peptides which comprises each the T cell exposed motif, or a subset thereof; generating an array of alternative peptides that are not present in the tumor, wherein each peptide in the array comprises the amino acids of one of the T cell exposed motifs, and in which the amino acids not lying within the T cell exposed motif are substituted to change the predicted MHC binding affinity; selecting a group of one or more selected peptides from the array of alternative peptides which have a desired predicted binding affinity for one or more of the subject's MHC alleles and then further selecting those peptides which have desirable characteristics for formulation and delivery, and synthesizing the group of one or more selected peptides, or nucleic acids encoding the selected peptides.

In some preferred embodiments a further selective criterion is applied to ensure that the selected peptides are in proteins which are expressed in the tumor. In such embodiments a determination is made of the ratio of the DNA encoding a gene of interest in the tumor biopsy and the RNA transcribing that gene locus and thus expressed as protein in the biopsy. In some embodiments the criteria applied are that the DNA encoding the mutant amino acid is present in at least 10% of the DNA reads for that gene in the biopsy and the RNA is transcribed from at least 10% of that DNA read count. In yet other embodiments the RNA is transcribed from at least 20% of the DNA reads for that gene. In further embodiments the DNA encoding the mutant amino acid is present in at least 3% of the DNA reads for that gene in the biopsy and the RNA is transcribed from at least 10% of the DNA read count.

In some embodiments, the MHC alleles are MHC type I and the T cell response is a CD8+ response. In some preferred embodiments, the MHC alleles are MHC type II and the T cell response is a CD4+ response. In some embodiments, the selected peptides are 8 to 10 amino acids long, while in other embodiments the selected peptides are 11-22 amino acids long. In other preferred embodiments the selected peptides are chosen to stimulate a cytotoxic T cell response, whereas in other embodiments the selected peptides are chosen to stimulate a CD4+T helper response. In some instances, both CD8+ and CD4+ stimulating peptides are chosen and are administered together. Here some peptides, or the peptides encoded by nucleic acids, are selected to bind MHC I alleles while others are selected to bind MHC II alleles.

By altering the amino acids in the positions flanking the T cell exposed motif, i.e. those amino acid positions which comprise the groove exposed motif, it is possible to select a peptide of desired binding affinity for a particular MHC allele. The intended application determines what is the desired binding affinity. In the case of T cell stimulation to target a cancer peptide the desired affinity is that which stimulates an active response and, hence, a peptide is selected to have a binding affinity of less than 500 nanomolar, less than 200 nanomolar, or less than 100 nanomolar. However, a higher binding affinity of less than 50 nanomolar is more desired when the goal is to bring about T cell exhaustion or anergy or to stimulate a T regulatory response. Thus, the present invention provides methods to select peptides of a particular predicted binding affinity to a particular MHC allele. As the goal is to provide T cells that can target a T cell exposed motif that is bound and presented in the natural tumor setting, in some embodiments it is desirable to select peptides carrying a T cell exposed motif of interest that in the natural amino acid context, in vivo, is predicted to be bound by the corresponding MHC alleles at an affinity of less than 500 nanomolar.

It is desirable that T cells stimulated by the methods described herein are targeted to epitopes in the tumor but also to mitigate the chance of an adverse effect through inadvertent targeting of a normal protein in the human proteome. Therefore, in embodiments described herein, peptides are selected which comprise T cell exposed motifs that are either absent from the normal human proteome or that occur only in low numbers. In some embodiments peptides are selected in which the T cell exposed motif they comprise occur only in less than ten other protein contexts in the human proteome. Further, in preferred embodiments the peptides are selected such that, when there is an identical T cell exposed motif in the human proteome, it is not found in the context of amino acid flanks that result in a predicted binding affinity of less than 200 nM to the MHC alleles of the particular subject, thus mitigating the possibility of presentation and recognition by the stimulated T cells

Methods provided herein are to select peptides to stimulate T cell responses to target peptides than comprise an amino acid of interest, or more than one amino acids, that are the result of a mutation in a tumor, referred to herein as “mutated amino acids”. Thus, the amino acid of interest is the differentiator between a normal protein and a tumor protein and by specifically targeting T cell exposed motifs that comprise the mutated amino acid of interest, the T cell response can be tumor specific in its effects. In some cases the mutated amino acids arise as the product of a missense mutation, where one amino acid is replaced by another as the result of a non-synonymous codon change. In other instances, the mutated amino acid, or amino acids, of interest results from an insertion, deletion, splice, or frame shift. In yet other embodiments the mutated amino acids are a combination of amino acids not normally found in normal cells but which are juxtaposed at the bridge junction arising from the fusion of two genes or partial genes.

In some cases, the mutation, and the resultant T cell exposed motif that is created, is unique to the individual subject. But in other embodiments the mutation is one which occurs commonly in certain cancer oncogenes and tumor suppressors which exhibit “hotspots” at which mutations occur frequently with deleterious effects in multiple individuals or in multiple cancer types. In some preferred embodiments the target peptides which are mutated are from proteins in the group represented by the symbols EGFR, H3.3, IDH, BRAF, TP53, PTEN, ERBB2, PIK3CA and KRAS, although these are considered non-limiting examples. In yet other embodiments the mutations are gene fusions that create novel amino acid motifs at their junctions, not found in either of the constituent proteins. In some preferred embodiments the gene fusions are those commonly associated with cancers and include, but are not limited to, KIAA1549-BRAF and EML4-ALK. In other embodiments other gene fusions are found which may include, but are not limited to, DNAJB1-PRKCA, BCR-ABL1, ETV6-RUNX1, FGFR3-TACC3, TMPRSS2-ERG and BRD3/4-NUT. Some gene fusions occur in the same junction position or a few junction positions on each occurrence; other fusions and fusion junctions are unique to each individual. The present invention therefore provides a method for identifying and targeting novel T cell exposed motifs created at such junctions.

One example of a splice variant that produces a unique amino acid motif is the deletion of exons 2-7 in EGFR to produce the EGFRvIII variant, although other instances of unique junction motifs arise as the result of splice variants and fusions and so these examples are not limiting. In some particular embodiments provided in the present invention, the commonly mutated T cell exposed motif sequences are provided for the above referenced EGFR, H3.3, IDH, BRAF, TP53, PTEN, ERBB2, PIK3CA, KRAS, EGFRVIII, KIAA1549-BRAF and EML4-ALK. Peptides which comprise these T cell exposed motifs, but not limited to these, are the basis for design of the selected peptides with desired binding affinity to the alleles of a particular subject, in which amino acids have been replaced in the groove exposed position to bring about the desired binding affinity. In addition, examples of such selected peptides are provided for each of these proteins, with peptides each selected to have a desired binding affinity for several different alleles. For each combination of allele and T cell motif, several hundred peptide options are generated and down-selected based on binding and other criteria relevant to manufacturing and formulation such as stability, solubility and propensity to aggregate. Therefore, the example sequences of selected peptides that are provided are considered non-limiting. In some particular instances it is not possible to design MHC II binding peptides which overlap the selected MHC I binding peptides and for these examples are provided of adjacent naturally occurring peptides which have at least a moderate affinity for MHC II to function as CD4+ helpers.

In some embodiments, the methods provided herein are used to design a selected peptide that will stimulate a T cell response to a target peptide in a protein that is encoded by a gene present at high copy number in an individual affected by cancer. In yet other embodiments the methods are used to generate T cell response to a protein the expression of which is upregulated in a subject affected by cancer. In yet further embodiments the methods are used to generate a T cell response to a protein that is a tumor associated antigen that is not mutated but is upregulated or present in increased copy number in a tumor, for administration alongside peptides designed to target tumor specific mutations.

In some preferred embodiments, the present invention provides methods to select an array of one or more peptides for inclusion in a personalized composition to stimulate T cell responses, wherein the selection is conducted to facilitate formulation. In some preferred embodiments, the present invention further provides methods of formulation for parenteral and non-parenteral delivery.

A particular challenge of manufacturing and delivering peptide immunogens is that the composition is determined by the tumor specific T cell epitope in the tumor protein based on the site of a mutation and cannot be changed. This may result in a peptide which is more or less soluble or stable in a particular carrier. Furthermore, a neoepitope vaccine typically comprises multiple peptides with different characteristics. However more flexibility is available to change the amino acids that are not located in the T cell exposed motif provided they are selected to provide an appropriate binding affinity.

Administration of peptide vaccines to cancer patients has been achieved by many methods. In some instances, peptides have been applied to autologous dendritic cells in vitro and the dendritic cells, or the T cells that they have contacted in vitro, transfused back into the patient. In other instances, the peptides have been encoded in RNA or DNA sequences and delivered in vitro or in vivo. Intradermal delivery is also a route of administration of choice. While cancer vaccines have typically been administered in an acute treatment phase, it is also important to consider the long-term maintenance of an effective tumor antigen specific T cell repertoire to avoid recurrence of immune evasion resulting in progression or metastasis of the tumor. Consideration therefore needs to be given to specific and stable delivery formulations which can be administered over the long term, and in some cases for life, and which are more acceptable to the subject. In some embodiments the present invention provides methods for formulation for parenteral delivery by several routes, including intradermal. In yet other embodiments the invention provides methods to deliver peptide vaccines non-parenterally, including orally.

In preferred embodiments the selected peptides are selected to achieve a desired solubility, stability and to reduce the probability of aggregation of the peptides during formulation. In some embodiments the average of the first principal components of the peptides comprised in the peptides are used as an index of the polarity of the peptide. In most preferred embodiments the peptides are selected for inclusion in the selected array of peptides when the index of polarity is less than 1; in further preferred embodiments the peptides are selected when the index of polarity is less than or equal to 2. As these indices are derived from the first principal component, they are unitless. A second characteristic which in some embodiments is applied as a criterion for selection is the log P, and in particular embodiments the log P of the octanol:water partition coefficient. In some preferred embodiments the desired solubility of the peptides is achieved by inclusion among those amino acids not in the T cell exposed motif of the amino acids arginine, lysine, glutamic or aspartic acid. Stability of the peptide is another important consideration in the manufacturability and formulation of the selected peptides. To achieve greater stability, in some embodiments those amino acids most prone to oxidation are excused from the groove exposed motif. Thus, in preferred embodiments methionine, tryptophan, histidine, cysteine and tyrosine are excluded from the groove exposed positions. In yet other embodiments those amino acids most prone to deamidation are avoided in the groove exposed position, leaving the T cell exposed positions in the peptide intact. Thus, the amino acids asparagine and glutamine are preferably excluded from the groove exposed positions of the selected peptides. In especially preferred embodiments aggregation caused by disulfide bond formation is avoided by exclusion of cysteine residues from the groove exposed positions.

Smaller peptides are more easily formulated and delivered to the subject. Therefore in desirable embodiments the selected peptides have a molecular weight of less than 4000 daltons; in yet further embodiments the preferred molecular weight is between 1500 and 4000 daltons and in the most preferred embodiments the molecular weight of each peptide is less than 1500 daltons.

In embodiments of the invention the group of selected peptides are administered to the subject from whose biopsy the T cell exposed motifs comprising mutated amino acids were identified, in order to stimulate a T cell response in that subject.

In further embodiments, the methods provided herein include the implementation of an assay to monitor the response to a particular selected peptide which has been designed to create a desired binding affinity to a particular MHC allele. The assaying of the immune response may be conducted by an Elispot assay or T cell repertoire analysis or by other in vitro assay methods.

In some preferred embodiments, the present invention provides a personalized vaccination regimen for administration to particular individual subjects with cancer, wherein the methods to select peptides that comprise an amino acid of interest and which have a desired binding affinity to binding to that subjects MHC alleles are applied to select a group of such selected peptides to include in a vaccine regimen. It will be apparent to those skilled in the art that such peptides may be delivered as peptides or as nucleic acids that encode them. Such a vaccine regimen is unique to the subject and the particular combination of MHC alleles and tumor specific mutations that the individual subject carries in their tumor.

In some embodiments, the selected peptides targeting tumor specific mutations include some which are drawn from those designed to encompass common mutations in the proteins EGFR, H3.3, IDH, BRAF, TP53, PTEN, ERBB2, PIK3CA, KRAS, EGFRVIII, KIAA1549-BRAF and EML4-ALK, and which comprise the T cell exposed motifs associate with such mutations. In preferred embodiments such peptides are embodied in the sequences provided herein. These examples are, however, are not intended to be limiting.

In some embodiments, in addition to selected peptides targeting T cell exposed motifs comprising tumor specific mutated amino acids, the vaccination regimen may also incorporate peptides selected to stimulate T cell responses to tumor associated proteins for administration alongside peptides selected to stimulate responses to tumor specific mutations. It may also incorporate naturally occurring peptides from the tumor protein that comprises the mutated amino acids.

In some embodiments the administration of a personalized vaccine as provided by the methods described herein, may be accompanied by a different immunotherapy intervention. Such an immunotherapy intervention may include, but is not limited to the administration of a checkpoint inhibitor drug. The immunotherapy intervention may be administered contemporaneously with the vaccine or at a later date.

The vaccination comprising the selected synthetic peptides may be administered to the subject parenterally, including but not limited to by intradermal or subcutaneous route. In yet other embodiments the vaccine comprising the selected synthetic peptides may be administered to the subject non-parenterally. In such a non-parenteral administration the routes of administration include but are not limited to intranasal, pulmonary inhalation, rectal, and oral. Oral administration may be used to apply the vaccine peptides to the buccal or pharyngeal mucosa or to deliver sublingually. In preferred embodiments the goal of oral administration is to deliver the selected synthetic peptide array to the gastrointestinal mucosa. To achieve this in some embodiments the peptides are formulated in a coated tablet. In most preferred embodiments the selected peptide array is encased in an enteric coated capsule. Such capsule may be soft or hard. The enteric capsule may be sued to deliver peptides in various forms other than a simple mixture to facilitate passage into the intestinal mucosa. Thus in preferred embodiments the enteric coated capsule contains peptides formulated in a particulate form, including but not limited to in glucan particles. Alternatively, the enteric capsule may comprise more complex formulations including lipid drug delivery systems including but not limited to lipid nanoparticles, emulsions, self-emulsifying drug delivery systems, nanocapsules and liposomes. In yet other preferred embodiments the peptides may be comprised in nanoparticles, a hydrogel, a mucoadhesive patch, and a microneedle patch, each serving to assist the passage of the peptide though the mucus layer and into proximity of the mucosal surface of the intestine. Microneedles are also a means of delivery intradermally, where in some preferred embodiments the peptides are delivered through the epidermis and into the dermis by means of a microneedle patch. In yet other embodiments the intradermal delivery is accomplished by an automated injection device with multiple needles calibrated to deliver precisely to the dermis.

Formulation of the peptides may also comprise an adjuvant, including but not limited to those from the group comprising Freund's (complete and incomplete), mineral gels such as aluminum hydroxide, surface active substances such as lysolecithin, Lipid A analogues (e.g. poly I:.C), pluronic polyols, polyanions, peptides, oil emulsions, CpG, C type lectin ligands, CD1d ligands (e.g. a-galactosylceramide), squalene, squalene emulsions, liposomes, imidazoquinolines (e.g. imiquimod), keyhole limpet hemocyanins, dinitrophenol, various cytokines and locally applied proinflammatory agents. In some embodiments an adjuvant such as, but not limited to, granulocyte stimulating factor, is administered some time before the administration of the peptide vaccine. In some embodiments the peptides are lyophilized. In such an embodiment, and indeed also when not lyophilized, the peptides may be accompanied by a pharmaceutically acceptable excipient.

In some embodiments the peptides in the vaccination regimen may be divided into separate subgroups for delivery according to their polarity, whereas in yet other embodiments they may be groups based on their octanol:water partition coefficient.

While many of the methods described herein focus on the selection and design of T cell stimulating peptides to target T cell motifs that comprise mutated amino acids in subjects affected by cancer, in some embodiments it is desirable to direct a T cell response to a tumor protein that is not mutated, but which is present in a tumor in increased in gene copy number or where the protein expression is upregulated in the tumor above that found in normal cells in the same tissue. The present invention therefore provides methods for targeting such proteins by identifying one or more epitopes of interest and determining the T cell exposed motif in each the epitope, determining the predicted binding affinity to the MHC alleles of the subject with cancer of the peptide that comprises the T cell exposed motif and generating an alternate array of peptides by substitution of amino acids not contained in the T cell exposed motifs, and selecting a group of such peptides having a desired predicted binding affinity for the MHC alleles of the subject affected by cancer and desired characteristics for formulation and delivery, and synthesizing the group of peptides, or nucleic acids that encode them.

Modulation of the T cell response by administration of a uniquely designed peptide vaccine is also an intervention suitable for management of other immunopathologies. In some preferred embodiments, the immunopathology that the subject is afflicted by is an allergy. In other preferred embodiments, the subject is afflicted by an autoimmune disease. In some other embodiments, the immunopathology arises as an adverse immune response to a biopharmaceutical protein. These examples are not intended to be limiting.

In some embodiments therefore, the present invention provides methods for treating an immunopathology in a subject, comprising designing a group of one or more T-cell epitope peptides, or nucleic acids encoding T cell epitope peptides, which have a desired predicted binding affinity for MHC alleles of the subject affected by the immunopathology, comprising the following steps: identifying a protein of interest comprising an epitope of interest that is causing the immunopathological T cell response; obtaining the sequence for the protein of interest and identifying the peptide comprising the epitope of interest; determining T cell exposed motifs in the epitope of interest; determining the predicted binding affinity to the subject's MHC alleles of peptides which comprise each the T cell exposed motif, or a subset thereof; generating an array of alternative peptides not present in the natural protein sequence, wherein each peptide in the array comprises the amino acids of one of the T cell exposed motifs, and in which one or more of the amino acids not within the T cell exposed motif are substituted to change the predicted MHC binding affinity; selecting a group of one or more selected peptides from the array of alternative peptides which have a desired predicted binding affinity for one or more of the subject's MHC alleles; synthesizing the group of one or more selected peptides, or nucleic acids encoding the selected peptides; and administering the group of one or more selected peptides, or nucleic acids encoding the selected peptides, to the subject affected by the immunopathology.

In some preferred embodiments of the methods in the invention for treating immunopathologies, the MHC alleles are MHC type I and the T cell response is a CD8+ response. In some preferred embodiments, the MHC alleles are MHC type II and the T cell response is a CD4+ response. In some preferred embodiments, the selected peptides are 9 or 10 amino acids long. In some preferred embodiments, the selected peptides are 13-20 amino acids long. In some preferred embodiments, the desired predicted binding affinity is less than 500 nanomolar. In some preferred embodiments, the desired predicted binding affinity is less than 200 nanomolar. In some preferred embodiments, the desired predicted binding affinity is less than 50 nanomolar. In some preferred embodiments, the desired predicted binding affinity is less than 20 nanomolar. In some embodiments the desired T cell response is up-regulatory; but in other instances the desired modulation of T cell responses in an immunopathology is achieved by a T regulatory response.

In some particular embodiments the allergy which afflicts the subject is an allergy to peanuts, to the Anisakis fish parasite worm, or to cats. In particular embodiments selected peptides and their sequences are provided which embody the T cell exposed motifs of these allergens and a set of selected peptides to induce anergy or exhaustion of the CD4+ helper responses to these allergens in individuals with particular exemplar MHC alleles.

In immunopathologies, including but not limited to autoimmunity, allergies and inflammation, an excessive immune response by T cells may drive the pathology. In such a situation the provision of a very high affinity MHC binding peptide may allow dampening of the T cell response by causing specific clones to become exhausted and anergic. As this is a clonal-specific intervention, the design of peptides which can bring about such modulation may be specific to the individual subject and to their HLA alleles. The need therefore arises to be able to select and formulate such selected and designed peptides in a way which facilitates their delivery for such immunopathologies.

When a synthetic peptide array is designed for treatment of the above noted immunopathologies, by creating alternative groove exposed motifs, the same considerations arise in determining manufacturability and formulation as arise in the design of a personal cancer vaccine. Therefore, the same considerations of solubility, stability and avoidance of aggregation apply in the case of a peptide vaccine to modulate the T cell response in an immunopathology other than cancer. Hence, the same preferred criteria of selection of peptides to conform to desired indices of polarity and log P in octanol:water apply as described above for a personal cancer vaccine. Furthermore the inclusion or omission of particular amino acids in the groove exposed motifs assist in achieving solubility, stability (including but not limited to avoidance of deamidation or oxidation) and avoidance of aggregation of peptides (including but not limited to by formation of disulfide bonds) in synthetic peptide vaccines for such immunopathologies.

The delivery systems for vaccine regimens for non-cancer immunopathologies are chosen according to each clinical condition, but as for cancer vaccines include a variety of both parenteral and non-parenteral routes. Among the non parenteral routes a preferred mode of delivery is per os to deliver vaccinal peptides to the gastrointestinal mucosa. Such delivery of a peptide vaccine for a non-cancer immunopathology in some preferred embodiments may include delivery by coated tablet or enteric coated capsule. In preferred embodiments the enteric coated capsules may contain lipid drug delivery systems, particulates or embodiments designed to facilitate mucosal contact, including but not limited to mucoadhesive patches, nanoparticles or microneedles. As with cancer vaccines, the synthetic peptide vaccine regimens for a non-cancer immunopathology may comprise peptides accompanied by adjuvants, excipients and may include peptides in a lyophilized form. As noted for peptides to stimulate T cells targeting cancer, smaller peptides are more easily formulated and delivered to the subject. Therefore in desirable embodiments targeting immunopathologies, the selected peptides have a molecular weight of less than 4000 daltons; in yet further embodiments the preferred molecular weight is between 1500 and 4000 daltons and in the most preferred embodiments the molecular weight of each peptide is less than 1500 daltons.

The considerations in design of a vaccination regimen for an immunopathology based on personalized selection of peptides, or their encoding nucleic acids, carrying T cell exposed motifs of interest are similar to those for a personalized cancer vaccine. Hence, the invention provides methods that in one embodiment groups peptides based on their polarity or in a second embodiment based on their partition coefficient in octanol:water. Furthermore, the invention provides for the delivery of a personalized peptide vaccine to a subject affected by an immunopathology via several routes including parenterally, including but not limited to intradermally and subcutaneously, or via a non-parenteral route including but not limited to intranasal, pulmonary inhalation, rectal, and oral routes. Embodiments of the oral routes include the buccal, pharyngeal and sublingual routes as well as to the gastrointestinal tract. The vaccine composition may be delivered in a coated capsule or in a enteric coated capsule. In yet other embodiments the vaccine composition may be delivered to the subject with an immunopathology in a lipid drug delivery system selected from the group consisting of lipid nanoparticles, emulsions, self-emulsifying drug delivery systems, nanocapsules and liposomes. Alternatively in further embodiments delivery is in a particulate form, including but not limited to, in glucan particles. In preferred embodiments delivery is accomplished in a nanoparticle system, a hydrogel system, a mucoadhesive patch, and a microneedle. Particularly preferred embodiments include a microneedle patch or delivery via a multi needle injection device. The immunopathology personal vaccine composition may comprise an adjuvant and may be formulated with a pharmaceutically acceptable excipient. In some alternative embodiments the peptide vaccine composition may be lyophilized or spray dried.

In a further embodiment, the invention provides a database of selected peptides each designed to provide T cell stimulation for a particular combination of amino acid and target peptide of interest and for a particular MHC allele. In some embodiments the database collates selected peptides with predicted binding affinity of less than 200 nanomolar to the corresponding MHC molecule; in yet other embodiments the predicted binding affinity recorded in the database is <100 nM, in yet further embodiments it is <50 nM.

In some preferred embodiments the database provides selected peptides for any possible amino acid missense mutation in a set of over 100 proteins, in other instances it provides selected peptides responsive to target peptides in a set of over 1000 proteins, and in yet other instances for over 5000 proteins. The database comprises the selected peptide sequences for the common mutations in the proteins detailed above: EGFR, H3.3, IDH, BRAF, TP53, PTEN, ERBB2, PIK3CA, KRAS, EGFRVIII, KIAA1549-BRAF and EML4-ALK and embodied in sequences provided herein, but also provides selected peptides to target a T cell response to other mutations in these and other proteins. In some preferred embodiments the database includes all possible mutations in a set of oncogenes and tumor suppressors. In further embodiments the database is expanded to include selected peptides for target T cell exposed motifs arising from other mutations and allele combinations in proteins other than oncogenes and suppressor proteins, up to and including the whole human proteome. The database has the utility of accelerating the design of a vaccine regimen by enabling a “look up” of a particular target peptide:MHC allele combination, without the need to compute the binding affinity of peptides in the parent protein and design a customized peptide every time a new mutation-allele combination arises.

In some further preferred embodiments, the present invention provides methods for producing a personalized composition to treat a subject with cancer comprising designing a group of one or more tumor-specific T-cell stimulating peptides, or nucleic acids encoding T cell stimulating peptides, which have a desired predicted binding affinity for the MHC alleles of the subject, comprising the following steps: obtaining a biopsy of the subject's tumor and a normal tissue sample; obtaining DNA sequences from the tumor biopsy and normal tissue and RNA sequences from the tumor biopsy obtaining sequences for proteins in the biopsy; identifying proteins from the biopsy containing mutated amino acids and the peptide comprising each of the mutated amino acids; determining T cell exposed motifs which comprise mutated amino acids in each of the proteins; determining the predicted binding affinity to the subject's MHC alleles of peptides which comprises each of the T cell exposed motifs, or a subset thereof; generating an array of alternative peptides not present in the tumor, wherein each peptide in the array comprises the amino acids of one of the T cell exposed motifs, and in which the amino acids not within the T cell exposed motif are substituted to change the predicted MHC binding affinity; selecting a group of one or more selected peptides from the array of alternative peptides which have a desired predicted binding affinity for one or more of the subject's MHC alleles; selecting from the array of alternative peptides those peptides in which those amino acids not located within the T cell exposed motif provide desired characteristics for formulation and delivery; and synthesizing the group of one or more selected peptides, or nucleic acids encoding the selected peptides.

In some preferred embodiments, the methods further comprise: determining the fraction of the DNA in the tumor biopsy comprising genes that encode each of the proteins containing mutated amino acids, and the fraction of RNA transcribed from that gene locus and expressing the protein containing mutated amino acids; and selecting from the proteins containing mutated amino acids in the biopsy those which are present in at least 10% of the DNA in the biopsy and expressed in at least 10% of the RNA transcribed from that gene locus in the biopsy; and generating the array of alternative peptides from these selected proteins.

In some preferred embodiments, the methods further comprise: determining the fraction of the DNA in the tumor biopsy comprising genes that encode each of the proteins containing mutated amino acids and the fraction of RNA transcribed from that gene locus and expressing the protein containing mutated amino acids; selecting from the proteins containing mutated amino acids in the biopsy those which are present in at least 3% of the DNA in the biopsy and expressed in at least 10% of the RNA transcribed from that gene locus in the biopsy; and generating the array of alternative peptides from these selected proteins.

In some preferred embodiments, the methods further comprise: determining the fraction of the DNA in the tumor biopsy comprising genes that encode each of the proteins containing mutated amino acids and the fraction of RNA transcribed from that gene locus and expressing the protein containing mutated amino acids; selecting from the proteins containing mutated amino acids in the biopsy those which are present in at least 10% of the DNA in the biopsy and expressed in at least 20% of the RNA transcribed from that gene locus in the biopsy; and generating the array of alternative peptides from these selected proteins.

In some preferred embodiments, the MHC alleles are MHC I alleles. In some preferred embodiments, the selected peptides are 8 to 10 amino acids in length. In some preferred embodiments, the MHC alleles are MHC II alleles. In some preferred embodiments, the selected peptides are from 11 to 22 amino acids in length. In some preferred embodiments, the T cell response is a cytotoxic T cell response. In some preferred embodiments, the T cell response is a T helper response. In some preferred embodiments, the group of selected peptides, or nucleic acids encoding the selected peptides, comprise peptides which bind an MHC I allele and peptides which bind an MHC II allele.

In some preferred embodiments, the desired binding affinity to an MHC allele is less than 500 nM. In some preferred embodiments, the desired binding affinity to an MHC allele is less than 200 nM. In some preferred embodiments, the desired binding affinity to an MHC allele is less than 100 nM. In some preferred embodiments, the desired binding affinity to an MHC allele is less than 50 nM. In some preferred embodiments, each of the peptides identified in the biopsy as comprising a mutated amino acid in the step of identifying proteins from the biopsy containing mutated amino acids and the peptide comprising each of the mutated amino acids is bound to one or more of the subject's MHC alleles with an affinity that is higher than 500 nanomolar.

In some preferred embodiments, the T cell exposed motif comprising the mutated amino acid is absent from the normal human proteome. In some preferred embodiments, the T cell exposed motif comprising the mutated amino acid occurs in less than 10 other protein sequence contexts in the human proteome. In some preferred embodiments, the T cell exposed motif comprising the mutated amino acid occurs in less than 10 other protein sequence contexts in the human proteome that have a predicted binding to the subject's MHC of <200 nM.

In some preferred embodiments, the mutated amino acids comprise a substituted amino acid that is a product of a missense mutation. In some preferred embodiments, the mutated amino acids comprise the product of insertion or deletion of one or more amino acids. In some preferred embodiments, the mutated amino acids comprise a new sequence that is the product of an in-frame or out of frame nucleotide mutation. In some preferred embodiments, the T cell exposed motif comprise a new sequence that is the product of a fusion of two genes.

In some preferred embodiments, the methods further comprise administering the group of one or more selected peptides, or nucleic acids encoding the selected peptides, to a subject affected by cancer.

In some preferred embodiments, the protein comprising the target peptide that comprises the mutated amino acid of interest has gene symbols selected from the group consisting of EGFR, EGFRVIII, H3.3, IDH, BRAF, TP53, PTEN, ERBB2, PIK3CA and KRAS. In some preferred embodiments, the protein comprising the target peptide that comprises the mutated amino acid of interest has gene symbols selected from the group consisting of KIAA1549-BRAF and EML4-ALK.

In some preferred embodiments, the protein is EGFRvIII and the target peptide comprises the T cell exposed motifs of any of SEQ ID NOS: 6-10. In some preferred embodiments, the protein is EGFRvIII and the group of one or more selected peptides, or nucleic acids encoding the selected peptides are selected from the group consisting of SEQ ID NOS: 11-50 and 51-75 and combinations thereof.

In some preferred embodiments, the protein is EGFR and the target peptide comprises the T cell exposed motifs of SEQ ID NOS: 98-119 and combinations thereof. In some preferred embodiments, the protein is EGFR and the group of one or more selected peptides, or nucleic acids encoding the selected peptides are selected from the group consisting of SEQ ID NOS: 120-177 and combinations thereof.

In some preferred embodiments, the protein is H3.3 and the target peptide comprises the T cell exposed motifs of SEQ ID NOS: 181-190 and combinations thereof. In some preferred embodiments, the protein is H3.3 and the group of one or more selected peptides, or nucleic acids encoding the selected peptides are selected from the group consisting of SEQ ID NOS: 201-287 and combinations thereof. In some preferred embodiments, the selected peptides are co-administered to the subject with one or more peptides selected from the group consisting of SEQ ID NOs: 288-292 and combinations thereof.

In some preferred embodiments, the protein is IDH and the target peptide comprises the T cell exposed motifs of SEQ ID NOS: 294-298 and 344-348 and combinations thereof. In some preferred embodiments, the protein is IDH and the group of one or more selected peptides, or nucleic acids encoding the selected peptides are selected from the group consisting of SEQ ID NOS: 304-343 and 354-391 and combinations thereof.

In some preferred embodiments, the protein is BRAF and the target peptide comprises the T cell exposed motifs of SEQ ID NOS: 397-401 and 441-443 and combinations thereof. In some preferred embodiments, the protein is BRAF and the group of one or more selected peptides, or nucleic acids encoding the selected peptides are selected from the group consisting of SEQ ID NOS: 402-437 and 444-463 and combinations thereof.

In some preferred embodiments, the protein is TP53 and the target peptide comprises the T cell exposed motifs of SEQ ID NOS: 468-495 and 621-641 and combinations thereof. In some preferred embodiments, the protein is TP53 and the group of one or more selected peptides, or nucleic acids encoding the selected peptides are selected from the group consisting of SEQ ID NOS: 520-620 and 645-704 and combinations thereof.

In some preferred embodiments, the protein is PTEN and the target peptide comprises the T cell exposed motifs of SEQ ID NOS: 705-712 and 791-797 and combinations thereof. In some preferred embodiments, the protein is PTEN and the group of one or more selected peptides, or nucleic acids encoding the selected peptides are selected from the group consisting of SEQ ID NOS: 721-790 and 805-812 and combinations thereof. In some preferred embodiments, the selected peptides are co-administered to the subject with one or more peptides selected from the group consisting of SEQ ID NOs: 802-804 and combinations thereof.

In some preferred embodiments, the protein is ERBB2 and the target peptide comprises the T cell exposed motifs of SEQ ID NOS: 813-824 and 919-930 and combinations thereof. In some preferred embodiments, the protein is ERBB2 and the group of one or more selected peptides, or nucleic acids encoding the selected peptides are selected from the group consisting of SEQ ID NOS: 835-918 and 943-1009 and combinations thereof.

In some preferred embodiments, the protein is PIK3CA and the target peptide comprises the T cell exposed motifs of SEQ ID NOS: 1010-1019 and 1097-1108 and combinations thereof. In some preferred embodiments, the protein is PIK3CA and the group of one or more selected peptides, or nucleic acids encoding the selected peptides are selected from the group consisting of SEQ ID NOS: 1030-1096 and 1121-1168 and combinations thereof.

In some preferred embodiments, the protein is KRAS and the target peptide comprises the T cell exposed motifs of SEQ ID NOS: 2269-2285 and 2342-2357 and combinations thereof. In some preferred embodiments, the protein is KRAS and the group of one or more selected peptides, or nucleic acids encoding the selected peptides are selected from the group consisting of SEQ ID NOS: 2309-2341 and 2374-2477 and combinations thereof.

In some preferred embodiments, the protein is a fusion protein KIAA1549-BRAF and the target peptide comprises the T cell exposed motifs of SEQ ID NOS: 1378-1388 and 1478-1483 and combinations thereof. In some preferred embodiments, the protein is a fusion protein KIAA1549-BRAF and group of one or more selected peptides, or nucleic acids encoding the selected peptides are selected from the group consisting of SEQ ID NOS: 1400-1477 and 1490-1519 and combinations thereof.

In some preferred embodiments, the protein is a fusion protein EML4-ALK and the group of one or more selected peptides, or nucleic acids encoding the selected peptides are selected from the group consisting of SEQ ID NOS: 1400-1477 and 1490-1519 and combinations thereof. In some preferred embodiments, the protein is a fusion protein EML4-ALK and the target peptide comprises the T cell exposed motifs of SEQ ID NOS: 1520-1550 and 1720-1745 and combinations thereof.

In some preferred embodiments, the target peptide comprising a mutated amino acid of interest is in a protein encoded by a gene that is present at increased copy number in an individual subject afflicted by cancer. In some preferred embodiments, the target peptide comprising a mutated amino acid of interest is in a protein the expression of which is upregulated in an individual subject afflicted by cancer.

In some preferred embodiments, the desired characteristics for formulation and delivery are selected from the group consisting of solubility, stability, and reduced aggregation and combinations thereof. In some preferred embodiments, the desired characteristic of solubility is achieved by selecting those amino acids not located in the T cell exposed motifs to increase the polarity of the peptide. In some preferred embodiments, the polarity of the peptide is increased by selecting peptides in which the index of polarity determined by the average of the first principal component of the amino acids in the peptide is less than or equal to 1. In some preferred embodiments, the polarity of the peptide is increased by selecting peptides in which the index of polarity determined by the average of the first principal component of the amino acids in the peptide is less than or equal to 2. In some preferred embodiments, the desired characteristic of solubility is achieved by selecting amino acids not located in the T cell exposed motifs to provide an average log P of the peptide for octanol:water of less than or equal to −2.0. In some preferred embodiments, the desired characteristic of solubility is achieved by selecting amino acids not located within the T cell exposed motif from the group comprising one or more of arginine, lysine, aspartic acid and glutamic acid. In some preferred embodiments, the desired characteristic of stability is achieved by selecting amino acids not located within the T cell exposed motif to reduce oxidation and deamidation. In some preferred embodiments, the amino acids not located within the T cell exposed motif are selected to exclude methionine, tryptophan, histidine, cysteine and tyrosine. In some preferred embodiments, the amino acids not located within the T cell exposed motif are selected to exclude asparagine and glutamine. In some preferred embodiments, the desired characteristic of reduced aggregation is achieved by selecting amino acids not located within the T cell exposed motif to exclude cysteine.

In some preferred embodiments, the selected peptides have a molecular weight less than 4000 daltons. In some preferred embodiments, the selected peptides have a molecular weight of 1500-4000 daltons. In some preferred embodiments, the selected peptides have a molecular weight less than 1500 daltons.

In some preferred embodiments, at least 2 peptides that bind to MHC I alleles or nucleic acids encoding the peptides that bind to MHC I alleles are selected for synthesis and/or coadministration to the subject. In some preferred embodiments, at least 5 peptides that bind to MHC I alleles or nucleic acids encoding the peptides that bind to MHC I alleles are selected for synthesis and/or coadministration to the subject. In some preferred embodiments, at least 10 peptides that bind to MHC I alleles or nucleic acids encoding the peptides that bind to MHC I alleles are selected for synthesis and/or coadministration to the subject. In some preferred embodiments, at least 15 peptides that bind to MHC I alleles or nucleic acids encoding the peptides that bind to MHC I alleles are selected for synthesis and/or coadministration to the subject. In some preferred embodiments, at least 20 peptides that bind to MHC I alleles or nucleic acids encoding the peptides that bind to MHC I alleles are selected for synthesis and/or coadministration to the subject. In some preferred embodiments, not more than 50 peptides that bind to MHC I alleles or nucleic acids encoding the peptides that bind to MHC I alleles are selected for synthesis and/or coadministration to a subject in a given round of vaccination.

In some preferred embodiments, at least 2 peptides that bind to MHC II alleles or nucleic acids encoding the peptides that bind to MHC II alleles are selected for synthesis and/or coadministration to the subject. In some preferred embodiments, at least 5 peptides that bind to MHC II alleles or nucleic acids encoding the peptides that bind to MHC II alleles are selected for synthesis and/or coadministration to the subject. In some preferred embodiments, at least 10 peptides that bind to MHC II alleles or nucleic acids encoding the peptides that bind to MHC II alleles are selected for synthesis and/or coadministration to the subject. In some preferred embodiments, at least 15 peptides that bind to MHC II alleles or nucleic acids encoding the peptides that bind to MHC II alleles are selected for synthesis and/or coadministration to the subject. In some preferred embodiments, at least 20 peptides that bind to MHC II alleles or nucleic acids encoding the peptides that bind to MHC II alleles are selected for synthesis and/or coadministration to the subject. In some preferred embodiments, not more than 50 peptides that bind to MHC II alleles or nucleic acids encoding the peptides that bind to MHC II alleles are selected for synthesis and/or coadministration to a subject in a given round of vaccination.

In some preferred embodiments, the peptides (ore nucleic acids encoding the peptides) selected for synthesis and/or coadministration to the subject comprise a combination of peptides that bind to MHC I alleles and MHC II alleles according to the foregoing ranges (e.g., at least 5 peptides that bind MHC I alleles and at least 5 peptides that bind MHC II alleles, and so on).

In some preferred embodiments, from 2 to 50 peptides that bind to MHC I alleles or nucleic acids encoding the peptides that bind to MHC I alleles are selected for synthesis and/or coadministration to the subject. In some preferred embodiments, from 5 to 50 peptides that bind to MHC I alleles or nucleic acids encoding the peptides that bind to MHC I alleles are selected for synthesis and/or coadministration to the subject. In some preferred embodiments, from 10 to 50 peptides that bind to MHC I alleles or nucleic acids encoding the peptides that bind to MHC I alleles are selected for synthesis and/or coadministration to the subject. In some preferred embodiments, from 15 to 50 peptides that bind to MHC I alleles or nucleic acids encoding the peptides that bind to MHC I alleles are selected for synthesis and/or coadministration to the subject. In some preferred embodiments, from 20 to 50 peptides that bind to MHC I alleles or nucleic acids encoding the peptides that bind to MHC I alleles are selected for synthesis and/or coadministration to the subject.

In some preferred embodiments, from 2 to 50 peptides that bind to MHC II alleles or nucleic acids encoding the peptides that bind to MHC II alleles are selected for synthesis and/or coadministration to the subject. In some preferred embodiments, from 5 to 100 peptides that bind to MHC II alleles or nucleic acids encoding the peptides that bind to MHC II alleles are selected for synthesis and/or coadministration to the subject. In some preferred embodiments, from 10 to 50 peptides that bind to MHC II alleles or nucleic acids encoding the peptides that bind to MHC II alleles are selected for synthesis and/or coadministration to the subject. In some preferred embodiments, from 15 to 50 peptides that bind to MHC II alleles or nucleic acids encoding the peptides that bind to MHC II alleles are selected for synthesis and/or coadministration to the subject. In some preferred embodiments, from 20 to 50 peptides that bind to MHC II alleles or nucleic acids encoding the peptides that bind to MHC II alleles are selected for synthesis and/or coadministration to the subject. In some preferred embodiments, the peptides (ore nucleic acids encoding the peptides) selected for synthesis and/or coadministration to the subject comprise a combination of peptides that bind to MHC I alleles and MHC II alleles according to the foregoing ranges (e.g., from 5 to 50 peptides that bind MHC I alleles and from 5 to 50 peptides that bind MHC II alleles, and so on).

In some preferred embodiments, the MHC I allele is not A0201 or A2402.

In some preferred embodiments, the peptides or nucleic acids encoding the peptides have a combination of 2 or more or mutations selected from the group consisting of a missense mutation, an insertion mutation, a deletion mutation, an in-frame nucleotide mutation or out-of-frame nucleotide mutation.

In some preferred embodiments, the proteins from the biopsy containing mutated amino acids are not one of WT-1 or a BCR/ABL fusion.

In some preferred embodiments, the methods further comprise conducting an assay to monitor the T cell response in the individual subject. In some preferred embodiments, the assay is an Elispot. In some preferred embodiments, the assay is analysis of the T cell repertoire of the individual subject.

In some preferred embodiments, the present invention provides a vaccination regimen comprising administering a group of peptides selected according to the methods described above to a subject with cancer.

In some preferred embodiments, the group of peptides comprises one of the sequences selected from the group consisting of SEQ ID NOS: 11-50, 51-75, 120-177, 201-287, 304-343, 354-391, 402-437, 444-463, 520-620, 665-704, 721-790, 805-812, 835-918, 943-1009, 1030-1096, 1121-1168, 1400-1477, 1490-1519, 1581-1719, 1772-1821, 1984-2012, 2169-2172, 2309-2341, and 2374-2477. In some preferred embodiments, the group of peptides comprises at least 3 of the sequences selected from the group consisting of SEQ ID NOS: 11-50, 51-75, 120-177, 201-287, 304-343, 354-391, 402-437, 444-463, 520-620, 665-704, 721-790, 805-812, 835-918, 943-1009, 1030-1096, 1121-1168, 1400-1477, 1490-1519, 1581-1719, 1772-1821, 1984-2012, 2169-2172, 2309-2341, and 2374-2477. In some preferred embodiments, the group of peptides comprises at least 5 of the sequences selected from the group consisting of SEQ ID NOS: 11-50, 51-75, 120-177, 201-287, 304-343, 354-391, 402-437, 444-463, 520-620, 665-704, 721-790, 805-812, 835-918, 943-1009, 1030-1096, 1121-1168, 1400-1477, 1490-1519, 1581-1719, 1772-1821, 1984-2012, 2169-2172, 2309-2341, and 2374-2477.

In some preferred embodiments, the group of peptides comprises one of the sequences selected from the group consisting of SEQ ID NOS: 6-10, 98-119, 181-190, 294-298, 344-348, 397-401, 441-443, 468-495, 621-641, 705-712, 791-797, 813-824, 919-930, 1010-1019, 1097-1108, 1378-1388, 1478-1483, 1520-1550, 1720-1745, 2174-2168, 2269-2285 and 2342-2357. In some preferred embodiments, the group of peptides comprises at least 3 of the sequences selected from the group consisting of SEQ ID NOS: 6-10, 98-119, 181-190, 294-298, 344-348, 397-401, 441-443, 468-495, 621-641, 705-712, 791-797, 813-824, 919-930, 1010-1019, 1097-1108, 1378-1388, 1478-1483, 1520-1550, 1720-1745, 2174-2168, 2269-2285 and 2342-2357. In some preferred embodiments, the group of peptides comprises at least 5 of the sequences selected from the group consisting of SEQ ID NOS: 6-10, 98-119, 181-190, 294-298, 344-348, 397-401, 441-443, 468-495, 621-641, 705-712, 791-797, 813-824, 919-930, 1010-1019, 1097-1108, 1378-1388, 1478-1483, 1520-1550, 1720-1745, 2174-2168, 2269-2285 and 2342-2357.

In some preferred embodiments, the group of peptides comprises alternative peptides selected according to the methods described above, and further comprises peptides which occur naturally in a tumor associated protein. In some preferred embodiments, the group of peptides comprises alternative peptides selected according to the methods described above, and further comprises other peptides which occur naturally in the tumor protein that comprises the mutated amino acids.

In some preferred embodiments, the vaccination is accompanied by administration of an immunotherapy intervention. In some preferred embodiments, the immunotherapy intervention is a checkpoint inhibitor drug.

In some preferred embodiments, the vaccine is administered to the subject parenterally. In some preferred embodiments, the vaccine is administered intradermally or subcutaneously. In some preferred embodiments, the vaccine is administered to the subject by a non-parenteral route. In some preferred embodiments, the non-parenteral route is selected from the group consisting of intranasal, pulmonary inhalation, rectal, and oral routes. In some preferred embodiments, the oral route is selected from the group consisting of buccal, pharyngeal and sublingual routes. In some preferred embodiments, the oral route is a gastrointestinal route. In some preferred embodiments, the vaccine is delivered as a coated tablet. In some preferred embodiments, the vaccine is delivered as an enteric coated capsule. In some preferred embodiments, the peptides are delivered in a lipid drug delivery system selected from the group consisting of lipid nanoparticles, emulsions, self-emulsifying drug delivery systems, nanocapsules and liposomes. In some preferred embodiments, the peptides are delivered in a particulate form. In some preferred embodiments, the particulate is a glucan particle. In some preferred embodiments, the peptides are formulated for delivery via a system selected from the group consisting of a nanoparticle system, a hydrogel system, a mucoadhesive patch, and a microneedle. In some preferred embodiments, the vaccine is delivered in a microneedle patch. In some preferred embodiments, the vaccine is delivered by a multi-needle delivery device.

In some preferred embodiments, the peptides in the vaccination regimen are administered with an adjuvant. In some preferred embodiments, the vaccination is preceded by administration of an adjuvant. In some preferred embodiments, the peptides are administered with a pharmaceutically acceptable excipient. In some preferred embodiments, the peptides are lyophilized. In some preferred embodiments, the group of peptides is divided into subgroups based on their polarity. In some preferred embodiments, the group of peptides is divided into subgroups based on their partition coefficient in octanol and water.

In some preferred embodiments, the present invention provides methods for treating a subject with cancer, comprising: designing a group of one or more T-cell epitope peptides, or nucleic acids encoding T cell epitope peptides, which have a desired predicted binding affinity for MHC alleles of the subject, comprising the following steps: identifying a protein of interest in the subject's tumor that is not mutated but that is encoded by a gene present at an increased copy number, or the expression of the protein of interest is upregulated; obtaining the sequence for the protein of interest and identifying a peptide comprising one or more epitopes of interest that is predicted to induce a T cell response to cells of the tumor; determining T cell exposed motifs in the epitope or epitopes of interest; determining the predicted binding affinity to the subject's MHC alleles of peptides which comprise each the T cell exposed motif, or a subset thereof; generating an array of alternative peptides not present in the natural protein sequence, wherein each peptide in the array comprises the amino acids of one of the T cell exposed motifs, and in which one or more of the amino acids not within the T cell exposed motif are substituted to change the predicted MHC binding affinity; selecting a group of one or more selected peptides from the array of alternative peptides which have a desired predicted binding affinity for one or more of the subject's MHC alleles; selecting from the array of alternative peptides those peptides in which those amino acids not located within the T cell exposed motif provide a desired characteristics for formulation and delivery; synthesizing the group of one or more selected peptides, or nucleic acids encoding the selected peptides; and administering the selected peptides or nucleic acids to the subject. In some preferred embodiments, the present invention provides methods for treating an immunopathology in a subject, comprising: designing a group of one or more T-cell epitope peptides, or nucleic acids encoding T cell epitope peptides, which have a desired predicted binding affinity for MHC alleles of the subject, comprising the following steps: identifying a protein of interest comprising an epitope of interest that is causing, or suspected of causing, the immunopathological T cell response; obtaining the sequence for the protein of interest and identifying the peptide comprising the epitope of interest; determining T cell exposed motifs in the epitope of interest; determining the predicted binding affinity to the subject's MHC alleles of peptides which comprise each the T cell exposed motif, or a subset thereof; generating an array of alternative peptides not present in the natural protein sequence, wherein each peptide in the array comprises the amino acids of one of the T cell exposed motifs, and in which one or more of the amino acids not within the T cell exposed motif are substituted to change the predicted MHC binding affinity; selecting a group of one or more selected peptides from the array of alternative peptides which have a desired predicted binding affinity for one or more of the subject's MHC alleles; selecting from the array of alternative peptides those peptides in which those amino acids not located within the T cell exposed motif provide a desired characteristics for formulation and delivery; synthesizing the group of one or more selected peptides, or nucleic acids encoding the selected peptides; and administering the selected peptides or nucleic acids to the subject.

In some preferred embodiments, the individual subject is afflicted by an autoimmune disease. In some preferred embodiments, the individual subject is afflicted by an allergy. In some preferred embodiments, the protein of interest comprising an epitope of interest is an allergen selected from the group comprising plant, insect, animal, parasite and fungal proteins. In some preferred embodiments, the individual subject is affected by an adverse immune response to a biopharmaceutical protein. In some preferred embodiments, the individual subject is afflicted by an infection or at risk of being infected.

In some preferred embodiments, the alternative peptide is selected to have a binding affinity to an MHC of less than 500 nM. In some preferred embodiments, the alternative peptide is selected to have a binding affinity to an MHC of less than 200 nM. In some preferred embodiments, the alternative peptide is selected to have a binding affinity to an MHC of less than 50 nM. In some preferred embodiments, the alternative peptide is selected to have a binding affinity to an MHC of less than 20 nM.

In some preferred embodiments, the selected peptides are 9 or 10 amino acids long. In some preferred embodiments, the selected peptides are 13-20 amino acids long.

In some preferred embodiments, the MHC alleles are MHC type I and the T cell response is a CD8+ response. In some preferred embodiments, the MHC alleles are MHC type II and the T cell response is a CD4+ response. In some preferred embodiments, the T cell response is a T regulatory response.

In some preferred embodiments, when treating an immunopathology, the protein is peanut allergen ara h2 and the target peptide comprises any of the T cell exposed motifs of SEQ ID NOS: 1822-1828. In some preferred embodiments, the protein is peanut allergen ara h2 and the group of one or more selected peptides, or nucleic acids encoding the selected peptides are selected from the group consisting of SEQ ID NOS: 1862-1881 and combinations thereof. In some preferred embodiments, the protein is Anisakis major allergen ani-s-1 and the target peptide comprises any of the T cell exposed motifs of SEQ ID NOS: 1829-1839. In some preferred embodiments, the protein is Anisakis major allergen ani-s-1 and the group of one or more selected peptides, or nucleic acids encoding the selected peptides are selected from the group consisting of SEQ ID NOS: 1882-1922 and combinations thereof. In some preferred embodiments, the protein is Felis catus major allergen 1 and the target peptide comprises the T cell exposed motifs of any of SEQ ID NOS: 1840-1841. In some preferred embodiments, the protein is Felis catus major allergen 1 and the group of one or more selected peptides, or nucleic acids encoding the selected peptides are selected from the group consisting of SEQ ID NOS: 1923-1925 and combinations thereof.

In some preferred embodiments, the desired characteristics for formulation and delivery are selected from the group consisting of solubility, stability, and reduced aggregation and combinations thereof. In some preferred embodiments, the desired characteristic of solubility is achieved by selecting those amino acids not located in the T cell exposed motifs to increase the polarity of the peptide. In some preferred embodiments, the polarity of the peptide is increased by selecting peptides in which the index of polarity determined by the average of the first principal component of the amino acids in the peptide is less than or equal to 1. In some preferred embodiments, the polarity of the peptide is increased by selecting peptides in which the index of polarity determined by the average of the first principal component of the amino acids in the peptide is less than or equal to 2. In some preferred embodiments, the desired characteristic of solubility is achieved by selecting amino acids not located in the T cell exposed motifs to provide an average log P of the peptide for octanol:water of less than or equal to −2.0. In some preferred embodiments, the desired characteristic of solubility is achieved by selecting amino acids not located within the T cell exposed motif from the group comprising one or more of arginine, lysine, aspartic acid and glutamic acid. In some preferred embodiments, the desired characteristic of stability is achieved by selecting amino acids not located within the T cell exposed motif to reduce oxidation and deamidation. In some preferred embodiments, the amino acids not located within the T cell exposed motif are selected to exclude methionine, tryptophan, histidine, cysteine and tyrosine. In some preferred embodiments, the amino acids not located within the T cell exposed motif are selected to exclude asparagine and glutamine. In some preferred embodiments, the desired characteristic of reduced aggregation is achieved by selecting amino acids not located within the T cell exposed motif to exclude cysteine.

In some preferred embodiments, the selected peptides have a molecular weight less than 4000 daltons. In some preferred embodiments, the selected peptides have a molecular weight of 1500-4000 daltons. In some preferred embodiments, the selected peptides have a molecular weight less than 1500 daltons.

In some preferred embodiments, at least 2 peptides that bind to MHC I alleles or nucleic acids encoding the peptides that bind to MHC I alleles are selected for synthesis and/or coadministration to the subject. In some preferred embodiments, at least 5 peptides that bind to MHC I alleles or nucleic acids encoding the peptides that bind to MHC I alleles are selected for synthesis and/or coadministration to the subject. In some preferred embodiments, at least 10 peptides that bind to MHC I alleles or nucleic acids encoding the peptides that bind to MHC I alleles are selected for synthesis and/or coadministration to the subject. In some preferred embodiments, at least 15 peptides that bind to MHC I alleles or nucleic acids encoding the peptides that bind to MHC I alleles are selected for synthesis and/or coadministration to the subject. In some preferred embodiments, at least 20 peptides that bind to MHC I alleles or nucleic acids encoding the peptides that bind to MHC I alleles are selected for synthesis and/or coadministration to the subject. In some preferred embodiments, not more than 50 peptides that bind to MHC I alleles or nucleic acids encoding the peptides that bind to MHC I alleles are selected for synthesis and/or coadministration to a subject in a given round of vaccination.

In some preferred embodiments, at least 2 peptides that bind to MHC II alleles or nucleic acids encoding the peptides that bind to MHC II alleles are selected for synthesis and/or coadministration to the subject. In some preferred embodiments, at least 5 peptides that bind to MHC II alleles or nucleic acids encoding the peptides that bind to MHC II alleles are selected for synthesis and/or coadministration to the subject. In some preferred embodiments, at least 10 peptides that bind to MHC II alleles or nucleic acids encoding the peptides that bind to MHC II alleles are selected for synthesis and/or coadministration to the subject. In some preferred embodiments, at least 15 peptides that bind to MHC II alleles or nucleic acids encoding the peptides that bind to MHC II alleles are selected for synthesis and/or coadministration to the subject. In some preferred embodiments, at least 20 peptides that bind to MHC II alleles or nucleic acids encoding the peptides that bind to MHC II alleles are selected for synthesis and/or coadministration to the subject. In some preferred embodiments, not more than 50 peptides that bind to MHC II alleles or nucleic acids encoding the peptides that bind to MHC II alleles are selected for synthesis and/or coadministration to a subject in a given round of vaccination.

In some preferred embodiments, the peptides (ore nucleic acids encoding the peptides) selected for synthesis and/or coadministration to the subject comprise a combination of peptides that bind to MHC I alleles and MHC II alleles according to the foregoing ranges (e.g., at least 5 peptides that bind MHC I alleles and at least 5 peptides that bind MHC II alleles, and so on).

In some preferred embodiments, from 2 to 50 peptides that bind to MHC I alleles or nucleic acids encoding the peptides that bind to MHC I alleles are selected for synthesis and/or coadministration to the subject. In some preferred embodiments, from 5 to 50 peptides that bind to MHC I alleles or nucleic acids encoding the peptides that bind to MHC I alleles are selected for synthesis and/or coadministration to the subject. In some preferred embodiments, from 10 to 50 peptides that bind to MHC I alleles or nucleic acids encoding the peptides that bind to MHC I alleles are selected for synthesis and/or coadministration to the subject. In some preferred embodiments, from 15 to 50 peptides that bind to MHC I alleles or nucleic acids encoding the peptides that bind to MHC I alleles are selected for synthesis and/or coadministration to the subject. In some preferred embodiments, from 20 to 50 peptides that bind to MHC I alleles or nucleic acids encoding the peptides that bind to MHC I alleles are selected for synthesis and/or coadministration to the subject.

In some preferred embodiments, from 2 to 50 peptides that bind to MHC II alleles or nucleic acids encoding the peptides that bind to MHC II alleles are selected for synthesis and/or coadministration to the subject. In some preferred embodiments, from 5 to 100 peptides that bind to MHC II alleles or nucleic acids encoding the peptides that bind to MHC II alleles are selected for synthesis and/or coadministration to the subject. In some preferred embodiments, from 10 to 50 peptides that bind to MHC II alleles or nucleic acids encoding the peptides that bind to MHC II alleles are selected for synthesis and/or coadministration to the subject. In some preferred embodiments, from 15 to 50 peptides that bind to MHC II alleles or nucleic acids encoding the peptides that bind to MHC II alleles are selected for synthesis and/or coadministration to the subject. In some preferred embodiments, from 20 to 50 peptides that bind to MHC II alleles or nucleic acids encoding the peptides that bind to MHC II alleles are selected for synthesis and/or coadministration to the subject. In some preferred embodiments, the peptides (ore nucleic acids encoding the peptides) selected for synthesis and/or coadministration to the subject comprise a combination of peptides that bind to MHC I alleles and MHC II alleles according to the foregoing ranges (e.g., from 5 to 50 peptides that bind MHC I alleles and from 5 to 50 peptides that bind MHC II alleles, and so on).

In some preferred embodiments, the methods further comprise administering the group of one or more selected peptides, or nucleic acids encoding the selected peptides to a subject affected by an immunopathology or cancer.

In some preferred embodiments, the present invention further provides vaccination regimens comprising administering a group of peptides selected according to the method as described above to a subject with an immunopathology or cancer. In some preferred embodiments, the group of peptides is divided into subgroups based on their polarity. In some preferred embodiments, the group of peptides is divided into subgroups based on their partition coefficient in octanol and water. In some preferred embodiments, the vaccine is administered to the subject parenterally. In some preferred embodiments, the vaccine is administered intradermally or subcutaneously. In some preferred embodiments, the vaccine is administered to the subject by a non-parenteral route. In some preferred embodiments, the non-parenteral route is selected from the group consisting of intranasal, pulmonary inhalation, rectal, and oral routes. In some preferred embodiments, the oral route is selected from the group consisting of buccal, pharyngeal and sublingual routes. In some preferred embodiments, the oral route is a gastrointestinal route. In some preferred embodiments, the vaccine is delivered as a coated tablet. In some preferred embodiments, the vaccine is delivered as an enteric coated capsule. In some preferred embodiments, the peptides are delivered in a lipid drug delivery system selected from the group consisting of lipid nanoparticles, emulsions, self-emulsifying drug delivery systems, nanocapsules and liposomes. In some preferred embodiments, the peptides are delivered in a particulate form. In some preferred embodiments, the particulate is a glucan particle. In some preferred embodiments, the peptides are formulated for delivery via a system selected from the group consisting of a nanoparticle system, a hydrogel system, a mucoadhesive patch, and a microneedle. In some preferred embodiments, the vaccine is delivered in a microneedle patch. In some preferred embodiments, the vaccine is delivered by a multi-needle delivery device. In some preferred embodiments, the peptides are administered with an adjuvant. In some preferred embodiments, the vaccination is preceded by administration of an adjuvant. In some preferred embodiments, the peptides are administered with a pharmaceutically acceptable excipient. In some preferred embodiments, the peptides are lyophilized. In some preferred embodiments, the peptides are spray-dried.

In some preferred embodiments, the present invention provides a database or non-transitory computer readable medium comprising tumor specific peptides comprising mutated amino acids, T cell exposed motifs comprising the mutated amino acids, and sequences of selected alternative peptides, or nucleic acids encoding alternative peptides selected according to any of the methods described above, wherein the database comprises alternative peptides selected to elicit a T cell response to target peptides in at least 100 proteins, and the database comprises alternative peptides each selected to bind to one of at least 8 MHC alleles. In some preferred embodiments, the alternative peptides are selected to bind MHC I alleles with a desired binding affinity. In some preferred embodiments, the alternative peptides are selected to bind MHC II alleles with a desired binding affinity. In some preferred embodiments, the desired binding affinity is less than 200 nM. In some preferred embodiments, the desired binding affinity is less than 100 nM. In some preferred embodiments, the desired binding affinity is less than 50 nM. In some preferred embodiments, the database comprises alternative peptides selected to elicit a T cell response to target peptides in at least 1000 proteins. In some preferred embodiments, the database comprises alternative peptides selected to elicit a T cell response to target peptides in at least 5000 proteins. In some preferred embodiments, the database comprises alternative peptides each selected to bind to one of at least 20 MHC alleles. In some preferred embodiments, the database comprises alternative peptides each selected to bind to one of at least 40 MHC alleles. In some preferred embodiments, the target peptides are in proteins selected from the group comprising oncogenes and tumor suppressor proteins. In some preferred embodiments, the target peptides are in proteins selected from the group comprising allergens and proteins that may induce autoimmunity. In some preferred embodiments, the database comprises 10 or more peptides comprising the T cell exposed motifs of SEQ ID NOS: 6-10, 98-119, 181-190, 294-298, 344-348, 397-401, 441-443, 468-495, 621-641, 705-712, 791-797, 813-824, 919-930, 1010-1019, 1097-1108, 1378-1388, 1478-1483, 1520-1550, 1720-1745, 2174-2168, 2269-2285 and 2342-2357. In some preferred embodiments, the database comprises 10 or more of the peptides of SEQ ID NOS: 11-50, 51-75, 120-177, 201-287, 304-343, 354-391, 402-437, 444-463, 520-620, 665-704, 721-790, 805-812, 835-918, 943-1009, 1030-1096, 1121-1168, 1400-1477, 1490-1519, 1581-1719, 1772-1821, 1984-2012, 2169-2172, 2309-2341, and 2374-2477.

### DESCRIPTION OF THE FIGURES

FIG. 1: EGFR and EGFRvIII Overview of MHC binding, B cell epitopes and topology. The X axis indicates the index position of sequential peptides with single amino acid displacement. The Y axis indicates predicted binding affinity of each peptide in standard deviation units for the protein. The red line shows the permuted average predicted MHC-IA and B (62 alleles) binding affinity of sequential 9-mer peptides with single amino acid displacement. The blue line shows the permuted average predicted MHC-II DRB allele (24 most common human alleles) binding affinity of sequential 15-mer peptides. Orange lines show the predicted probability of B-cell receptor binding for an amino acid centered in each sequential 9-mer peptide. Low numbers for MHC data represent high binding affinity, whereas low numbers equate to high B cell receptor contact probability. Ribbons (red: MHC-I, blue: MHC-II) indicate the 10% highest predicted MHC affinity binding. Orange ribbons indicate the top 25% predicted probability B-cell binding. Horizontal dotted lines demarcate the top 5% of binding affinity for the protein (red MHC I, blue MHC II).

FIG. 2: Relatively few of the MHC I A alleles have moderate or high binding in positions which expose the mutant motifs most commonly found in EGFR

FIG. 3: H3.3 K 27 M Overview of MHC binding, B cell epitopes and topology. Legend as for FIG. 1.

FIG. 4: IDH Overview of MHC binding, B cell epitopes and topology. Legend as for FIG. 1.

FIG. 5: BRAF Overview of MHC binding, B cell epitopes and topology. Legend as for FIG. 1.

FIG. 6: TP53 Overview of MHC binding, B cell epitopes and topology. Legend as for FIG. 1.

FIG. 7: PTEN Overview of MHC binding, B cell epitopes and topology. Legend as for FIG. 1.

FIG. 8: ERBB2 Overview of MHC binding, B cell epitopes and topology. Legend as for FIG. 1.

FIG. 9: PIK3CA Overview of MHC binding, B cell epitopes and topology. Legend as for FIG. 1.

FIG. 10: KRAS Overview of MHC binding, B cell epitopes and topology. Legend as for FIG. 1.

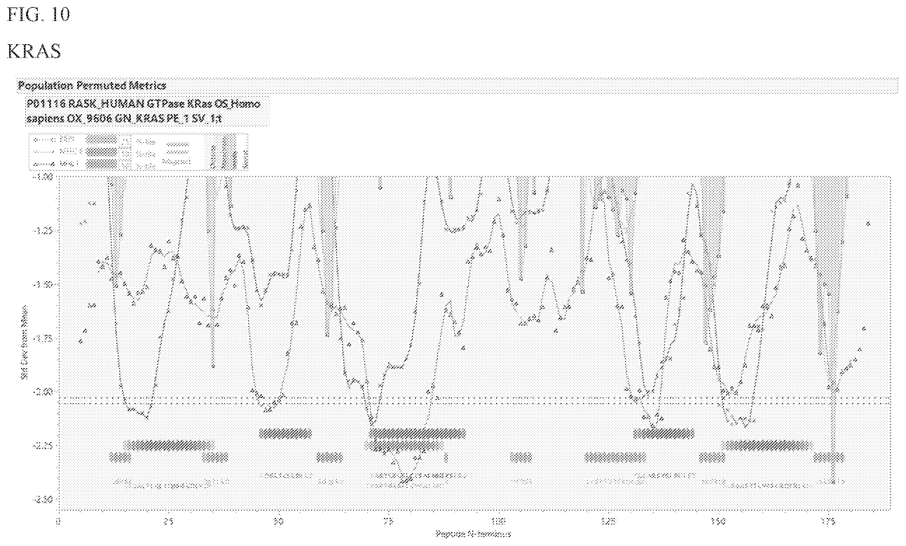

FIG. 11: Overview of fusion between C12orf49 and MDM2 showing parent proteins and fusion. Purple line indicates point of fusion in N and C partners and in the fusion. Legend otherwise as for FIG. 1.

FIG. 12: Overview of fusion between HSP90B1 and MDM2 showing parent proteins and fusion. Purple line indicates point of fusion in N and C partners and in the fusion. Legend otherwise as for FIG. 1.

FIG. 13: Overview of fusion between SBF2 and MDM2 showing parent proteins and fusion. Purple line indicates point of fusion in N and C partners and in the fusion. Legend otherwise as for FIG. 1.

FIG. 14: Bivariate fit showing correlation of index of polarity, as determined by the average of first principal component, and log P octanol:water for example set of peptides selected as candidate alternative peptides for a set of mutated proteins and allele A2902.

FIG. 15: Shows selection from an array of alternative peptides generated for allele A2902. in Panel A the unselected array comprises 304,804 unique peptides which bind above average to allele A2902 and carry a T cell exposed motif in the central pentamer that comprises a mutant amino acid in one of 15 mutated tumor proteins. In Panel B the selection has been constrained by polarity and binding affinity and exclusion of cysteine in the groove exposed positions. In Panel C the further criteria of exclusion of methionine and inclusion of arginine and lysine are applied.

FIG. 16: Starting from 15 proteins with 75 different T cell exposed motifs each comprising a tumor mutated amino acid, 304,804 alternative unique peptides were generated with above average binding for A2902. In Panel A, a selection was made for binding between −2.0 and −2.15 SD below the mean and a polarity less than 1 and no addition of C or M in the flanking regions, resulting in 4099 unique alternative peptides across 70 TCEM. In Panel B a further criterion of inclusion of R was added, resulting in 1725 unique alternative peptides across 69 T cell exposed motifs.

FIG. 17: Integrated genome browser analysis of PTEN G129V mutation. Track (a) contains the indicators for variants relative to the hg38 reference and the relevant annotations. Track (b) contains the read statistics for Normal DNA exomes from PBMC, (c) read statistics for tumor exomes and, (d) mRNA read statistics. In each case the bar graph portion of the track represents the fraction of the sequence having a different base at that genomic coordinate. In the left panel the lower portion of the track is squished to show all 166 reads of the exomes and 153 reads of the mRNA. In this case the tumor exome mutant frequency was 47% G→T. The gray background color is used to represent identity to hg38. Tumor.

FIG. 18: Integrated genome browser analysis of UPF1 S227L mutation. Layout is the same as in FIG. 17 of PTEN G129V. In this case the read track is expanded to show the C→T base change at the genomic coordinate. Exome mutant frequency was 32% and the frequency in the mRNA was 48% and the read depth for the region was a similar to the PTEN region.

### DEFINITIONS

As used herein, the term “genome” refers to the genetic material (e.g., chromosomes) of an organism or a host cell.

As used herein, the term “proteome” refers to the entire set of proteins expressed by a genome, cell, tissue or organism. A “partial proteome” refers to a subset the entire set of proteins expressed by a genome, cell, tissue or organism. Examples of “partial proteomes” include, but are not limited to, transmembrane proteins, secreted proteins, and proteins with a membrane motif. Human proteome refers to all the proteins comprised in a human being. Multiple such sets of proteins have been sequenced and are accessible at the InterPro international repository (on the world wide web at ebi.ac.uk/interpro). Human proteome is also understood to include those proteins and antigens thereof which may be over-expressed in certain pathologies, or expressed in a different isoforms in certain pathologies. Hence, as used herein, ‘tumor associated antigens’ are considered part of the normal human proteome. “Proteome” may also be used to describe a large compilation or collection of proteins, such as all the proteins in an immunoglobulin collection or a T cell receptor repertoire, or the proteins which comprise a collection such as the allergome, such that the collection is a proteome which may be subject to analysis. All the proteins in a bacteria or other microorganism are considered its proteome.

As used herein, the terms “protein,” “polypeptide,” and “peptide” refer to a molecule comprising amino acids joined via peptide bonds. In general “peptide” is used to refer to a sequence of 40 or less amino acids and “polypeptide” is used to refer to a sequence of greater than 40 amino acids.

As used herein, the term, “synthetic polypeptide,” “synthetic peptide” and “synthetic protein” refer to peptides, polypeptides, and proteins that are produced by a recombinant process (i.e., expression of exogenous nucleic acid encoding the peptide, polypeptide or protein in an organism, host cell, or cell-free system) or by chemical synthesis.

As used herein, the term “protein of interest” refers to a protein encoded by a nucleic acid of interest. It may be applied to any protein to which further analysis is applied or the properties of which are tested or examined. Similarly, as used herein, “target protein” may be used to describe a protein of interest that is subject to further analysis.

As used herein the term “amino acid of interest” refers to an amino acid which sets the protein apart from other sequences of the same protein, for instance by being the product of a mutation, indel, splice or fusion event, or the amino acid attracts attention as it is a salient feature in a particular T cell epitope.

As used herein “mutated amino acids” refers to an amino acid or an amino acid combination that is the result of a mutation, indel, splice or fusion event and that is distinct from normal sequences of the same protein. Hence “mutated amino acid” refers to an amino acid that has changed from the normal amino acid in that context, e.g., R273C in TP53 referred to as a missense mutation. It also refers to the de novo juxtaposition of amino acids arising from a deletion or splice or fusion or insertion.

A “target peptide” as used herein is one to which it is desired to direct an immune response.

As used herein “peptidase” refers to an enzyme which cleaves a protein or peptide. The term peptidase may be used interchangeably with protease, proteinases, oligopeptidases, and proteolytic enzymes. Peptidases may be endopeptidases (endoproteases), or exopeptidases (exoproteases). The the term peptidase would also include the proteasome which is a complex organelle containing different subunits each having a different type of characteristic scissile bond cleavage specificity. Similarly, the term peptidase inhibitor may be used interchangeably with protease inhibitor or inhibitor of any of the other alternate terms for peptidase.

As used herein, the term “exopeptidase” refers to a peptidase that requires a free N-terminal amino group, C-terminal carboxyl group or both, and hydrolyses a bond not more than three residues from the terminus. The exopeptidases are further divided into aminopeptidases, carboxypeptidases, dipeptidyl-peptidases, peptidyl-dipeptidases, tripeptidyl-peptidases and dipeptidases.

As used herein, the term “endopeptidase” refers to a peptidase that hydrolyses internal, alpha-peptide bonds in a polypeptide chain, tending to act away from the N-terminus or C-terminus. Examples of endopeptidases are chymotrypsin, pepsin, papain and cathepsins. A very few endopeptidases act a fixed distance from one terminus of the substrate, an example being mitochondrial intermediate peptidase. Some endopeptidases act only on substrates smaller than proteins, and these are termed oligopeptidases. An example of an oligopeptidase is thimet oligopeptidase. Endopeptidases initiate the digestion of food proteins, generating new N- and C-termini that are substrates for the exopeptidases that complete the process. Endopeptidases also process proteins by limited proteolysis. Examples are the removal of signal peptides from secreted proteins (e.g. signal peptidase I,) and the maturation of precursor proteins (e.g. enteropeptidase, furin,). In the nomenclature of the Nomenclature Committee of the International Union of Biochemistry and Molecular Biology (NC-IUBMB) endopeptidases are allocated to sub-subclasses EC 3.4.21, EC 3.4.22, EC 3.4.23, EC 3.4.24 and EC 3.4.25 for serine-, cysteine-, aspartic-, metallo- and threonine-type endopeptidases, respectively. Endopeptidases of particular interest are the cathepsins, and especially cathepsin B, L and S known to be active in antigen presenting cells.

As used herein, the term “immunogen” refers to a molecule which stimulates a response from the adaptive immune system, which may include responses drawn from the group comprising an antibody response, a B cell response, a cytotoxic T cell response, a T helper response, and a T cell or B cell memory response. An immunogen may stimulate an upregulation of the immune response with a resultant inflammatory response, or may result in down regulation or immunosuppression. Thus the T-cell response may be a T regulatory response. An immunogen also may stimulate a B-cell response and lead to an increase in antibody titer. Another term used herein to describe a molecule or combination of molecules which stimulate an immune response is “antigen”.

As used herein, the term “native” (or wild type) when used in reference to a protein refers to proteins encoded by the genome of a cell, tissue, or organism, other than one manipulated to produce synthetic proteins.

As used herein the term “epitope” refers to a peptide sequence which elicits an immune response, from either T cells or B cells or antibody

As used herein, the term “B-cell epitope” refers to a polypeptide sequence that is recognized and bound by a B-cell receptor. A B-cell epitope may be a linear peptide or may comprise several discontinuous sequences which together are folded to form a structural epitope. Such component sequences which together make up a B-cell epitope are referred to herein as B-cell epitope sequences. Hence, a B-cell epitope may comprise one or more B-cell epitope sequences. Hence, a B cell epitope may comprise one or more B-cell epitope sequences. A linear B-cell epitope may comprise as few as 2-4 amino acids or more amino acids.

“B cell core peptides” or “core pentamer” when used herein refers to the central 5 amino acid peptide in a predicted B cell epitope sequence. The B cell epitope may be evaluated by predicting the binding of across a series of 9-mer windows, the core pentamer then is the central pentamer of the 9-mer window

As used herein, the term “predicted B-cell epitope” refers to a polypeptide sequence that is predicted to bind to a B-cell receptor by a computer program, for example, as described in PCT US2011/029192, PCT US2012/055038, US2014/014523, and PCT US2015/039969, each of which is incorporated herein by reference in its entirety, and in addition by Bepipred (Larsen, et al., Immunome Research 2:2, 2006.) and others as referenced by Larsen et al (ibid) (Hopp T et al PNAS 78:3824-3828, 1981; Parker J et al, Biochem. 25:5425-5432, 1986). A predicted B-cell epitope may refer to the identification of B-cell epitope sequences forming part of a structural B-cell epitope or to a complete B-cell epitope.

As used herein, the term “T-cell epitope” refers to a polypeptide sequence which when bound to a major histocompatibility protein molecule provides a configuration recognized by a T-cell receptor. Typically, T-cell epitopes are presented bound to a MHC molecule on the surface of an antigen-presenting cell.

As used herein, the term “predicted T-cell epitope” refers to a polypeptide sequence that is predicted to bind to a major histocompatibility protein molecule by the neural network algorithms described herein, by other computerized methods, or as determined experimentally.

As used herein, the term “major histocompatibility complex (MHC)” refers to the MHC Class I and MHC Class II genes and the proteins encoded thereby. Molecules of the MHC bind small peptides and present them on the surface of cells for recognition by T-cell receptor-bearing T-cells. The MHC is both polygenic (there are several MHC class I and MHC class II genes) and polyallelic or polymorphic (there are multiple alleles of each gene). The terms MHC-I, MHC-II, MHC-1 and MHC-2 are variously used herein to indicate these classes of molecules. Included are both classical and nonclassical MHC molecules. An MHC molecule is made up of multiple chains (alpha and beta chains) which associate to form a molecule. The MHC molecule contains a cleft or groove which forms a binding site for peptides. Peptides bound in the cleft or groove may then be presented to T-cell receptors. The term “MHC binding region” refers to the groove region of the MHC molecule where peptide binding occurs.

As used herein, a “MHC I binding groove” refers to the structure of an MHC I molecule that binds to a peptide. The peptide that binds to the MHC I binding groove may be from about 8 amino acids to about 11 amino acids in length, but typically comprises a 9-mer. The amino acid positions in the peptide that binds to the groove are numbered based on a central core of 9 amino acids numbered 1-9 from N terminal to C terminal.

As used herein, a “MHC II binding groove” refers to the structure of an MHC molecule that binds to a peptide. The peptide that binds to the MHC II binding groove may be from about 11 amino acids to about 23 amino acids in length, but typically comprises a 15-mer. The amino acid positions in the peptide that binds to the groove are numbered based on a central core of 9 amino acids numbered 1-9, and positions outside the 9 amino acid core numbered as negative (N terminal) or positive (C terminal). Hence, in a 15mer the amino acid binding positions are numbered from −3 to +3 or as follows: −3, −2, −1, 1, 2, 3, 4, 5, 6, 7, 8, 9, +1, +2, +3.

As used herein, the term “haplotype” refers to the HLA alleles found on one chromosome and the proteins encoded thereby. Haplotype may also refer to the allele present at any one locus within the MHC. Each class of MHC-Is represented by several loci: e.g., HLA-A (Human Leukocyte Antigen-A), HLA-B, HLA-C, HLA-E, HLA-F, HLA-G, HLA-H, HLA-J, HLA-K, HLA-L, HLA-P and HLA-V for class I and HLA-DRA, HLA-DRB1-9, HLA-, HLA-DQA1, HLA-DQB1, HLA-DPA1, HLA-DPB1, HLA-DMA, HLA-DMB, HLA-DOA, and HLA-DOB for class II. The terms “HLA allele” and “MHC allele” are used interchangeably herein. HLA alleles are listed at hla.alleles.org/nomenclature/naming.html, which is incorporated herein by reference.

The MHCs exhibit extreme polymorphism: within the human population there are, at each genetic locus, a great number of haplotypes comprising distinct alleles—the IMGT/HLA database release (February 2010) lists 948 class I and 633 class II molecules, many of which are represented at high frequency (>1%). MHC alleles may differ by as many as 30-aa substitutions. Different polymorphic MHC alleles, of both class I and class II, have different peptide specificities: each allele encodes proteins that bind peptides exhibiting particular sequence patterns.

The naming of new HLA genes and allele sequences and their quality control is the responsibility of the WHO Nomenclature Committee for Factors of the HLA System, which first met in 1968, and laid down the criteria for successive meetings. This committee meets regularly to discuss issues of nomenclature and has published 19 major reports documenting firstly the HLA antigens and more recently the genes and alleles. The standardization of HLA antigenic specifications has been controlled by the exchange of typing reagents and cells in the International Histocompatibility Workshops. The IMGT/HLA Database collects both new and confirmatory sequences, which are then expertly analyzed and curated before been named by the Nomenclature Committee. The resulting sequences are then included in the tools and files made available from both the IMGT/HLA Database and at hla.alleles.org.

Each HLA allele name has a unique number corresponding to up to four sets of digits separated by colons. See e.g., hla.alleles.org/nomenclature/naming.html which provides a description of standard HLA nomenclature and Marsh et al., Nomenclature for Factors of the HLA System, 2010 Tissue Antigens 2010 75:291-455. HLA-DRB1*13:01 and HLA-DRB1*13:01:01:02 are examples of standard HLA nomenclature. The length of the allele designation is dependent on the sequence of the allele and that of its nearest relative. All alleles receive at least a four digit name, which corresponds to the first two sets of digits, longer names are only assigned when necessary. The digits before the first colon describe the type, which often corresponds to the serological antigen carried by an allele, The next set of digits are used to list the subtypes, numbers being assigned in the order in which DNA sequences have been determined. Alleles whose numbers differ in the two sets of digits must differ in one or more nucleotide substitutions that change the amino acid sequence of the encoded protein. Alleles that differ only by synonymous nucleotide substitutions (also called silent or non-coding substitutions) within the coding sequence are distinguished by the use of the third set of digits. Alleles that only differ by sequence polymorphisms in the introns or in the 5′ or 3′ untranslated regions that flank the exons and introns are distinguished by the use of the fourth set of digits. In addition to the unique allele number there are additional optional suffixes that may be added to an allele to indicate its expression status. Alleles that have been shown not to be expressed, ‘Null’ alleles have been given the suffix ‘N’. Those alleles which have been shown to be alternatively expressed may have the suffix ‘L’, ‘S’, ‘C’, ‘A’ or ‘Q’. The suffix ‘L’ is used to indicate an allele which has been shown to have ‘Low’ cell surface expression when compared to normal levels. The ‘S’ suffix is used to denote an allele specifying a protein which is expressed as a soluble ‘Secreted’ molecule but is not present on the cell surface. A ‘C’ suffix to indicate an allele product which is present in the ‘Cytoplasm’ but not on the cell surface. An ‘A’ suffix to indicate ‘Aberrant’ expression where there is some doubt as to whether a protein is expressed. A ‘Q’ suffix when the expression of an allele is ‘Questionable’ given that the mutation seen in the allele has previously been shown to affect normal expression levels.

In some instances, the HLA designations used herein may differ from the standard HLA nomenclature just described due to limitations in entering characters in the databases described herein. As an example, DRB1_0104, DRB1*0104, and DRB1-0104 are equivalent to the standard nomenclature of DRB1*01:04. In most instances, the asterisk is replaced with an underscore or dash and the semicolon between the two digit sets is omitted.

As used herein, the term “polypeptide sequence that binds to at least one major histocompatibility complex (MHC) binding region” refers to a polypeptide sequence that is recognized and bound by one or more particular MHC binding regions as predicted by the neural network algorithms described herein or as determined experimentally.

As used herein the terms “canonical” and “non-canonical” are used to refer to the orientation of an amino acid sequence. Canonical refers to an amino acid sequence presented or read in the N terminal to C terminal order; non-canonical is used to describe an amino acid sequence presented in the inverted or C terminal to N terminal order.

As used herein, the term “allergen” refers to an antigenic substance capable of producing immediate hypersensitivity and includes both synthetic as well as natural immunostimulant peptides and proteins. Allergen includes but is not limited to any protein or peptide catalogued in the Structural Database of Allergenic Proteins database (one the world wide web at fermi.utmb.edu/SDAP/index.html).

As used herein, the term “transmembrane protein” refers to proteins that span a biological membrane. There are two basic types of transmembrane proteins. Alpha-helical proteins are present in the inner membranes of bacterial cells or the plasma membrane of eukaryotes, and sometimes in the outer membranes. Beta-barrel proteins are found only in outer membranes of Gram-negative bacteria, cell wall of Gram-positive bacteria, and outer membranes of mitochondria and chloroplasts.

As used herein, the term “affinity” refers to a measure of the strength of binding between two members of a binding pair, for example, an antibody and an epitope and an epitope and a MHC-I or II haplotype. K_{d} is the dissociation constant and has units of molarity. The affinity constant is the inverse of the dissociation constant. An affinity constant is sometimes used as a generic term to describe this chemical entity. It is a direct measure of the energy of binding. The natural logarithm of K is linearly related to the Gibbs free energy of binding through the equation ΔG₀=−RT LN(K) where R=gas constant and temperature is in degrees Kelvin. Affinity may be determined experimentally, for example by surface plasmon resonance (SPR) using commercially available Biacore SPR units (GE Healthcare) or in silico by methods such as those described herein in detail. Affinity may also be expressed as the ic50 or inhibitory concentration 50, that concentration at which 50% of the peptide is displaced. Likewise ln(ic50) refers to the natural log of the ic50.

The term “K_{off}”, as used herein, is intended to refer to the off rate constant, for example, for dissociation of an antibody from the antibody/antigen complex, or for dissociation of an epitope from an MHC haplotype.

The term “K_{d}”, as used herein, is intended to refer to the dissociation constant (the reciprocal of the affinity constant “Ka”), for example, for a particular antibody-antigen interaction or interaction between an epitope and an MHC haplotype.

As used herein, the terms “strong binder” and “strong binding” and “High binder” and “high binding” or “high affinity” refer to a binding pair or describe a binding pair that have an affinity of greater than 2×10⁷M⁻¹ (equivalent to a dissociation constant of 50 nM Kd)

As used herein, the term “moderate binder” and “moderate binding” and “moderate affinity” refer to a binding pair or describe a binding pair that have an affinity of from 2×10⁷M⁻¹ to 2× 10⁶M⁻¹.

As used herein, the terms “weak binder” and “weak binding” and “low affinity” refer to a binding pair or describe a binding pair that have an affinity of less than 2×10⁶M⁻¹ (equivalent to a dissociation constant of 500 nM Kd)

Binding affinity may also be expressed by the standard deviation from the mean binding found in the peptides making up a protein. Hence a binding affinity may be expressed as “−1σ” or <−1σ, where this refers to a binding affinity of 1 or more standard deviations below the mean. A common mathematical transformation used in statistical analysis is a process called standardization wherein the distribution is transformed from its standard units to standard deviation units where the distribution has a mean of zero and a variance (and standard deviation) of 1. Because each protein comprises unique distributions for the different MHC alleles standardization of the affinity data to zero mean and unit variance provides a numerical scale where different alleles and different proteins can be compared. Analysis of a wide range of experimental results suggest that a criterion of standard deviation units can be used to discriminate between potential immunological responses and non-responses. An affinity of 1 standard deviation below the mean was found to be a useful threshold in this regard and thus approximately 15% (16.2% to be exact) of the peptides found in any protein will fall into this category.

The terms “specific binding” or “specifically binding” when used in reference to the interaction of an antibody and a protein or peptide or an epitope and an MHC haplotype means that the interaction is dependent upon the presence of a particular structure (i.e., the antigenic determinant or epitope) on the protein; in other words the antibody is recognizing and binding to a specific protein structure rather than to proteins in general. For example, if an antibody is specific for epitope “A,” the presence of a protein containing epitope A (or free, unlabeled A) in a reaction containing labeled “A” and the antibody will reduce the amount of labeled A bound to the antibody.

As used herein, the term “antigen binding protein” refers to proteins that bind to a specific antigen. “Antigen binding proteins” include, but are not limited to, immunoglobulins, including polyclonal, monoclonal, chimeric, single chain, and humanized antibodies, Fab fragments, F(ab′)2 fragments, and Fab expression libraries. Various procedures known in the art are used for the production of polyclonal antibodies. For the production of antibody, various host animals can be immunized by injection with the peptide corresponding to the desired epitope including but not limited to rabbits, mice, rats, sheep, goats, etc.

“Adjuvant” as used herein encompasses various adjuvants that are used to enhance the immunological response, depending on the host species, including but not limited to Freund's (complete and incomplete), mineral gels such as aluminum hydroxide, surface active substances such as lysolecithin, Lipid A analogues (e.g. poly I:.C), pluronic polyols, polyanions, peptides, oil emulsions, CpG, C type lectin ligands, CD1d ligands (e.g. a-galactosylceramide), squalene, squalene emulsions, liposomes, imidazoquinolines (e.g. imiquimod), keyhole limpet hemocyanins, dinitrophenol, and potentially useful human adjuvants such as BCG (Bacille Calmette-Guerin) and Corynebacterium parvum. In other embodiments a cytokine may be co-administered, including but not limited to interferon gamma or stimulators thereof, interleukin 12, or granulocyte stimulating factor. In other embodiments the peptides or their encoding nucleic acids may be co-administered with a local inflammatory agent, either chemical or physical. Examples include, but are not limited to, heat, infrared light, and proinflammatory drugs, including but not limited to imiquimod.

As used herein “immunoglobulin” means the distinct antibody molecule secreted by a clonal line of B cells; hence when the term “100 immunoglobulins” is used it conveys the distinct products of 100 different B-cell clones and their lineages.

As used herein, the terms “computer memory” and “computer memory device” refer to any storage media readable by a computer processor. Examples of computer memory include, but are not limited to, RAM, ROM, computer chips, digital video disc (DVDs), compact discs (CDs), hard disk drives (HDD), and magnetic tape.

As used herein, the term “computer readable medium” refers to any device or system for storing and providing information (e.g., data and instructions) to a computer processor. Examples of computer readable media include, but are not limited to, DVDs, CDs, hard disk drives, magnetic tape and servers for streaming media over networks.

As used herein, the terms “processor” and “central processing unit” or “CPU” are used interchangeably and refer to a device that is able to read a program from a computer memory (e.g., ROM or other computer memory) and perform a set of steps according to the program.

As used herein, the term “support vector machine” refers to a set of related supervised learning methods used for classification and regression. Given a set of training examples, each marked as belonging to one of two categories, an SVM training algorithm builds a model that predicts whether a new example falls into one category or the other.

As used herein, the term “classifier” when used in relation to statistical processes refers to processes such as neural nets and support vector machines.

As used herein “neural net”, which is used interchangeably with “neural network” and sometimes abbreviated as NN, refers to various configurations of classifiers used in machine learning, including multilayered perceptrons with one or more hidden layer, support vector machines and dynamic Bayesian networks. These methods share in common the ability to be trained, the quality of their training evaluated, and their ability to make either categorical classifications of non-numeric data or to generate equations for predictions of continuous numbers in a regression mode. Perceptron as used herein is a classifier which maps its input x to an output value which is a function of x, or a graphical representation thereof.

As used herein, the term “principal component analysis”, or as abbreviated “PCA”, refers to a mathematical process which reduces the dimensionality of a set of data (Wold, S., Sjorstrom, M., and Eriksson, L., Chemometrics and Intelligent Laboratory Systems 2001. 58: 109-130.; Multivariate and Megavariate Data Analysis Basic Principles and Applications (Parts I&II) by L. Eriksson, E. Johansson, N. Kettaneh-Wold, and J. Trygg, 2006 2^{nd} Edit. Umetrics Academy). Derivation of principal components is a linear transformation that locates directions of maximum variance in the original input data, and rotates the data along these axes. For n original variables, n principal components are formed as follows: The first principal component is the linear combination of the standardized original variables that has the greatest possible variance. Each subsequent principal component is the linear combination of the standardized original variables that has the greatest possible variance and is uncorrelated with all previously defined components. Further, the principal components are scale-independent in that they can be developed from different types of measurements. The application of PCA generates numerical coefficients (descriptors). The coefficients are effectively proxy variables whose numerical values are seen to be related to underlying physical properties of the molecules. A description of the application of PCA to generate descriptors of amino acids and by combination thereof peptides is provided in PCT US2011/029192 incorporated herein by reference in its entirety. Unlike neural nets PCA do not have any predictive capability. PCA is deductive not inductive.

As used herein, the term “vector” when used in relation to a computer algorithm or the present invention in relation to an amino acid sequence, refers to the mathematical properties of the amino acid sequence.

As used herein, the term “vector,” when used in relation to recombinant DNA technology, refers to any genetic element, such as a plasmid, phage, transposon, cosmid, chromosome, retrovirus, virion, etc., which is capable of replication when associated with the proper control elements and which can transfer gene sequences between cells. Thus, the term includes cloning and expression vehicles, as well as viral vectors. “Viral vector” as used herein includes but is not limited to adenoviral vectors, adeno-associated viral vectors, lentiviral vectors, retroviral vectors, poliovirus vectors, measles virus vectors, flavivirus vectors, poxvirus vectors, and other viral vectors which may be used to deliver a peptide or nucleic acid sequence to a host cell.

As used herein, the term “host cell” refers to any eukaryotic cell (e.g., mammalian cells, avian cells, amphibian cells, plant cells, fish cells, insect cells, yeast cells), and bacteria cells, and the like, whether located in vitro or in vivo (e.g., in a transgenic organism).

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro, including oocytes and embryos.

The term “isolated” when used in relation to a nucleic acid, as in “an isolated oligonucleotide” refers to a nucleic acid sequence that is identified and separated from at least one contaminant nucleic acid with which it is ordinarily associated in its natural source. Isolated nucleic acids are nucleic acids present in a form or setting that is different from that in which they are found in nature. In contrast, non-isolated nucleic acids are nucleic acids such as DNA and RNA that are found in the state in which they exist in nature.

The terms “in operable combination,” “in operable order,” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

A “subject” is an animal such as vertebrate, preferably a mammal such as a human, a bird, or a fish. Mammals are understood to include, but are not limited to, murines, simians, humans, bovines, ovines, cervids, equines, porcines, canines, felines etc.).

An “effective amount” is an amount sufficient to effect beneficial or desired results. An effective amount can be administered in one or more administrations,

As used herein, the term “purified” or “to purify” refers to the removal of undesired components from a sample. As used herein, the term “substantially purified” refers to molecules, either nucleic or amino acid sequences, that are removed from their natural environment, isolated or separated, and are at least 60% free, preferably 75% free, and most preferably 90% free from other components with which they are naturally associated. An “isolated polynucleotide” is therefore a substantially purified polynucleotide.

As used herein “Complementarity Determining Regions” (CDRs) are those parts of the immunoglobulin variable chains which determine how these molecules bind to their specific antigen. Each immunoglobulin variable region typically comprises three CDRs and these are the most highly variable regions of the molecule. T cell receptors also comprise similar CDRs and the term CDR may be applied to T cell receptors.

“Somatic hypermutation” (SHM), as used herein refers to the process by which variability in the immunoglobulin variable region is generated during the proliferation of individual B-cells responding to an immune stimulus. SHM occurs in the complementarity determining regions.

“Immunoglobulin germline” is used herein to refer to the variable region sequences encoded in the inherited germline genes and which have not yet undergone any somatic hypermutation. Each individual carries and expresses multiple copies of germline genes for the variable regions of heavy and light chains. These undergo somatic hypermutation during affinity maturation. Information on the germline sequences of immunoglobulins is collated and referenced on the world wide web at imgt.org [1]. “Germline family” as used herein refers to the 7 main gene groups, catalogued at IMGT, which share similarity in their sequences and which are further subdivided into subfamilies.

“Affinity maturation” is the molecular evolution that occurs during somatic hypermutation during which unique variable region sequences generated that are the best at targeting and neutralizing and antigen become clonally expanded and dominate the responding cell populations.

As used herein, the term “motif” refers to a characteristic sequence of amino acids forming a distinctive pattern.

“Germline motif” as used herein describes the amino acid subsets that are found in germline immunoglobulins. Germline motifs comprise both Groove Exposed Motifs and T Cell Exposed Motifs found in the variable regions of immunoglobulins which have not yet undergone somatic hypermutation.

“pMHC” Is used to describe a complex of a peptide bound to an MHC molecule. In many instances a peptide bound to an MHC-I will be a 9-mer or 10-mer however other sizes of 7-11 amino acids may be thus bound. Similarly MHC-II molecules may form pMHC complexes with peptides of 15 amino acids or with peptides of other sizes from 11-23 amino acids. The term pMHC is thus understood to include any short peptide bound to a corresponding MHC.

The term “Groove Exposed Motif” (GEM) as used herein refers to a subset of amino acids within a peptide that binds to an MHC molecule; the GEM comprises those amino acids which are turned inward towards the groove formed by the MHC molecule and which play a significant role in determining the binding affinity. In the case of human MHC-I the GEM amino acids are typically (1, 2, 3, 9). In the case of MHC-II molecules two formats of GEM are most common comprising amino acids (−3, 2, −1, 1, 4, 6, 9, +1, +2, +3) and (−3, 2, 1, 2, 4, 6, 9, +1, +2, +3) based on a 15-mer peptide with a central core of 9 amino acids numbered 1-9 and positions outside the core numbered as negative (N terminal) or positive (C terminal).

“T-cell exposed motif” (also where abbreviated TCEM), as used herein, refers to the sub-set of amino acids in a peptide bound in a MHC molecule which are directed outwards and exposed to a T-cell binding to the pMHC complex. A T-cell binds to a complex molecular space-shape made up of the outer surface MHC of the particular HLA allele and the exposed amino acids of the peptide bound within the MHC. Hence any T-cell recognizes a space shape or receptor which is specific to the combination of HLA and peptide. The amino acids which comprise the TCEM in an MHC-I binding peptide (TCEM I) typically comprise positions 4, 5, 6, 7, 8 of a 9-mer. The amino acids which comprise the TCEM in an MHC-II binding peptide typically comprise 2, 3, 5, 7, 8 (TCEM IIA) or −1, 3, 5, 7, 8 (TCEM IIB) based on a 15-mer peptide with a central core of 9 amino acids numbered 1-9 and positions outside the core numbered as negative (N terminal) or positive (C terminal). As indicated under pMHC, the peptide bound to an MHC may be of other lengths and thus the numbering system here is considered a non-exclusive example of the instances of 9-mer and 15 mer peptides.

As used herein “histotope” refers to the outward facing surface of the MHC molecules which surrounds the T cell exposed motif and in combination with the T cell exposed motif serves as the binding surface for the T cell receptor.

As used herein the T cell receptor refers to the molecules exposed on the surface of a T cell which engage the histotope of the MHC and the T cell exposed motif of a peptide bound in the MHC. The T cell receptor comprises two protein chains, known as the alpha and beta chain in 95% of human T cells and as the delta and gamma chains in the remaining 5% of human T cells. Each chain comprises a variable region and a constant region. Each variable region comprises three complementarity determining regions or CDRs

“Regulatory T-cell” or “Treg” as used herein, refers to a T-cell which has an immunosuppressive or down-regulatory function. Regulatory T-cells were formerly known as suppressor T-cells. Regulatory T-cells come in many forms but typically are characterized by expression CD4+, CD25, and Foxp3. Tregs are involved in shutting down immune responses after they have successfully eliminated invading organisms, and also in preventing immune responses to self-antigens or autoimmunity.

“uTOPE™ analysis” as used herein refers to the computer assisted processes for predicting binding of peptides to MHC and predicting cathepsin cleavage, described in, e.g., PCT US2011/029192, PCT US2012/055038, US2014/014523, PCT US2015/039969, PCT US2017/021781, US Publ. No. 20130330335, US Publ. No. 20160132631, US Publ. No. 20170039314, US Publ. No 20170161430, US Publ. No. 20190070255, PCT US2020/037206, U.S. Pat. Nos. 10,706,955 and 10,755,801, each of which is incorporated by reference herein in its entirety.

“Framework region” as used herein refers to the amino acid sequences within an immunoglobulin variable region which do not undergo somatic hypermutation.

“Isotype” as used herein refers to the related proteins of particular gene family. Immunoglobulin isotype refers to the distinct forms of heavy and light chains in the immunoglobulins. In heavy chains there are five heavy chain isotypes (alpha, delta, gamma, epsilon, and mu, leading to the formation of IgA, IgD, IgG, IgE and IgM respectively) and light chains have two isotypes (kappa and lambda). Isotype when applied to immunoglobulins herein is used interchangeably with immunoglobulin “class”.

“Isoform” as used herein refers to different forms of a protein which differ in a small number of amino acids. The isoform may be a full length protein (i.e., by reference to a reference wild-type protein or isoform) or a modified form of a partial protein, i.e., be shorter in length than a reference wild-type protein or isoform.

“Class switch recombination” (CSR) as used herein refers to the change from one isotype of immunoglobulin to another in an activated B cell, wherein the constant region associated with a specific variable region is changed, typically from IgM to IgG or other isotypes.

“Immunostimulation” as used herein refers to the signaling that leads to activation of an immune response, whether the immune response is characterized by a recruitment of cells or the release of cytokines which lead to suppression of the immune response. Thus, immunostimulation refers to both upregulation or down regulation.

“Up-regulation” as used herein refers to an immunostimulation which leads to cytokine release and cell recruitment tending to eliminate a non self or exogenous epitope. Such responses include recruitment of T cells, including effectors such as cytotoxic T cells, and inflammation. In an adverse reaction upregulation may be directed to a self-epitope.

“Down regulation” as used herein refers to an immunostimulation which leads to cytokine release that tends to dampen or eliminate a cell response. In some instances such elimination may include apoptosis of the responding T cells.

“Frequency class” or “frequency classification” as used herein is used to describe logarithmic based bins or subsets of amino acid motifs or cells. When applied to the counts of TCEM motifs found in a given dataset of peptides a logarithmic (log base 2) frequency categorization scheme was developed to describe the distribution of motifs in a dataset. As the cellular interactions between T-cells and antigen presenting cells displaying the motifs in MHC molecules on their surfaces are the ultimate result of the molecular interactions, using a log base 2 system implies that each adjacent frequency class would double or halve the cellular interactions with that motif. Thus, using such a frequency categorization scheme makes it possible to characterize subtle differences in motif usage as well as providing a comprehensible way of visualizing the cellular interaction dynamics with the different motifs. Hence a Frequency Class 2, or FC 2 means 1 in 4, a Frequency class 10 or FC 10 means 1 in 2¹⁰ or 1 in 1024. In other embodiments the frequency classification of the TCEM motif in the reference dataset is described by the quantile score of the TCEM in the reference dataset. Quantile scores are used, but is not limited to, applications where the reference dataset is the human proteome or a microbial proteome. “Frequency class” or “frequency classification” may also be applied to cellular clonotypic frequency where it refers to subgroups or bins defined by logarithmic based groupings, whether log base 2 or another selected log base.

A “rare TCEM” as used herein is a T cell Exposed Motif which is completely missing in the human proteome or present in up to only five instances in the human proteome.

“Adverse immune response” as used herein may refer to (a) the induction of immunosuppression when the appropriate response is an active immune response to eliminate a pathogen or tumor or (b) the induction of an upregulated active immune response to a self-antigen or (c) an excessive up-regulation unbalanced by any suppression, as may occur for instance in an allergic response.

“Clonotype” as used herein refers to the cell lineage arising from one unique cell. In the particular case of a B cell clonotype it refers to a clonal population of B cells that produces a unique sequence of IGV. The number of B cells that express that sequence varies from singletons to thousands in the repertoire of an individual. In the case of a T cell it refers to a cell lineage which expresses a particular TCR. A clonotype of cancer cells all arise from one cell and carry a particular mutation or mutations or the derivates thereof. The above are examples of clonotypes of cells and should not be considered limiting.

“T cell receptor” as used herein is the unique combination of receptors on a clonotype of T cells that engage an epitope and is abbreviated to “TCR”

As used herein “epitope mimic” or “TCEM mimic” is used to describe a peptide which has an identical or overlapping TCEM, but may have a different GEM. Such a mimic occurring in one protein may induce an immune response directed towards another protein which carries the same TCEM motif. This may give rise to autoimmunity or inappropriate responses to the second protein. Epitope mimic may also be used to refer to a B cell epitope which comprises the same pentamer motif that binds to a B cell receptor or antibody.

“Cytokine” as used herein refers to a protein which is active in cell signaling and may include, among other examples, chemokines, interferons, interleukins, lymphokines, granulocyte colony-stimulating factor tumor necrosis factor and programmed death proteins.

“MHC subunit chain” as used herein refers to the alpha and beta subunits of MHC molecules. A MHC II molecule is made up of an alpha chain which is constant among each of the DR, DP, and DQ variants and a beta chain which varies by allele. The MHC I molecule is made up of a constant beta macroglobulin and a variable MHC A, B or C chain.

As used here in “virome” comprises the viruses present in a human subject, latently chronically or during acute infection, or a sub set thereof made up of viruses of a particular taxonomic group or of the viruses located in a particular tissue or organ.

“Immunoglobulinome” as used herein refers to the total complement of immunoglobulins produced and carried by any one subject.

As used herein “allergome” refers to all proteins which may give rise to allergies. This includes proteins recorded in allergen datasets such as that represented on the world wide web at at allergome.com, allergenonline.org, comparedatabase.org/, and allergen.org as well as included in Uniprot, Swiss prot, etc.

As used herein the term “repertoire” is used to describe a collection of molecules or cells making up a functional unit or whole. Thus, as one non limiting example, the entirely of the B cells or T cells in a subject comprise its repertoire of B cells or T cells. The entirety of all immunoglobulins expressed by the B cells are its immunoglobulinome or the repertoire of immunoglobulins. A collection of proteins or cell clonotypes which make up a tissue sample, an individual subject or a microorganism may be referred to as a repertoire.

“Splice variant” as used herein refers to different proteins that are expressed from one gene as the result of inclusion or exclusion of particular exons of a gene in the final, processed messenger RNA produced from that gene or that is the result of cutting and re-annealing of RNA or DNA.

“TRAV” as used herein refers to the T cell receptor alpha variable region family or allele subgroups and “TRBV” refers to T cell receptor beta variable region family or allele subgroups as described in IMGT (one the world wide web at imgt.org/IMGTrepertoire/Proteins/index.php#C and imgt.org/IMGTrepertoire/Proteins/taballeles/human/TRA/TRAV/Hu_TRAVall.html. TRAV comprises at least 41 subgroups, with some having sub-subgroups. TRBV comprises at least 30 subgroups. Most combinations of alpha and beta variable region subgroups are encountered. “hTRAV” refers to human TRAV.

As used here in a “receptor bearing cell” is any cell which carries a ligand binding recognition motif on its surface. In some particular instances a receptor bearing cell is a B cell and its surface receptor comprises an immunoglobulin variable region, the immunoglobulin variable region comprising both heavy and light chains which make up the receptor. In other particular instances a receptor bearing cell may be a T cell which bears a receptor made up of both alpha and beta chains or both delta and gamma chains. Other examples of a receptor bearing cell include cells which carry other ligands such as, in one particular non limiting example, a programmed death protein of which there are multiple isoforms.

As used herein the term “bin” refers to a quantitative grouping and a “logarithmic bin” is used to describe a grouping according to the logarithm of the quantity.

As used herein “immunotherapy intervention” is used to describe any deliberate modification of the immune system including but not limited to through the administration of therapeutic drugs or biopharmaceuticals, radiation, T cell therapy, application of engineered T cells, which may include T cells linked to cytotoxic, chemotherapeutic or radiosensitive moieties, checkpoint inhibitor administration, cytokine or recombinant cytokine or cytokine enhancer, including but not limited to a IL-15 agonist, microbiome manipulation, vaccination, B or T cell depletion or ablation, or surgical intervention to remove any immune related tissues.

As used herein “immunomodulatory intervention” refers to any medical or nutritional treatment or prophylaxis administered with the intent of changing the immune response or the balance of immune responsive cells. Such an intervention may be delivered parenterally or orally or via inhalation. Such intervention may include, but is not limited to, a vaccine including both prophylactic and therapeutic vaccines, a biopharmaceutical, which may be from the group comprising an immunoglobulin or part thereof, a T cell stimulator, checkpoint inhibitor, or suppressor, an adjuvant, a cytokine, a cytotoxin, receptor binder, an enhancer of NK (natural killer) cells, an interleukin including but not limited to variants of IL15, superagonists, and a nutritional or dietary supplement. The intervention may also include radiation or chemotherapy to ablate a target group of cells. The impact on the immune response may be to stimulate or to down regulate.

“Checkpoint inhibitor” or “checkpoint blockade” as used herein refers to a type of drug that blocks certain proteins made by some types of immune system cells, such as T cells, and some cancer cells. These proteins help keep immune responses in check and can keep T cells from killing cancer cells. When these proteins are blocked, the “brakes” on the immune system are released and T cells are able to kill cancer cells better. Examples of checkpoint proteins found on T cells or cancer cells include, but are not limited to, PD-1/PD-L1 and CTLA-4/B7-1/B7-2.

As used herein the “cluster of differentiation” proteins refers to cell surface molecules providing targets for immunophenotyping of cells. The cluster of differentiation is also known as cluster of designation or classification determinant and may be abbreviated as CD. Examples of CD proteins include those listed on the world wide web at www.uniprot.org/docs/cdlist.

As used herein “microbiome” refers to the constellation of commensal microorganisms found within the human or other host body, inhabiting sites such as the gastrointestine, skin the urogenital tract, the oral cavity, the upper respiratory tract. While most frequently referring to bacteria, the microbiome also may include the viruses in these sites, referred to as the “virome”, or commensal fungi.

“Pattern” as used herein means a characteristic or consistent distribution of data points.

As used herein a “frequency pattern” is a data set that displays the frequency of TCEMs in a repertoire of proteins from a proteome associated with an individual subject as compared to the frequency of those TCEMs in a reference database. Particular TCEMs, or groups of TCEMs, within the subject's repertoire may occur at the same, lower or higher frequencies than the corresponding TCEMs in the reference database. The frequency pattern allows identification and categorization of unique TCEMs and/or patterns of TCEMs (i.e., unique features of unique TCEM features). The term “frequency pattern” as used herein is also used to describe the distribution of cellular clonotypes within a repertoire of cells from an individual subject, as compared to the frequency of the cellular clonotypes in a reference database. Particular clonotypes, or groups of clonotypes, within the subject's repertoire may occur at the same, lower or higher frequencies than the corresponding cellular clonotypes in the reference database. The frequency pattern allows identification and categorization of unique patterns of clonotypes. In some embodiments, a “frequency class” or “frequency classification” is assigned to a TCEM motif or to a cellular clonotype based on its frequency as described elsewhere herein.

As used herein “clonotype” is a line of cells derived from a committed or fully differentiated progenitor. In the case of T cells and somatic cells other than B cells, a clonotype of cells has a common genotype, i.e. comprises a common nucleotide sequence. Clonotypes with different nucleotide sequences may express a protein of identical amino acid sequence as a result of different codon utilization. Hence multiple genotypes may lead to a shared phenotype among such clonotypes. In B cells, somatic mutation results in a differentiated cell line comprising a nucleotide sequence that expresses antibodies of one isotype and variable region sequence; this is a B cell clonotype.

As used herein “clonotypic diversity” refers to the distribution of the total number of cells in a repertoire among all unique clonotypes in a repertoire. Hence, if a repertoire has 1 million cells, but these comprise 400,000 of clonotype 1 and 600,000 of clonotype 2, the repertoire has a low clonotypic diversity. If the 1 million cells are distributed as 10 each of 100,000 unique clonotypes the repertoire has a high clonotypic diversity.

As used herein “many to one” describes a relationship in which one protein or peptide sequence is encoded be many different synonymous nucleotide sequences.

As used herein “presentome” refers to the peptides bound in MHC and presented on the surface of antigen presented cells. Mass spectroscopy detects some but not all peptides which are part of the presentome.

“Neoantigen” as used herein refers to a novel epitope motif or antigen created as the result of introduction of a mutation into an amino acid sequence. Thus, a neoantigen differentiates a wildtype protein from its mutant-bearing tumor protein homolog, when such mutant is presented to T cells or B cells.

As used herein “tumor mutations” refers to all nucleotide or amino acid mutations detected in a tumor. In some cases the tumor mutations are commonly found within many patients with a particular tumor type. In other cases tumor mutations may be unique to a specific patient.

“Tumor specific antigen” or “tumor specific epitope” or “tumor specific mutated peptide” is used herein to designate an epitope or antigen or peptide that comprises a mutated amino acid and differentiates a mutated tumor protein from its unmutated wildtype homologue. Thus, a neoantigen is one type of tumor specific antigen. A “tumor specific T cell stimulating peptide” is a peptide that comprises a tumor specific epitope and which, when bound in an MHC molecule, engages a T cell receptor leading to stimulation of the T cell bearing that TCR. The combination of tumor specific antigens is almost always unique to a particular tumor in a particular subject.

“Tumor associated antigen” or “tumor associated protein’ as used herein refers to an antigen found in a protein that is not mutated or changed from a normal sequence in a tumor but which may be expressed on the surface of a tumor cell and may be expressed at higher levels in a tumor.

As used herein “driver” mutations are those which arise very early in tumorigenesis and are causally associated with the early steps of cell dysregulation. Driver mutations are shared by all clonal offspring arising from the initial tumor cells and offer some additional fitness benefit to the clonal line within its microenvironment. In contrast passenger mutations are those somatic mutations which arise during the differentiation of the tumor and which offer no particular benefit of fitness to the cell. Passengers may serve as biomarkers on tumor cells and may enable some immune evasion. Passenger mutations may differ at different time points in its development and among different parts of a tumor or among metastases. “Driver and passenger” are terms largely interchangeable with “trunk and branch” mutations.

“Bespoke peptides” or “bespoke vaccine” as used herein refers to a peptide or neoantigen or a combination of peptides, or nucleic acid encoding peptides, that are tailored or personalized specifically for an individual patient, taking into account that patient's HLA alleles and mutations. A bespoke peptide or bespoke vaccine is also referred to herein as a “personalized peptide”, “personalized peptide vaccine”, “personalized neoepitope vaccine” or “personalized vaccine”.

“Heteroclitic peptide” as used herein refers to a peptide in which one or more amino acid has been substituted with another to alter its engagement with its ligands.

As used herein “TCGA” refers to The Cancer Genome Atlas (on the world wide web at cancer.gov/about-nci/organization/ccg/research/structural-genomics/tcga) (.)

As used herein a “polyhydrophobic amino acid” refers to a short chain of natural amino acids which are hydrophobic. Examples include, but are not limited to, leucines, isoleucines or tryptophans where these are assembled in multimers of 5-15 repeats of any one such amino acid. As a non-limiting example, a poly leucine comprising 8 leucines would be an example of a polyhydrophobic amino acid.

“Lipid drug delivery system” or LDDS as used herein is a generic term which encompasses lipid nanoparticles, emulsions, self-emulsifying drug delivery systems, nanocapsules and liposomes, wherein molecules of a drug active product is encased or partially encased in lipid.

A “lipid core peptide system”, as used herein, refers to subunit vaccine comprising a lipoamino acid (LAA) moiety which allows the stimulation of immune activity. A combination of T cell stimulating epitopes or T and B cell stimulating epitopes are linked to a LAA. Multiple different constructs can be created with of different spatial orientation or LAA lengths (e.g. C12 2-amino-D,L-dodecanoic acid or C16, 2-amino-D,L-hexadecanoic acid,). When dissolved in a standard phosphate buffer LCP particles form and the particles facilitate uptake by antigen presenting cells. Different LAA chain lengths lead to different particle sizes.

As used herein, the term “cleavage site octomer” refers to the 8 amino acids located four each side of the bond at which a peptidase cleaves an amino acid sequence. Cleavage site octomer is abbreviated as CSO. “Cathepsin cleavage site octomer” is used herein where the peptidase is a cathepsin.

As used herein “compounding pharmacy” has the meaning defined in sections 503A and 503B of the Federal Food, Drug, and Cosmetic Act

As used herein, a “BAM” file is a compressed binary version of a Sequence Alignment File “SAM” file wherein the nucleotides are aligned to a reference genome. A “BAM slice” is a subset of the entire genome defined by genome coordinates. The HLA locus is located on Chromosome 6. In one particular instance a BAM slice is defined to contain just the HLA locus.

“Immunopathology” when used herein describes an abnormality of the immune system or of the immune response. An immunopathology may affect B-cells and their lineage causing qualitative or quantitative changes in the production of immunoglobulins. Immunopathologies may alternatively affect T-cells and result in abnormal T-cell responses. Immunopathologies may also affect the antigen presenting cells. An immunopathology may be manifest as an excessive immune response or a deficient immune response to a particular antigen. Immunopathologies may be the result of neoplasia of the cells of the immune system. Immunopathology is also used to describe diseases mediated by the immune system such as autoimmune diseases and allergies. Representative autoimmune diseases include, but are not limited to rheumatoid arthritis, diabetes type I and type II, Ankylosing Spondylitis, Atopic allergy, Atopic Dermatitis, Autoimmune cardiomyopathy, Autoimmune enteropathy, Autoimmune hemolytic anemia, Autoimmune hepatitis, Autoimmune inner ear disease, Autoimmune lymphoproliferative syndrome, Autoimmune peripheral neuropathy, Autoimmune pancreatitis, Autoimmune polyendocrine syndrome, Autoimmune progesterone dermatitis, Autoimmune thrombocytopenia purpura, Autoimmune uveitis, Bullous Pemphigoid, Castleman's disease, Celiac disease, Cogan syndrome, Cold agglutinin disease, Crohns Disease, Dermatomyositis, Eosinophilic fasciitis, Gastrointestinal pemphigoid, Goodpasture's syndrome, Graves' disease, Guillain-Barré syndrome, Anti-ganglioside Hashimoto's encephalitis, Hashimoto's thyroiditis, Systemic Lupus erythematosus, Miller-Fisher syndrome, Mixed Connective Tissue Disease, Myasthenia gravis, Narcolepsy, Pemphigus vulgaris, Polymyositis, Primary biliary cirrhosis, Psoriasis, Psoriatic Arthritis, Relapsing polychondritis, Sjögren's syndrome, Temporal arteritis, Ulcerative Colitis, Vasculitis, and Wegener's granulomatosis. An allergy is a form of immunopathology. An allergy may result from exposure to epitopes of, among other sources, plant, animal, environmental, or microbial origin. An adverse immune response to an exogenous agent such as a biopharmaceutical protein introduced into a subject is a form of immunopathology. An immunopathology may render an individual more susceptible to an infectious disease through an insufficient immune response.

“Antigen presenting cell” as used herein refers to cells which are capable of presentation of peptides to T cells bound to MHC molecules. This includes but is not limited to the so called “professional” antigen presenting cells comprising but not limited to dendritic cells, B cells, and macrophages, but also the so called non-professional antigen presenting cells which carry MHC molecules.

“Parenteral” as used herein refers to any direct injection into the body, including but not limited to intradermal, subcutaneous, intramuscular, intraperitoneal and intravenous injection.

“Non parenteral” as used herein refers to delivery per os to any point in the gastrointestinal tract, to the mucosa of the upper and lower respiratory tract, rectal mucosa or genitourinary tract. Topical application to the skin is also non parenteral

“Partition coefficient” as used herein, and abbreviated as P, is the particular ratio of the concentrations of a solute between the two solvents (a biphase of liquid phases), specifically for un-ionized solutes. The logarithm of the ratio, expressed as “log P” is used as a metric of the partition coefficient. When one of the solvents is water and the other is a non-polar solvent, then the log P value is a measure of lipophilicity or hydrophobicity.

The “distribution coefficient” and its logarithm “log D”, is the ratio of the sum of the concentrations of all forms of the compound (ionized plus un-ionized) in each of the two phases, one essentially always aqueous. As such, it depends on the pH of the aqueous phase, and log D=log P for non-ionizable compounds at any pH. For measurements of distribution coefficients, the pH of the aqueous phase is buffered to a specific value such that the pH is not significantly perturbed by the introduction of the compound. The value of each log D is then determined as the logarithm of a ratio of the sum of the experimentally measured concentrations of the solute's various forms in one solvent, to the sum of such concentrations of its forms in the other solvent.

As used herein “stability” when applied to amino acids and peptides refers to the absence of degradation of the amino acids and peptides into a non-biological chemical entity during storage and handling.

As used herein “aggregation” when applied to amino acids and peptides refers to the agglomeration of molecules such that they are not processed appropriately by biological systems

“Glucan particle” as defined herein refers to a particle comprising glucan from Saccharomyces as described by Soto et al and Huang et al [2, 3]

“Index of polarity” as used herein is calculated as the average of the first principal component (PC1) of the constituent amino acids is used. The PC of each amino acid are shown in Table 1.

“Originating peptide” as used herein refers to a naturally occurring peptide, whether mutated or not, which comprises a T cell exposed motif and an amino acid of interest therein, that is used as the basis for designing a peptide with desired binding affinity for a particular MHC allele.

“Proposed peptide” as used herein refers to the peptide with desired binding affinity for a particular MHC allele which is designed by changing the amino acids not in the T cell exposed motif and then selected from a list of such peptides for potential inclusion in a vaccination regimen.

As used herein, the term “motif” refers to a characteristic sequence of amino acids forming a distinctive pattern, this may also be expressed as an “amino acid motif”. A “pentamer motif’ is a combination of five amino acids, either contiguous to each other or separated by one or more other amino acids

“HUGO” as used herein refers to the Human Genome Organisation Gene Nomenclature Committee at the European Bioinformatics Institute (on the world wide web at genenames.org (_) which assigns a name and an approved gene symbol to each gene. Examples of HUGO gene names included herein are EGFR (Epidermal growth factor receptor), H3.3 or H33 (Histone H3.3), IDH (isocitrate dehydrogenase), BRAF (Serine/threonine-protein kinase B-raf), TP53 (Cellular tumor antigen p53), PTEN (Phosphatidylinositol 3,4,5-trisphosphate 3-phosphatase and dual-specificity protein phosphatase), ERBB2 (Receptor tyrosine-protein kinase erbB-2), PIK3CA (Phosphatidylinositol 4,5-bisphosphate 3-kinase catalytic subunit alpha isoform), and KRAS (GTPase KRas). Other examples which are found in fusion proteins mentioned herein are KIAA1549-BRAF (UPF0606 protein KIAA1549 fused to Serine/threonine-protein kinase B-raf) and EML4-ALK (Echinoderm microtubule-associated protein-like 4 fused to ALK tyrosine kinase receptor).

“EGFRvIII” as used herein refers to the common variant #3 of EGFR in which exons 2-7 are deleted.

“Upregulated” when used herein to refer to the expression of a gene or a protein denotes a level of expression above that in a normal quiescent cell.

“Copy number” when used in relation to a gene refers to the number of copies of an individual chromosomal sequence that encodes one or more genes.

### DESCRIPTION OF THE INVENTION

T cell mediated immune responses are the product of many factors unique to each individual. These include the immunogenetics of an individual subject, the T cell repertoire of the individual having been conditioned by prior epitope exposures, and the unique nature of any given epitope to which a T cell response is targeted. Both cancer and an array of immunopathologies can therefore be regarded as “personal diseases” in which the selection and design of an immunotherapeutic intervention must take into consideration the unique nexus of these factors in the individual.

There is increasing evidence that a variety of T cell immunotherapies can be successful in halting the progression of cancer [4]. Whereas in early days of cancer immunotherapy, the focus was only on tumor-associated antigens, current focus is now towards proteins comprising specific mutations in cancer cells, so called tumor-specific antigens or tumor neoantigens [5-8]. The fundamental goal in identifying and targeting mutations specific to the tumor is to differentiate normal from tumor tissue and hence eliminate tumor cells while leaving normal cells unharmed. A second current focus, and often combined strategy, is the application of checkpoint inhibitors and other immunomodulatory interventions to unleash T cell responses.

Tumor specific antigens comprise both those common to many cancers, and those which are unique to any single patient and which may change over the life of a tumor. Generally, the higher the mutational load, the more infiltrating T cells and the more inflamed a tumor, the greater probability of a check-point inhibitor leading to a successful T cell driven elimination of the tumor cells. Mutational load tends to differ between cancer types; some such as melanoma and colorectal cancers have a high mutational frequency. Others such as glioblastoma are notoriously low in mutational numbers.

Several recent publications have reported promising, but mixed, results in the development of personalized vaccines for melanoma [9, 10], lung cancer and glioblastoma [12, 13]. These have employed from 1 to 20 different neoantigens. Increasing the number of neoepitopes incorporated in a vaccine allows for a multipronged attack on the tumor using multiple alleles and multiple antigens derived from different proteins. Mutations continue to arise in tumors as they develop, with antigens gained or lost in the process. There may also be heterogeneity of mutations within a tumor and the mutational landscape may not be fully reflected in the sequencing of a biopsy. Hence a high number of cytotoxic “hits” is desirable rather than depending on only one or two antigen targets [8]. A goal of the present invention is to maximize the number of tumor specific epitopes which can be targeted by T cells responding to peptides presented by a particular patient's alleles.

The goal of T cell immunotherapy has been primarily to activate CD8+ cytotoxic T cells which will target tumor cells, but also to stimulate CD4+T helper cells to enhance CD8+ responses. Stimulation of CD4+T helper cells may also enhance B cell responses. Selection of peptides for use as neoepitopes has followed several paths. As a starting point, given the diversity of the human genome, it is desirable to compare sequences of proteins in tumor biopsies with a normal tissue from the same patient [14]. However, reference human genomes are frequently used as comparators to determine mutation sites. Practitioners have then used several approaches to select peptides for use, or for encoding in RNA or DNA for administration. In some instances peptides have been selected based on mass spectroscopy [15, 16]; in yet others predictive algorithms, most often NetMHC Pan [17], was used to select peptides [9, 10, 13]. In one instance, both approaches were reported, but in this particular case none of the mutated peptides were detected by mass spectroscopy [12].

In cancer many “personal factors” come into play. First, mutations arise that cause disrupted metabolic pathways resulting in the characteristic features of cancer: ongoing proliferation, evasion of growth suppressors, cellular replicative immortality, resistance to cell death and dysregulation of cell energetics, with associated angiogenesis and metastasis [18]. Each tumor comprises multiple genomic mutations. Some are silent mutations (synonymous) which do not change amino acid coding and have no consequence; others result in amino acid changes. Each tumor has a unique combination and number of mutated proteins. In many cases mutations are stochastic and thus unique to the individual. However, some proteins are more prone to mutations than others and have particular locations at which such mutations are more likely to occur.

An initial mutation (trunk mutation or driver mutation) may be followed by many more mutations (branch or passenger mutations), each stochastic. Thus, the initial genomic aberration is personal, the combination of unique tumor proteins is personal, and various therapeutic interventions may be prescribed based on this pattern. Each cell comprising a mutated protein is then subject to surveillance by the immune system, which may result in elimination of the cancer cell, or its escape through immune evasion or by inducing anergy or immune suppression [19]. As the immune surveillance depends on an individual patient's combination of HLA alleles, this is also personal. The presence of cognate T cells which can participate in the process of immune surveillance is determined by the individual's prior immune exposure and T cell repertoire. So this too is personal. Our findings show that mutations present in tumor proteins by the time of clinical diagnosis have developed several means of camouflage from immune surveillance and elimination, and that strategies to overcome such camouflage must be employed to achieve effective immunotherapy. The present invention provides such strategies by devising means to expose and present the tumor specific peptides to T cell recognition and effective elimination by T cells and by utilizing the B cell epitopes also exposed.

This invention provides a method for maximizing the immune response to mutated tumor specific proteins, either by means of stimulation of dendritic cells or T cells in vitro followed by administration of these cells to a patient, or by means of administration of a neoantigen vaccine in which de novo peptides, or their encoding nucleic acids, have been designed to ensure an appropriate level of binding affinity to a particular cancer patient's MHC alleles. Neoantigen selection from mutated tumor proteins is often limited by poor binding to a patient's MHC alleles. This invention overcomes this limitation by providing methods to design novel peptides, not found in the tumor protein, which bind a patient's alleles with a desired binding affinity while still retaining the tumor-specific T cell exposed motif needed to stimulate T cells cognate for the tumor mutation. The invention provides methods to design personalized neoantigen peptides for a particular patient based on that patient's alleles and unique mutations and to group these peptides into a vaccination regimen.

Mutations take many forms. As noted, some are silent as they result in no change in the amino acid composition. More common are missense mutations which change a codon to that of a different amino acid. Insertions and deletions may occur in frame, adding new potential epitopes or removing some. Out of frame codon insertions or deletions may generate novel strings of amino acids, until a stop codon is encountered. Splicing of genes may delete one or more exons. Fusion of two genes or partial genes may generate a novel fusion product with new functional characteristics, as well as a unique sequence at the bridge junction, which potentially provides a novel tumor specific target. Some gene fusions occur at common sites and are repeatedly associated with particular cancers. Others are unique to an individual subject. In another embodiment, therefore, the invention provides a method for designing an array of peptides which enable tumor-specific targeting of the junction sites created by insertions, deletions and fusions.

In one particular embodiment the invention provides specific peptides which may be used to target EGFRvIII, a common oncogenic deletion mutant of epidermal growth factor receptor found in multiple cancers as well as for the common fusion proteins KIAA1549-BRAF, found particularly in in low-grade pediatric gliomas and EML4-ALK a common finding in non small cell lung cancer but also in many other cancers. These examples are not considered limiting as other gene fusion products are commonly identified associated with certain cancer types. Examples include, but are not considered limiting, DNAJB1-PRKCA in fibrolamellar hepatocarcinoma [20], BCR-ABL1- in chronic myeloid leukemia and ETV6-RUNX1 in acute lymphoblastic leukemia [21], FRFR3-TACC3 in glioblastoma [22, 23], TMPRSS2-ERG in prostate adenocarcinoma [24], and BRD3/4-NUT fusions in midline carcinomas [25]. In some cases the fusion junctions are consistent from one tumor to another; in others several common fusions sites are identified (e.g. BCR-ABL1); however, in yet others the fusion junction locations are unique and vary between subjects.

In addition, novel epitopes may arise as the result of gene fusions that are unique to an individual. While the presence of some gene fusion products are common to most tumor cells, individual subjects may carry unique fusions. This may arise particularly when there is replication of gene fragments, for instance as in chromothripsis. The present invention therefore provides a method for identifying and targeting such individual fusion bridge sequences.

### Frequent Mutation Sites

While the majority of mutations are stochastic, certain protein have a propensity to acquire mutations at particular sequence locations. Furthermore, mutations at some sequence locations have a greater propensity to evade immune surveillance. The invention therefore addresses both tumor specific mutations which are personal to a specific individual cancer patient and also those mutations which appear repeatedly in the same protein in cancers of different types in different subjects.

In some embodiments, therefore, the present invention enables selection of a group of peptides that will elicit T cells to respond to mutations that are found in a given protein in multiple cancers, including cancers arising from different tissues. Such an array of peptides is selected based on the presence of T cell exposed motifs that match those in commonly mutated proteins but also on their binding to any of an extended list of alleles that may be carried by any cancer patient who has a cancer with the common mutation. In one particular embodiment, the sequences of peptides suitable to stimulate T cells targeting common mutations in, EGFR, H3.3, IDH, BRAF, TP53, PTEN,ERBB2, PIK3CA and KRAS as well as for the common fusion proteins KIAA1549-BRAF and EML4-ALK are provided for individuals carrying any of multiple different MHC I or 4 MHC II alleles.

By addressing these mutational hotspots in several common oncogenes and tumor suppressors and providing examples of personalized T cell stimulatory peptides designed to produce binding to a specific set of MHC alleles we demonstrate that a bank of such peptides can be designed in advance for such common mutations and which are then ready to use when a subject presents to a clinician with that mutation and for their particular MHC alleles. Thus, the invention provides for the design a priori of a database of selected peptides designed to stimulate T cell responses to any mutation that may occur in a particular list of oncogenes and suppressors for subjects of any combination of MHC alleles. In addition, it provides for a “look-up database,” which catalogues peptides designed to target mutations in oncogenes and suppressors, to be expanded to a database that covers all stochastic missense mutations which can arise in any protein in the human proteome and within subjects of diverse MHC alleles. The utility of this is that is accelerates the process of providing a vaccine tailored to the mutational landscape of an individual subject once sequencing of the tumor and comparative normal tissue is available from a biopsy, but without the need to individually compute the binding affinities of the peptides that encompass the mutated amino acid of interest and generate and select alternative peptides, a process that in practice can take several days and delay initiating treatment.

In one embodiment, therefore, the invention embodies a method to create a group of peptides, not found in the original mutated protein, which are capable of stimulating T cells specific to the individual tumor-bearing subject and which target the mutations in proteins unique to those in the tumor of that subject. Such a group of peptides is selected to bind to MHC alleles carried by that subject.

### Non Mutated Sequences in Tumors

In addition to the proteins characterized by mutations, tumors also may comprise non-mutated proteins which comprise appropriate T cell target epitopes. These may be unique to an individual and characterized by upregulation of gene or protein expression or increased copy number in the absence of mutation. Where such non-mutated proteins are characteristic of the tumor compared to normal tissue and their targeting will not produce adverse effects in normal tissue, they may be appropriate targets to include in a composition designed to stimulate T cell responses alongside those which target tumor specific mutations.

### Heteroclitic Peptides

There has been interest for some time in evaluating how substitution of amino acids can change the immunogenicity of peptides. Such peptides are known as heteroclitic peptides. Substitution of amino acids have been examined in the MHC binding positions and in the T cell exposed motifs [26-31]. In cancer studies these efforts have been directed to tumor associated antigens, which are normally occurring non-mutated proteins that are found associated with a tumor. Here, modification of the natural peptide to increase immunogenicity has allowed breaking of immune tolerance towards such natural self-proteins. Dyall showed that, following modification of amino acids at position 2 of a 9-mer, the same T cell receptors were bound in the natural and heteroclitic peptide. In two mouse models heteroclitic peptides could bring about tumor regression, whereas the natural peptide did not induce such a response [31]. A number of cancer vaccines that incorporate heteroclitic peptides have been developed to target tumor associated antigens, including those targeting melanoma [32-35], prostate cancer and tumors comprising Wilms tumor protein WT-1 (see also U.S. Ser. No. 10/815,273, U.S. Ser. No. 10/221,224).

In these examples efforts have been focused on binding to one or two single MHC I alleles, typically A0201 and A2402 and single peptides carrying single amino acid substitutions. Notably interest in the impact of heteroclitic peptides has been focused almost exclusively on MHC I binding peptides to produce cytotoxic lymphocytes, and not on MHC II/CD4+ binding peptides, with the exception of WT-1. Furthermore, heteroclitic peptides have not been described or applied where both CD8+ and CD4+ responses are sought together.

Meanwhile others have examined the differences in clonal T cell binding that may occur when changes are made in the binding pocket amino acids of long peptides which, when fitted into a MHC I groove, protrude in different configurations as pocket position amino acids are interchanged [29, 30][38]. Such bulged heteroclitic peptides may engage different TCR from the natural counterpart peptides.

Others investigators have examined changing amino acids lying in the T cell exposed motif to try to enhance immunogenicity. Cavalluzo showed that T cell cross reactivity could be maintained when changes were made to position 4 of a 9 mer peptide [39]. Zirlik examined increased immunogenicity by changing exposed amino acids [27]. These studies reinforce the concept that there are multiple T cell clones that will bind any particular pMHC combination, each with varying degrees of affinity, provided that the substituted amino acid has similar physical properties to that it replaces [40, 41].

The focus of the work on heteroclitic peptides has been on single naturally occurring tumor associated peptides and teaches away from addressing the complex of unique mutated tumor specific peptides that arise in each tumor and each individual. The goals are different. In the case of tumor associated antigen, the need addressed is to increase immunogenicity and break tolerance of a naturally occurring epitope. The overwhelming emphasis in the work on tumor associated antigens has been to provide epitopes to stimulate a cytotoxic lymphocyte response and does not address the role of the CD4+ response in supporting a CD8+ memory. In the case of a tumor specific mutation the need is to ensure that the mutation is actually exposed to the T cell immune response. This requires overcoming evasion caused by preferential hiding of mutations in MHC groove binding pocket positions, or evasion by lack of sufficient MHC binding to the subject's alleles. As our observations show that tumor specific mutations are often those which are rare in, or are absent from, the human proteome there may also be a need to stimulate rare T cell clones; de facto this is the inverse from seeking to overcome immune tolerance to a pre-existing natural peptide. The present invention, therefore, seeks to address the needs created by unique subject and tumor specific mutations. It provides a method for modifying binding for tumor specific peptides each encompassing a mutated amino acid (or junction of) and providing an array of peptides to address each active target in a subject with a personal combination of MHC alleles. This process needs to be conducted in a clinically relevant timeframe in order to stimulate both a CD8+ and CD4+ response. It further provides methods for selecting among the peptides which can achieve these goals for each tumor specific mutation to provide an array of peptides that facilitate manufacturing and delivery to that patient.

### Vaccination

The T cell stimulating peptides described and selected in this invention may be deployed in several ways. In some embodiments they can be used in vitro to prime dendritic cells which upon administration to the tumor-bearing subject will stimulate T cells. In other embodiments the peptides may be used in vitro to stimulate T cells, whether the T cells are from the tumor bearing subject or from an allele matched donor. The stimulated T cells are then administered to the subject. In preferred embodiments the groups of T cell stimulating peptides designed and selected by the methods of the invention are used as a vaccine administered to the tumor bearing subject. In some embodiments, instead of applying the peptides as a vaccine, nucleic acids encoding the peptides are administered to the subject, wherein the nucleic acids may be RNA or DNA.

Having identified an array of T cell stimulating peptides which are suitable to target the mutated tumor protein in the particular tumor-bearing subject of known MHC alleles, the present invention then embodies the design of a vaccination regimen. In one such embodiment the group of selected peptides is administered at one time. In an alternate embodiment the group of peptides may be divided into multiple subgroups which are administered at different time points. In one embodiment the invention provides for organizing the subgroups to ensure that several T cell exposed motifs are targeted in each subgroup and that the peptides depend on several different alleles for presentation. As motifs which are rare in the human proteome may offer an advantage in stimulating T cells and specifically targeting a tumor, one embodiment provides for prioritizing the peptide subgroup composition according to the frequency classification of the T cell exposed motif that each peptide carries relative to its frequency in the human proteome or human immunoglobulinome. In a preferred embodiment, the rare motifs are included in the early subgroups.

In embodiments of this invention, a vaccine is provided comprising peptides which carry T cell exposed motifs found in the tumor, but in which flanking amino acids have been substituted with others to change the binding of the peptide to optimize to a desired binding to the subject's MHC alleles. In some embodiments the vaccine is delivered to the subject parenterally, in other embodiments delivery is intradermal or transdermal. In other embodiments the delivery is non-parenteral, which may include but is not limited to delivery per os to the buccal, sublingual, pharyngeal or gastroineststinal mucosa. In yet other embodiments the non-parenteral delivery is to other mucosae, including but not limited to the mucosa of the respiratory or genitourinary tract or per rectum.

In some embodiments, vaccination is accompanied by an adjuvant. In some embodiments an adjuvant is incorporated into the solution comprising the neoantigen peptides. When vaccine is delivered transdermally, a particular embodiment is to accompany delivery by a local proinflammatory agent, whether physical, such as, but not limited to, heat, infrared light or friction, or by administration of a proinflammatory drug or cream.

### Checkpoint Inhibitor and Other Immunotherapeutic Interventions

Checkpoint inhibitor drugs prevent or delay the termination of T cell responses. In some embodiments the present invention provides for the administration of a checkpoint inhibitor with the vaccine or, in a preferred embodiment, following a peptide vaccine as described herein, or a nucleic acid vaccine encoding peptides. As another embodiment, when the vaccine is administered in multiple subgroups of peptides over time the checkpoint inhibitor may be reapplied after each or some of the subgroups of peptides. Furthermore, there are other immunomodulatory and immunotherapeutic interventions which extend the T cell responses, including but not limited to NK cells, IL-15, and other superagonists. In a further embodiment the present invention provides for the administration of other such interventions intended to extend or enhance T cell responses with the vaccine or, in a preferred embodiment, following the vaccine.

Checkpoint inhibitors are not always predictable in their efficacy; despite remarkable benefits to some patients, the percentage of patients who benefit is still low, on average about 20%. There is an effort to define better biomarkers to predict the outcome of checkpoint inhibitor therapy [42-44]. Furthermore, a wide variety of adverse off-target effects have been reported following checkpoint inhibitor treatment [45]. The issue underlying both problems is that checkpoint inhibitors are indiscriminate and will unleash whatever T cells the patient has at the time of administration, whether or not they are targeting the tumor or self-antigens. Combination of neoantigen vaccination with checkpoint inhibitor blockade has been shown to elicit T cells specific of the neoantigens and has been combined with neoantigen vaccines in several of the above referenced studies. Thus, one goal of the present invention is to maximize the number of tumor-targeting T cells which are dis-inhibited by checkpoint inhibitor administration, while also focusing on those T cells which do not target critical self-antigens. This has the potential to greatly increase the efficacy of checkpoint blockade therapy. Other immunomodulatory interventions have been designed to extend T cell responses, including but not limited to NK cells, IL-15, and other superagonists. In a further embodiment the present invention provides for the administration of such other immunotherapeutic interventions intended to extend T cell responses with the vaccine or, in a preferred embodiment, following the vaccine.

There is therefore a need to facilitate the selection of peptides suitable for use in neoantigen vaccines and to maximize the number and immunogenicity of peptides that are applied. This can then also be used to enhance the benefits of checkpoint inhibitor blockade.

### Determination of HLA Alleles and Determination of Binding

Determination of the subject's HLA alleles are a necessary prerequisite to designing a peptide of suitable HLA binding affinity for that individual. Therefore, in some embodiments the HLA alleles of the subject are determined from the whole exome sequence which is also used to determine the tumor mutations.

Methods for precisely predicting MHC binding, identifying and analyzing T cell exposed motifs and generating peptides with altered binding affinity are provided in the following co-pending applications, all of which are incorporated herein by reference in their entirety: PCT US2011/029192, PCT US2012/055038, US2014/014523, PCT US2015/039969, PCT US2017/021781, US Publ. No. 20130330335, US Publ. No. 20160132631, US Publ. No. 20170039314, US Publ. No 20170161430, US Publ. No. 20190070255, PCT US2020/037206, U.S. Pat. Nos. 10,706,955 and 10,755,801.

### DNA Sequencing

In some preferred embodiments, mutated proteins in biopsy samples are identified by sequencing the genome, proteome or transcriptome of cells from the biopsy. The present invention is not limited to any particular method of obtaining sequences of mutated in a biopsy. A variety of sequencing methods are readily available to those of ordinary skill in the art.

In some preferred embodiments, the present invention utilizes nucleic acid sequencing techniques. The nucleic acid sequences are preferably converted in silico to protein sequences from the identification of mutated amino acids and peptides comprising the mutated amino acids.

In some embodiments, the sequencing is Second Generation (a.k.a. Next Generation or Next-Gen), Third Generation (a.k.a. Next-Next-Gen), or Fourth Generation (a.k.a. N3-Gen) sequencing technology including, but not limited to, pyrosequencing, sequencing-by-ligation, single molecule sequencing, sequence-by-synthesis (SBS), semiconductor sequencing, massive parallel clonal, massive parallel single molecule SBS, massive parallel single molecule real-time, massive parallel single molecule real-time nanopore technology, etc. Morozova and Marra provide a review of some such technologies in Genomics, 92: 255 (2008), herein incorporated by reference in its entirety. Those of ordinary skill in the art will recognize that because RNA is less stable in the cell and more prone to nuclease attack experimentally RNA is usually reverse transcribed to DNA before sequencing.

DNA sequencing techniques include fluorescence-based sequencing methodologies (See, e.g., Birren et al., Genome Analysis: Analyzing DNA, 1, Cold Spring Harbor, N.Y.; herein incorporated by reference in its entirety). In some embodiments, the sequencing is automated sequencing. In some embodiments, the sequencing is parallel sequencing of partitioned amplicons (PCT Publication No: WO2006084132 to Kevin McKernan et al., herein incorporated by reference in its entirety). In some embodiments, the sequencing is DNA sequencing by parallel oligonucleotide extension (See, e.g., U.S. Pat. No. 5,750,341 to Macevicz et al., and U.S. Pat. No. 6,306,597 to Macevicz et al., both of which are herein incorporated by reference in their entireties). Additional examples of sequencing techniques include the Church polony technology (Mitra et al., 2003, Analytical Biochemistry 320, 55-65; Shendure et al., 2005 Science 309, 1728-1732; U.S. Pat. Nos. 6,432,360, 6,485,944, 6,511,803; herein incorporated by reference in their entireties), the 454 picotiter pyrosequencing technology (Margulies et al., 2005 Nature 437, 376-380; US 20050130173; herein incorporated by reference in their entireties), the Solexa single base addition technology (Bennett et al., 2005, Pharmacogenomics, 6, 373-382; U.S. Pat. Nos. 6,787,308; 6,833,246; herein incorporated by reference in their entireties), the Lynx massively parallel signature sequencing technology (Brenner et al. (2000). Nat. Biotechnol. 18:630-634; U.S. Pat. Nos. 5,695,934; 5,714,330; herein incorporated by reference in their entireties), and the Adessi PCR colony technology (Adessi et al. (2000). Nucleic Acid Res. 28, E87; WO 00018957; herein incorporated by reference in its entirety).

Next-generation sequencing (NGS) methods share the common feature of massively parallel, high-throughput strategies, with the goal of lower costs in comparison to older sequencing methods (see, e.g., Voelkerding et al., Clinical Chem., 55: 641-658, 2009; MacLean et al., Nature Rev. Microbiol., 7: 287-296; each herein incorporated by reference in their entirety). NGS methods can be broadly divided into those that typically use template amplification and those that do not. Amplification-requiring methods include pyrosequencing commercialized by Roche as the 454 technology platforms (e.g., GS 20 and GS FLX), Life Technologies/Ion Torrent, the Solexa platform commercialized by Illumina, GnuBio, and the Supported Oligonucleotide Ligation and Detection (SOLID) platform commercialized by Applied Biosystems. Non-amplification approaches, also known as single-molecule sequencing, are exemplified by the HeliScope platform commercialized by Helicos BioSciences, and emerging platforms commercialized by VisiGen, Oxford Nanopore Technologies Ltd., and Pacific Biosciences, respectively.

In pyrosequencing (Voelkerding et al., Clinical Chem., 55: 641-658, 2009; MacLean et al., Nature Rev. Microbiol., 7: 287-296; U.S. Pat. Nos. 6,210,891; 6,258,568; each herein incorporated by reference in its entirety), template DNA is fragmented, end-repaired, ligated to adaptors, and clonally amplified in-situ by capturing single template molecules with beads bearing oligonucleotides complementary to the adaptors. Each bead bearing a single template type is compartmentalized into a water-in-oil microvesicle, and the template is clonally amplified using a technique referred to as emulsion PCR. The emulsion is disrupted after amplification and beads are deposited into individual wells of a picotitre plate functioning as a flow cell during the sequencing reactions. Ordered, iterative introduction of each of the four dNTP reagents occurs in the flow cell in the presence of sequencing enzymes and luminescent reporter such as luciferase. In the event that an appropriate dNTP is added to the 3′ end of the sequencing primer, the resulting production of ATP causes a burst of luminescence within the well, which is recorded using a CCD camera. It is possible to achieve read lengths greater than or equal to 400 bases, and 10⁶ sequence reads can be achieved, resulting in up to 500 million base pairs (Mb) of sequence.

In the Solexa/Illumina platform (Voelkerding et al., Clinical Chem., 55: 641-658, 2009; MacLean et al., Nature Rev. Microbiol., 7: 287-296; U.S. Pat. Nos. 6,833,246; 7,115,400; 6,969,488; each herein incorporated by reference in its entirety), sequencing data are produced in the form of shorter-length reads. In this method, single-stranded fragmented DNA is end-repaired to generate 5′-phosphorylated blunt ends, followed by Klenow-mediated addition of a single A base to the 3′ end of the fragments. A-addition facilitates addition of T-overhang adaptor oligonucleotides, which are subsequently used to capture the template-adaptor molecules on the surface of a flow cell that is studded with oligonucleotide anchors. The anchor is used as a PCR primer, but because of the length of the template and its proximity to other nearby anchor oligonucleotides, extension by PCR results in the “arching over” of the molecule to hybridize with an adjacent anchor oligonucleotide to form a bridge structure on the surface of the flow cell. These loops of DNA are denatured and cleaved. Forward strands are then sequenced with reversible dye terminators. The sequence of incorporated nucleotides is determined by detection of post-incorporation fluorescence, with each fluor and block removed prior to the next cycle of dNTP addition. Sequence read length ranges from 36 nucleotides to over 250 nucleotides, with overall output exceeding 1 billion nucleotide pairs per analytical run.

Sequencing nucleic acid molecules using SOLID technology (Voelkerding et al., Clinical Chem., 55: 641-658, 2009; MacLean et al., Nature Rev. Microbiol., 7: 287-296; U.S. Pat. Nos. 5,912,148; 6,130,073; each herein incorporated by reference in their entirety) also involves fragmentation of the template, ligation to oligonucleotide adaptors, attachment to beads, and clonal amplification by emulsion PCR. Following this, beads bearing template are immobilized on a derivatized surface of a glass flow-cell, and a primer complementary to the adaptor oligonucleotide is annealed. However, rather than utilizing this primer for 3′ extension, it is instead used to provide a 5′ phosphate group for ligation to interrogation probes containing two probe-specific bases followed by 6 degenerate bases and one of four fluorescent labels. In the SOLID system, interrogation probes have 16 possible combinations of the two bases at the 3′ end of each probe, and one of four fluors at the 5′ end. Fluor color, and thus identity of each probe, corresponds to specified color-space coding schemes. Multiple rounds (usually 7) of probe annealing, ligation, and fluor detection are followed by denaturation, and then a second round of sequencing using a primer that is offset by one base relative to the initial primer. In this manner, the template sequence can be computationally re-constructed, and template bases are interrogated twice, resulting in increased accuracy. Sequence read length averages 35 nucleotides, and overall output exceeds 4 billion bases per sequencing run.

In certain embodiments, sequencing is nanopore sequencing (see, e.g., Astier et al., J. Am. Chem. Soc. 2006 Feb. 8; 128(5):1705-10, herein incorporated by reference). The theory behind nanopore sequencing has to do with what occurs when a nanopore is immersed in a conducting fluid and a potential (voltage) is applied across it. Under these conditions a slight electric current due to conduction of ions through the nanopore can be observed, and the amount of current is exceedingly sensitive to the size of the nanopore. As each base of a nucleic acid passes through the nanopore, this causes a change in the magnitude of the current through the nanopore that is distinct for each of the four bases, thereby allowing the sequence of the DNA molecule to be determined.

In certain embodiments, sequencing is HeliScope by Helicos BioSciences (Voelkerding et al., Clinical Chem., 55: 641-658, 2009; MacLean et al., Nature Rev. Microbiol., 7: 287-296; U.S. Pat. Nos. 7,169,560; 7,282,337; 7,482,120; 7,501,245; 6,818,395; 6,911,345; 7,501,245; each herein incorporated by reference in their entirety). Template DNA is fragmented and polyadenylated at the 3′ end, with the final adenosine bearing a fluorescent label. Denatured polyadenylated template fragments are ligated to poly(dT) oligonucleotides on the surface of a flow cell. Initial physical locations of captured template molecules are recorded by a CCD camera, and then label is cleaved and washed away. Sequencing is achieved by addition of polymerase and serial addition of fluorescently-labeled dNTP reagents. Incorporation events result in fluor signal corresponding to the dNTP, and signal is captured by a CCD camera before each round of dNTP addition. Sequence read length ranges from 25-50 nucleotides, with overall output exceeding 1 billion nucleotide pairs per analytical run.

The Ion Torrent technology is a method of DNA sequencing based on the detection of hydrogen ions that are released during the polymerization of DNA (see, e.g., Science 327(5970): 1190 (2010); U.S. Pat. Appl. Pub. Nos. 20090026082, 20090127589, 20100301398, 20100197507, 20100188073, and 20100137143, incorporated by reference in their entireties for all purposes). A microwell contains a template DNA strand to be sequenced. Beneath the layer of microwells is a hypersensitive ISFET ion sensor. All layers are contained within a CMOS semiconductor chip, similar to that used in the electronics industry. When a dNTP is incorporated into the growing complementary strand a hydrogen ion is released, which triggers a hypersensitive ion sensor. If homopolymer repeats are present in the template sequence, multiple dNTP molecules will be incorporated in a single cycle. This leads to a corresponding number of released hydrogens and a proportionally higher electronic signal. This technology differs from other sequencing technologies in that no modified nucleotides or optics are used. The per-base accuracy of the Ion Torrent sequencer is ˜99.6% for 50 base reads, with ˜100 Mb to 100Gb generated per run. The read-length is 100-300 base pairs. The accuracy for homopolymer repeats of 5 repeats in length is ˜98%. The benefits of ion semiconductor sequencing are rapid sequencing speed and low upfront and operating costs.

In some embodiments, sequencing is the technique developed by Stratos Genomics, Inc. and involves the use of Xpandomers. This sequencing process typically includes providing a daughter strand produced by a template-directed synthesis. The daughter strand generally includes a plurality of subunits coupled in a sequence corresponding to a contiguous nucleotide sequence of all or a portion of a target nucleic acid in which the individual subunits comprise a tether, at least one probe or nucleobase residue, and at least one selectively cleavable bond. The selectively cleavable bond(s) is/are cleaved to yield an Xpandomer of a length longer than the plurality of the subunits of the daughter strand. The Xpandomer typically includes the tethers and reporter elements for parsing genetic information in a sequence corresponding to the contiguous nucleotide sequence of all or a portion of the target nucleic acid. Reporter elements of the Xpandomer are then detected. Additional details relating to Xpandomer-based approaches are described in, for example, U.S. Pat. Pub No. 20090035777, entitled “High Throughput Nucleic Acid Sequencing by Expansion,” filed Jun. 19, 2008, which is incorporated herein in its entirety. Other emerging single molecule sequencing methods include real-time sequencing by synthesis using a VisiGen platform (Voelkerding et al., Clinical Chem., 55: 641-58, 2009; U.S. Pat. No. 7,329,492; U.S. patent application Ser. No. 11/671,956; U.S. patent application Ser. No. 11/781,166; each herein incorporated by reference in their entirety) in which immobilized, primed DNA template is subjected to strand extension using a fluorescently-modified polymerase and florescent acceptor molecules, resulting in detectible fluorescence resonance energy transfer (FRET) upon nucleotide addition.

In other preferred embodiments, the present invention utilizes protein sequencing techniques. In some embodiments, proteins may be sequenced by Edman degradation. See, e.g., Edman and Begg (1967). “A protein sequenator”. Eur. J. Biochem. 1 (1): 80-91; Alterman and Hunziker (2011) Amino Acid Analysis: Methods and Protocols. Humana Press. ISBN 978-1-61779-444-5. In other embodiments, mass spectrometry techniques are utilized to sequence proteins. See, e.g., Shevchenko et al., (2006) “In-gel digestion for mass spectrometric characterization of proteins and proteomes”. Nature Protocols. 1 (6): 2856-60; Gundry et al., (2009) “Preparation of proteins and peptides for mass spectrometry analysis in a bottom-up proteomics workflow” Current Protocols in Molecular Biology. Chapter 10: Unit10.25.

### Tumor Fraction and RNA Expression.

In order to effectively target an immune response to any individual tumor-specific mutation, a minimum of two initial conditions must be fulfilled: The mutation must be present in the DNA of a sufficient fraction of tumor cells and the DNA encoding the mutation must be transcribed into RNA and expressed as a protein. The tissue fraction comprising the mutant DNA can vary with the precision of resection of the biopsy and the relative composition of tumor to stromal tissue. It may also vary between metastases. In some instances, the fraction of the tumor biopsy comprising the mutation may be very high, represented in over 35% or over 50% of all cell DNA. In other instances, it may be lower, from 1-2% to 10%. In preferred embodiments the targeted mutations are selected from those mutant genes represented in at least 10% of the tumor DNA. In other embodiments a mutation is selected from those mutant genes represented in at least 3 to 5% of the tumor DNA.

The RNA expression is an indicator of whether the gene is transcribed and hence actually targetable as a protein. Bulk RNA content of the tumor is enumerated and for each protein is normalized for the number of total reads of RNA detected in the biopsy sample and the length of the RNA transcript as a metric for gene expression. The number of RNA transcripts varies widely between proteins and overall the bulk RNA frequencies can be described as a log-normal distribution. If the gene is being expressed by both parental chromosomes, the relative expression of the normal and mutant allele for a mutated proteins should be correlated to the DNA mutation frequency. Allele specific expression has been shown to occur in tumors. Such a situation would be manifest with the parental chromosomes being expressed at differential rates and would lead to RNA mutant frequencies that differ from the frequency in the DNA. The mutation:normal ratio of expressed RNA compared to the DNA in the tumor fraction is an indicator of this. In some embodiments the DNA mutation occurs in only one chromosome and if expression of the protein is effected solely or predominantly from the other chromosome, the mutant protein is not expressed or is under represented thus rendering it an ineffective target.

To effectively target a tumor specific mutation it is necessary to establish that the protein is indeed being expressed from the parental chromosome containing the mutant gene. Methods for determination of the RNA fraction are described in Example 19 below. Thus in preferred embodiments peptides comprising mutant amino acids are selected from those proteins that are expressed and for which the RNA mutant: normal fraction is at least 10% of the corresponding DNA tumor:normal fraction and in yet further preferred embodiments the RNA fraction at least 20%.

### Considerations in Selection of Tumor Specific T Cell Antigens

The present invention provides a method for maximizing the number of opportunities to mount a cytotoxic T cell attack on a tumor which carries mutated proteins. In one embodiment the invention provides a method for generating a peptide or an array of peptides that carry the same T cell exposed motifs that are found in the tumor specific proteins, but wherein the peptide or peptides in the array are not present in the tumor, but rather are created by substitution of flanking amino acids to optimize the binding affinity of the peptides to the alleles of a particular tumor-bearing subject. Further embodiments of the invention then enable the selection of a group of peptides so created, which when synthesized, are capable of stimulating tumor specific T cells of the tumor-bearing subject. In particular embodiments these peptides may be encoded in nucleic acid sequences, which may be RNA or DNA. In some embodiments the peptides in the array generated are of 8 to 10 amino acids long. In such embodiments the T cell response stimulated is as the result of binding to MHC I molecules and the response by CD8+ T cells. In other embodiments the peptides in the array generated are 15 amino acids long or from 11-22 amino acids long. In such embodiments the T cell response stimulated is as the result of binding to MHC II molecules and the response by CD4+ T cells. In yet other instances the peptides may be longer, up to about 35 amino acids and may encompass both CD8+ and CD4+ stimulating peptides. In yet other embodiments the T cell response stimulated is as the result of a combination of peptides that stimulate CD8+ and CD4+ responses.

In particular embodiments a single peptide capable of stimulating tumor specific T cells of the tumor-bearing subject may be selected. In other instances, up to 5 peptides maybe selected. In another desired embodiment a group of selected peptides in the array capable of stimulating tumor specific T cells of the tumor-bearing subject comprises at least 5 unique peptides not found in the tumor; in other embodiments the array encompasses at least 20 unique peptides, while in further embodiments the array has more than 60 unique peptides not found in the tumor. Each peptide carries a T cell exposed motif that is shared with the tumor protein at a position that includes the mutated amino acid in the T cell exposed motif. In some embodiments the group of peptides has at least 5 different T cell exposed motifs; in other embodiments the group of selected peptides comprises at least 10 different T cell exposed motifs. In yet other embodiments the group of selected peptides comprises at least 50 different T cell exposed motifs. In some particular embodiments the flanking amino acids of the peptides are selected so each peptide group has peptides collectively predicted to bind to at least 2 different MHC alleles carried by the tumor bearing subject. In other embodiments the flanking amino acids of the peptides are selected so each peptide group has peptides collectively predicted to bind to at least 4 different MHC alleles carried by the tumor bearing subject. In some embodiments a group of peptides created by substitution of the flanking amino acids of one or more T cell exposed motif to optimize binding to MHC allele of an individual subject may be combined in an array with naturally occurring neoepitope peptides. In some embodiments peptides are selected to bind to one MHC allele while avoiding excessively high affinity binding to another HLA allele.

The signal strength stimulating T cells as the result of presentation of peptides to T cells depends in part on the affinity of the peptide to the MHC. In some cases a very high affinity may be sought; in others a moderately high affinity. It is therefore useful to be able to select peptides of a desired affinity, but which are still present the same T cell exposed motif. In one embodiment of the invention therefore, the invention enables the selection of peptides that bind better than 99% of other peptides in the mutant protein; in other embodiments the invention enables selection of peptides binding better than 95% of other peptides in the mutant protein, while in further instances selection of peptides with a binding affinity of about 85% or better is enabled. Described in a different way, in one embodiment the invention enables selection of peptides which are predicted to bind at concentrations of less than 20 nanomolar, and in other embodiments at less than 50 nanomolar, less than 200 nanomolar or at less than 500 nanomolar concentrations.

### T Cell Exposed Motifs

The goal of stimulating a cytotoxic T cell response to a tumor is to specifically and differentially destroy the tumor cells while leaving normal cells intact. It follows that to drive a T cell response specific to the cancer, the T cell receptor must recognize an epitope unique to the tumor. Thus, the mutated amino acid must be located in the exposed pentameric motif exposed to the T cell receptor. When a mutated amino acid is located in a pocket or groove exposed motif, it may or may not affect binding affinity, but it is hidden from the T cell receptor and cannot elicit tumor-specific T cell responses. In some instances, the natural binding affinity of the mutated peptide and its neighboring peptides in the affected protein may give rise to better binding in positions which do not expose the mutated amino acid. In some cases, so-called neoepitope peptides have been selected which do not, in fact, differentiate tumor and normal T cell exposed motifs [11, 47]. In the present invention we seek to maximize use of the T cell exposed motifs containing mutant amino acids, and hence focus the T cell response on these differentiating epitopes, and likewise subsequent expansion of this response as the result of administration of checkpoint inhibitors.

The invention provides peptides to stimulate T cells which will target the mutant protein displaying the same T cell exposed motifs. For this to happen the peptides from the mutant protein in the tumor need to be naturally presented at some level by the MHC alleles of the subject. Therefore, another embodiment of the present invention provides for selection of peptides from the initial array which have a sufficient binding affinity to the subject's MHC alleles to allow some presentation. In particular, therefore, the selection of peptides is down-selected to remove targets which are in the lower 50% of probability of presentation by the subject's MHC, i.e. those with less than the mean binding affinity for the protein from which their T cell exposed motif is derived. This is the reason why in the Examples below not all five T cell exposed motifs created by any single amino acid mutation are necessarily represented.

### Peptide Binding Affinity

Many investigators have considered how to identify peptides in mutated tumor proteins which bind to a patient's MHC alleles. Some have employed mass spectrometry to identify the “presentome” of peptides bound and presented to T cells [15]. However, this has the bias of identifying very high affinity peptides. In some cases, the peptides containing mutant amino acids were never detected by mass spectroscopy [12].

It is unlikely that the highest binding peptides are those which will actually generate the best cytotoxic T cell response. Indeed, evidence in other settings suggests that an intermediate binding affinity may be most effective in stimulating a T cell response and good memory T cells [48]. Low affinity peptides may initiate a CD8+ response but this is not sustained [49]. Furthermore, also drawing on experience in an anti-microbial setting, an active interferon gamma response is also needed to trigger the development of T memory cells [50]. Strength of T cell receptor-pMHC binding may be a factor in determining whether the T cell response to a tumor leads to T cell exhaustion and tolerance [19].

Analysis of the predicted MHC binding of peptides comprising mutations among proteins documented in the TCGA shows no statistical difference in overall predicted binding affinity between mutant and wildtype homolog. However, for TCEM I there is a significant impact when the mutant amino acid lies in positions 2 or 9 of a 9mer. Overall, based on analysis of proteins with mutations recorded in TCGA, the MHC I binding affinity of the peptides containing the T cell exposed motif which become mutated is very low; about 22 micromolar, which is more than 40×lower than the 500 nanomolar that is the consensus T cell stimulatory level. This indicates that such peptides are overall not highly likely to naturally elicit an effective and sustained cytotoxic T cell response and memory, and hence points to the advantage of designing peptides which can alter such presentation.

In one embodiment, the present invention enables the design of peptides presenting the T cell exposed motif of interest with a range of MHC binding affinities, allowing for selection of very high affinity binders or intermediate binding affinity to the alleles of a particular patient with the goal of stimulating and effective cytotoxic response.

### Frequency Characteristics of Peptides Generated by Mutations in Cancer

Comparison of the frequency distribution of the T cell exposed motifs in peptides comprising mutations (for TCEM I cognate for MHC I molecules), among those documented in the TCGA, reveals that those comprising mutated amino acids are motifs that occur less commonly in the human proteome than their wildtype homologues. Overall, the mutant peptides are biased towards those that are rare or even completely absent in the human proteome; the comparator here being all T cell exposed motif in all peptides of all isoforms of human proteins, approximately 88,000 proteins. The mutational event that inserts a new amino acid in the T cell exposed motif consistently produces T cell exposed motif that are much more rare as compared to the wildtype T cell exposed motif.

### Cross Presentation of MHC I and II Binding Peptides

While the primary focus is on stimulating a cytotoxic T cell response, driven by CD8+ T cells, such a response is enhanced and helped by the simultaneous stimulation of a CD4+T helper response. This may be particularly important to the development of a population of memory T cells which can ensure ongoing surveillance and elimination of cancer cells. In some instances, a naturally occurring T helper response may be driven from the native mutated protein. In the present invention we also describe how a tumor specific T helper response can be stimulated by peptides designed to have a high binding affinity to the patient's MHC II alleles and to target T cell exposed motifs which comprise the mutated amino acid. Therefore, in one embodiment the invention provides for designing 15mer peptides by maintaining the TCEM II and varying the flanking sequences.

### Maximizing Targeting of Mutations and Stimulation of Cytotoxic T Cell Responses

The combination of these factors: low binding affinity of mutated peptides and rare T cell exposed motif category reduces the chance of a strong natural cytotoxic response. Mutations detected in proteins in tumor biopsies are the “surviving mutations” which have escaped immune surveillance and have not been effectively eliminated after they occur, and so continue to be propagated in the tumor. In one embodiment, the present invention reverses this balance and provides strongly binding peptides which comprise the rare T cell exposed motif and are thus likely to elicit a strong cytotoxic response. Each of the peptides is designed to provide such conditions for a specific patient allele. If a patient is homozygous for any one of their MHC loci, this is detrimental as it limits the number of T cell clones which can be stimulated by the tumor mutations, likely reducing the chances of tumor elimination. Some cancer patients are further handicapped in stimulating the development of effective cytotoxic T cell responses to tumors due to low numbers of mutations.

In some embodiments, therefore, the present invention provides methods to maximize the utilization of available tumor specific antigens to generate effective cytotoxic T cell response that can bring about elimination of the tumor cells. This is achieved by identifying the T cell exposed motif containing the mutant amino acids and generating an array of peptides which combine these T cell exposed motifs with an array of different flanking amino acids of varying predicted binding affinity to enable selection of appropriate high binding peptides. In the case of TCEM I located in a 9-mer comprising 5 exposed amino acids flanked by 4 groove exposed amino acids, for each T cell exposed motif there is a maximum of 204 or 160,000 possible variant amino acid combinations in the groove exposed position. In some embodiments, an array of 1000 peptides is created by random amino acid substitution in the groove exposed positions, in other embodiments an array of 10,000 peptides is likewise created, and in further embodiments a 50,000 peptide array is created. In the case of TCEM II to create peptides binding differentially to MHC II, we consider a 15 mer in which exposed positions 2, 3, 5, 7, 8 or −1,3,5,7, 8 are kept constant, as all other amino acids in the peptide that are presumed to be involved in the binding affinity are changed by random substitution to create arrays of 1000, 5,000 or 10,000 peptides. In both cases the array sizes cited here are examples that are considered non limiting.

In each case, both MHC I and MHC II, the TCEM is maintained identical to the mutated peptides in the native mutated protein and all TCEM which comprise a mutated amino acid are selected as the basis for generation of binding variants.

In further steps embodied in this invention, the initial array of peptides generated by amino acid substitution is then filtered to remove any duplicate peptides, and in some preferred embodiments peptides predicted to be of low solubility are removed by assigning a score to the polarity of their constituent amino acids. The peptides are then selected to be suitable for the specific patient and his/her combination of MHC I and MHC II alleles. In preferred embodiments all alleles are typed, including MHC I A, MHC I B, MHC I C, and MHC II DRB, DP and DQ loci. In one embodiment, the predicted affinity of the peptides in the native mutant protein is reviewed to determine the probability that a particular peptide would be bound by one or more of the patient's MHC alleles, albeit with a low affinity, and hence presented for T cell recognition. As the goal is to stimulate or “train” T cells to target the specific mutated T cell exposed motifs (TCEM) in the tumor, these must be exposed to T cell recognition to enable targeting of tumor cells. In one embodiment we identify each of the TCEM-allele combinations in each native mutant protein which binds with an affinity greater than the mean for the comprising protein. Such TCEM are targetable by T cells which are also specific to that MHC allele histotope. TCEM-allele combinations which have a predicted binding affinity above the mean are set aside as unlikely to ever be presented. For this subset of “presentable” TCEM-allele combinations, we then assess the array of randomly generated peptides, filtered for binding and solubility, and identify a peptide for each TCEM-allele combination with a desired predicted binding affinity. In some embodiments, the peptide with maximum predicted binding affinity for each allele may be chosen. This may be a peptide that binds at 2.5 or 3 or more standard deviation units below the mean for peptides in the protein (i.e., higher affinity). Such a high binding peptide would be comparable to those detected as part of the presentome by mass spectroscopy and equivalent to approximately <20 nM to 100 nM, depending on the protein context. In preferred embodiments, peptides are chosen with high, but not excessive predicted binding affinity, keeping in mind the probability that this may be more likely to stimulate an effective cytotoxic response and memory and mitigate against T cell exhaustion. Such a binding affinity may be from 1-2 standard deviation units below the mean for peptides in the protein, typically equivalent to 100-500 nM. Overall, the invention embodies the ability to select for a desired binding affinity and can be considered “tunable” to that selected binding affinity for each patient allele.

Given that each mutated protein has 5 possible TCEM I and TCEM II which exposed the mutated amino acid, in a patient who, for example, has 6 known MHC I alleles and 4 known MHC II alleles, there is a maximum of 30 possible high binding peptides for CD8+ stimulation and 20 for CD4+ stimulation for every known mutated protein. This may be reduced, sometimes by half, due to filtering of non-presented TCEM but still offers a vastly greater number of ways to stimulate T cells which will target the TCEM of interest that depending on natural binding peptides. Simply put, if a binding peptide does not exist, we will create one and if a poor binder is found the affinity is improved by modification of the MHC groove exposed amino acids. The novel peptide thus created will stimulate T cells bearing TCR specific to the tumor.

In some embodiments the novel peptides are used in vitro to stimulate dendritic cells or T cells. In some embodiments such cells are of autologous source, in yet other embodiments they are obtained from allele-matched donors. Stimulated cells are then administered to the cancer patient to passively provide an active T cell population or to provide dendritic cells presenting the TCEM of interest which can stimulate T cells in the patient. In yet other embodiments the peptides are used as components of a peptide vaccine. In yet other embodiments the peptides are applied as a fusion with antibody sequences. In further embodiments the peptides may be encoded in RNA or DNA for administration.

In desired embodiments, therefore, the process described above yields a unique array of peptides for a particular patient, enabling stimulation of T cells targeting the maximum possible TCEM specific to that patient's tumor-specific mutations and mutated proteins, by presentation of peptides of selected binding affinity in each of the known alleles the patient carries, and the peptides further selected to be soluble. This is a panel of peptides which can then be deployed to stimulate T cells in vivo and in vitro by application in a number of different formats.

As further illustrated in the Examples, this invention may be applied in two ways, to design and apply bespoke neoantigen vaccines for individual patients and to provide ready-to-go multi-cancer neoantigen arrays for neoantigens found commonly in many cancers.

### Design of Personal Neoantigen Vaccines

In a preferred embodiment the present invention allows the rapid design of a personalized immunotherapeutic intervention designed for each cancer patient based on their HLA alleles and particular set of mutations. In some applications of this embodiment the mutations are unique to one patient. This intervention becomes feasible as soon as sequencing of a tumor biopsy and HLA typing is available and can be rapidly computed. In some embodiments the process of sequencing a biopsy may be repeated several times in the course of treatment and the selection of peptides updated based on detection of new mutations. In some preferred embodiments the invention provides an immunotherapy solution for patients who have few proteins with known mutations, for example, but not limited to, glioblastoma patients, who would otherwise be limited to only one neoantigen per protein and possibly no neoantigens with appropriate HLA binding. The preferred embodiment of the present invention is to provide the maximum number of T cell stimulating peptides which will result in targeting of every possible TCEM in which the mutant amino acid occurs and by utilizing every possible HLA. In a further embodiment of the invention the peptides are down-selected to those which will target TCEM presented in vivo and those which are less likely to cause adverse targeting of other human proteins. In an extension of this preferred embodiment, the selected stimulatory peptides may be grouped to provide a series of vaccinations or treatments which allow the utilization of all available alleles the patient carries, while not causing competition for peptide presentation in any one group of peptides.

In some embodiments the selected peptides are applied to dendritic cells in vitro which are then administered to the patient to stimulate T cells. In yet other embodiments the selected peptides are applied in vitro to stimulate a population of T cells which are administered to the patient. In yet other embodiments the peptides, or nucleic acids encoding them are administered directly to the patient in one or more groups spaced over time. In particular embodiments the selected peptides may be encoded in nucleic acid sequences, which may be RNA or DNA

### Neoantigen Array for Common Mutations in Multiple Cancers

Recognizing that many cancers share common mutations in certain proteins, an embodiment of the present invention provides an array of pre-computed and designed peptides which will provide high affinity binding peptides, or nucleic acids that encode them, for the common mutations in commonly mutated proteins shared by many cancers. In preferred embodiments, the proteins with common mutations which are pre-computed and have designed peptides include but are not limited to EGFR, H3.3, IDH, BRAF, TP53, PTEN, ERBB2, PIK3CA and KRAS

In some proteins, and in the particular case of EGFRvIII, in addition to the common amino acid substitution mutations, insertion-deletions are also common in many types of cancer. Some cancers are associated with the presence common fusion proteins, including but not limited to KIAA1549-BRAF and EML4-ALK. In a further embodiment of the invention, we therefore also provide a method of selecting an array of peptides which can serve as tumor specific T cell stimulating peptides for these common deletions and fusions. This is an approach which can be applied wherever a deletion or fusion creates a novel amino acid motif at the junction or deletion site and thus the examples for EGFRVIII, KIAA1549-BRAF and EML4-ALK are not considered limiting.

In preferred embodiments one or more the pre-computed and designed high affinity peptide from common mutated proteins are applied in the treatment of cancers, including but not limited to adrenocortical carcinoma, bladder urothelial carcinoma, breast adenocarcinoma, cervical squamous cell carcinoma, cholangiocarcinoma, colon carcinoma, lymphoid neoplasm diffuse large b-cell lymphoma, esophageal carcinoma, glioblastoma multiforme, head and neck squamous cell carcinoma, kidney chromophobe, kidney renal clear cell carcinoma, kidney renal papillary cell carcinoma, acute myeloid leukemia, chronic myelogenous leukemia, brain lower grade glioma, liver hepatocellular carcinoma, lung adenocarcinoma, lung squamous cell carcinoma, mesothelioma, ovarian serous carcinoma, pancreatic adenocarcinoma, pheochromocytoma and paraganglioma, prostate adenocarcinoma, rectal carcinoma, sarcoma, skin cutaneous melanoma, stomach adenocarcinoma, testicular germ cell tumors, thyroid carcinoma, thymoma, uterine corpus endometrial carcinoma, uterine carcinosarcoma, uveal melanoma. In preferred embodiments the precomputed and designed peptides included in the array are designed to have high binding for any one of the following alleles A_0101, A_0201, A_0202, A_0203, A_0206, A_0211, A_0212, A_0216, A_0217, A_0219, A_0250, A_0301, A_0801, A_1101, A_2301, A_2402, A_2403, A_2501, A_2601, A_2602, A_2603, A_2902, A_3001, A_3002, A_3101, A_3201, A_3301, A_6801, A_6802, A_6901, A_8001, B_0702, B_0801, B_0802, B_0803, B_1501, B_1502, B_1503, B_1509, B_1517, B_1542, B_1801, B_2703, B_2705, B_3501, B_3801, B_3901, B_4001, B_4002, B_4402, B_4403, B_4501, B_4506, B_4601, B_4801, B_5101, B_5301, B_5401, B_5701, B_5801, B_7301, B_8301, C_0303, C_0401, C_0501, C_0602, C_0702, C_1203, C_1402, C_1502, DPA1_0103-DPB1_0201, DPA1_0201-DPB1_0101, DPA1_0201-DPB1_0501, DPA1_0301-DPB1_0401, DPA1_0301-DPB1_0402, DPB1_0101, DPB1_0201, DPB1_0301, DPB1_0401, DPB1_0402, DPB1_0501, DPB1_1401, DPB1_2001, DQA1_0101-DQB1_0501, DQA1_0102-DQB1_0501, DQA1_0102-DQB1_0502, DQA1_0102-DQB1_0602, DQA1_0103-DQB1_0603, DQA1_0104-DQB1_0503, DQA1_0201-DQB1_0202, DQA1_0201-DQB1_0301, DQA1_0201-DQB1_0303, DQA1_0201-DQB1_0402, DQA1_0301-DQB1_0302, DQA1_0303-DQB1_0402, DQA1_0401-DQB1_0402, DQA1_0501-DQB1_0201, DQA1_0501-DQB1_0301, DQA1_0501-DQB1_0302, DQA1_0501-DQB1_0303, DQA1_0501-DQB1_0402, DQA1_0601-DQB1_0402, DQB1_0201-, DQB1_0202-, DQB1_0301-, DQB1_0302-, DQB1_0402-, DQB1_0501-, DQB1_0502-, DQB1_0503-, DQB1_0602-, DRB1_0101, DRB1_0101 C30S mutant, DRB1_0301, DRB1_0401, DRB1_0404, DRB1_0405, DRB1_0701, DRB1_0801, DRB1_0802, DRB1_0901, DRB1_1001, DRB1_1101, DRB1_1201, DRB1_1301, DRB1_1302, DRB1_1454, DRB1_1501, DRB1_1602, DRB3_0101, DRB3_0202, DRB3_0301, DRB4_0101, DRB4_0103, DRB5_0101. Additional alleles may be added to this list as training sets become available and thus this allele list is not considered limiting. In preferred embodiments, as soon as a patient is identified as carrying a common mutation in a tumor, and his or her HLA typing is known, one or more peptides from the pre-computed ready-to-go array is selected and used in vitro to provide dendritic cells that stimulate T cells on administration to the patient, stimulate T cells which are administered to the patient, or is administered as a component of a peptide vaccination regimen or vaccination with nucleic acids encoding the peptides. In a further embodiment the TCEM matches which can give rise to off-target cytotoxic effects are also precomputed for all potential allele binding situations, enabling risk analysis of peptide use for each patient based on their allele combination.

Neoantigen Based Interventions Combined with Additional Immunotherapies

Application of the bespoke and multi-cancer designed peptides described in the prior sections may, in some embodiments, be combined with other cancer immunotherapies. In some embodiments the peptides or their encoding nucleic acids may be used in vitro to prime dendritic cells or stimulate T cells, or as vaccines in conjunction with drugs targeting upregulated cancer-expressed proteins, biopharmaceuticals binding to tumors, CAR T therapies, radiotherapy, chemotherapy and other clinical interventions. In preferred embodiments the combined chemotherapy should not lead to lymphodepletion. In one particular embodiment the application of the designed peptides or encoding nucleic acids to stimulate dendritic cells or T cells administered to the patient may be combined with a check point inhibitor blockade. In other preferred embodiments, the methods of the present invention comprise administering an immune checkpoint inhibitor to a subject following administration of a multi peptide vaccine or nucleic acid vaccine encoding the peptides. Checkpoint inhibitors act by blocking the inhibition of T cell responses or blocking the termination of a T cell response, thereby unleashing continuing T cell actions. The present invention is applied to ensure that the appropriate tumor targeting T cells are present prior to administration of such a check point blockade. In preferred embodiments, therefore, the peptides designed by the present invention are applied prior to a checkpoint blockade. Suitable checkpoint inhibitors include, but are not limited to, antigen binding proteins that inhibit immune checkpoints, for example by PD-1, PD-L1 or CTLA-4. Suitable checkpoint inhibitors include, but are not limited to, Pembrolizumab, Nivolumab, Ipilimumab, Atezolizumab, Durvalumab, REGN2810 (Anti-PD-1), BMS-936558 (Anti-PD-1), SHR1210 (Anti-PD-1), KN035 (Anti-PD-L1), IBI308 (Anti-PD-1), PDR001 (Anti-PD-1), BGB-A317 (Anti-PD-1), BCD-100 (Anti-PD-1), and JS001 (Anti-PD-1). Other immunomodulatory interventions having the effect of enhancing or extending cellular immune function include but are not limited to ALT-803 and N-803 (IL-15), and haNK, tank and other NK cells.

### Utilization of Designed Peptides

In some embodiments the present invention will yield an array of many peptides suitable for enhancing the CD8+ response of a particular patient to his/her mutated tumor proteins and a list of many peptides suitable for enhancing a CD4+ helper response to these proteins. In some particular embodiments the number of peptides designed to bind MHC and stimulate T cells in a particular patient may be up to 5, in others it is about 20, in yet others it is over 100 and in yet others over 200 peptides. In some embodiments the peptide array will include those which bind to 1 allele, 2 alleles or up to 6 MHC I alleles and others which bind 1, 2 or up to 6 MHC II alleles. In order to optimize the application of the peptides and maximize the use of binding alleles while minimizing competition for binding at any single administration, a further embodiment of the present invention is to prioritize and group the peptides for sequential administration. In a preferred embodiment the peptides may be grouped into subgroups of about 5, in other embodiments subgroups of about 10 are preferred, and in yet other embodiments subgroups of about 20 are preferred and in further embodiments larger groups are preferred. The subgroups may combine both MHC I and MHC II binding peptides. Some peptides may be repeated in several subgroups. In some embodiments where vaccination regimens comprise sequential administration of a subset of selected peptides, each peptide administration may be followed by check point inhibitor treatment. In some embodiments, consideration is given to whether particular TCEM encompassed in the peptides in each group are rare or common TCEM in the human proteome or immunoglobulinome. In some preferred embodiments priority is given to inclusion of peptides that comprise rare TCEM. In each instance where a peptide is mentioned above, this may also refer to the application of a nucleic acid encoding the peptide. In preferred embodiments peptides that have TCEM matches in certain human proteins are excluded from consideration, where stimulating a T cell response which may target the human proteins may result in an adverse effect. In yet another embodiment, where transcription levels of the mutated proteins in a tumor are known, peptides may be prioritized based on their transcription level to increase the chance of successful targeting of tumor cells.

### Selection of Peptides for Formulation and Administration

Administration of peptide vaccines to cancer patients has to date been achieved by several methods. In some instances, peptides have been applied to autologous dendritic cells in vitro and the dendritic cells transfused back into the patient. In some instances, the peptides have been encoded in RNA or DNA sequences and delivered in vitro or in vivo. Intradermal delivery is also a delivery route of choice. While cancer vaccines, both from tumor associated proteins and neoepitope vaccines have typically been administered in an acute treatment phase, it is also important to consider the long-term maintenance of an effective tumor antigen specific T cell repertoire to avoid recurrence of immune evasion resulting in progression or metastasis of the tumor. Consideration therefore needs to be given to delivery formulations which can be administered over the long term, and in some cases for many years of life, and which are more acceptable to the subject. In some embodiments, therefore, the present invention provides methods for formulation for parenteral delivery by several routes, including intradermal. In yet other embodiments the invention provides methods to deliver such peptide vaccines non-parenterally, including but not limited to orally.

Selection of a peptide may be personalized according to the individual subject's tumor mutations and HLA, and selected to optimize the binding of the peptide to their MHC molecules while still presenting the tumor specific motif comprising the mutant amino acid or amino acids to the T cells. This may be achieved by maintaining the T cell exposed motif, which engages the T cell receptor and comprises the tumor specific mutation, but changing amino acids in the flanking pocket or groove exposed positions which determine binding to the MHC molecular groove (See, e.g., PCT US2020/037206, which is incorporated by reference herein in its entirety) and as briefly described in Examples below. As many different combinations of amino acids placed in the groove exposed positions may achieve the objective of binding to a particular HLA within a desired range of predicted binding affinity, the opportunity arises to select from among the potential candidate groove exposed motifs. In the present invention we provide methods of selection among possible groove exposed motifs based on various criteria which facilitate formulation, manufacturability and uptake by antigen presenting cells in vivo.

When a personalized neoepitope vaccine is designed for an individual following the exome sequencing of a tumor biopsy collected at surgery, there is typically urgency in making the vaccine rapidly available for administration. In an ideal situation the goal is to have the vaccine available for administration within a month post-surgery. Furthermore, in many embodiments a neoepitope vaccine comprises multiple peptides, each with different physicochemical characteristics. It is therefore desirable to be able to predict the performance of each peptide in formulation, manufacturability and uptake rapidly and consistently. The present invention provides a method for selection of groove exposed motifs that accomplishes the goal of a desired MHC binding affinity and enhanced performance in formulation, manufacturability and uptake.

### Considerations in Formulation

Peptides are a rapidly growing class of pharmaceutical products and cancer vaccines and immunopathology interventions comprising peptides share many of the same challenges as peptides delivered for other reasons.

Two broad sets of challenges exist in formulation; these are in stability and solubility [51]. Chemical changes such as oxidation and deamidation comprise one source of instability problems. Peptides comprising methionine, tryptophan, histidine, cystine and tyrosine are most prone to oxidation, whereas those comprising asparagine and glutamine are prone to deamidation. In one embodiment therefore, to reduce oxidation, peptides are selected in which amino acids from the group comprising methionine, tryptophan, histidine, cystine and tyrosine are excluded in the groove exposed motif. In yet another embodiment, to reduce deamidation, peptides are selected in which asparagine and glutamine are not present in the groove exposed motif. Exclusion of cysteine has the additional benefit in reducing cross linking between peptides by formation of disulfide bonds.

Physical challenges to stability include the formation of aggregates or micelles, adsorption to surfaces and denaturation due to extremes of temperature or pH. Various strategies have been developed to mitigate each of these including, but not limited to, the use of surfactants and lower concentrations, adjusting salt concentrations and pH (to reduce aggregations), polymer excipients such as polysorbate 80, selection of appropriate containers (to mitigate adsorption), addition of salts or metal ions and control of pH (to reduce denaturation), addition of buffers, selection of storage temperature (to reduce hydrolysis) and addition of antioxidants and chelating agents (to reduce oxidation).

Biological challenges to stability include enzymatic degradation and intestinal permeability. Strategies have been developed to mitigate both the above including but not limited to the use of enteric drug delivery systems and permeation enhancers. To overcome the enzymatic and pH-dependent degradation of peptides in the stomach, in addition to permeability issues and the potential for degradation via first pass metabolism, formulation strategies, such as enzymatic activity inhibitors, permeation enhancers, enteric coatings, and carrier molecules, can be employed

Solubility of peptides is at a minimum at the isoelectric point. Hence optimization of pH, salt concentration and ionic strength are non-limiting examples of approaches to improve solubility. An assessment of peptide solubility in aqueous solvents can be made by determining the polarity and the partition coefficient. A determination of the octanol:water partition co-efficient is another useful guide as it could predict the molecules solubility and permeability. In some embodiments therefore peptides are selected based on their index of polarity. In other embodiments the selection is based on the partition coefficient or the log thereof (log P), and in preferred embodiments the partition coefficient of octanol:water.

In some particular embodiments peptides are selected to include highly polar amino acids in their groove exposed motif positions. In some particularly preferred embodiments peptides are selected to include amino acids selected from the group comprising arginine, lysine, glutamic acid or aspartic acid in the groove exposed motifs.

Stabilizing excipients may be included in the formulation including, but not limited to polysorbate 20, polysorbate 80 and sodium dodecyl sulfate, pluronic 107, polyethylene glycol, dextran, hydroxyethyl starch, ascorbic acid, salts of sulfurous acid, and thiols, ethylene glycol, glycerol, glucose, mannitol. Lyophilization is a common mode of preservation of peptides and during lyophilization additional excipients are protective, examples include, but are not limited to, sodium phosphate, monobasic monohydrate, mannitol and sucrose. Spray drying is another form of preservation which may be employed. Here, an aqueous peptide solution is transformed into a powder by “atomizing” (transform the liquid into very small droplets of 10-500 μm diameter) the peptide solution through a nozzle into a chamber together with a hot gas in order to remove the liquid. This method is comparable to lyophilization and additionally, during spray drying fine and dense particles are formed which are less static than the particles formed during lyophilization. Stabilization during the drying process is achieved using excipients that can form hydrogen-bonds with the peptides. Sugars such as trehalose, raffinose, or dextran are commonly used as matrix formers. Due to their high glass transition temperatures they can offer excellent long-term storage stability. Combinations of amino acids such as histidine, glycine, proline and arginine, or divalent metal ions such as zinc, can be added to maintain the structure, reduce aggregation, and minimize chemical. In addition, surfactants, such as polysorbates or pluronics, offer protection at the liquid-air interface during the atomization stage, preventing aggregation or denaturation.

Therefore, in some embodiments peptides are formulated with one or more stabilizing excipients.

### Delivery of Peptide Vaccines

Peptides in isolation are not readily taken up by antigen presenting cells without the addition of an adjuvant. In some cases, the adjuvant effect is a function of the form in which peptides are administered, including, but not limited to, when peptides are delivered as an emulsion, particulate, liposome, virosome, or glucan particle. In other instances a peptide vaccine may be administered with an adjuvant selected from the following non-limiting examples: lipid A analogues (e.g. poly I:.C), imidazoquinolines 9e.g. imiquimod), CpG, saponins, C type lectin ligands, CD1d ligands 9 e.g. a-galactosylceramide), aluminum salts (e.g. aluminum hydroxide), emulsions (e.g. MF59), and many variants thereof [12, 52]. Adjuvants may act in many ways, by enhancing antigen uptake by antigen presenting cells, by activating toll receptors, activating inflammasomes, enhancing immune cell recruitment and by increasing presentation of antigen to T cells [53]. A further adjuvant used with neoantigen peptides has been granulocyte stimulating factor [12]. Combinations of adjuvants may be used together. In the case of peptide vaccines, enhancing antigen uptake by antigen presenting cells, both professional and non professional, is the most critical function of an adjuvant.

Peptide vaccines may be delivered to the subject to be vaccinated by parenteral or non parenteral routes. The most common parenteral route for a neoepitope vaccine is intradermally or subcutaneously. This takes advantage of the high population of antigen presenting cells and particularly dendritic cells in the dermis.

Delivery of peptides by non-parenteral routes presents some additional challenges. Non-parenteral routes include delivery to mucosal surfaces of the respiratory tract by intranasal or pulmonary delivery, rectal delivery, and per os, whether as sublingual films, buccal mucosal application, or delivery to the gastrointestinal tract. Each location brings different challenges in peptide formulation.

For oral delivery to the intestinal mucosa, in addition to solubility and stability, safe passage to the desired point in the intestine and permeability allowing access to antigen presenting cells are important considerations. The regional specialization in the intestinal immune system is complex, but the most desired location of delivery of a peptide vaccine is to the small intestine, where dendritic cells are present and where mucosal epithelial cells also serve as antigen presenting cells expressing MHC II molecules and presentation of antigenic peptides to the gastrointestinal lymphatic tissue (GALT) [54-56]. Therefore, in some embodiments the peptides are formulated for enteric delivery and in most preferred embodiments the formulation is designed to deliver the peptides to the mucosa of the duodenum and ileum. Protection from gastric enzymes and physical stresses may in some embodiments be by formulation in tablet form or, in preferred embodiments, as a capsule with an enteric coating. Many materials known to those skilled in the art may be used as enteric coating including but not limited to waxes, fatty acids, cellulose, polymers etc. Within the enteric coated capsules peptides may be formulated to enhance permeation into the mucosa. The barriers to permeation include passage through the mucus layer, motility, enzyme digestion, all of which must be overcome before uptake by antigen presenting cells and presentation to T cells can occur. In some embodiments therefore the peptides are formulated in particulate form as nanoparticles, as gels, encased in biodegradable microneedles, or placed in mucoadhesive patches. In a particularly preferred embodiment, the peptides are encased in a lipid drug delivery system. In preferred embodiments the lipid drug delivery system comprises a solid lipid nanoparticle, an emulsion or microemulsion, a self-emulsifying drug delivery system [57], a nanocapsule or a liposome. In further preferred embodiments particulate size is maintained at less than or equal to 200 nm to facilitate uptake. While lipid drug delivery systems may be used as a formulation within an enteric delivery systems including enteric capsules, or tablets capsule, they may also be used other routes of delivery, including but not limited to other mucosal routes (rectal, buccal, sublingual, intranasal, inhalation) and parenteral routes including but not limited to intradermal subcutaneous and intraperitoneal.

### Molecular Weight

Peptides comprising neoepitopes and in particular those which have been designed to provide personalized groove exposed motifs are short. Peptides selected from naturally occurring sequences in a tumor may be up to about 25-30 amino acids. Peptides which are personalized by designing the groove exposed motifs to optimize HLA binding are typically up to 15 or 16 amino acids for MHC II binding peptides and 8-10 amino acids for MHC I binding peptides.

Peptides selected by the methods described herein therefore have a low molecular weight. In some embodiments the selected vaccinal peptides are under or equal to 4000 Da. In preferred embodiments the molecular weight of each selected vaccinal peptide is less than or equal to 2000 Da; in a highly preferred embodiment the peptide molecular weight is less than or equal to 1500 Da.

### Treatment of Other Immunopathologies

Immunopathologies are also personal diseases as they depend on the conjunction of an antigen exposure, HLA alleles and T cell repertoire based on prior epitope exposure that is unique to the individual patient.

Modified epitopes can also play a role in modulation of other immunopathologies, outside the field of oncology. This includes, but is not limited to, applications in autoimmune diseases, allergies and inflammation where the problem is not an insufficient T cell stimulation, but rather an overexuberant response. Provision of a very high affinity binding peptide can serve to exhaust or diminish the T cell response to the particular T cell exposed motif in question and thereby diminish CD4+ T cell help or a CD8+ cytotoxic response and ameliorate the pathogenesis of the disease. In each case the peptides are customized to ensure binding appropriate the HLA alleles of the individual patient.

Autoimmune diseases in which such an approach may be useful include, but are not limited to rheumatoid arthritis, diabetes type I and type II, Ankylosing Spondylitis, Atopic allergy, Atopic Dermatitis, Autoimmune cardiomyopathy, Autoimmune enteropathy, Autoimmune hemolytic anemia, Autoimmune hepatitis, Autoimmune inner ear disease, Autoimmune lymphoproliferative syndrome, Autoimmune peripheral neuropathy, Autoimmune pancreatitis, Autoimmune polyendocrine syndrome, Autoimmune progesterone dermatitis, Autoimmune thrombocytopenia purpura, Autoimmune uveitis, Bullous Pemphigoid, Castleman's disease, Celiac disease, Cogan syndrome, Cold agglutinin disease, Crohns Disease, Dermatomyositis, Eosinophilic fasciitis, Gastrointestinal pemphigoid, Goodpasture's syndrome, Graves' disease, Guillain-Barré syndrome, Anti-ganglioside Hashimoto's encephalitis, Hashimoto's thyroiditis, Systemic Lupus erythematosus, Miller-Fisher syndrome, Mixed Connective Tissue Disease, Myasthenia gravis, Narcolepsy, Pemphigus vulgaris, Polymyositis, Primary biliary cirrhosis, Psoriasis, Psoriatic Arthritis, Relapsing polychondritis, Sjögren's syndrome, Temporal arteritis, Ulcerative Colitis, Vasculitis, and Wegener's granulomatosis.

Allergic responses which may benefit from immunomodulation by design of personal peptides of modified binding include but are not limited to allergies to plant, animal, insect, arachnoid materials, parasites, and other environmental materials comprising allergen epitopes. Allergies may result from airborne or gastrointestinal exposure or from skin contact.

In some instances, an immunopathology can arise as the result of an adverse response to a therapeutic agent administered to a subject. In some cases the therapeutic is a biopharmaceutical protein.

In each case an individual subject afflicted by an autoimmune disease or allergen may be typed as to their HLA alleles and a peptide array designed specifically for that person to provide peptides that exhaust the T cell response. Examples of such customized peptides for three particular allergens are shown in Example 24.

It follows therefore that, as a personalized peptide array can be designed for an individual affected by an immunopathology other than cancer, that similar considerations as all those discussed above arise in the selection of groove exposed motifs based not only on achieving a desired predicted binding affinity for a subjects individual HLA alleles, but also to facilitate formulation, manufacturing and delivery.

### EXAMPLES

### Example 1: Selection of Mutant Peptides and Generation of Better Binding Peptides

The development of vaccines and stimulants for dendritic cells and T cells in vitro to comprise multiple peptides with a selected desired affinity for the patient's alleles builds on methods previously described to precisely predict MHC binding, identify and analyze T cell exposed motifs and generate peptides with altered binding affinity (See PCT Appl. US14/41523, PCT Appl. US15/39969, and PCT Appl US17/21781, all of which are incorporated herein by reference in their entirety).

### Identification of Relevant Peptide Positions.

In order for a T cell to differentially target a tumor cell expressing a mutated protein, the mutated amino acid has to be located in a position “visible” or exposed to the T cell receptor and not hidden in the pocket or groove exposed positions that determine binding. A first step in designing a multi peptide vaccine or stimulant panel is therefore to identify those peptide positions which expose the mutated amino acid. For MHC I this means the mutant amino acid must be at positions 4, 5, 6, 7 or 8 of a 9-mer peptide and for MHC II at positions 2, 3, 5, 7, 8 of the 9-mer core of a 15 mer. This identifies TCEM IIA; TCEM IIB positions are at −1, 3, 5, 7, 8. We first calculated the predicted binding affinity of all sequential peptide positions in the mutant protein and then selected those peptides with relevant TCEM comprising mutated amino acids.

A T cell is only able to target a TCEM if that motif is presented in the host from the naturally occurring mutant peptide. Mutant TCEM that lie in peptides that are extremely unlikely to ever be presented are thus poor targets. We therefore filtered the TCEM to identify those which have some likelihood of exposure in the host, limiting to those whose predicted binding affinity is greater than the mean for the protein. This is not an absolute requirement but maximizes the potential for a successful targeting.

For each of the selected peptides comprising a mutant TCEM, a bank of peptides was generated by randomly varying the flanking amino acids, and recalculating the new binding affinity for each allele of interest. For a 9-mer with a pentamer exposed TCEM, this implies up to 160,000 (20⁴) different peptides could be generated, each with a different binding affinity. For practical purposes a bank of 1000 or up to 10,000 peptides is usually sufficient to provide peptides within the range of binding affinity desired. For MHC II we opted to vary only those amino acids outside the core 9 mer peptide comprising the TCEM, as the intercalated amino acids which are in pocket (groove exposed) positions affect binding but may also influence the positioning of the exposed amino acids.

A further practical consideration is solubility of the peptide. A score was generated based on the polarity of the constituent amino acids and only peptides likely to be soluble were put forward as candidates. Sufficient peptides can be generated to prevent this from becoming a limitation.

For a group of 5 proteins each with one mutation and a patient with 4 known alleles therefore a maximum number of allele TCEM combinations is 5 TCEM×5 proteins×4 alleles or 100 possible ways to stimulate T cells which will uniquely target those proteins. This is reduced by the TCEM of low probability of natural presentation.

### Example 2: Selection of Personalized Simulated Peptides

The process described in Example 1 generates a selection of peptides of different binding affinity for each combination of mutant-containing-TCEM and patient allele. Peptides are then selected which have a desired predicted binding affinity. We have discussed the relevance of binding affinity on T cell phenotype in the Description above. As peptides of many different binding affinities are provided, the desired affinity may be selected. In the subsequent examples wherever T cell stimulation is the desired outcome we have opted to focus on peptides with predicted binding affinity at about 2 standard deviations below the mean of the protein, placing them at about the 95^{th} percentile; i.e. the top 5% binders, but not higher, because conceivably very high affinity peptide could lead to immunosuppression or exhaustion. In contrast, to induce anergy to allergens a higher binding affinity of greater than 2.5 standard deviation units below the mean, and ideally about 3 standard deviation units is desired.

Utilization of the available peptides may depend on the intended use as a neoepitope vaccine or in vitro stimulant of dendritic cells and T cells to be administered to the patient.

Peptides may be selected to use in groups that target the maximum number of combinations of allele and TCEM in any one application. One desired aspect is to ensure not all peptides administered at any one time as a multi-neoepitope vaccine target the same allele, thus competing with each other for space in MHC and presentation. When dendritic cells and T cells are targeted in vitro it may be desirable to provide as many combinations as possible.

### Example 3: Determination of HLA Haplotypes Determined from Whole Exome Sequences

A ‘BAM slice’ of the exome file containing the HLA locus (GRch38=chr6:29722700-33143300) was used. The principles outlined for the Optitype which focuses on the read matches to exons 2 and 3 of the MHC molecules was used in conjunction with the magicBLAST aligner. magicBLAST has features that are particularly suited for this type of application. Optitype has been shown to be one of the most accurate methods but only has prediction capabilities for MHC I and thus teaches away from MHC II typing. This general approach was modified as follows to provide MHC II typing also.

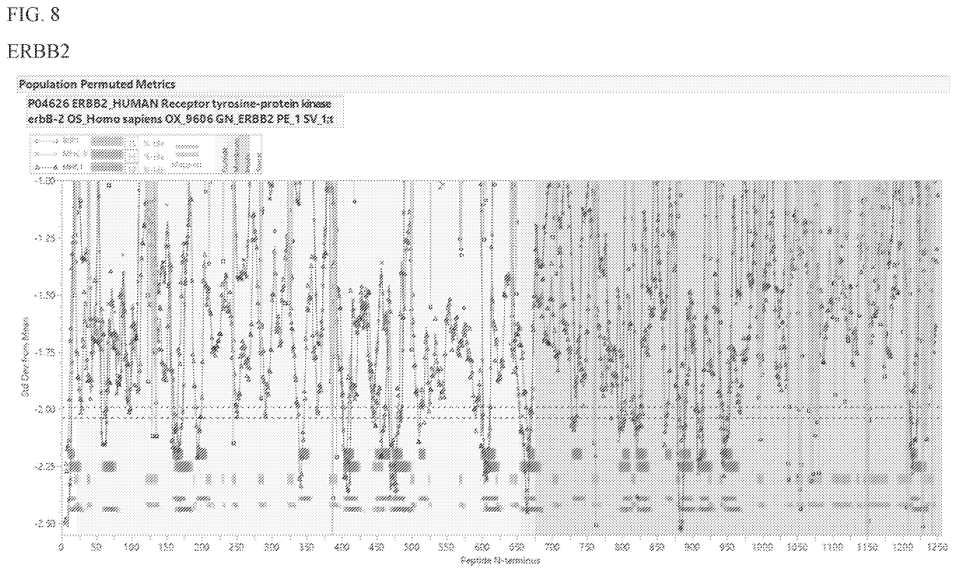

The BAM formatted ‘slice’ was converted to a fastq split read format required by magicBLAST using tools from GATK (Broad Institute). A special magicBLAST database for both MHC I and MHC II needed for the alignment process was created from the IMGT HLA sequence database (imgt.org). Exons 2 and 3 are each 270 nucleotides and code for the amino acid variations that form the basis of the different HLA haplotypes. A matrix 540×N (N=number of reads) was created and was used to tally the 100% read match at each nucleotide position produced by magicBLAST. The magicBLAST 100% alignment statistics in the matrix were then tallied across all reads and matched to the different MHC genotypes. Whereas Optitype uses a special integer linear programming approach with the hit matrix to assign the best fit HLA, we demonstrated that a simple tally of the hits in the matrix are adequate to clearly identify the haplotype of the exome data. FIG. 8 shows an example of the output.

### Example 4: EGFRvIII Splice Variant in Glioblastoma

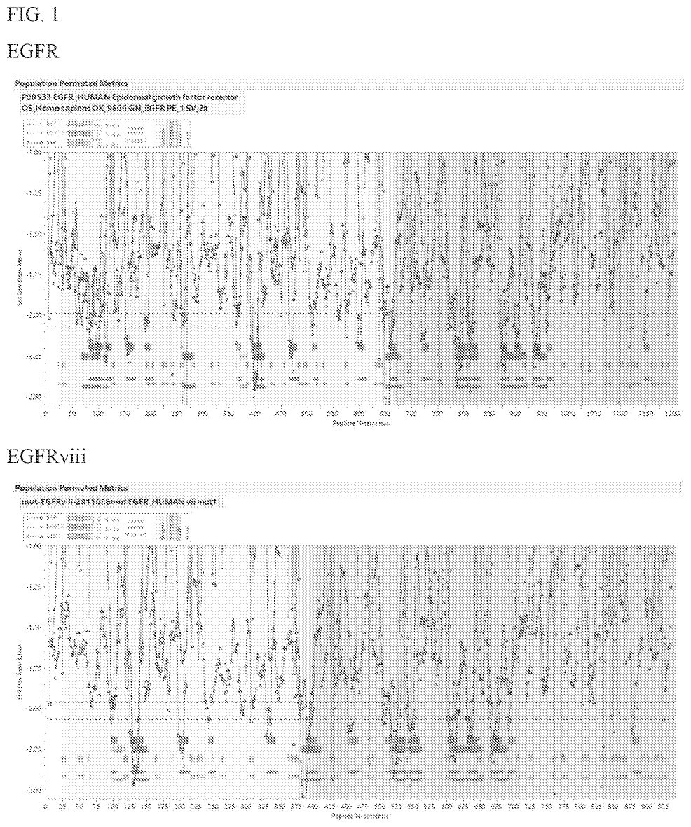

EGFR is a transmembrane protein with a transmembrane domain located at positions 646-667 relative to its N terminal and a signal peptide. EGFR is amplified in over half of primary glioblastoma cases [61]. The EGFRvIII splice variant, (isoform i, NP_001333870) occurs in a large percentage of all EGFR amplified of all glioblastomas. EGFRvIII arises as a result of splice errors that omit exons 2-7 and remove amino acids 2-273 of the mature protein, while generating a novel glycine at the junction site. This results in the potential exposure in a tumor of novel T cell exposed motifs in peptides bound in MHC I and engaging CD8+ T cells, with the novel exposed motifs for MHC I bound peptides being the amino acid pentamers EEKKG (SEQ ID 6), EKKGN (SEQ ID 7), KKGNY (SEQ ID 8), KGNYV (SEQ ID 9), and GNYVV (SEQ ID 10). As shown in FIG. 1, while there is a moderate level of predicted MHC I binding, and the creation of a small non-dominant B cell epitope, there is essentially no moderate or high affinity MHC II binding in the proximity of the splice site tumor specific T cell exposed motifs. In this example, we demonstrate that by making changes in the amino acids flanking the tumor specific T cell exposed motifs of EGFRvIII, it is possible to create many novel synthetic peptides with a predicted higher affinity MHC I binding for the tumor specific motif, increasing the possibility of stimulating a CD8+ T cell clone for a large number of potential allele-T cell exposed motif combinations. Table 1 provides examples of such peptides for MHC A, B and C, alleles which would bind at approximately 2 standard deviation units below the mean, equivalent to approximately 50-100 nmolar, meaning that these would compete for binding in the top 13% binding of all peptides in EGFRvIII. While 2 or 3 examples are provided for each allele-TCEM combination, such examples are not considered limiting but were extracted from a more extensive list with similar properties. An important factor determining whether such novel CD8+ would act on the tumor is, however, that natural presentation of the same T cell exposed motif should occur with sufficient frequency to allow the newly stimulated CD8+ to engage and act on the tumor peptides in vivo. The preferred combinations of alleles and T cell exposed motifs are thus those which are in the top 50% of natural binding affinity when competing with all peptides in the protein and more preferably in the top 35%. In Table 1 we therefore show the predicted natural binding and the predicted binding of the newly generated synthetic peptides that comprise the same T cell exposed motif, indicating which are the most preferred combinations. For any individual patient carrying the EGFRvIII variant, a selection of which T cell exposed motif to target is made based on those most likely to be better exposed when bound by that patient's MHC alleles in vivo.

The maturation of CD8+ cells is enhanced by the presence of CD4+ helper T cells. In the case of EGFRvIII the very low binding affinity of DRB alleles carrying the novel tumor specific motifs makes it difficult to use natural peptides or even synthetic peptides designed to stimulate T cells that engage the tumor specific T cell exposed motifs. The EGFRvIII variant does, however, carry a sequence adjacent to the splice site, comprising 15 mer peptides with index positions of 97 to 105 and 127-140 that are predicted to be high binders for a multiplicity of MHC II alleles. These peptides would be naturally presented by such MHC II alleles if present and can thus provide CD4+ help to the desired CD8. Specifically, in Table 2 we show synthetic peptides which would bind certain indicated MHC II alleles and which can provide CD4+ help to the above referenced synthetic CD8+ targeting peptides. While one or more 15mer peptide may be selected as a MHC II binding peptide for a subject of a known allele, a longer synthetic peptide comprising two or more sequential 15mers from those shown in Table 2 can also be administered as a longer peptide of from about 16 to about 22 amino acids as indicated in the bottom two lines, which are provided as non-limiting examples. When co-administered with peptides designed to stimulate CD8+ responses to EGFRvIII such MHC II binding peptides are selected to enhance the response.

TABLE 1
Bespoke peptides designed for illustrative MHC I alleles to target
the tumor specific motifs in EGFRvIII

| | | | | | | | | Originating | synthetic |
|---|---|---|---|---|---|---|---|---|---|
| | originating | SEQ ID | proposed | SEQ ID | TCEM | SEQ ID | | natural | peptide |
| pos | peptide | NO.: | peptide | NO.: | core | NO.: | Allele | peptide affinity | affinity |
| 23 | RALEEKKGN | 1 | ESLEEKKGK | 11 | EEKKG | 6 | A6801 | −0.92 | −2.02 |
| 23 | RALEEKKGN | 1 | EAAEEKKGK | 12 | EEKKG | 6 | A6801 | −0.92 | −2.02 |
| 23 | RALEEKKGN | 1 | DTQEEKKGR | 13 | EEKKG | 6 | A6801 | −0.92 | −2.02 |
| 23 | RALEEKKGN | 1 | KTKEEKKGL | 14 | EEKKG | 6 | B0702 | −1.23 | −2.02 |
| 23 | RALEEKKGN | 1 | RIREEKKGY | 15 | EEKKG | 6 | B0702 | −1.23 | −2.02 |
| 23 | RALEEKKGN | 1 | HWFEEKKGR | 16 | EEKKG | 6 | B2705 | −0.25 | −2.01 |
| 23 | RALEEKKGN | 1 | RDFEEKKGE | 17 | EEKKG | 6 | B2705 | −0.25 | −2.01 |
| 23 | RALEEKKGN | 1 | VAEEEKKGL | 18 | EEKKG | 6 | B3501 | −0.53 | −2.01 |
| 23 | RALEEKKGN | 1 | RAVEEKKGI | 19 | EEKKG | 6 | B3501 | −0.53 | −2.01 |
| 23 | RALEEKKGN | 1 | KSFEEKKGL | 20 | EEKKG | 6 | B5701 | −0.05 | −2.06 |
| 23 | RALEEKKGN | 1 | RAGEEKKGY | 21 | EEKKG | 6 | B5701 | −0.05 | −2.02 |
| 24 | ALEEKKGNY | 2 | TLSEKKGNI | 22 | EKKGN | 7 | A0201 | −0.41 | −2.01 |
| 24 | ALEEKKGNY | 2 | RLTEKKGNL | 23 | EKKGN | 7 | A0201 | −0.41 | −2.01 |
| 24 | ALEEKKGNY | 2 | QTKEKKGNL | 24 | EKKGN | 7 | B0801 | −0.75 | −2.01 |
| 24 | ALEEKKGNY | 2 | KFREKKGNA | 25 | EKKGN | 7 | B0801 | −0.75 | −2.01 |
| 24 | ALEEKKGNY | 2 | GPREKKGNA | 26 | EKKGN | 7 | B0801 | −0.75 | −2.01 |
| 24 | ALEEKKGNY | 2 | QEMEKKGNY | 27 | EKKGN | 7 | B1501 | −1.92 | −2.02 |
| 24 | ALEEKKGNY | 2 | YEQEKKGNF | 28 | EKKGN | 7 | B1501 | −1.92 | −2.05 |
| 24 | ALEEKKGNY | 2 | LDGEKKGNM | 29 | EKKGN | 7 | B1501 | −1.92 | −2.05 |
| 24 | ALEEKKGNY | 2 | LASEKKGNY | 30 | EKKGN | 7 | B3501 | −0.96 | −2.02 |
| 24 | ALEEKKGNY | 2 | LLYEKKGNW | 31 | EKKGN | 7 | B3501 | −0.96 | −2.02 |
| 24 | ALEEKKGNY | 2 | KANEKKGNF | 32 | EKKGN | 7 | B5701 | −0.27 | −2.08 |
| 24 | ALEEKKGNY | 2 | SSPEKKGNF | 33 | EKKGN | 7 | B5701 | −0.27 | −2.07 |
| 25 | LEEKKGNYV | 3 | GWGKKGNYA | 34 | KKGNY | 8 | A0202 | −0.90 | −2.01 |
| 25 | LEEKKGNYV | 3 | KWQKKGNYL | 35 | KKGNY | 8 | A0202 | −0.90 | −2.01 |
| 25 | LEEKKGNYV | 3 | ELAKKGNYW | 36 | KKGNY | 8 | A2402 | −0.37 | −2.01 |
| 25 | LEEKKGNYV | 3 | SKFKKGNYF | 37 | KKGNY | 8 | A2402 | −0.37 | −2.01 |
| 25 | LEEKKGNYV | 3 | LTLKKGNYR | 38 | KKGNY | 8 | A2601 | 0.41 | −2.01 |
| 25 | LEEKKGNYV | 3 | TPYKKGNYY | 39 | KKGNY | 8 | A2601 | 0.41 | −2.01 |
| 25 | LEEKKGNYV | 3 | VEAKKGNYT | 40 | KKGNY | 8 | B4402 | −1.39 | −2.02 |
| 25 | LEEKKGNYV | 3 | ADRKKGNYF | 41 | KKGNY | 8 | B4402 | −1.39 | −2.01 |
| 25 | LEEKKGNYV | 3 | KSFKKGNYV | 42 | KKGNY | 8 | B5101 | −0.56 | −2.02 |
| 25 | LEEKKGNYV | 3 | VPTKKGNYA | 43 | KKGNY | 8 | B5101 | −0.56 | −2.01 |
| 26 | EEKKGNYVV | 4 | LRHKGNYVW | 44 | KGNYV | 9 | A2402 | −0.46 | −2.01 |
| 26 | EEKKGNYVV | 4 | LPPKGNYVL | 45 | KGNYV | 9 | A2402 | −0.46 | −2.01 |
| 26 | EEKKGNYVV | 4 | WLRKGNYVV | 46 | KGNYV | 9 | B0801 | −0.48 | −2.11 |
| 26 | EEKKGNYVV | 4 | FLRKGNYVL | 47 | KGNYV | 9 | B0801 | −0.48 | −2.03 |
| 26 | EEKKGNYVV | 4 | GETKGNYVK | 48 | KGNYV | 9 | B4402 | −0.99 | −2.02 |
| 26 | EEKKGNYVV | 4 | TEIKGNYVE | 49 | KGNYV | 9 | B4402 | −0.99 | −2.02 |
| 27 | EKKGNYVVT | 5 | RPGGNYVVF | 50 | GNYVV | 10 | A2403 | −0.25 | −2.02 |
| 27 | EKKGNYVVT | 5 | EREGNYVVL | 51 | GNYVV | 10 | A2403 | −0.25 | −2.01 |
| 27 | EKKGNYVVT | 5 | RPRGNYVVA | 52 | GNYVV | 10 | B0801 | −0.40 | −2.02 |
| 27 | EKKGNYVVT | 5 | CACGNYVVI | 53 | GNYVV | 10 | B0801 | −0.40 | −2.01 |
| 27 | EKKGNYVVT | 5 | ARDGNYVVS | 54 | GNYVV | 10 | B2705 | −0.34 | −2.06 |
| 27 | EKKGNYVVT | 5 | QKAGNYVVS | 55 | GNYVV | 10 | B2705 | −0.34 | −2.06 |
| 27 | EKKGNYVVT | 5 | YPAGNYVVA | 56 | GNYVV | 10 | B5101 | −0.15 | −2.02 |
| 27 | EKKGNYVVT | 5 | LSRGNYVVV | 50 | GNYVV | 10 | B5101 | −0.15 | −2.01 |

TABLE 2
Synthetic peptides from EGFRvIII with binding affinity to MHC II alleles to
provide CD4+help-showing illustrative DRB alleles

| index | | SEQ | nDRB1_ | nDRB1_ | nDRB1_ | nDRB1_ | nDRB1_ | nDRB1_ |
|---|---|---|---|---|---|---|---|---|
| position | peptide | ID NO.: | 0101 | 0301 | 0401 | 0701 | 1101 | 1501 |
| 97 | SISGDLHILPVAFRG | 51 | −1.50 | 0.56 | 0.68 | −1.60 | 0.25 | −1.61 |
| 98 | ISGDLHILPVAFRGD | 52 | −0.89 | 0.12 | −0.78 | −0.96 | −0.89 | −1.49 |
| 99 | SGDLHILPVAFRGDS | 53 | −1.56 | 0.47 | −0.73 | −0.34 | −1.13 | 1.39 |
| 100 | GDLHILPVAFRGDSF | 54 | −1.73 | −0.17 | −2.34 | −1.38 | −1.21 | −0.82 |
| 101 | DLHILPVAFRGDSFT | 55 | −2.25 | −0.57 | −2.98 | −1.36 | −1.74 | −1.59 |
| 102 | LHILPVAFRGDSFTH | 56 | −1.51 | −1.21 | −1.58 | −1.53 | −1.53 | −0.89 |
| 103 | HILPVAFRGDSFTHT | 57 | −1.20 | −0.91 | −0.39 | −1.06 | −1.46 | −0.86 |
| 104 | ILPVAFRGDSFTHTP | 58 | −0.80 | −1.19 | −1.21 | −0.84 | −0.53 | −0.71 |
| 105 | LPVAFRGDSFTHTPP | 59 | −0.82 | −1.42 | −1.04 | 0.22 | −0.60 | −0.29 |
| 127 | ILKTVKEITGFLLIQ | 60 | −0.89 | 0.00 | −0.60 | −2.19 | 0.10 | −1.60 |
| 128 | LKTVKEITGFLLIQA | 61 | −1.33 | −0.84 | −0.32 | −2.04 | 0.41 | −0.96 |
| 129 | KTVKEITGFLLIQAW | 62 | −0.34 | 0.25 | −1.20 | −1.48 | 0.09 | −0.89 |
| 130 | TVKEITGFLLIQAWP | 63 | −1.24 | 0.79 | −0.46 | −1.03 | 0.12 | −1.45 |
| 131 | VKEITGFLLIQAWPE | 64 | −1.87 | −0.07 | −0.57 | −1.28 | −1.29 | −1.64 |
| 132 | KEITGFLLIQAWPEN | 65 | −0.99 | −0.46 | −1.10 | −0.87 | −1.16 | −3.12 |
| 133 | EITGFLLIQAWPENR | 66 | −1.35 | −1.66 | −1.87 | −1.49 | −2.10 | −2.91 |
| 134 | ITGFLLIQAWPENRT | 67 | −2.18 | −1.33 | −1.46 | −0.83 | −2.66 | −2.73 |
| 135 | TGFLLIQAWPENRTD | 68 | −1.27 | −2.40 | −1.44 | −1.41 | −3.72 | −2.85 |
| 136 | GFLLIQAWPENRTDL | 69 | −1.56 | −1.18 | −1.88 | −0.69 | −3.29 | −1.46 |
| 137 | FLLIQAWPENRTDLH | 70 | −0.88 | −1.83 | −2.34 | −1.69 | −2.81 | −1.48 |
| 138 | LLIQAWPENRTDLHA | 71 | −0.36 | −1.17 | −0.47 | −0.12 | −1.32 | −0.74 |
| 139 | LIQAWPENRTDLHAF | 72 | −0.18 | −1.80 | −0.92 | −0.86 | −0.76 | −0.32 |
| 140 | IQAWPENRTDLHAFE | 73 | −0.02 | −1.67 | 0.00 | −0.07 | −0.92 | −0.04 |
| 101 | DLHILPVAFRGDSFTHT | 74 | | | | | | |
| 132 | KEITGFLLIQAWPENRTDLH | 75 | | | | | | |

### Example 5: Design of Peptides to Target Common EGFR Mutants

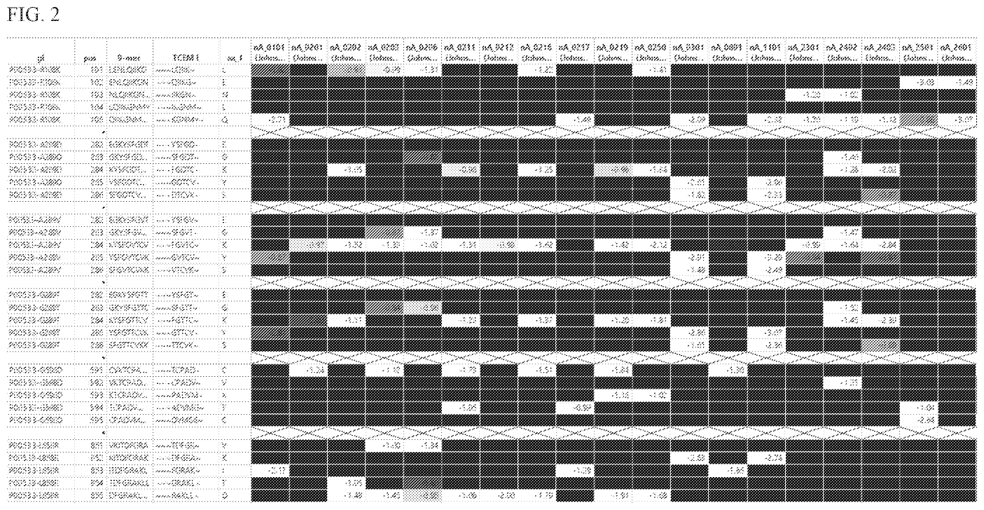

While the EGFR viii mutation is typically seen only in glioblastoma and related brain tumors, other cancers exhibit aberrations of EGFR and several common mutations are described, while other stochastic mutations may also arise. In glioblastoma about 25% cases have mutations in the extracellular domain of EGFR including A289V/D/T, R108K, and G598D. Conversely in lung cancer the most common mutation of EGFR is L858R [61, 62]. Hence the need frequently arises to address these common mutations. Each of these mutations creates a novel amino acid motif which allows tumor specific targeting of T cells. Relatively few of the MHC I A alleles have moderate or high binding in positions which expose the mutant motifs, and some have excessively high binding which may result in anergy or exhaustion (e.g. A1101) (FIG. 2). Where natural binding falls in a stimulatory range, immunization with the neoepitope in its natural form may result in an effective cytotoxic response. In Table 3 we show examples of how modification of the flanking sequences can be used to generate a peptide with the same T cell exposed motif but also an appropriate binding affinity to generate a cytotoxic response. This is shown for a subset of MHC I A alleles but such examples should not be considered limiting, as the same approach can be applied to other A alleles or to MHC IB or IC alleles.

Table 3 shows peptides which have T cell exposed motifs spanning the common EGFR mutations and in which natural binding affinity is appropriate; it will be noted that relatively few alleles have high affinity natural binding to the mutant T cell exposed motifs, reflecting one reason tumor motifs may achieve immune evasion.

Table 3 shows bespoke peptides as examples of peptides designed for exemplar alleles to provide binding that will allow them to be presented to these alleles where natural binding is insufficient to competitively stimulate a new T cell clone. As these examples were selected from a large array for illustrative purposes, and other peptides with differing binding affinity or for other alleles could have been selected, these examples are considered non limiting.

TABLE 3
Bespoke peptides to provide CD8+ targeting of common
tumor specific mutant EGFR T cell exposed motifs

| | | | | | | | | Binding | | | | |
|---|---|---|---|---|---|---|---|---|---|---|---|---|
| | | | | | | | | affinity of | | | Binding | |
| | | | | | | | | proposed | | | affinity of | |
| | | origi- | SEQ | | SEQ | | SEQ | peptide | | | originating | |
| | | nating | ID | proposed | ID | | ID | A_0202 | A_2601 | po- | peptide | |
| Mutant | pos | peptide | NO.: | peptide | NO.: | TCEM I | NO.: | mean | mean | larity | A 0202 | A 2601 |
| R108K | 101 | LENLQIIKG | 76 | TTQLQIIKM | 120 | ~~~LQIIK~ | 98 | | −2.02 | 0.73 | −0.91 | |
| R108K | 101 | LENLQIIKG | 76 | DTTLQIIKL | 121 | ~~~LQIIK~ | 98 | | −2.01 | 0.77 | −0.91 | |
| R108K | 101 | LENLQIIKG | 76 | LRALQIIKA | 122 | ~~~LQIIK~ | 98 | −2.01 | | 1.22 | −0.91 | −2.01 |
| R108K | 101 | LENLQIIKG | 76 | WKSLQIIKV | 123 | ~~~LQIIK~ | 98 | −2.01 | | 1.15 | −0.91 | −2.01 |
| R108K | 103 | NLQIIKGNM | 77 | ELRIIKGNY | 124 | ~~~IIKGN~ | 99 | | −2.02 | −0.15 | −0.55 | |
| R108K | 103 | NLQIIKGNM | 77 | ELAIIKGNW | 125 | ~~~IIKGN~ | 99 | | −2.01 | 0.98 | −0.55 | |
| R108K | 103 | NLQIIKGNM | 77 | NWSIIKGNL | 126 | ~~~IIKGN~ | 99 | −2.03 | | 0.75 | −0.55 | −2.03 |
| R108K | 103 | NLQIIKGNM | 77 | LWDIIKGNG | 127 | ~~~IIKGN~ | 99 | −2.01 | | 0.77 | −0.55 | −2.01 |
| R108K | 104 | LQIIKGNMY | 78 | DPLIKGNMY | 128 | ~~~IKGNM~ | 100 | | −2.03 | 0.23 | −0.13 | |
| R108K | 104 | LQIIKGNMY | 78 | ELGIKGNMW | 129 | ~~~IKGNM~ | 100 | | −2.01 | 0.53 | −0.13 | |
| R108K | 104 | LQIIKGNMY | 78 | QWEIKGNML | 130 | ~~~IKGNM~ | 100 | −2.02 | | 0.18 | −0.13 | −2.02 |
| A289D | 282 | EGKYSFGDT | 79 | KVTYSFGDY | 130 | ~~~YSFGD~ | 101 | | −2.02 | −0.05 | | −0.44 |
| A289D | 282 | EGKYSFGDT | 79 | YTYYSFGDI | 131 | ~~~YSFGD~ | 101 | | −2.02 | 1.19 | | −0.44 |
| A289D | 283 | GKYSFGDTC | 80 | YKYSFGDTI | 132 | ~~~SFGDT~ | 102 | −2.03 | | 0.16 | −0.52 | |
| A289D | 283 | GKYSFGDTC | 80 | EYCSFGDTI | 133 | ~~~SFGDT~ | 102 | −2.03 | | 0.22 | −0.52 | |
| A289D | 284 | KYSFGDTCV | 81 | YYVFGDTCI | 134 | ~~~FGDTC~ | 103 | −1.88 | | 1.96 | −1.05 | |
| A289D | 284 | KYSFGDTCV | 81 | YYTFGDTCV | 135 | ~~~FGDTC~ | 103 | −1.84 | | 1.07 | −1.05 | |
| A289D | 285 | YSFGDTCVK | 82 | YSVGDTCVY | 136 | ~~~GDTCV~ | 104 | | −2.02 | 0.66 | | −0.37 |
| A289D | 285 | YSFGDTCVK | 82 | KVVGDTCVY | 137 | ~~~GDTCV~ | 104 | | −2.01 | 0.46 | | −0.37 |
| A289D | 286 | SFGDTCVKK | 83 | YVCDTCVKV | 138 | ~~~DTCVK~ | 105 | −2.02 | | 0.78 | −0.31 | |
| A289D | 286 | SFGDTCVKK | 83 | IYTDTCVKS | 139 | ~~~DTCVK~ | 105 | −2.01 | | −0.23 | −0.31 | |
| G289T | 282 | EGKYSFGTT | 84 | EGVYSFGTE | 140 | ~~~YSFGT~ | 106 | | −2.02 | −0.27 | | −0.61 |
| G289T | 282 | EGKYSFGTT | 84 | DVVYSFGTT | 141 | ~~~YSFGT~ | 106 | | −2.01 | 0.78 | | −0.61 |
| G289T | 283 | GKYSFGTTC | 85 | EYDSFGTTV | 142 | ~~~SFGTT~ | 107 | −2.00 | | −0.38 | −0.51 | |
| G289T | 283 | GKYSFGTTC | 85 | IIGSFGTTS | 143 | ~~~SFGTT~ | 107 | −2.00 | | 1.20 | −0.51 | |
| G289T | 284 | KYSFGTTCV | 86 | YYVFGTTCV | 144 | ~~~FGTTC~ | 108 | −2.08 | | 2.27 | −1.11 | |
| G289T | 284 | KYSFGTTCV | 86 | YYIFGTTCI | 145 | ~~~FGTTC~ | 108 | −2.00 | | 2.68 | −1.11 | |
| G289T | 285 | YSFGTTCVK | 87 | EVVGTTCVV | 146 | ~~~GTTCV~ | 109 | | −2.05 | 1.33 | | −0.37 |
| G289T | 285 | YSFGTTCVK | 87 | VTGGTTCVY | 147 | ~~~GTTCV~ | 109 | | −2.01 | 0.94 | | −0.37 |
| G598D | 591 | CVKTCPADV | 88 | ETCTCPADT | 148 | ~~~TCPAD~ | 110 | | −2.01 | −1.23 | | −0.71 |
| G598D | 591 | CVKTCPADV | 88 | TTETCPADY | 149 | ~~~TCPAD~ | 110 | | −2.01 | −1.18 | | −0.71 |
| G598D | 591 | CVKTCPADV | 88 | KYKTCPADV | 150 | ~~~TCPAD~ | 110 | −2.01 | | −1.18 | −0.45 | |
| G598D | 591 | CVKTCPADV | 88 | GYDTCPADV | 151 | ~~~TCPAD~ | 110 | −2.01 | | −0.46 | −0.45 | |
| G598D | 595 | CPADVMGEN | 89 | EGVDVMGEK | 152 | ~~~DVMGE~ | 111 | | −2.00 | −1.32 | | −0.64 |
| G598D | 595 | CPADVMGEN | 89 | DKIDVMGEY | 153 | ~~~DVMGE~ | 111 | | −2.00 | −0.78 | | −0.64 |
| G598V | 591 | CVKTCPAVV | 90 | TSGTCPAVY | 154 | ~~~TCPAV~ | 112 | | −2.03 | 0.43 | | −0.86 |
| G598V | 591 | CVKTCPAVV | 90 | DSSTCPAVY | 155 | ~~~TCPAV~ | 112 | | −2.03 | −0.29 | | −0.86 |
| G598V | 591 | CVKTCPAVV | 90 | KYITCPAVG | 156 | ~~~TCPAV~ | 112 | −2.01 | | 0.88 | −0.69 | |
| G598V | 591 | CVKTCPAVV | 90 | EYSTCPAVV | 157 | ~~~TCPAV~ | 112 | −2.01 | | 0.58 | −0.69 | |
| G598V | 592 | VKTCPAVVM | 91 | DCCCPAVVY | 158 | ~~~CPAVV~ | 113 | | −2.02 | 1.46 | | −0.34 |
| G598V | 592 | VKTCPAVVM | 91 | VVKCPAVVY | 159 | ~~~CPAVV~ | 113 | | −2.01 | 1.98 | | −0.34 |
| G598V | 592 | VKTCPAVVM | 91 | TIKCPAVVV | 160 | ~~~CPAVV~ | 113 | −2.06 | | 1.75 | −0.38 | |
| G598V | 592 | VKTCPAVVM | 91 | GIECPAVVV | 161 | ~~~CPAVV~ | 113 | −2.03 | | 1.93 | −0.38 | |
| G598V | 593 | KTCPAVVMG | 92 | KYKPAVVMI | 162 | ~~~PAVVM~ | 114 | −2.03 | | 1.14 | −0.44 | |
| G598V | 593 | KTCPAVVMG | 92 | KYKPAVVMV | 163 | ~~~PAVVM~ | 114 | −2.02 | | 0.94 | −0.44 | |
| G598V | 595 | CPAVVMGEN | 93 | EVGVVMGEK | 164 | ~~~VVMGE~ | 115 | | −2.03 | −0.12 | | −0.41 |
| G598V | 595 | CPAVVMGEN | 93 | EDVVVMGEY | 165 | ~~~VVMGE~ | 115 | | −2.02 | 0.32 | | −0.41 |
| L858R | 851 | VKITDFGRA | 94 | EVSTDFGRK | 166 | ~~~TDFGR~ | 116 | | −2.03 | −1.95 | | −0.34 |
| L858R | 851 | VKITDFGRA | 94 | GVETDFGRY | 167 | ~~~TDFGR~ | 116 | | −2.00 | −0.71 | | −0.34 |
| L858R | 851 | VKITDFGRA | 94 | TYGTDFGRG | 168 | ~~~TDFGR~ | 116 | −2.01 | | −0.86 | −0.68 | |
| L858R | 851 | VKITDFGRA | 94 | IIKTDFGRI | 169 | ~~~TDFGR~ | 116 | −2.01 | | 0.66 | −0.68 | |
| L858R | 853 | ITDFGRAKL | 95 | DTIFGRAKI | 170 | ~~~FGRAK~ | 117 | | −2.02 | 0 | | −0.05 |
| L858R | 853 | ITDFGRAKL | 95 | EVSFGRAKI | 171 | ~~~FGRAK~ | 117 | | −2.01 | −0.31 | | −0.05 |
| L858R | 853 | ITDFGRAKL | 95 | YYTFGRAKI | 172 | ~~~FGRAK~ | 117 | −1.98 | | 0.50 | −0.64 | |
| L858R | 853 | ITDFGRAKL | 95 | YYVFGRAKI | 173 | ~~~FGRAK~ | 117 | −1.94 | | 1.19 | −0.64 | |
| L858R | 854 | TDFGRAKLL | 96 | SIGGRAKLV | 174 | ~~~GRAKL~ | 118 | −2.01 | | 0.18 | −1.05 | |
| L858R | 854 | TDFGRAKLL | 96 | EYYGRAKLI | 175 | ~~~GRAKL~ | 118 | −2.01 | | −0.04 | −1.05 | |
| L858R | 855 | DFGRAKLLG | 97 | KYTRAKLLI | 176 | ~~~RAKLL~ | 119 | −2.01 | | 0.23 | −1.48 | |
| L858R | 855 | DFGRAKLLG | 97 | GYGRAKLLV | 177 | ~~~RAKLL~ | 119 | −2.01 | | 0.75 | −1.48 | |

### Example 6: Histone 3.3 Variants

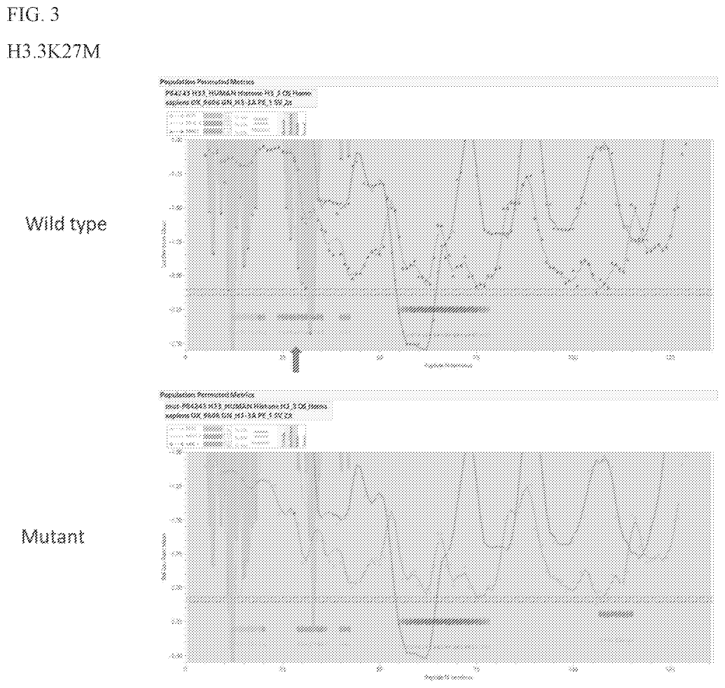

A particularly common mutation found in glioma and glioblastoma is the missense mutation in Histone 3.3 (P84243, H33) that replaces a lysine at position 28 with a methionine (although commonly referred to in the literature as the K27M variant). The resulting sequence thus comprises the peptide ATKAARMSA (SEQ ID NO.: 178) instead of ATKAARKSA (SEQ ID NO.: 179). As shown in FIG. 3, this mutation lies in a region with overall poor predicted MHC I and MHC II binding affinity and also coincides with a predicted B cell epitope. The binding to MHC I and MHC II is little changed between wild type and mutant. The poor MHC binding is a possible explanation for the escape of this mutant from immune surveillance.

While peptide approaches have been proposed, these have been restricted to subjects carrying HLA A0201 and one particular peptide RMSAPSTGGV (SEQ ID NO.: 180) [63, 64] (see also U.S. Pat. No. 10,441,644). While this peptide has a moderately high predicted binding affinity to A0201, the location of the mutant methionine at position 2 means that this amino acid is preferentially hidden in a pocket position of the MHC groove (groove exposed position) and thus unlikely to stimulate a T cell response that will effectively differentiate tumor and normal tissue. In the present invention, by using modifications of the groove exposed motif of peptides in which the mutant methionine is exposed in the T cell exposed motif, we identify other peptides which are capable of directing a CD8+ response to the tumor and for other HLA alleles.

Possible approaches were examined to direct a T cell response to the tumor specific T cell exposed motifs. For MHC I the peptides KAARM, AARMS, ARMSA, RMSAP, and MSAPS (SEQ ID NOS: 181-185) are the T cell exposed motifs which distinguish the mutant from the normal wildtype and thus can stimulate a CD8+ tumor specific response. From MHC II the corresponding exposed tumor specific motifs are ATxAxRM, TKxAxMS, AAxMxAP, RMxAxST, MSxPxTG (SEQ ID NOS: 186-190) where x indicates an amino acid hidden in the groove exposed or pocket position.

Synthetic peptides were designed by maintaining the T cell exposed motifs constant and substituting other amino acids in the groove exposed or pocket positions to achieve a desired binding to a particular allele of interest. Not all alleles would naturally bind and present to T cells the peptides that expose the T cell exposed motifs that are tumor specific (ie those comprising the mutant methionine in an exposed position). Creating such peptides of desired affinity proved more feasible for 9 mers to engage MHC I alleles than for 15 mers to engage MHC II alleles. Furthermore, because not all peptides bearing these T cell exposed motifs are competitive with other peptides in the protein in their binding to the MHC molecules. For those which carry the T cell exposed motif and which are in the top 50% of naturally competitive binders, synthetic peptides were designed which have a higher binding affinity, with the intent of stimulating T cells that then bind to the naturally presented peptides that share the same T cell exposed motifs. These are shown in Table 4A.

Where natural binding to MHC II alleles is found to be insufficient, CD4+ help can be provided by providing synthetic copies of naturally occurring MHC II binding peptides which lie in proximity to, but not actually overlapping the mutant amino acid position. Several sequential 15 mer peptides with appropriate MHC II binding affinity are shown in Table 4B and the optimal peptide may be selected from among these based on binding to the individual's alleles. Alternatively, an extended peptide comprising more than one sequential 15mer of those shown in Table 4B may be selected for many different MHC II alleles and administered as a synthetic peptide of from about 16 to 22 amino acids long. The MHC II binding peptides, whether 15 amino acids or longer, are co-administered with the synthetic MHC I binding peptides of Table 4A.

TABLE 4A
Peptides designed to provide tumor specific
targeting of CD8+ to mutant H3.3 for illustrative alleles

| | | | | | | | | Natural | Synthetic |
|---|---|---|---|---|---|---|---|---|---|
| | | SEQ | | | | SEQ | | peptide | peptide |
| | Originating | ID | proposed | | TCEM | ID | | binding | binding |
| pos | peptide | NO.: | peptide | | core | NO.: | Allele | affinity | affinity |
| | | | | SEQ ID | | | | | |
| | | | | NO.: of | | | | | |
| | | | | proposed | | | | | |
| | | | | peptides | | | | | |
| 21 | LATKAARMS | 191 | KATKAARMY | 201 | KAARM | 181 | A0101 | −0.13 | −2.03 |
| 21 | LATKAARMS | 191 | FAQKAARMR | 202 | KAARM | 181 | A0101 | −0.13 | −2.02 |
| 21 | LATKAARMS | 191 | TQRKAARMY | 203 | KAARM | 181 | B5701 | −0.20 | −2.01 |
| 21 | LATKAARMS | 191 | LKKKAARMY | 204 | KAARM | 181 | B5701 | −0.20 | −2.01 |
| 21 | LATKAARMS | 191 | MRRKAARMG | 205 | KAARM | 181 | C0401 | −1.29 | −2.04 |
| 21 | LATKAARMS | 191 | RRRKAARMS | 206 | KAARM | 181 | C0401 | −1.29 | −1.99 |
| 22 | ATKAARMSA | 192 | PVEAARMSR | 207 | AARMS | 182 | A0101 | −0.17 | −2.03 |
| 22 | ATKAARMSA | 192 | RPDAARMSR | 208 | AARMS | 182 | A0101 | −0.17 | −2.02 |
| 22 | ATKAARMSA | 192 | KAKAARMSL | 209 | AARMS | 182 | B0702 | −1.01 | −2.01 |
| 22 | ATKAARMSA | 192 | RGKAARMSA | 210 | AARMS | 182 | B0702 | −1.01 | −2.01 |
| 22 | ATKAARMSA | 192 | EQEAARMSY | 211 | AARMS | 182 | B1501 | −0.14 | −2.01 |
| 22 | ATKAARMSA | 192 | GTRAARMSY | 212 | AARMS | 182 | B1501 | −0.14 | −2.00 |
| 23 | TKAARMSAP | 193 | ARLARMSAK | 213 | ARMSA | 183 | C0702 | −1.04 | −2.02 |
| 23 | TKAARMSAP | 193 | HRRARMSAA | 214 | ARMSA | 183 | C0702 | −1.04 | −2.01 |
| 23 | TKAARMSAP | 193 | SSEARMSAR | 215 | ARMSA | 183 | A2601 | −0.30 | −2.05 |
| 23 | TKAARMSAP | 193 | EPDARMSAQ | 216 | ARMSA | 183 | A2601 | −0.30 | −2.04 |
| 23 | TKAARMSAP | 193 | KSRARMSAL | 217 | ARMSA | 183 | B0702 | −0.11 | −2.02 |
| 23 | TKAARMSAP | 193 | RTRARMSAV | 218 | ARMSA | 183 | B0702 | −0.11 | −2.01 |
| 23 | TKAARMSAP | 193 | QRDARMSAL | 219 | ARMSA | 183 | B0801 | −0.54 | −2.01 |
| 23 | TKAARMSAP | 193 | KPGARMSAA | 220 | ARMSA | 183 | B0801 | −0.54 | −2.01 |
| 23 | TKAARMSAP | 193 | SRRARMSAA | 221 | ARMSA | 183 | B2705 | −1.52 | −2.01 |
| 23 | TKAARMSAP | 193 | ARAARMSAD | 222 | ARMSA | 183 | B2705 | −1.52 | −2.01 |
| 23 | TKAARMSAP | 193 | RARARMSAK | 223 | ARMSA | 183 | C0401 | −0.99 | −2.02 |
| 23 | TKAARMSAP | 193 | RAHARMSAS | 224 | ARMSA | 183 | C0401 | −0.99 | −2.01 |
| 24 | KAARMSAPS | 194 | RLDRMSAPQ | 225 | RMSAP | 184 | A0101 | −0.15 | −2.03 |
| 24 | KAARMSAPS | 194 | FQERMSAPR | 226 | RMSAP | 184 | A0101 | −0.15 | −2.02 |
| 24 | KAARMSAPS | 194 | KLTRMSAPT | 227 | RMSAP | 184 | A0201 | −0.01 | −2.02 |
| 24 | KAARMSAPS | 194 | RQIRMSAPA | 228 | RMSAP | 184 | A0201 | −0.01 | −2.00 |
| 24 | KAARMSAPS | 194 | REMRMSAPR | 229 | RMSAP | 184 | A0301 | −0.56 | −2.01 |
| 24 | KAARMSAPS | 194 | EHRRMSAPK | 230 | RMSAP | 184 | A0301 | −0.56 | −2.01 |
| 24 | KAARMSAPS | 194 | NLIRMSAPR | 231 | RMSAP | 184 | A1101 | −0.18 | −2.02 |
| 24 | KAARMSAPS | 194 | RQYRMSAPK | 232 | RMSAP | 184 | A1101 | −0.18 | −2.01 |
| 24 | KAARMSAPS | 194 | DQKRMSAPI | 233 | RMSAP | 184 | A3201 | −0.12 | −2.02 |
| 24 | KAARMSAPS | 194 | RVKRMSAPL | 234 | RMSAP | 184 | A3201 | −0.12 | −2.01 |
| 24 | KAARMSAPS | 194 | RSSRMSAPR | 235 | RMSAP | 184 | A6801 | −0.03 | −2.01 |
| 24 | KAARMSAPS | 194 | EQTRMSAPR | 236 | RMSAP | 184 | A6801 | −0.03 | −2.01 |
| 24 | KAARMSAPS | 194 | RPRRMSAPD | 237 | RMSAP | 184 | B0702 | −0.55 | −2.02 |
| 24 | KAARMSAPS | 194 | KPSRMSAPG | 238 | RMSAP | 184 | B0702 | −0.55 | −2.01 |
| 24 | KAARMSAPS | 194 | QTRRMSAPA | 239 | RMSAP | 184 | B0801 | −0.07 | −2.02 |
| 24 | KAARMSAPS | 194 | RDRRMSAPI | 240 | RMSAP | 184 | B0801 | −0.07 | −2.02 |
| 24 | KAARMSAPS | 194 | DDARMSAPY | 241 | RMSAP | 184 | B1501 | −0.02 | −2.02 |
| 24 | KAARMSAPS | 194 | KERRMSAPF | 242 | RMSAP | 184 | B1501 | −0.02 | −2.02 |
| 24 | KAARMSAPS | 194 | RRYRMSAPY | 243 | RMSAP | 184 | C0401 | −0.22 | −2.01 |
| 24 | KAARMSAPS | 194 | TLRRMSAPK | 244 | RMSAP | 184 | C0401 | −0.22 | −2.01 |
| 25 | AARMSAPST | 195 | LEEMSAPSP | 245 | MSAPS | 185 | A0101 | −0.22 | −2.02 |
| 25 | AARMSAPST | 195 | KAAMSAPSY | 246 | MSAPS | 185 | A0101 | −0.22 | −2.01 |
| 25 | AARMSAPST | 195 | RETMSAPSK | 247 | MSAPS | 185 | A0301 | −0.50 | −2.02 |
| 25 | AARMSAPST | 195 | RTRMSAPSR | 248 | MSAPS | 185 | A0301 | −0.50 | −2.01 |
| 25 | AARMSAPST | 195 | DTGMSAPSR | 249 | MSAPS | 185 | A2601 | −0.18 | −2.04 |
| 25 | AARMSAPST | 195 | ESTMSAPSR | 250 | MSAPS | 185 | A2601 | −0.18 | −2.03 |
| 25 | AARMSAPST | 195 | RPSMSAPSS | 251 | MSAPS | 185 | B0702 | −1.65 | −2.02 |
| 25 | AARMSAPST | 195 | DPKMSAPSA | 252 | MSAPS | 185 | B0702 | −1.65 | −2.01 |
| 25 | AARMSAPST | 195 | KTKMSAPSM | 253 | MSAPS | 185 | B1501 | −0.04 | −2.03 |
| 25 | AARMSAPST | 195 | KAQMSAPSY | 254 | MSAPS | 185 | B1501 | −0.04 | −2.03 |
| 25 | AARMSAPST | 195 | ASVMSAPSF | 255 | MSAPS | 185 | B5701 | −0.15 | −2.09 |
| 25 | AARMSAPST | 195 | RAQMSAPSY | 256 | MSAPS | 185 | B5701 | −0.15 | −2.04 |
| 25 | AARMSAPST | 195 | LFKMSAPSP | 257 | MSAPS | 185 | C0401 | −0.17 | −2.14 |
| 25 | AARMSAPST | 195 | RRFMSAPSA | 258 | MSAPS | 185 | C0401 | −0.17 | −1.94 |

MHC II

| | | | | Column 6 | | | | | |
|---|---|---|---|---|---|---|---|---|---|
| 18 | RKQLATKAARMSAPS | 196 | IIMGATKAARMGIGL | 259 | AT~A~RM | 186 | DQA1_0201- | −0.74 | −1.53 |
| | | | | | | | DQB1_0301 | | |
| 18 | RKQLATKAARMSAPS | 196 | YILAATKAARMVAIV | 260 | AT~A~RM | 186 | DQA1_0201- | −0.74 | −1.48 |
| | | | | | | | DQB1_0301 | | |
| 18 | RKQLATKAARMSAPS | 196 | ARALATKAARMASPV | 261 | AT~A~RM | 186 | DQA1_0501- | −0.22 | −1.22 |
| | | | | | | | DQB1_0301 | | |
| 18 | RKQLATKAARMSAPS | 196 | SLALATKAARMSALM | 262 | AT~A~RM | 186 | DQA1_0501- | −0.22 | −1.17 |
| | | | | | | | DQB1_0301 | | |
| 18 | RKQLATKAARMSAPS | 196 | QLLIATKAARMSVDV | 263 | AT~A~RM | 186 | DRB1_0401 | −0.12 | −1.90 |
| 18 | RKQLATKAARMSAPS | 196 | TMIIATKAARMFARK | 264 | AT~A~RM | 186 | DRB1_0401 | −0.12 | −1.71 |
| 18 | RKQLATKAARMSAPS | 196 | DRLRATKAARMGEAA | 265 | AT~A~RM | 186 | DRB1_1602 | −0.56 | −2.02 |
| 18 | RKQLATKAARMSAPS | 196 | GRITATKAARMEMQI | 266 | AT~A~RM | 186 | DRB1_1602 | −0.56 | −2.08 |
| 19 | KQLATKAARMSAPST | 197 | KFLRTKAARMSTYDA | 267 | TK~A~MS | 187 | DRB1_0401 | −0.53 | −2.01 |
| 19 | KQLATKAARMSAPST | 197 | PKFRTKAARMSTFPR | 268 | TK~A~MS | 187 | DRB1_0401 | −0.53 | −2.03 |
| 21 | LATKAARMSAPSTGG | 198 | FSIAAARMSAPIAIM | 269 | AA~M~AP | 188 | DQA1_0201- | −1.37 | −1.94 |
| | | | | | | | DQB1_0301 | | |
| 21 | LATKAARMSAPSTGG | 198 | AALGAARMSAPGGAA | 270 | AA~M~AP | 188 | DQA1_0501- | −2.07 | −1.99 |
| | | | | | | | DQB1_0301 | | |
| 21 | LATKAARMSAPSTGG | 198 | GVAAAARMSAPAGGV | 271 | AA~M~AP | 188 | DQA1_0501- | −2.07 | −2.12 |
| | | | | | | | DQB1_0301 | | |
| 21 | LATKAARMSAPSTGG | 198 | AKKYAARMSAPTRKG | 272 | AA~M~AP | 188 | DRB1_1602 | −2.35 | −2.01 |
| 21 | LATKAARMSAPSTGG | 198 | KRATAARMSAPCATH | 273 | AA~M~AP | 188 | DRB1_1602 | −2.35 | −2.01 |
| 23 | TKAARMSAPSTGGVK | 199 | FSILRMSAPSTIGTP | 274 | RM~A~ST | 189 | DQA1_0201- | −0.72 | −1.17 |
| | | | | | | | DQB1_0301 | | |
| 23 | TKAARMSAPSTGGVK | 199 | LSYIRMSAPSTLAGL | 275 | RM~A~ST | 189 | DQA1_0201- | −0.72 | −1.16 |
| | | | | | | | DQB1_0301 | | |
| 23 | TKAARMSAPSTGGVK | 199 | SIAARMSAPSTAGSL | 276 | RM~A~ST | 189 | DQA1_0201- | −1.21 | −1.55 |
| | | | | | | | DQB1_0301 | | |
| 23 | TKAARMSAPSTGGVK | 199 | AITLRMSAPSTAGAL | 277 | RM~A~ST | 189 | DQA1_0501- | −1.21 | −1.55 |
| | | | | | | | DQB1_0301 | | |
| 23 | TKAARMSAPSTGGVK | 199 | RARLRMSAPSTIAQG | 278 | RM~A~ST | 189 | DRB1_0101 | −0.35 | −2.01 |
| 23 | TKAARMSAPSTGGVK | 199 | TRAFRMSAPSTLADQ | 279 | RM~A~ST | 189 | DRB1_0101 | −0.35 | −2.01 |
| 24 | KAARMSAPSTGGVKK | 200 | EAMPMSAPSTGVAVG | 280 | MS~P~TG | 190 | DQA1_0201- | −1.36 | −2.01 |
| | | | | | | | DQB1_0301 | | |
| 24 | KAARMSAPSTGGVKK | 200 | QGFLMSAPSTGGLAD | 281 | MS~P~TG | 190 | DQA1_0201- | −1.36 | −2.02 |
| | | | | | | | DQB1_0301 | | |
| 24 | KAARMSAPSTGGVKK | 200 | APAAMSAPSTGASSK | 282 | MS~P~TG | 190 | DQA1_0501- | −1.56 | −2.00 |
| | | | | | | | DQB1_0301 | | |
| 24 | KAARMSAPSTGGVKK | 200 | LPSFMSAPSTGTDSK | 283 | MS~P~TG | 190 | DQA1_0501- | −1.56 | −2.01 |
| | | | | | | | DQB1_0301 | | |
| 24 | KAARMSAPSTGGVKK | 200 | EAILMSAPSTGIASE | 284 | MS~P~TG | 190 | DRB1_0101 | −0.41 | −2.01 |
| 24 | KAARMSAPSTGGVKK | 200 | RPRIMSAPSTGLETA | 285 | MS~P~TG | 190 | DRB1_0101 | −0.41 | −2.01 |
| 24 | KAARMSAPSTGGVKK | 200 | AGRRMSAPSTGQGYC | 286 | MS~P~TG | 190 | DRB1_1602 | −0.98 | −2.02 |
| 24 | KAARMSAPSTGGVKK | 200 | KRMRMSAPSTGGDLI | 287 | MS~P~TG | 190 | DRB1_1602 | −0.98 | −2.04 |

TABLE 4B
Synthetic Peptides in H3.3 which
provide CD4+ help to tumor specific CD8+ T cells

| | index position | | | | |
|---|---|---|---|---|---|
| | 57 | 58 | 59 | 60 | 61 |
| | Peptide | | | | |
| | KSTELLIRKLPFQRL | STELLIRKLPFQRLV | TELLIRKLPFQRLVR | ELLIRKLPFQRLVRE | LLIRKLPFQRLVREI |
| | SEQ ID NO.: | | | | |
| | SEQ ID NO.: 288 | SEQ ID NO.: 289 | SEQ ID NO.: 290 | SEQ ID NO.: 291 | SEQ ID NO.: 292 |
| Allele | predicted binding affinity in standard deviation units | | | | |
| nDRB1_0101 | | −1.49 | | −1.74 | −1.32 |
| nDRB1_0301 | −1.64 | −2.46 | −2.20 | −1.30 | |
| nDRB1_0401 | | −1.12 | | −1.92 | −1.22 |
| nDRB1_0404 | −2.46 | −1.34 | −1.21 | −1.45 | −2.71 |
| nDRB1_0405 | | −1.89 | −2.04 | −1.99 | −2.29 |
| nDRB1_0701 | −1.80 | −1.69 | −1.02 | −1.77 | −2.77 |
| nDRB1_0801 | −1.50 | −2.50 | −1.88 | −1.58 | −2.78 |
| nDRB1_0802 | | | −1.75 | −2.22 | |
| nDRB1_0901 | | −1.31 | | | −1.94 |
| nDRB1_1001 | −1.40 | | | | −1.12 |
| nDRB1_1101 | −2.64 | −2.18 | −1.52 | −1.87 | −2.69 |
| nDRB1_1201 | −1.23 | −1.65 | −1.21 | −1.42 | −1.44 |
| nDRB1_1301 | −1.39 | −1.84 | −1.29 | −2.31 | −2.90 |
| nDRB1_1302 | −1.63 | | | −1.76 | −1.13 |
| nDRB1_1454 | −2.15 | −2.41 | −2.21 | −1.75 | −2.54 |
| nDRB1_1501 | −1.84 | −2.16 | −1.98 | −2.93 | −1.66 |
| nDRB1_1602 | | −1.62 | | | |
| nDRB3_0101 | | −2.98 | −1.55 | −2.05 | −1.37 |
| nDRB3_0202 | | −1.25 | −2.25 | −2.32 | |
| nDRB3_0301 | −1.68 | −1.95 | | −2.15 | |
| nDRB4_0101 | −1.56 | −1.49 | −2.83 | −2.11 | |
| nDRB4_0103 | −1.65 | −1.32 | −1.47 | −1.65 | −2.97 |
| nDRB5_0101 | −2.57 | −1.38 | −2.38 | −2.48 | −1.75 |

### Example 7: Bespoke Peptides Targeting Common Mutations in IDH1

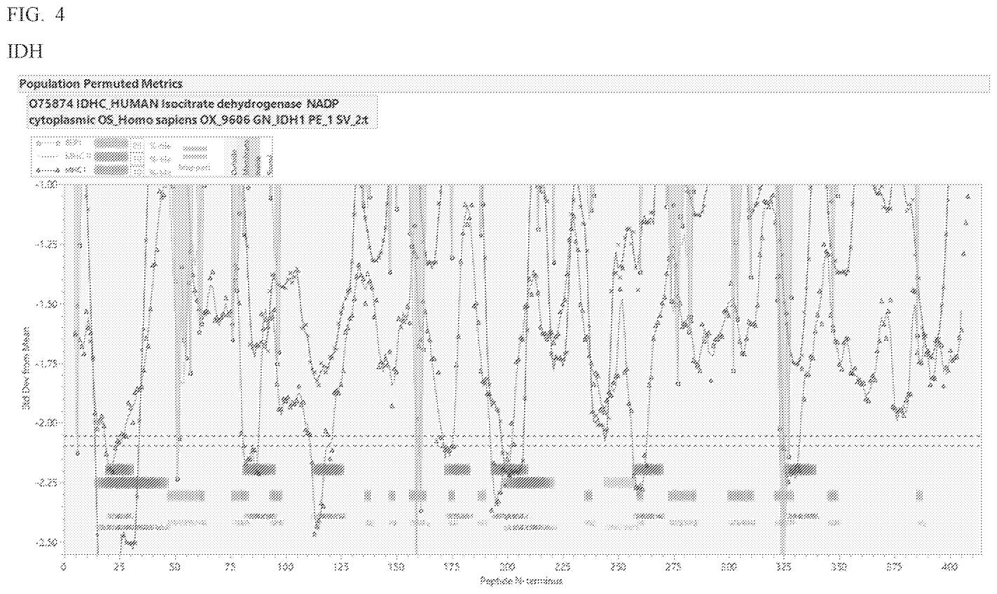

Isocitrate dehydrogenase IDH1, encoded by sequence 075874, is commonly mutated in gliomas and glioblastomas. FIG. 4 provides an overview of the immunome features. The most frequent mutation is R132H is associated with loss of conversion of isocitrate to alpha-ketoglutarate and increased risk of hypermethylation. leading to genetic instability which may trigger other mutations [65]. Mutations in isocitrate dehydrogenase enzyme isoform 1 (IDH1) and, to a lesser extent in isoform 2 (IDH2) genes have been identified in a large proportion of diffuse astrocytomas (70-90%), oligodendrogliomas (69-94%), oligoastrocytomas (78-100%), and secondary glioblastomas (82-88%). Peptides have been identified in the vicinity of the mutation in IDH which can stimulate CD4+ cells and produce a tumor suppressive effect [66, 67] (see also U.S. Ser. No. 10/161,940). In the present invention we identify tumor specific epitopes in IDH R132H that can target an array of CD8+ cells in subjects of differing HLA alleles and also provide examples of personalized peptides with altered groove exposed motifs to optimize binding for particular HLAs.

The R132H mutation produces novel tumor specific class I T cell exposed motifs ˜˜˜IIIGH˜(SEQ ID NO.: 294), ˜˜˜IIGHH˜(SEQ ID NO.: 295), ˜˜˜IGHHA˜(SEQ ID NO.: 296), ˜˜˜GHHAY˜(SEQ ID NO.: 297), ˜˜˜HHAYG˜(SEQ ID NO.: 298). For a few MHC I alleles, namely A0201, A0202, A0203, A0206, A0211, A0212, A0216 and A0250 the 9-mer peptide IIIGHHAYG (SEQ ID NO.: 293) encompassing ˜˜˜GHHAY˜(SEQ ID NO.: 297) provides a predicted binding affinity of a suitable range to stimulate T cells, approximating to 100-200 nmolar. For these alleles the natural peptide may provide a suitable immunogen. In this case the natural 15mer VKPIIIGHHAYGDQY (SEQ ID NO.: 341) may be administered to provide CD4+ help, albeit at a more moderate binding affinity.

For other alleles and to increase the array of alleles the generation of designed flanking regions to the above cited T cell exposed motifs is desirable. Tables 5 and 6 provide examples of such bespoke peptides for an illustrative set of alleles. These examples are considered non limiting as the same approach can be applied for other alleles and multiple peptide options exist for each allele.

TABLE 5
Exemplars of peptides designed to provide high binding
affinity for various MHC I alleles to the tumor specific T cell
exposed motifs of IDH R132H

| | | SEQ | | SEQ | | SEQ | | Proposed | original | |
|---|---|---|---|---|---|---|---|---|---|---|
| | originating | ID | proposed | ID | TCEM | ID | | peptide | peptide | |
| pos | peptide | NO.: | peptide | NO.: | core | NO.: | Allele | affinity | affinity | polarity |
| 125 | VKPIIIGHH | 299 | HGDIIIGHK | 304 | IIIGH | 294 | A0101 | −2.02 | −0.27 | 0.07 |
| 125 | VKPIIIGHH | 299 | KADIIIGHK | 305 | IIIGH | 294 | A0101 | −2.01 | −0.27 | −0.23 |
| 125 | VKPIIIGHH | 299 | EWKIIIGHI | 306 | IIIGH | 294 | A2402 | −2.04 | −0.69 | 1.84 |
| 125 | VKPIIIGHH | 299 | KYTIIIGHL | 307 | IIIGH | 294 | A2402 | −2.02 | −0.69 | 1.97 |
| 125 | VKPIIIGHH | 299 | NVEIIIGHR | 308 | IIIGH | 294 | A6801 | −2.01 | −0.60 | 0.56 |
| 125 | VKPIIIGHH | 299 | YNAIIIGHK | 309 | IIIGH | 294 | A6801 | −2.01 | −0.60 | 0.99 |
| 125 | VKPIIIGHH | 299 | ARPIIIGHN | 310 | IIIGH | 294 | B2705 | −2.03 | −0.48 | 0.74 |
| 125 | VKPIIIGHH | 299 | KKMIIIGHA | 311 | IIIGH | 294 | B2705 | −2.02 | −0.48 | 0.90 |
| 126 | KPIIIGHHA | 300 | QSDIIGHHR | 312 | IIGHH | 295 | A0101 | −2.02 | −0.15 | −1.28 |
| 126 | KPIIIGHHA | 300 | NGDIIGHHS | 313 | IIGHH | 295 | A0101 | −2.01 | −0.15 | −0.71 |
| 126 | KPIIIGHHA | 300 | NGQHIGHHI | 314 | IIGHH | 295 | B5701 | −2.01 | −0.11 | 0.56 |
| 126 | KPIIIGHHA | 300 | RGNIIGHHR | 315 | IIGHH | 295 | B5701 | −2.00 | −0.11 | −1.14 |
| 127 | PIIIGHHAY | 301 | YSDIGHHAK | 316 | IGHHA | 296 | A0101 | −2.04 | −1.20 | −1.25 |
| 127 | PIIIGHHAY | 301 | KGDIGHHAL | 317 | IGHHA | 296 | A0101 | −2.03 | −1.20 | −0.60 |
| 127 | PIIIGHHAY | 301 | EYLIGHHAF | 318 | IGHHA | 296 | A2402 | −2.08 | −0.68 | 1.35 |
| 127 | PIIIGHHAY | 301 | DWRIGHHAF | 319 | IGHHA | 296 | A2402 | −2.01 | −0.68 | 0.23 |
| 127 | PIIIGHHAY | 301 | EVLIGHHAP | 320 | IGHHA | 296 | A2601 | −2.02 | −1.53 | 0.80 |
| 127 | PIIIGHHAY | 301 | QPFIGHHAY | 321 | IGHHA | 296 | A2601 | −2.02 | −1.53 | 0.81 |
| 127 | PIIIGHHAY | 301 | EINIGHHAK | 322 | IGHHA | 296 | A6801 | −2.01 | −0.70 | −0.95 |
| 127 | PIIIGHHAY | 301 | IADIGHHAK | 323 | IGHHA | 296 | A6801 | −2.01 | −0.70 | −0.43 |
| 127 | PIIIGHHAY | 301 | QRKIGHHAT | 324 | IGHHA | 296 | B2705 | −2.02 | −0.09 | −1.87 |
| 127 | PIIIGHHAY | 301 | SRIIGHHAS | 325 | IGHHA | 296 | B2705 | −2.02 | −0.09 | −0.38 |
| 127 | PIIIGHHAY | 301 | AELIGHHAN | 326 | IGHHA | 296 | B4402 | −2.01 | −0.29 | −0.14 |
| 127 | PIIIGHHAY | 301 | GEYIGHHAE | 327 | IGHHA | 296 | B4402 | −2.00 | −0.29 | −0.90 |
| 127 | PIIIGHHAY | 301 | QAKIGHHAY | 328 | IGHHA | 296 | B5701 | −2.05 | −1.10 | −0.65 |
| 127 | PIIIGHHAY | 301 | EQSIGHHAW | 329 | IGHHA | 296 | B5701 | −2.02 | −1.10 | −0.57 |
| 128 | IIIGHHAYG | 302 | LDDGHHAYA | 330 | GHHAY | 297 | A0101 | −2.03 | −0.25 | −0.82 |
| 128 | IIIGHHAYG | 302 | RADGHHAYA | 331 | GHHAY | 297 | A0101 | −2.01 | −0.25 | −1.50 |
| 128 | IIIGHHAYG | 302 | AVTGHHAYP | 332 | GHHAY | 297 | A2601 | −2.01 | −0.68 | 0.17 |
| 128 | IIIGHHAYG | 302 | NPGGHHAYF | 333 | GHHAY | 297 | A2601 | −2.01 | −0.68 | −0.06 |
| 128 | IIIGHHAYG | 302 | NIRGHHAYV | 334 | GHHAY | 297 | B0801 | −2.06 | −0.99 | −0.20 |
| 128 | IIIGHHAYG | 302 | VPRGHHAYL | 335 | GHHAY | 297 | B0801 | −2.05 | −0.99 | 0.28 |
| 129 | IIGHHAYGD | 303 | FTDHHAYGT | 336 | HHAYG | 298 | A0101 | −2.03 | −0.41 | −0.47 |
| 129 | IIGHHAYGD | 303 | DLQHHAYGY | 337 | HHAYG | 298 | A0101 | −2.03 | −0.41 | −0.38 |
| 129 | IIGHHAYGD | 303 | KRAHHAYGW | 338 | HHAYG | 298 | A2402 | −2.01 | −0.13 | −1.02 |
| 129 | IIGHHAYGD | 303 | RRTHHAYGW | 339 | HHAYG | 298 | A2402 | −2.01 | −0.13 | −1.21 |
| 129 | IIGHHAYGD | 303 | QGMHHAYGR | 340 | HHAYG | 298 | A6801 | −2.03 | −0.23 | −1.05 |
| 129 | IIGHHAYGD | 303 | DGIHHAYGR | 341 | HHAYG | 298 | A6801 | −2.03 | −0.23 | −1.00 |
| 129 | IIGHHAYGD | 303 | EEGHHAYGK | 342 | HHAYG | 298 | B4402 | −2.01 | −0.13 | −2.38 |
| 129 | IIGHHAYGD | 303 | FEIHHAYGT | 343 | HHAYG | 298 | B4402 | −2.01 | −0.13 | 0.46 |

NOTE:
Binding is shown in standard deviation units comparing all peptides within the protein. While actual affinity varies between proteins a value of -1 SD equates to 100-200 nanomolar

TABLE 6
Exemplars of peptides designed to provide high binding
affinity for various MHC II alleles to the tumor specific T cell
exposed motifs of IDH R132H

| | | SEQ | | SEQ | | SEQ | | Affinity | Afinity | |
|---|---|---|---|---|---|---|---|---|---|---|
| | originating | ID | proposed | ID | TCEM | ID | | proposed | original | po- |
| pos | peptide | NO.: | peptide | NO.: | core | NO.: | Allele | peptide | peptide | larity |
| 122 | SGWVKPIIIGHHAYG | 349 | PMKFKPIIIGHPKGN | 354 | KP~I~GH | 344 | DRB1_0401 | −2.01 | −0.68 | 0.18 |
| 122 | SGWVKPIIIGHHAYG | 349 | LLLRKPIIIGHAKRS | 355 | KP~I~GH | 344 | DRB1_0401 | −2.01 | −0.68 | 0.57 |
| 122 | SGWVKPIIIGHHAYG | 349 | IVWFKPIIIGHKRDA | 356 | KP~I~GH | 344 | DRB1_0801 | −2.01 | −0.83 | 1.06 |
| 122 | SGWVKPIIIGHHAYG | 349 | WVLWKPIIIGHKEQA | 357 | KP~I~GH | 344 | DRB1_0801 | −2.00 | −0.83 | 1.14 |
| 122 | SGWVKPIIIGHHAYG | 349 | YQPWKPIIIGHGEKH | 358 | KP~I~GH | 344 | DRB1_1101 | −2.02 | −1.31 | −0.10 |
| 122 | SGWVKPIIIGHHAYG | 349 | SMIPKPIIIGHNKWT | 359 | KP~I~GH | 344 | DRB1_1101 | −2.01 | −1.31 | 0.75 |
| 122 | SGWVKPIIIGHHAYG | 349 | NLTIKPIIIGHLTRN | 360 | KP~I~GH | 344 | DRB1_1501 | −2.02 | −1.31 | 0.79 |
| 122 | SGWVKPIIIGHHAYG | 349 | PFRIKPIIIGHRIQT | 361 | KP~I~GH | 344 | DRB1_1501 | −2.02 | −1.31 | 0.83 |
| 123 | GWVKPIIIGHHAYGD | 350 | TDLMPIIIGHHDKIH | 362 | PI~I~HH | 345 | DRB1_0401 | −2.00 | −1.49 | 0.58 |
| 123 | GWVKPIIIGHHAYGD | 350 | KGIYPIIIGHHHGQL | 363 | PI~I~HH | 345 | DRB1_0401 | −2.00 | −1.49 | 1.01 |
| 123 | GWVKPIIIGHHAYGD | 350 | LNIFPIIIGHHKNNK | 364 | PI~I~HH | 345 | DRB1_0801 | −2.01 | −0.61 | 0.54 |
| 123 | GWVKPIIIGHHAYGD | 350 | YIKQPIIIGHHAKTF | 365 | PI~I~HH | 345 | DRB1_0801 | −2.01 | −0.61 | 0.83 |
| 123 | GWVKPIIIGHHAYGD | 350 | ATFQPIIIGHHPKQP | 366 | PI~I~HH | 345 | DRB1_1101 | −2.01 | −1.14 | 0.39 |
| 123 | GWVKPIIIGHHAYGD | 350 | GWIEPIIIGHHQQNM | 367 | PI~I~HH | 345 | DRB1_1101 | −2.00 | −1.14 | 0.79 |
| 123 | GWVKPIIIGHHAYGD | 350 | YHILPIIIGHHSQNN | 368 | PI~I~HH | 345 | DRB1_1501 | −2.00 | −1.01 | 0.80 |
| 123 | GWVKPIIIGHHAYGD | 350 | GGIIPIIIGHHEDRS | 369 | PI~I~HH | 345 | DRB1_1501 | −2.00 | −1.01 | 0.34 |
| 125 | VKPIIIGHHAYGDQY | 351 | PKVIIIGHHAYAKGK | 370 | II~H~AY | 346 | DRB1_0401 | −2.02 | −1.35 | 0.14 |
| 125 | VKPIIIGHHAYGDQY | 351 | QYQIIIGHHAYEQGA | 371 | II~H~AY | 346 | DRB1_0401 | −2.01 | −1.35 | 0.15 |
| 125 | VKPIIIGHHAYGDQY | 351 | GPLIIIGHHAYKEQF | 372 | II~H~AY | 346 | DRB1_0801 | −2.01 | −1.59 | 0.93 |
| 125 | VKPIIIGHHAYGDQY | 351 | HFLMIIGHHAYEDNA | 373 | II~H~AY | 346 | DRB1_0801 | −2.01 | −1.59 | 0.72 |
| 125 | VKPIIIGHHAYGDQY | 351 | NIWAIIGHHAYWDEG | 374 | II~H~AY | 346 | DRB1_1101 | −2.01 | −1.01 | 0.84 |
| 125 | VKPIIIGHHAYGDQY | 351 | KNYMIIGHHAYYEKS | 375 | II~H~AY | 346 | DRB1_1101 | −2.00 | −1.01 | −0.40 |
| 125 | VKPIIIGHHAYGDQY | 351 | KQTRIIGHHAYILLQ | 376 | II~H~AY | 346 | DRB1_1501 | −2.02 | −0.87 | 0.55 |
| 125 | VKPIIIGHHAYGDQY | 351 | KPDIIIGHHAYKDFE | 377 | II~H~AY | 346 | DRB1_1501 | −2.01 | −0.87 | −0.44 |
| 127 | PIIIGHHAYGDQYRA | 352 | PWIFGHHAYGDKSGL | 378 | GH~A~GD | 347 | DRB1_0401 | −2.14 | −1.45 | 0.44 |
| 127 | PIIIGHHAYGDQYRA | 352 | FLILGHHAYGDNKSM | 379 | GH~A~GD | 347 | DRB1_0401 | −2.13 | −1.45 | 0.54 |
| 127 | PIIIGHHAYGDQYRA | 352 | LMFWGHHAYGDEIYF | 380 | GH~A~GD | 347 | DRB1_0801 | −1.80 | −0.83 | 1.66 |
| 127 | PIIIGHHAYGDQYRA | 352 | IFFWGHHAYGDIKKD | 381 | GH~A~GD | 347 | DRB1_0801 | −1.48 | −0.83 | 0.31 |
| 127 | PIIIGHHAYGDQYRA | 352 | AKFWGHHAYGDLSRP | 382 | GH~A~GD | 347 | DRB1_1101 | −2.05 | −1.00 | −0.32 |
| 127 | PIIIGHHAYGDQYRA | 352 | KFIFGHHAYGDYDKK | 383 | GH~A~GD | 347 | DRB1_1101 | −2.04 | −1.00 | −0.79 |
| 127 | PIIIGHHAYGDQYRA | 352 | TAYVGHHAYGDWYYI | 384 | GH~A~GD | 347 | DRB1_1501 | −2.01 | −0.76 | 0.99 |
| 127 | PIIIGHHAYGDQYRA | 352 | ELIFGHHAYGDWFLI | 385 | GH~A~GD | 347 | DRB1_1501 | −1.87 | −0.76 | 2.10 |
| 128 | IIIGHHAYGDQYRAT | 353 | KRVFHHAYGDQAITL | 386 | HH~Y~DQ | 348 | DRB1_0401 | −2.01 | −0.52 | −0.07 |
| 128 | IIIGHHAYGDQYRAT | 353 | GRLFHHAYGDQLLDL | 387 | HH~Y~DQ | 348 | DRB1_0401 | −2.01 | −0.52 | 0.52 |
| 128 | IIIGHHAYGDQYRAT | 353 | MFFYHHAYGDQIKDN | 388 | HH~Y~DQ | 348 | DRB1_0801 | −2.02 | −0.20 | −0.13 |
| 128 | IIIGHHAYGDQYRAT | 353 | LYYFHHAYGDQQATD | 389 | HH~Y~DQ | 348 | DRB1_0801 | −2.02 | −0.20 | −0.29 |
| 128 | IIIGHHAYGDQYRAT | 353 | TVLRHHAYGDQWYKS | 390 | HH~Y~DQ | 348 | DRB1_1101 | −2.02 | −0.42 | −0.67 |
| 128 | IIIGHHAYGDQYRAT | 353 | IKMLHHAYGDQLDDT | 391 | HH~Y~DQ | 348 | DRB1_1101 | −2.01 | −0.42 | −0.59 |

NOTE:
Binding is shown in standard deviation units comparing all peptides within the protein. While actual affinity varies between proteins a value of -1 SD equates to 100-200 nanomolar

### Example 8: Bespoke Peptides Targeting Common Mutations in BRAF

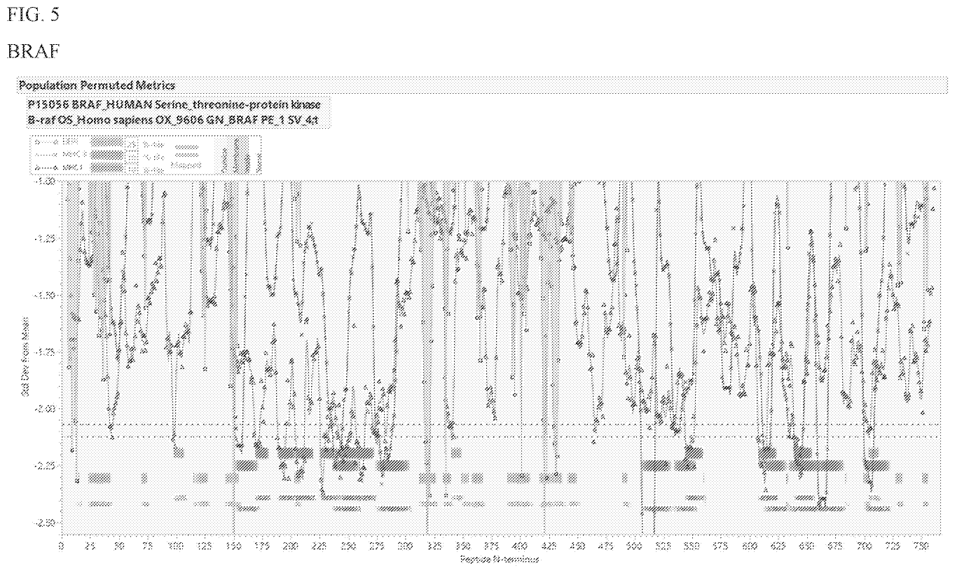

BRAF (Serine/threonine-protein kinase B-raf) exemplified by sequence P15056 is one of the most commonly mutated proteins in cancer. FIG. 5 provides an overview of the immunome features. BRAF is thought to function in regulating the MAP kinase/ERKs signaling pathway, which affects cell division, differentiation, and secretion. BRAF mutations have been associated with various cancers, including non-Hodgkin lymphoma, colorectal cancer, malignant melanoma, thyroid carcinoma, non-small cell lung carcinoma, and adenocarcinoma of the lung. There are several particularly common mutations in BRAF but the most common is V600E. Mutation V600M, G and K are less common.

Natural binding of MHC I A alleles to the T cell expose motif which would be tumor specific in the V600E mutation is very sparse, with no alleles having an optimum binding and very even moderate binding. Furthermore the adjacent peptides on the C terminal side of the mutation which would place the mutant E in a pocket position, or “out of frame” have an extremely high binding affinity for both A and B alleles, which would tend to favor binding preferentially in that position, hiding the mutant amino acid. The same is true for some MHC I A and B alleles for the V600M mutant. Thus the desirable approach is to create peptides with the mutant amino acid in the T cell exposed position and with modified amino acids in the groove exposed positions binding to stimulate T cell clones that can target the tumor specifically. Table 7 provides examples for exemplar selected alleles, using the method described in Examples 1 and 2. As many different peptides can be designed with flanking regions that produce the desired binding affinity and solubility, these examples shown are provided as illustrative but non-limiting examples. Table 8 includes examples of MHC II DRB binding peptides which can provide CD4. It is noted that the naturally occurring peptide GSHQFEQLSGSILWM (SEQ ID NO.: 464) provides a suitable predicted binding affinity for DRB1_0701 and could be used in this form. In addition naturally occurring adjacent peptides have suitable natural binding affinity and could provide CD4+ help, albeit not embodying the tumor specific amino acid motif. This short sequence of peptides comprises SGSILWMAPEVIRMQ (SEQ ID NO.: 465), GSILWMAPEVIRMQD (SEQ ID NO.: 466) and SILWMAPEVIRMQDK (SEQ ID NO.: 467).

TABLE 7
Exemplars of peptides designed to provide high binding
affinity for various MHC I alleles to the tumor specific T cell
exposed motifs of BRAF V600E

| | | | | | | | | | Predicted | Predicted | |
|---|---|---|---|---|---|---|---|---|---|---|---|
| | | | SEQ | | SEQ | | SEQ | | affinity | affinity | |
| | | Originating | ID | proposed | ID | TCEM | ID | | proposed | originating | po- |
| gi | pos | peptide | NO: | peptide | NO.: | core | NO.: | Allele | peptide | peptide | larity |
| P15056-600E | 604 | WSGSHQFEQ | 392 | KLVSHQFEY | 402 | SHQFE | 397 | A1101 | −2.02 | −0.39 | −0.04 |
| P15056-600E | 604 | WSGSHQFEQ | 392 | SIDSHQFER | 403 | SHQFE | 397 | A1101 | −2.02 | −0.39 | −1.78 |
| P15056-600E | 604 | WSGSHQFEQ | 392 | AAASHQFEF | 404 | SHQFE | 397 | B1501 | −2.11 | −0.17 | 0.18 |
| P15056-600E | 604 | WSGSHQFEQ | 392 | SSKSHQFEF | 405 | SHOFE | 397 | B1501 | −2.00 | −0.17 | −1.39 |
| P15056-600E | 604 | WSGSHQFEQ | 392 | GAISHQFER | 406 | SHQFE | 397 | B5701 | −2.02 | −0.54 | −0.82 |
| P15056-600E | 604 | WSGSHQFEQ | 392 | HSPSHQFEL | 407 | SHQFE | 397 | B5701 | −2.01 | −0.54 | −0.70 |
| P15056-600E | 605 | SGSHQFEQL | 393 | KLYHQFEQS | 408 | HQFEQ | 398 | A0201 | −2.03 | −0.13 | −1.02 |
| P15056-600E | 605 | SGSHQFEQL | 393 | AMDHQFEQA | 409 | HQFEQ | 398 | A0201 | −2.02 | −0.13 | −1.05 |
| P15056-600E | 605 | SGSHQFEQL | 393 | FPQHQFEQP | 410 | HQFEQ | 398 | B0702 | −1.98 | −0.48 | −0.65 |
| P15056-600E | 605 | SGSHQFEQL | 393 | IPLHQFEQY | 411 | HQFEQ | 398 | B0702 | −2.04 | −0.48 | 0.75 |
| P15056-600E | 605 | SGSHQFEQL | 393 | SSTHQFEQF | 412 | HQFEQ | 398 | B1501 | −2.05 | −0.45 | −0.95 |
| P15056-600E | 605 | SGSHQFEQL | 393 | KVPHQFEQY | 413 | HQFEQ | 398 | B1501 | −2.00 | −0.45 | −0.93 |
| P15056-600E | 605 | SGSHQFEQL | 393 | LSSHQFEQF | 414 | HQFEQ | 398 | A2601 | −2.01 | −0.83 | −0.06 |
| P15056-600E | 605 | SGSHQFEQL | 393 | DSFHQFEQL | 415 | HQFEQ | 398 | A2601 | −2.01 | −0.83 | −0.43 |
| P15056-600E | 605 | SGSHQFEQL | 393 | PIHHQFEQC | 416 | HQFEQ | 398 | B0801 | −2.07 | −1.43 | −0.31 |
| P15056-600E | 605 | SGSHQFEQL | 393 | ELAHQFEQG | 417 | HQFEQ | 398 | B0801 | −2.04 | −1.43 | −0.91 |
| P15056-600E | 605 | SGSHQFEQL | 393 | LALHQFEQM | 418 | HQFEQ | 398 | B5701 | −2.03 | −0.96 | −1.00 |
| P15056-600E | 605 | SGSHQFEQL | 393 | GEEHQFEQW | 419 | HQFEQ | 398 | B5701 | −2.03 | −0.96 | −1.72 |
| P15056-600E | 606 | GSHQFEQLS | 394 | SAKQFEQLR | 420 | QFEQL | 399 | A1101 | −2.03 | −0.32 | −1.64 |
| P15056-600E | 606 | GSHQFEQLS | 394 | GEAQFEQLK | 421 | QFEQL | 399 | A1101 | −2.02 | −0.32 | −1.36 |
| P15056-600E | 606 | GSHQFEQLS | 394 | EVKQFEQLK | 422 | QFEQL | 399 | A3001 | −2.02 | −1.00 | −1.57 |
| P15056-600E | 606 | GSHQFEQLS | 394 | RVDQFEQLG | 423 | QFEQL | 399 | A3001 | −2.02 | −1.00 | −0.91 |
| P15056-600E | 607 | SHQFEQLSG | 395 | DFKFEQLSA | 424 | FEQLS | 400 | A3001 | −2.02 | −0.31 | −0.37 |
| P15056-600E | 607 | SHQFEQLSG | 395 | GGRFEQLSN | 425 | FEQLS | 400 | A3001 | −2.02 | −0.31 | −1.19 |
| P15056-600E | 607 | SHQFEQLSG | 395 | YKLFEQLSI | 426 | FEQLS | 400 | B0801 | −2.02 | −0.69 | 1.18 |
| P15056-600E | 607 | SHQFEQLSG | 395 | YLGFEQLSA | 427 | FEQLS | 400 | B0801 | −2.04 | −0.69 | 1.18 |
| P15056-600E | 608 | HQFEQLSGS | 396 | IMHEQLSGS | 428 | EQLSG | 401 | A0201 | −2.02 | −0.48 | −0.10 |
| P15056-600E | 608 | HQFEQLSGS | 396 | KIEEQLSGL | 429 | EQLSG | 401 | A0201 | −2.02 | −0.48 | −0.62 |
| P15056-600E | 608 | HQFEQLSGS | 396 | DEAEQLSGF | 430 | EQLSG | 401 | B1501 | −2.04 | −0.77 | −1.15 |
| P15056-600E | 608 | HQFEQLSGS | 396 | FYLEQLSGF | 431 | EQLSG | 401 | B1501 | −2.04 | −0.77 | 1.89 |
| P15056-600E | 608 | HQFEQLSGS | 396 | TTKEQLSGS | 432 | EQLSG | 401 | A3001 | −2.02 | −0.53 | −2.07 |
| P15056-600E | 608 | HQFEQLSGS | 396 | RSQEQLSGA | 433 | EQLSG | 401 | A3001 | −2.01 | −0.53 | −2.08 |
| P15056-600E | 608 | HQFEQLSGS | 396 | EAVEQLSGS | 434 | EQLSG | 401 | A2601 | −2.01 | −0.84 | −1.04 |
| P15056-600E | 608 | HQFEQLSGS | 396 | NQREQLSGY | 435 | EQLSG | 401 | A2601 | −2.01 | −0.84 | −2.06 |
| P15056-600E | 608 | HQFEQLSGS | 396 | SLGEQLSGA | 436 | EQLSG | 401 | B0801 | −2.08 | −0.97 | −0.29 |
| P15056-600E | 608 | HQFEQLSGS | 396 | MNVEQLSGL | 437 | EQLSG | 401 | B0801 | −2.09 | −0.97 | 0.52 |

NOTE:
Binding is shown in standard deviation units comparing all peptides within the protein. While actual affinity varies between proteins a value of -1 SD equates to 100-200 nanomolar

TABLE 8
Exemplars of peptides designed to provide high binding
affinity for various MHC II alleles to the tumor specific T cell
exposed motifs of BRAF V600E

| | | | | | | | | | Pre- | Predicted | |
|---|---|---|---|---|---|---|---|---|---|---|---|
| | | | | | | | | | dicted | affinity | |
| | | | SEQ | | SEQ | | SEQ | | affinity | origi- | |
| | | originating | ID | proposed | ID | TCEM | ID | | proposed | nating | po- |
| gi | pos | peptide | NO.: | peptide | NO.: | core | NO.: | Allele | peptide | peptide | larity |
| P15056-600E | 604 | WSGSHQFEQLSGSIL | 438 | IHKFHQFEQLSPPSQ | 444 | HQ~E~LS | 441 | DRB1_0401 | −2.07 | −1.02 | −0.47 |
| P15056-600E | 604 | WSGSHQFEQLSGSIL | 438 | PHFIHQFEQLSGFAM | 445 | HQ~E~LS | 441 | DRB1_0401 | −2.04 | −1.02 | 1.17 |
| P15056-600E | 604 | WSGSHQFEQLSGSIL | 438 | YRYYHQFEQLSMPQV | 446 | HQ~E~LS | 441 | DRB1_0701 | −2.07 | −0.41 | 0.09 |
| P15056-600E | 604 | WSGSHQFEQLSGSIL | 438 | HMMFHQFEQLSKLQL | 447 | HQ~E~LS | 441 | DRB1_0701 | −2.01 | −0.41 | 0.69 |
| P15056-600E | 604 | WSGSHQFEQLSGSIL | 438 | WLPYHQFEQLSSPIP | 448 | HQ~E~LS | 441 | DRB1_1501 | −2.05 | −0.14 | 0.91 |
| P15056-600E | 604 | WSGSHQFEQLSGSIL | 438 | FFECHQFEQLSWLSV | 449 | HQ~E~LS | 441 | DRB1_1501 | −2.07 | −0.14 | 1.34 |
| P15056-600E | 606 | GSHQFEQLSGSILWM | 439 | SSWPFEQLSGSTGDN | 450 | FE~L~GS | 442 | DRB1_0401 | −2.08 | −0.90 | −0.94 |
| P15056-600E | 606 | GSHQFEQLSGSILWM | 439 | GPGMFEQLSGSTELF | 451 | FE~L~GS | 442 | DRB1_0401 | −2.07 | −0.90 | 0.49 |
| P15056-600E | 606 | GSHQFEQLSGSILWM | 439 | LEVFFEQLSGSSLAN | 452 | FE~L~GS | 442 | DRB1_0701 | −2.14 | −2.11 | 0.75 |
| P15056-600E | 606 | GSHQFEQLSGSILWM | 439 | RRILFEQLSGSCVGL | 453 | FE~L~GS | 442 | DRB1_0701 | −2.08 | −2.11 | 0.76 |
| P15056-600E | 606 | GSHQFEQLSGSILWM | 439 | WYDIFEQLSGSNAPS | 454 | FE~L~GS | 442 | DRB1-1101 | −2.11 | −0.44 | 0.04 |
| P15056-600E | 606 | GSHQFEQLSGSILWM | 439 | LVFIFEQLSGSNWRA | 455 | FE~L~GS | 442 | DRB1-1101 | −2.09 | −0.44 | 1.27 |
| P15056-600E | 606 | GSHQFEQLSGSILWM | 439 | RGAYFEQLSGSVMFS | 456 | FE~L~GS | 442 | DRB1_1501 | −2.00 | −1.65 | 0.52 |
| P15056-600E | 606 | GSHQFEQLSGSILWM | 439 | FYFYFEQLSGSLDSG | 457 | FE~L~GS | 442 | DRB1_1501 | −2.06 | −1.65 | 0.98 |
| P15056-600E | 607 | SHQFEQLSGSILWMA | 440 | PPLFEQLSGSIPICM | 458 | EQ~S~SI | 443 | DRB1_0401 | −2.01 | −1.19 | 1.61 |
| P15056-600E | 607 | SHQFEQLSGSILWMA | 440 | YPRFEQLSGSIKIIG | 459 | EQ~S~SI | 443 | DRB1_0401 | −2.01 | −1.19 | 0.45 |
| P15056-600E | 607 | SHQFEQLSGSILWMA | 440 | PTGQEQLSGSIIVIF | 460 | EQ~S~SI | 443 | DRB1_0701 | −2.11 | −1.83 | 1.11 |
| P15056-600E | 607 | SHQFEQLSGSILWMA | 440 | PKVLEQLSGSIFVGF | 461 | EQ~S~SI | 443 | DRB1_0701 | −1.99 | −1.83 | 1.37 |
| P15056-600E | 607 | SHQFEQLSGSILWMA | 440 | IFHFEQLSGSIFILI | 462 | EQ~S~SI | 443 | DRB1_1501 | −2.02 | −0.40 | 2.87 |
| P15056-600E | 607 | SHQFEQLSGSILWMA | 440 | RFRFEQLSGSILSLT | 463 | EQ~S~SI | 443 | DRB1_1501 | −2.07 | −0.40 | 0.56 |

NOTE:
Binding is shown in standard deviation units comparing all peptides within the protein. While actual affinity varies between proteins a value of -1 SD equates to 100-200 nanomolar

### Example 9: Bespoke Peptides Targeting Common Mutations in TP53

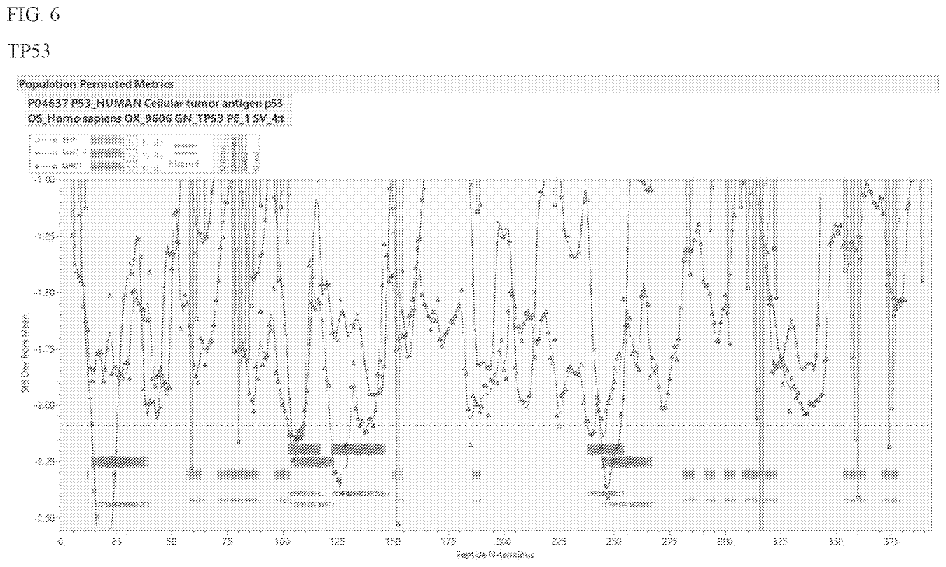

TP53 is the most commonly mutated protein in cancers [68, 69]. Such mutations are present in over half of all cancers [70]. TP 53 is a tumor suppressor whose function is to respond to stress and to induces numerous cellular responses including cell cycle arrest to restore genetic integrity, or apoptosis. Most mutations of TP53 occur in the central DNA binding domain of the protein between positions 102 and 292, disrupting its function and allowing genetic instability and greater risk of tumor progression by removing its proapoptotic function. While there are many unique stochastic mutations in TP53, there are also several which are most commonly recognized, these are: R175H, R273C, R248Q, R273H, R248W, R282W. While many other TP53 mutations are also found in individual tumors, the frequency of the above common mutations means that peptides can be pre-designed and prepared “ready to go” for these mutations for a variety of common alleles. TP53 is characterized by the sequence P04637 at Uniprot, although multiple other isoforms are recognized. FIG. 6 provides an overview of the immunome features Tables 9 and 10 below shows bespoke peptides designed and selected for a set of example alleles.

TABLE 9
Bespoke peptides designed for common mutations of TP53 for MHC I alleles

| | | | | | | | | | Predicted | Predicted |
|---|---|---|---|---|---|---|---|---|---|---|
| | | origi- | SEQ | | SEQ | | SEQ | | affinity | affinity |
| TP53 | | nating | ID | proposed | ID | TCEM | ID | | originating | proposed |
| mutant | pos | peptide | NO.: | peptide | NO.: | core | NO.: | Allele | peptide | peptide |
| P04637-R175H | 168 | HMTEVVRHC | 496 | PLQEVVRHA | 520 | EVVRH | 468 | A0201 | −1.79 | −1.98 |
| P04637-R175H | 168 | HMTEVVRHC | 496 | GQTEVVRHL | 521 | VVRHC | 468 | B1501 | −0.96 | −0.84 |
| P04637-R175H | 169 | MTEVVRHCP | 497 | WVRVVRHCI | 522 | VVRHC | 469 | A0201 | −0.04 | −2.10 |
| P04637-R175H | 169 | MTEVVRHCP | 497 | TAVVVRHCP | 523 | VVRHC | 469 | A1101 | −0.50 | −2.09 |
| P04637-R175H | 169 | MTEVVRHCP | 497 | PVKVVRHCA | 524 | VVRHC | 469 | B5701 | −0.59 | −0.25 |
| P04637-R175H | 170 | TEVVRHCPH | 498 | GLPVRHCPR | 525 | VRHCP | 470 | A1101 | −0.37 | −1.99 |
| P04637-R175H | 170 | TEVVRHCPH | 498 | VVGVRHCPM | 526 | VRHCP | 470 | A2601 | −0.55 | −2.06 |
| P04637-R175H | 170 | TEVVRHCPH | 498 | LFGVRHCPG | 527 | VRHCP | 470 | B0801 | −0.18 | −2.01 |
| P04637-R175H | 170 | TEVVRHCPH | 498 | NLWVRHCPP | 528 | VRHCP | 470 | B1501 | −0.63 | −0.89 |
| P04637-R175H | 170 | TEVVRHCPH | 498 | AGPVRHCPH | 529 | VRHCP | 470 | B5701 | −0.18 | −1.44 |
| P04637-R175H | 171 | EVVRHCPHH | 499 | KLERHCPHG | 530 | RHCPH | 471 | A0201 | −0.08 | −2.03 |
| P04637-R175H | 171 | EVVRHCPHH | 499 | LSIRHCPHM | 531 | RHCPH | 471 | A2601 | −3.69 | −2.09 |
| P04637-R175H | 171 | EVVRHCPHH | 499 | LPKRHCPHF | 532 | RHCPH | 471 | B0801 | −0.46 | −2.03 |
| P04637-R175H | 171 | EVVRHCPHH | 499 | MRVRHCPHL | 533 | RHCPH | 471 | B1501 | −0.39 | −0.27 |
| P04637-R175H | 171 | EVVRHCPHH | 499 | PSERHCPHY | 534 | RHCPH | 471 | B5701 | −0.53 | −1.95 |
| P04637-R248Q | 241 | SCMGGMNQR | 500 | NAWGGMNQP | 535 | GGMNQ | 472 | A1101 | −1.18 | −2.00 |
| P04637-R248Q | 241 | SCMGGMNQR | 500 | EPVGGMNQN | 536 | GGMNQ | 472 | A2601 | −0.48 | −2.03 |
| P04637-R248Q | 241 | SCMGGMNQR | 500 | VMVGGMNQH | 537 | GGMNQ | 472 | B1501 | −0.88 | −1.55 |
| P04637-R248Q | 241 | SCMGGMNQR | 500 | SRAGGMNQF | 538 | GGMNQ | 472 | B5701 | −1.03 | −0.27 |
| P04637-R248Q | 242 | CMGGMNQRP | 501 | YNIGMNQRL | 539 | GMNQR | 473 | A0201 | −0.85 | −2.06 |
| P04637-R248Q | 242 | CMGGMNQRP | 501 | VAVGMNQRP | 540 | GMNQR | 473 | A1101 | −1.14 | −2.05 |
| P04637-R248Q | 242 | CMGGMNQRP | 501 | LVAGMNQRL | 541 | GMNQR | 473 | A2601 | −0.31 | −2.03 |
| P04637-R248Q | 242 | CMGGMNQRP | 501 | KMEGMNQRN | 542 | GMNQR | 473 | B1501 | −0.44 | −0.35 |
| P04637-R248Q | 243 | MGGMNQRPI | 502 | RLPMNQRPL | 543 | MNQRP | 474 | A0201 | −0.47 | −2.06 |
| P04637-R248Q | 243 | MGGMNQRPI | 502 | ELLMNQRPW | 544 | MNQRP | 474 | A2601 | −0.49 | −2.06 |
| P04637-R248Q | 243 | MGGMNQRPI | 502 | PPAMNQRPS | 545 | MNQRP | 474 | B0702 | −1.22 | −1.98 |
| P04637-R248Q | 243 | MGGMNQRPI | 502 | MLGMNQRPV | 546 | MNQRP | 474 | B0801 | −1.43 | −2.02 |
| P04637-R248Q | 243 | MGGMNQRPI | 502 | LDQMNQRPL | 547 | MNQRP | 474 | B5701 | −0.92 | −0.30 |
| P04637-R248Q | 244 | GGMNQRPIL | 503 | FSHNQRPIV | 548 | NQRPI | 475 | A0201 | −0.28 | −1.99 |
| P04637-R248Q | 244 | GGMNQRPIL | 503 | PVANQRPIP | 549 | NQRPI | 475 | A1101 | −0.05 | −2.02 |
| P04637-R248Q | 244 | GGMNQRPIL | 503 | HSLNQRPIL | 550 | NQRPI | 475 | A2601 | −0.38 | −2.00 |
| P04637-R248Q | 244 | GGMNQRPIL | 503 | QSQNQRPIK | 551 | NQRPI | 475 | A3001 | −0.22 | −2.05 |
| P04637-R248Q | 244 | GGMNQRPIL | 503 | SPVNQRPIS | 552 | NQRPI | 475 | B0702 | −1.67 | −2.03 |
| P04637-R248Q | 244 | GGMNQRPIL | 503 | PLTNQRPIL | 553 | NQRPI | 475 | B0801 | −1.34 | −2.06 |
| P04637-R248Q | 244 | GGMNQRPIL | 503 | PALNQRPIP | 554 | NQRPI | 475 | B1501 | −0.35 | −0.04 |
| P04637-R248Q | 244 | GGMNQRPIL | 503 | GLVNQRPIF | 555 | NQRPI | 475 | B5701 | −2.03 | −0.74 |
| P04637-R248W | 241 | SCMGGMNWR | 504 | TLPGGMNWR | 556 | GGMNW | 480 | A1101 | −1.66 | −2.03 |
| P04637-R248W | 241 | SCMGGMNWR | 504 | QLQGGMNWY | 557 | GGMNW | 480 | A2601 | −1.07 | −2.02 |
| P04637-R248W | 241 | SCMGGMNWR | 504 | EIKGGMNWT | 558 | GGMNW | 480 | A3001 | −0.26 | −2.02 |
| P04637-R248W | 241 | SCMGGMNWR | 504 | ACSGGMNWS | 559 | GGMNW | 480 | B1501 | −0.75 | −0.15 |
| P04637-R248W | 241 | SCMGGMNWR | 504 | PASGGMNWM | 560 | GGMNW | 480 | B5701 | −0.96 | −1.76 |
| P04637-R248W | 242 | CMGGMNWRP | 505 | KPAGMNWRL | 561 | GMNWR | 481 | A0201 | −1.57 | −2.06 |
| P04637-R248W | 242 | CMGGMNWRP | 505 | YPLGMNWRR | 562 | GMNWR | 481 | A1101 | −1.77 | −2.06 |
| P04637-R248W | 242 | CMGGMNWRP | 505 | GTFGMNWRF | 563 | GMNWR | 481 | A2601 | −0.51 | −1.99 |
| P04637-R248W | 242 | CMGGMNWRP | 505 | HMNGMNWRI | 564 | GMNWR | 481 | B1501 | −0.34 | −1.00 |
| P04637-R248W | 243 | MGGMNWRPI | 506 | VLDMNWRPG | 565 | MNWRP | 482 | A0201 | −0.83 | −2.09 |
| P04637-R248W | 243 | MGGMNWRPI | 506 | STAMNWRPL | 566 | MNWRP | 482 | A2601 | −1.77 | −1.98 |
| P04637-R248W | 243 | MGGMNWRPI | 506 | PPIMNWRPH | 567 | MNWRP | 482 | B0702 | −1.21 | −2.09 |
| P04637-R248W | 243 | MGGMNWRPI | 506 | QPTMNWRPF | 568 | MNWRP | 482 | B0801 | −2.26 | −2.01 |
| P04637-R248W | 243 | MGGMNWRPI | 506 | QKYMNWRPF | 569 | MNWRP | 482 | B1501 | −0.03 | −0.26 |
| P04637-R248W | 243 | MGGMNWRPI | 506 | PGGMNWRPF | 570 | MNWRP | 482 | B5701 | −1.10 | −3.64 |
| P04637-R248W | 244 | GGMNWRPIL | 507 | VTGNWRPIV | 571 | NWRPI | 483 | A0201 | −0.96 | −2.03 |
| P04637-R248W | 244 | GGMNWRPIL | 507 | TYGNWRPIK | 572 | NWRPI | 483 | A1101 | −0.54 | −1.98 |
| P04637-R248W | 244 | GGMNWRPIL | 507 | TVFNWRPIH | 573 | NWRPI | 483 | A2601 | −0.67 | −2.10 |
| P04637-R248W | 244 | GGMNWRPIL | 507 | SIKNWRPIK | 574 | NWRPI | 483 | A3001 | −0.10 | −2.04 |
| P04637-R248W | 244 | GGMNWRPIL | 507 | SPMNWRPIS | 575 | NWRPI | 483 | B0702 | −1.41 | −2.05 |
| P04637-R248W | 244 | GGMNWRPIL | 507 | PPDNWRPIV | 576 | NWRPI | 483 | B0801 | −1.73 | −2.04 |
| P04637-R248W | 244 | GGMNWRPIL | 507 | RESNWRPIF | 577 | NWRPI | 483 | B1501 | −0.10 | −1.55 |
| P04637-R248W | 244 | GGMNWRPIL | 507 | QTINWRPIP | 578 | NWRPI | 483 | B5701 | −2.63 | −0.71 |
| P04637-R273C | 266 | GRNSFEVCV | 508 | GSLSFEVCV | 579 | SFEVC | 484 | A0201 | −0.20 | −1.98 |
| P04637-R273C | 267 | RNSFEVCVC | 509 | PLVFEVCVT | 580 | FEVCV | 485 | A0201 | −0.18 | −1.97 |
| P04637-R273C | 267 | RNSFEVCVC | 509 | SVVFEVCVH | 581 | FEVCV | 485 | A2601 | −0.51 | −1.99 |
| P04637-R273C | 267 | RNSFEVCVC | 509 | SPVFEVCVS | 582 | FEVCV | 485 | B0702 | −0.05 | −2.00 |
| P04637-R273C | 267 | RNSFEVCVC | 509 | APMFEVCVC | 583 | FEVCV | 485 | B0801 | −1.32 | −2.05 |
| P04637-R273C | 267 | RNSFEVCVC | 509 | PIGFEVCVR | 584 | FEVCV | 485 | B1501 | −1.08 | −0.06 |
| P04637-R273C | 268 | NSFEVCVCA | 510 | VLGEVCVCP | 585 | EVCVC | 486 | A0201 | −1.20 | −2.10 |
| P04637-R273C | 268 | NSFEVCVCA | 510 | FVCEVCVCM | 586 | EVCVC | 486 | A2601 | −1.46 | −2.05 |
| P04637-R273C | 268 | NSFEVCVCA | 510 | DVREVCVCS | 587 | EVCVC | 486 | A3001 | −0.56 | −1.99 |
| P04637-R273C | 268 | NSFEVCVCA | 510 | PPWEVCVCS | 588 | EVCVC | 486 | B0702 | −0.44 | −2.07 |
| P04637-R273C | 268 | NSFEVCVCA | 510 | PAFEVCVCY | 589 | EVCVC | 486 | B1501 | −1.48 | −2.78 |
| P04637-R273C | 268 | NSFEVCVCA | 510 | DQCEVCVCF | 590 | EVCVC | 486 | B5701 | −0.77 | −1.02 |
| P04637-R273C | 269 | SFEVCVCAC | 511 | LIYVCVCAP | 591 | VCVCA | 487 | A0201 | −0.34 | −1.97 |
| P04637-R273C | 269 | SFEVCVCAC | 511 | LLRVCVCAA | 592 | VCVCA | 487 | B0801 | −1.53 | −2.00 |
| P04637-R273C | 269 | SFEVCVCAC | 511 | NITVCVCAH | 593 | VCVCA | 487 | B1501 | −0.08 | −0.79 |
| P04637-R273H | 266 | GRNSFEVHV | 512 | TLDSFEVHG | 594 | SFEVH | 488 | A0201 | −0.20 | −2.07 |
| P04637-R273H | 267 | RNSFEVHVC | 513 | KHCFEVHVI | 595 | FEVHV | 489 | A0201 | −0.30 | −2.00 |
| P04637-R273H | 267 | RNSFEVHVC | 513 | ESEFEVHVM | 596 | FEVHV | 489 | A2601 | −0.81 | −2.10 |
| P04637-R273H | 267 | RNSFEVHVC | 513 | YTRFEVHVL | 597 | FEVHV | 489 | B0702 | −0.04 | −1.99 |
| P04637-R273H | 267 | RNSFEVHVC | 513 | PLMFEVHVL | 598 | FEVHV | 489 | B0801 | −1.29 | −1.99 |
| P04637-R273H | 267 | RNSFEVHVC | 513 | ELQFEVHVY | 599 | FEVHV | 489 | B1501 | −0.82 | −2.26 |
| P04637-R273H | 268 | NSFEVHVCA | 514 | GMAEVHVCM | 600 | EVHVC | 490 | A0201 | −0.86 | −2.07 |
| P04637-R273H | 268 | NSFEVHVCA | 514 | EPSEVHVCY | 601 | EVHVC | 490 | A2601 | −0.91 | −2.09 |
| P04637-R273H | 268 | NSFEVHVCA | 514 | NAKEVHVCA | 602 | EVHVC | 490 | A3001 | −0.86 | −2.07 |
| P04637-R273H | 268 | NSFEVHVCA | 514 | SPMEVHVCT | 603 | EVHVC | 490 | B0702 | −0.62 | −2.08 |
| P04637-R273H | 268 | NSFEVHVCA | 514 | FMYEVHVCV | 604 | EVHVC | 490 | B1501 | −1.25 | −2.11 |
| P04637-R273H | 268 | NSFEVHVCA | 514 | ERWEVHVCN | 605 | EVHVC | 490 | B5701 | −0.79 | −0.22 |
| P04637-R273H | 269 | SFEVHVCAC | 515 | LICVHVCAT | 606 | VHVCA | 491 | A0201 | −0.10 | −2.09 |
| P04637-R273H | 269 | SFEVHVCAC | 515 | ETPVHVCAV | 607 | VHVCA | 491 | B0801 | −2.06 | −2.10 |
| P04637-R282W | 275 | CACPGRDWR | 516 | LLGPGRDWL | 608 | PGRDW | 492 | A0201 | −0.11 | −2.06 |
| P04637-R282W | 275 | CACPGRDWR | 516 | SLWPGRDWR | 609 | PGRDW | 492 | A1101 | −1.95 | −1.99 |
| P04637-R282W | 275 | CACPGRDWR | 516 | NPLPGRDWY | 610 | PGRDW | 492 | A2601 | −1.43 | −2.03 |
| P04637-R282W | 275 | CACPGRDWR | 516 | MNEPGRDWL | 611 | PGRDW | 492 | B5701 | −0.99 | −0.77 |
| P04637-R282W | 276 | ACPGRDWRT | 517 | KGHGRDWRS | 612 | GRDWR | 493 | A3001 | −0.44 | −1.99 |
| P04637-R282W | 277 | CPGRDWRTE | 518 | SMHRDWRTA | 613 | RDWRT | 494 | A0201 | −0.42 | −2.06 |
| P04637-R282W | 277 | CPGRDWRTE | 518 | TSLRDWRTR | 614 | RDWRT | 494 | A2601 | −0.92 | −2.08 |
| P04637-R282W | 277 | CPGRDWRTE | 518 | YPHRDWRTE | 615 | RDWRT | 494 | B0702 | −1.05 | −2.02 |
| P04637-R282W | 277 | CPGRDWRTE | 518 | QTNRDWRTF | 616 | RDWRT | 494 | B0801 | −1.45 | −2.11 |
| P04637-R282W | 278 | PGRDWRTEE | 519 | HAKDWRTEK | 617 | DWRTE | 495 | A3001 | −1.76 | −2.03 |
| P04637-R282W | 278 | PGRDWRTEE | 519 | PLEDWRTEL | 618 | DWRTE | 495 | B0801 | −0.72 | −2.11 |
| P04637-R282W | 278 | PGRDWRTEE | 519 | TLPDWRTEY | 619 | DWRTE | 495 | B1501 | −0.12 | −2.21 |
| P04637-R282W | 278 | PGRDWRTEE | 519 | CELDWRTEM | 620 | DWRTE | 495 | B5701 | −0.82 | −0.94 |

TABLE 10
Bespoke peptides designed for common mutations of TP53 for MHC II alleles

| | | | | | | | | | Predicted | Pre- | |
|---|---|---|---|---|---|---|---|---|---|---|---|
| | | | | | | | | | affinity | dicted | |
| | | | SEQ | | SEQ | | SEQ | | origi- | affinity | |
| | | originating | ID | proposed | ID | TCEM | ID | | nating | proposed | po- |
| gi | pos | peptide | NO.: | peptide | NO.: | core | NO.: | Allele | peptide | peptide | larity |
| P04637-R175H | 166 | SQHMTEVVRHCPHHE | 642 | GSFYTEVVRHCLLVL | 665 | TE~V~HC | 621 | DRB1_0701 | −0.07 | −2.02 | 1.78 |
| P04637-R175H | 170 | TEVVRHCPHHERCSD | 643 | LKFLRHCPHHERKVE | 666 | RH~P~HE | 622 | DRB1_1101 | −0.09 | −2.03 | −1.18 |
| P04637-R248Q | 239 | NSSCMGGMNQRPILT | 644 | VPRIMGGMNQRRRRG | 667 | MG~M~QR | 623 | DRB1_1101 | −0.08 | −2.05 | −1.49 |
| P04637-R248Q | 239 | NSSCMGGMNQRPILT | 644 | VLWLMGGMNQRPFLR | 668 | MG~M~QR | 623 | DRB1_1501 | −0.36 | −2.01 | 1.55 |
| P04637-R248Q | 241 | SCMGGMNQRPILTII | 645 | YPFTGMNQRPIYMLT | 669 | GM~Q~PI | 623 | DRB1_0701 | −0.87 | −2.08 | 1.04 |
| P04637-R248Q | 241 | SCMGGMNQRPILTII | 645 | TQMLGMNQRPIFQVM | 670 | GM~Q~PI | 623 | DRB1_1501 | −0.56 | −1.99 | 0.86 |
| P04637-R248Q | 243 | MGGMNQRPILTIITL | 646 | HKPLNQRPILTLCDV | 671 | NQ~P~LT | 624 | DRB1_0701 | −1.42 | −2.03 | 0.13 |
| P04637-R248Q | 243 | MGGMNQRPILTIITL | 646 | LEWPNQRPILTPNPE | 672 | NQ~P~LT | 624 | DRB1_1101 | −0.62 | −2.01 | −0.43 |
| P04637-R248Q | 243 | MGGMNQRPILTIITL | 646 | DDMTNQRPILTLTFA | 673 | NQ~P~LT | 624 | DRB1_1501 | −1.55 | −2.03 | 0.06 |
| P04637-R248Q | 244 | GGMNQRPILTIITLE | 647 | SPVYQRPILTIVTKH | 674 | QR~I~TI | 625 | DRB1_0401 | −0.58 | −2.04 | 0.47 |
| P04637-R248Q | 244 | GGMNQRPILTIITLE | 647 | EIPPQRPILTIGDIM | 675 | QR~I~TI | 625 | DRB1_0701 | −1.60 | −2.07 | 0.87 |
| P04637-R248Q | 244 | GGMNQRPILTIITLE | 647 | PHRPQRPILTILGSP | 676 | QR~I~TI | 625 | DRB1_1101 | −1.27 | −1.99 | 0.16 |
| P04637-R248Q | 244 | GGMNQRPILTIITLE | 647 | PLDWQRPILTITDLF | 677 | QR~I~TI | 625 | DRB1 1501 | −1.49 | −1.99 | 1.38 |
| P04637-R248W | 238 | CNSSCMGGMNWRPIL | 649 | YRWPCMGGMNWAQLG | 678 | CM~G~NW | 626 | DRB1_0701 | −0.10 | −1.97 | 0.98 |
| P04637-R248W | 239 | NSSCMGGMNWRPILT | 650 | LRPLMGGMNWRLKLY | 679 | MG~M~WR | 627 | DRB1_0401 | −0.12 | −2.00 | 1.18 |
| P04637-R248W | 239 | NSSCMGGMNWRPILT | 650 | EPKFMGGMNWRLFYA | 680 | MG~M~WR | 627 | DRB1_0701 | −0.24 | −2.01 | 0.91 |
| P04637-R248W | 239 | NSSCMGGMNWRPILT | 650 | RPDLMGGMNWRLYDR | 681 | MG~M~WR | 627 | DRB1_1501 | −1.36 | −1.99 | −0.48 |
| P04637-R248W | 241 | SCMGGMNWRPILTII | 651 | SEFVGMNWRPIFSLL | 682 | GM~W~PI | 628 | DRB1_0701 | −1.28 | −2.05 | 1.76 |
| P04637-R248W | 241 | SCMGGMNWRPILTII | 651 | EPMLGMNWRPINPGL | 683 | GM~W~PI | 628 | DRB1_1101 | −0.81 | −1.99 | 0.77 |
| P04637-R248W | 241 | SCMGGMNWRPILTII | 651 | PNLTGMNWRPIFLRP | 684 | GM~W~PI | 628 | DRB1_1501 | −1.20 | −2.08 | 0.88 |
| P04637-R248W | 243 | MGGMNWRPILTIITL | 652 | LPQFNWRPILTSASS | 685 | NW~P~LT | 629 | DRB1_0401 | −0.43 | −2.05 | 0.62 |
| P04637-R248W | 243 | MGGMNWRPILTIITL | 652 | DLRRNWRPILTPWTL | 686 | NW~P~LT | 629 | DRB1_0701 | −1.65 | −2.02 | 0.37 |
| P04637-R248W | 243 | MGGMNWRPILTIITL | 652 | PLFRNWRPILTHYNP | 687 | NW~P~LT | 629 | DRB1_1101 | −1.46 | −2.06 | 0.66 |
| P04637-R248W | 243 | MGGMNWRPILTIITL | 652 | KVNPNWRPILTAMFP | 688 | NW~P~LT | 629 | DRB1_1501 | −3.14 | −2.05 | 0.84 |
| P04637-R248W | 244 | GGMNWRPILTIITLE | 653 | IFKYWRPILTILMEM | 689 | WR~I~TI | 630 | DRB1_0401 | −1.49 | −2.05 | 2.45 |
| P04637-R248W | 244 | GGMNWRPILTIITLE | 653 | VVYMWRPILTIQSLR | 690 | WR~I~TI | 630 | DRB1_0701 | −2.33 | −2.08 | 1.85 |
| P04637-R248W | 244 | GGMNWRPILTIITLE | 653 | EARQWRPILTILPER | 691 | WR~I~TI | 630 | DRB1_1101 | −2.52 | −2.03 | 0.19 |
| P04637-R248W | 244 | GGMNWRPILTIITLE | 653 | MSSPWRPILTIFLKY | 692 | WR~I~TI | 630 | DRB1_1501 | −2.85 | −2.00 | 1.75 |
| P04637-R273C | 263 | NLLGRNSFEVCVCAC | 654 | PLYQRNSFEVCFWPG | 693 | RN~F~VC | 631 | DRB1_0701 | −0.81 | −2.07 | 0.82 |
| P04637-R273C | 264 | LLGRNSFEVCVCACP | 655 | PEMQNSFEVCVLFRI | 694 | NS~E~CV | 632 | DRB1_0701 | −0.54 | −1.97 | 0.98 |
| P04637-R273C | 268 | NSFEVCVCACPGRDR | 656 | GYSFVCVCACPFKID | 695 | VC~C~CP | 633 | DRB1_0401 | −0.66 | −2.09 | 1.60 |
| P04637-R273C | 268 | NSFEVCVCACPGRDR | 656 | TLPRVCVCACPIVLA | 696 | VC~C~CP | 633 | DRB1_0701 | −0.19 | −1.98 | 2.28 |
| P04637-R273C | 269 | SFEVCVCACPGRDRR | 657 | PMWMCVCACPGSGLE | 697 | CV~A~PG | 634 | DRB1_0401 | −0.91 | −1.98 | 1.50 |
| P04637-R273H | 263 | NLLGRNSFEVHVCAC | 658 | PLRFRNSFEVHITFH | 698 | RN~F~VH | 635 | DRB1_0701 | −1.38 | −2.02 | 0.52 |
| P04637-R273H | 264 | LLGRNSFEVHVCACP | 659 | TPMRNSFEVHVIVGV | 699 | NS~E~HV | 636 | DRB1_0701 | −0.14 | −1.99 | 0.89 |
| P04637-R273H | 268 | NSFEVHVCACPGRDR | 660 | LVHLVHVCACPADVP | 700 | VH~C~CP | 637 | DRB1_0401 | −0.51 | −2.08 | 1.79 |
| P04637-R273H | 269 | SFEVHVCACPGRDRR | 661 | PMLIHVCACPGKSSK | 701 | HV~A~PG | 638 | DRB1_0401 | −1.19 | −2.02 | 0.34 |
| P04637-R282W | 273 | RVCACPGRDWRTEEE | 662 | RPFVCPGRDWRTKPQ | 702 | CP~R~WR | 639 | DRB1_1101 | −0.20 | −2.01 | −1.21 |
| P04637-R282W | 275 | CACPGRDWRTEEENL | 663 | WPYYGRDWRTEAVKP | 703 | GR~W~TE | 640 | DRB1_1101 | −0.01 | −2.00 | −0.75 |
| P04637-R282W | 278 | PGRDWRTEEENLRKK | 664 | KHMYWRTEEENLGLP | 704 | WR~E~EN | 641 | DRB1_1101 | −0.76 | −2.02 | −0.91 |

### Example 10: Bespoke Peptides Targeting Common Mutations in PTEN

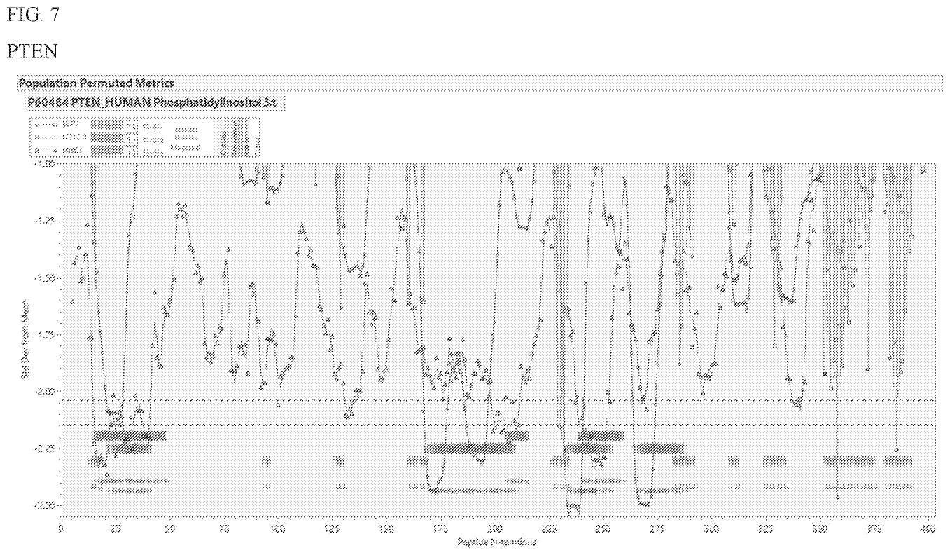

PTEN (phosphatase and tensin homologue) is another tumor suppressor, which negatively regulates the PI3K-AKT signaling pathway and thereby modulating cell cycle progression and cell survival [71]. PTEN is exemplified by the sequence P60484 in Uniprot. FIG. 7 provides an overview of the immunome features. Mutations of PTEN have been reported in glioblastoma multiforme, advanced prostate cancers, melanoma, endometrial cancer, and also in breast, head, neck, and thyroid cancers [68, 72, 73]. Among the most commonly recorded PTEN mutations in the Genome Data Commons are mutations within the P loop which is essential to the phosphatase function. These include the mutations R130Q and R130G. In Tables 11 and 12 we provide examples of bespoke peptides designed to have a desirable predicted binding affinity to example alleles and to target these two common mutations. Given the location of these particular mutations and the low binding affinity for MHC II alleles it is impossible to identify peptides that would be naturally presented at any level of affinity and also encompass the mutated amino acid. Hence, we also show for some alleles how an adjacent peptide can be selected to provide CD4+ help to the bespoke CD8+ targeting peptide.

TABLE 11
Bespoke peptides designed for common mutations of PTEN for MHC II alleles

| | | | | | | | | predicted | predicted | |
|---|---|---|---|---|---|---|---|---|---|---|
| | | | SEQ | | SEQ | | SEQ | binding | binding | |
| | | proposed | ID | originating | ID | TCEM | ID | proposed | original | |
| gi | pos | peptide | NO.: | peptide | NO.: | core | NO.: | peptide | peptide | Allele |
| P60484-R130G | 125 | LLNKGGTGG | 721 | KAGKGGTGV | 713 | KGGTG | 705 | −2.19 | −1.22 | A0201 |
| P60484-R130G | 125 | YPIKGGTGV | 722 | KAGKGGTGV | 713 | KGGTG | 705 | −2.01 | −1.22 | |
| P60484-R130G | 126 | FFIGGTGVA | 723 | AGKGGTGVM | 714 | GGTGV | 706 | −2.13 | −0.08 | |
| P60484-R130G | 126 | FQPGGTGVL | 724 | AGKGGTGVM | 714 | GGTGV | 706 | −2.13 | −0.08 | |
| P60484-R130G | 124 | CAMGKGGTY | 725 | CKAGKGGTG | 715 | GKGGT | 707 | −2.07 | −0.17 | A2601 |
| P60484-R130G | 124 | NSFGKGGTF | 726 | CKAGKGGTG | 715 | GKGGT | 707 | −2.01 | −0.17 | |
| P60484-R130G | 125 | IVLKGGTGV | 727 | KAGKGGTGV | 713 | KGGTG | 705 | −2.01 | −0.52 | |
| P60484-R130G | 125 | YVFKGGTGR | 728 | KAGKGGTGV | 713 | KGGTG | 705 | −2.03 | −0.52 | |
| P60484-R130G | 126 | DVGGGTGVR | 729 | AGKGGTGVM | 714 | GGTGV | 706 | −2.11 | −0.11 | |
| P60484-R130G | 126 | CIHGGTGVY | 730 | AGKGGTGVM | 714 | GGTGV | 706 | −2.08 | −0.11 | |
| P60484-R130G | 123 | NGKAGKGGQ | 731 | HCKAGKGGT | 716 | AGKGG | 708 | −1.99 | −1.40 | A3001 |
| P60484-R130G | 123 | SAKAGKGGP | 732 | HCKAGKGGT | 716 | AGKGG | 708 | −2.07 | −1.40 | |
| P60484-R130G | 126 | KVEGGTGVP | 733 | AGKGGTGVM | 714 | GGTGV | 706 | −2.07 | −0.68 | |
| P60484-R130G | 126 | KTPGGTGVK | 734 | AGKGGTGVM | 714 | GGTGV | 706 | −1.98 | −0.68 | |
| P60484-R130G | 123 | DPHAGKGGP | 735 | HCKAGKGGT | 716 | AGKGG | 708 | −2.02 | −1.54 | B0702 |
| P60484-R130G | 123 | NPHAGKGGY | 736 | HCKAGKGGT | 716 | AGKGG | 708 | −1.98 | −1.54 | |
| P60484-R130G | 124 | CPTGKGGTF | 737 | CKAGKGGTG | 715 | GKGGT | 707 | −2.04 | −0.17 | |
| P60484-R130G | 124 | IPQGKGGTW | 738 | CKAGKGGTG | 715 | GKGGT | 707 | −1.99 | −0.17 | |
| P60484-R130G | 125 | PAQKGGTGL | 739 | KAGKGGTGV | 713 | KGGTG | 705 | −2.07 | −2.12 | |
| P60484-R130G | 125 | YGKKGGTGA | 740 | KAGKGGTGV | 713 | KGGTG | 705 | −2.04 | −2.12 | |
| P60484-R130G | 126 | SPPGGTGVV | 741 | AGKGGTGVM | 714 | GGTGV | 706 | −2.04 | −1.35 | |
| P60484-R130G | 126 | DARGGTGVM | 742 | AGKGGTGVM | 714 | GGTGV | 706 | −1.99 | −1.35 | |
| P60484-R130G | 124 | MLAGKGGTV | 743 | CKAGKGGTG | 715 | GKGGT | 707 | −1.99 | −0.43 | B0801 |
| P60484-R130G | 124 | QLEGKGGTC | 744 | CKAGKGGTG | 715 | GKGGT | 707 | −2.07 | −0.43 | |
| P60484-R130G | 125 | NLKKGGTGV | 745 | KAGKGGTGV | 713 | KGGTG | 705 | −2.05 | −0.55 | |
| P60484-R130G | 125 | YPKKGGTGI | 746 | KAGKGGTGV | 713 | KGGTG | 705 | −2.05 | −0.55 | |
| P60484-R130G | 123 | LGSAGKGGM | 747 | HCKAGKGGT | 716 | AGKGG | 708 | −2.01 | −0.16 | B1501 |
| P60484-R130G | 123 | SQPAGKGGY | 748 | HCKAGKGGT | 716 | AGKGG | 708 | −2.01 | −0.16 | |
| P60484-R130G | 125 | VVRKGGTGY | 749 | KAGKGGTGV | 713 | KGGTG | 705 | −2.07 | −0.44 | |
| P60484-R130G | 125 | YGKKGGTGY | 750 | KAGKGGTGV | 713 | KGGTG | 705 | −2.00 | −0.44 | |
| P60484-R130G | 126 | NFRGGTGVY | 751 | AGKGGTGVM | 714 | GGTGV | 706 | −2.02 | −1.87 | |
| P60484-R130G | 126 | RFTGGTGVY | 752 | AGKGGTGVM | 714 | GGTGV | 706 | −2.03 | −1.87 | |
| P60484-R130G | 125 | YNEKGGTGF | 753 | KAGKGGTGV | 713 | KGGTG | 705 | −2.08 | −0.28 | B5701 |
| P60484-R130G | 125 | KCYKGGTGY | 754 | KAGKGGTGV | 713 | KGGTG | 705 | −2.14 | −0.28 | |
| P60484-R130G | 126 | KSLGGTGVL | 755 | AGKGGTGVM | 714 | GGTGV | 706 | −2.03 | −1.20 | |
| P60484-R130G | 126 | KAFGGTGVR | 756 | AGKGGTGVM | 714 | GGTGV | 706 | −2.14 | −1.20 | |
| P60484-R130Q | 125 | FNYKGQTGV | 757 | KAGKGQTGV | 717 | KGQTG | 709 | −2.11 | −0.94 | A0201 |
| P60484-R130Q | 125 | FMSKGQTGP | 758 | KAGKGQTGV | 717 | KGQTG | 709 | −2.19 | −0.94 | |
| P60484-R130Q | 126 | DLVGQTGVL | 759 | AGKGQTGVM | 718 | GQTGV | 710 | −2.05 | −0.11 | |
| P60484-R130Q | 126 | PMIGQTGVA | 760 | AGKGQTGVM | 718 | GQTGV | 710 | −2.14 | −0.11 | |
| P60484-R130Q | 125 | ETLKGQTGQ | 761 | KAGKGQTGV | 717 | KGQTG | 709 | −2.05 | −0.14 | A2601 |
| P60484-R130Q | 125 | LVVKGQTGP | 762 | KAGKGQTGV | 717 | KGQTG | 709 | −2.12 | −0.14 | |
| P60484-R130Q | 123 | ESKAGKGQN | 763 | HCKAGKGQT | 719 | AGKGQ | 711 | −2.03 | −1.52 | A3001 |
| P60484-R130Q | 123 | KFKAGKGQQ | 764 | HCKAGKGQT | 719 | AGKGQ | 711 | −1.99 | −1.52 | |
| P60484-R130Q | 125 | FVRKGQTGN | 765 | KAGKGQTGV | 717 | KGQTG | 709 | −2.02 | −0.11 | |
| P60484-R130Q | 125 | HVKKGQTGN | 766 | KAGKGQTGV | 717 | KGQTG | 709 | −2.06 | −0.11 | |
| P60484-R130Q | 126 | SKRGQTGVK | 767 | AGKGQTGVM | 718 | GQTGV | 710 | −1.98 | −0.21 | |
| P60484-R130Q | 126 | PARGQTGVV | 768 | AGKGQTGVM | 718 | GQTGV | 710 | −2.04 | −0.21 | |
| P60484-R130Q | 123 | YSRAGKGQI | 769 | HCKAGKGQT | 719 | AGKGQ | 711 | −2.06 | −0.91 | B0702 |
| P60484-R130Q | 123 | NPPAGKGQL | 770 | HCKAGKGQT | 719 | AGKGQ | 711 | −1.99 | −0.91 | |
| P60484-R130Q | 124 | GPEGKGQTA | 771 | CKAGKGQTG | 720 | GKGQT | 712 | −2.00 | −0.18 | |
| P60484-R130Q | 124 | FPLGKGQTA | 772 | CKAGKGQTG | 720 | GKGQT | 712 | −2.06 | −0.18 | |
| P60484-R130Q | 125 | MGSKGQTGI | 773 | KAGKGQTGV | 717 | KGQTG | 709 | −2.05 | −2.28 | |
| P60484-R130Q | 125 | YSMKGQTGI | 774 | KAGKGQTGV | 717 | KGQTG | 709 | −2.05 | −2.28 | |
| P60484-R130Q | 126 | YPAGQTGVG | 775 | AGKGQTGVM | 718 | GQTGV | 710 | −2.01 | −1.22 | |
| P60484-R130Q | 126 | VGRGQTGVL | 776 | AGKGQTGVM | 718 | GQTGV | 710 | −2.00 | −1.22 | |
| P60484-R130Q | 124 | ELFGKGQTV | 777 | CKAGKGQTG | 720 | GKGQT | 712 | −2.05 | −0.25 | B0801 |
| P60484-R130Q | 124 | FNKGKGQTI | 778 | CKAGKGQTG | 720 | GKGQT | 712 | −2.06 | −0.25 | |
| P60484-R130Q | 125 | QFKKGQTGV | 779 | KAGKGQTGV | 717 | KGQTG | 709 | −2.05 | −0.49 | |
| P60484-R130Q | 125 | LLKKGQTGC | 780 | KAGKGQTGV | 717 | KGQTG | 709 | −2.05 | −0.49 | |
| P60484-R130Q | 126 | KPQGQTGVV | 781 | AGKGQTGVM | 718 | GQTGV | 710 | −2.02 | −0.97 | |
| P60484-R130Q | 126 | LLSGQTGVV | 782 | AGKGQTGVM | 718 | GQTGV | 710 | −2.06 | −0.97 | |
| P60484-R130Q | 125 | MVGKGQTGY | 783 | KAGKGQTGV | 717 | KGQTG | 709 | −2.07 | −0.19 | B1501 |
| P60484-R130Q | 125 | DFMKGQTGY | 784 | KAGKGQTGV | 717 | KGQTG | 709 | −2.06 | −0.19 | |
| P60484-R130Q | 126 | DFCGQTGVY | 785 | AGKGQTGVM | 718 | GQTGV | 710 | −2.07 | −1.71 | |
| P60484-R130Q | 126 | PEIGQTGVF | 786 | AGKGQTGVM | 718 | GQTGV | 710 | −2.03 | −1.71 | |
| P60484-R130Q | 125 | AKLKGQTGF | 787 | KAGKGQTGV | 717 | KGQTG | 709 | −1.99 | −0.37 | B5701 |
| P60484-R130Q | 125 | ISIKGQTGL | 788 | KAGKGQTGV | 717 | KGQTG | 709 | −2.06 | −0.37 | |
| P60484-R130Q | 126 | NVIGQTGVF | 789 | AGKGQTGVM | 718 | GQTGV | 710 | −2.04 | −1.09 | |
| P60484-R130Q | 126 | DGYGQTGVL | 790 | AGKGQTGVM | 718 | GQTGV | 710 | −2.01 | −1.09 | |

TABLE 12
Bespoke peptides designed for common mutations of PTEN for MHC II alleles

| | | | | | | | | | Predicted | |
|---|---|---|---|---|---|---|---|---|---|---|
| | | | | | | | | | affinity | |
| | | | SEQ | | SEQ | | SEQ | | origi- | |
| | | proposed | ID | | ID | TCEM | ID | | nating | |
| gi | pos | peptide | NO.: | | NO.: | core | NO.: | | peptide | Allele |
| | | | | | | | | Pre- | | |
| | | | | | | | | dicted | | |
| | | | | origi- | | | | affinity | | |
| | | | | nating | | | | proposed | | |
| | | | | peptide | | | | peptide | | |
| P60484-R130G | 121 | KHTMKAGKGGTLFLS | 805 | AIHCKAGKGGTGVMI | 798 | KA~K~GT | 791 | −2.00 | −0.74 | DRB1_0701 |
| P60484-R130G | 121 | KYEFKAGKGGTYDYV | 806 | AIHCKAGKGGTGVMI | 798 | KA~K~GT | 791 | −2.01 | −0.74 | |
| P60484-R130G | 126 | KIYTGTGVMICLVAL | 807 | AGKGGTGVMICAYLL | 799 | GT~V~IC | 792 | −2.04 | −0.23 | |
| P60484-R130G | 126 | LTAPGTGVMICSFFI | 808 | AGKGGTGVMICAYLL | 799 | GT~V~IC | 792 | −2.03 | −0.23 | |
| P60484-R130Q | 121 | FSLMKAGKGQTMVLI | 809 | AIHCKAGKGQTGVMI | 800 | KA~K~QT | 793 | −2.07 | −0.34 | DRB1_0701 |
| P60484-R130Q | 121 | NSFLKAGKGQTLFSV | 810 | AIHCKAGKGQTGVMI | 800 | KA~K~QT | 793 | −2.04 | −0.34 | |
| P60484-R130Q | 126 | KYAEQTGVMICFGIF | 811 | AGKGQTGVMICAYLL | 801 | QT~V~IC | 794 | −2.04 | −0.40 | |
| P60484-R130Q | 126 | KTYEQTGVMICVPYA | 812 | AGKGQTGVMICAYLL | 801 | QT~V~IC | 794 | −2.02 | −0.40 | |
| | | | | Adjacent | | | | | | |
| | | | | non-mutated | | | | Pre- | | |
| | | | | peptides as | | | | dicted | | |
| | | | | CD4+helpers | | | | affinity | | |
| | | | | for some | | | | natural | | |
| | | | | alleles | | | | peptide | | |
| P60484 | 131 | | | TGVMICAYLLHRGKF | 802 | IC~Y~LH | 795 | −0.61 | | DRB1_0401 |
| P60484 | 132 | | | GVMICAYLLHRGKFL | 803 | CA~L~HR | 796 | −1.40 | | |
| P60484 | 133 | | | VMICAYLLHRGKFLK | 804 | AY~L~RG | 797 | −0.29 | | |
| P60484 | 131 | | | TGVMICAYLLHRGKF | 802 | IC~Y~LH | 795 | −1.63 | | DRB1_0802 |
| P60484 | 132 | | | GVMICAYLLHRGKFL | 803 | CA~L~HR | 796 | −1.28 | | |
| P60484 | 133 | | | VMICAYLLHRGKFLK | 804 | AY~L~RG | 797 | −2.14 | | |
| P60484 | 131 | | | TGVMICAYLLHRGKF | 802 | IC~Y~LH | 795 | −1.09 | | DRB1_1501 |
| P60484 | 132 | | | GVMICAYLLHRGKFL | 803 | CA~L~HR | 796 | −1.25 | | |
| P60484 | 133 | | | VMICAYLLHRGKFLK | 804 | AY~L~RG | 797 | −1.58 | | |

### Example 11: Bespoke Peptides Targeting Common Mutations in ERBB2

ERBB2, also known as HER2, is a tyrosine kinase that is part of several cell surface receptor complexes. It is commonly mutated in bladder, breast, colorectal and gastric cancers and in gliomas, as well as other cancers [74]. The canonical sequence is P04626. FIG. 8 provides an overview of the immunome features. Several monoclonal antibodies have been developed to target ERBB2[75]. Increasingly the role of mutations and amplification of ERBB2 in cancers is being recognized and alternative methods of targeting it are being sought, including T cell targeting. Missense mutations comprise about 70% of the mutations in ERBB2. Like EGFR, ERBB2 is a transmembrane protein and mutations are recorded in the extracellular domain, transmembrane domain and intracellular domain. Among the most common mutations observed in ERBB2 are S310F (extracellular), R678Q (juxta membrane) and V842I (intracellular). Tables 13 and 14 provide examples of peptides which embody these mutant amino acids within their T cell exposed motifs and in which the flanking groove exposed amino acids are selected to provide binding to selected alleles. The same approach could be applied to other ERBB2 mutations and to design peptides with a desired binding affinity for other alleles therefore the examples are considered non-limiting.

TABLE 13
Bespoke peptides designed for common mutations of ERBB2 for MHC I alleles

| | | | | | | | | | Predicted | |
|---|---|---|---|---|---|---|---|---|---|---|
| | | | | | | | | Predicted | binding | |
| | | | | | | | | binding | affinity | |
| | | | SEQ | origi- | SEQ | | SEQ | affinity | origi- | |
| | | proposed | ID | nating | ID | TCEM | ID | proposed | nating | |
| gi | pos | peptide | NO.: | peptide | NO.: | core | NO.: | peptide | peptide | Allele |
| P04626-R678Q | 671 | AAPLIKRQR | 835 | FGILIKRQQ | 825 | LIKRQ | 813 | -2.04 | -1.01 | A1101 |
| P04626-R678Q | 671 | PAVLIKRQP | 836 | FGILIKRQQ | 825 | LIKRQ | 813 | −2.00 | −1.01 | |
| P04626-R678Q | 672 | LGVIKRQQK | 837 | GILIKRQQQ | 826 | IKRQQ | 814 | −2.05 | −0.37 | |
| P04626-R678Q | 672 | ALPIKRQQK | 838 | GILIKRQQQ | 826 | IKRQQ | 814 | −2.09 | −0.37 | |
| P04626-R678Q | 673 | IVMKRQQQR | 839 | ILIKRQQQK | 827 | KRQQQ | 815 | −2.06 | −2.38 | |
| P04626-R678Q | 674 | LVLRQQQKP | 840 | LIKRQQQKI | 828 | RQQQK | 816 | −2.02 | −0.18 | A2601 |
| P04626-R678Q | 674 | FVTRQQQKP | 841 | LIKRQQQKI | 828 | RQQQK | 816 | −2.08 | −0.18 | |
| P04626-R678Q | 674 | FAWRQQQKI | 842 | LIKRQQQKI | 828 | RQQQK | 816 | −2.04 | −0.83 | B0702 |
| P04626-R678Q | 674 | WAPRQQQKL | 843 | LIKRQQQKI | 828 | RQQQK | 816 | −2.00 | −0.83 | |
| P04626-R678Q | 674 | SLPRQQQKV | 844 | LIKRQQQKI | 828 | RQQQK | 816 | −2.00 | −2.78 | B0801 |
| P04626-R678Q | 674 | FFDRQQQKG | 845 | LIKRQQQKI | 828 | RQQQK | 816 | −2.00 | −2.78 | |
| P04626-R678Q | 674 | AGMRQQQKF | 846 | LIKRQQQKI | 828 | RQQQK | 816 | −2.00 | −0.78 | B1501 |
| P04626-R678Q | 674 | YGLRQQQKY | 847 | LIKRQQQKI | 828 | RQQQK | 816 | −2.07 | −0.78 | |
| P04626-R678Q | 671 | WRFLIKRQF | 848 | FGILIKRQQ | 825 | LIKRQ | 813 | −2.00 | −1.83 | B5701 |
| P04626-R678Q | 671 | RPLLIKRQL | 849 | FGILIKRQQ | 825 | LIKRQ | 813 | −2.00 | −1.83 | |
| P04626-S310F | 303 | TLNTDVGFA | 850 | YLSTDVGFC | 829 | TDVGF | 817 | −2.02 | −1.89 | A0201 |
| P04626-S310F | 303 | RLPTDVGFG | 851 | YLSTDVGFC | 829 | TDVGF | 817 | −2.08 | −1.89 | |
| P04626-S310F | 305 | KLQVGFCTG | 852 | STDVGFCTL | 830 | VGFCT | 818 | −2.02 | −0.78 | |
| P04626-S310F | 305 | SLEVGFCTR | 853 | STDVGFCTL | 830 | VGFCT | 818 | −2.01 | −0.49 | A1101 |
| P04626-S310F | 305 | RNIVGFCTR | 854 | STDVGFCTL | 830 | VGFCT | 818 | −2.09 | −0.49 | |
| P04626-S310F | 303 | NQVTDVGFR | 855 | YLSTDVGFC | 829 | TDVGF | 817 | −2.01 | −1.12 | A2601 |
| P04626-S310F | 303 | LTTTDVGFP | 856 | YLSTDVGFC | 829 | TDVGF | 817 | −2.06 | −1.12 | |
| P04626-S310F | 305 | ETPVGFCTR | 857 | STDVGFCTL | 830 | VGFCT | 818 | −2.06 | −0.69 | |
| P04626-S310F | 305 | EIQVGFCTI | 858 | STDVGFCTL | 830 | VGFCT | 818 | −2.03 | −0.69 | |
| P04626-S310F | 306 | NSVGFCTLG | 859 | TDVGFCTLV | 831 | GFCTL | 819 | −2.02 | −1.45 | |
| P04626-S310F | 306 | DNAGFCTLP | 860 | TDVGFCTLV | 831 | GFCTL | 819 | −2.02 | −1.45 | |
| P04626-S310F | 304 | DTRDVGFCP | 861 | LSTDVGFCT | 832 | DVGFC | 820 | −2.03 | −0.13 | A3001 |
| P04626-S310F | 304 | PGRDVGFCP | 862 | LSTDVGFCT | 832 | DVGFC | 820 | −2.02 | −0.13 | |
| P04626-S310F | 305 | RADVGFCTP | 863 | STDVGFCTL | 830 | VGFCT | 818 | −2.09 | −0.14 | |
| P04626-S310F | 305 | GSRVGFCTQ | 864 | STDVGFCTL | 830 | VGFCT | 818 | −2.04 | −0.14 | |
| P04626-S310F | 303 | LLRTDVGFL | 865 | YLSTDVGFC | 829 | TDVGF | 817 | −2.06 | −0.17 | B0702 |
| P04626-S310F | 303 | DPKTDVGFM | 866 | YLSTDVGFC | 829 | TDVGF | 817 | −2.04 | −0.17 | |
| P04626-S310F | 305 | TPVVGFCTG | 867 | STDVGFCTL | 830 | VGFCT | 818 | −2.06 | −0.32 | |
| P04626-S310F | 305 | GPGVGFCTG | 868 | STDVGFCTL | 830 | VGFCT | 818 | −1.98 | −0.32 | |
| P04626-S310F | 303 | LYQTDVGFI | 869 | YLSTDVGFC | 829 | TDVGF | 817 | −1.98 | −1.34 | B0801 |
| P04626-S310F | 303 | MPDTDVGFV | 870 | YLSTDVGFC | 829 | TDVGF | 817 | −2.00 | −1.34 | |
| P04626-S310F | 305 | QPRVGFCTI | 871 | STDVGFCTL | 830 | VGFCT | 818 | −1.99 | −0.08 | |
| P04626-S310F | 305 | VGRVGFCTV | 872 | STDVGFCTL | 830 | VGFCT | 818 | −2.04 | −0.08 | |
| P04626-S310F | 303 | PVYTDVGFL | 873 | YLSTDVGFC | 829 | TDVGF | 817 | −2.05 | −0.29 | B1501 |
| P04626-S310F | 303 | WPPTDVGFF | 874 | YLSTDVGFC | 829 | TDVGF | 817 | −2.04 | −0.29 | |
| P04626-S310F | 304 | EITDVGFCM | 875 | LSTDVGFCT | 832 | DVGFC | 820 | −2.07 | −0.30 | |
| P04626-S310F | 304 | NSPDVGFCM | 876 | LSTDVGFCT | 832 | DVGFC | 820 | −2.07 | −0.30 | |
| P04626-S310F | 305 | ELSVGFCTM | 877 | STDVGFCTL | 830 | VGFCT | 818 | −2.06 | −1.01 | |
| P04626-S310F | 305 | DEVVGFCTY | 878 | STDVGFCTL | 830 | VGFCT | 818 | −2.02 | −1.01 | |
| P04626-S310F | 306 | PMKGFCTLV | 879 | TDVGFCTLV | 831 | GFCTL | 819 | −2.01 | −1.24 | |
| P04626-S310F | 306 | PQPGFCTLW | 880 | TDVGFCTLV | 831 | GFCTL | 819 | −2.07 | −1.24 | |
| P04626-S310F | 304 | VMKDVGFCW | 881 | LSTDVGFCT | 832 | DVGFC | 820 | −2.05 | −0.19 | B5701 |
| P04626-S310F | 304 | RARDVGFCR | 882 | LSTDVGFCT | 832 | DVGFC | 820 | −1.99 | −0.19 | |
| P04626-S310F | 305 | TGGVGFCTY | 883 | STDVGFCTL | 830 | VGFCT | 818 | −2.04 | −0.81 | |
| P04626-S310F | 305 | QGIVGFCTR | 884 | STDVGFCTL | 830 | VGFCT | 818 | −2.02 | −0.81 | |
| P04626-V8421 | 835 | NLYDVRLIS | 885 | YLEDVRLIH | 831 | DVRLI | 821 | −2.01 | −1.05 | A0201 |
| P04626-V8421 | 835 | TTHDVRLIV | 886 | YLEDVRLIH | 831 | DVRLI | 821 | −2.03 | −1.05 | |
| P04626-V8421 | 835 | RAEDVRLIR | 887 | YLEDVRLIH | 831 | DVRLI | 821 | −2.09 | −0.64 | A1101 |
| P04626-V8421 | 835 | THGDVRLIK | 888 | YLEDVRLIH | 831 | DVRLI | 821 | −2.08 | −0.64 | |
| P04626-V8421 | 836 | ADRVRLIHR | 889 | LEDVRLIHR | 832 | VRLIH | 822 | −2.07 | −1.29 | |
| P04626-V8421 | 836 | ADDVRLIHR | 890 | LEDVRLIHR | 832 | VRLIH | 822 | −2.00 | −1.29 | |
| P04626-V8421 | 835 | HSHDVRLIR | 891 | YLEDVRLIH | 831 | DVRLI | 821 | −2.05 | −0.56 | |
| P04626-V8421 | 835 | EIHDVRLIL | 892 | YLEDVRLIH | 831 | DVRLI | 821 | −1.99 | −0.56 | |
| P04626-V8421 | 837 | VVRRLIHRF | 893 | EDVRLIHRD | 833 | RLIHR | 823 | −2.05 | −1.10 | |
| P04626-V8421 | 837 | EGLRLIHRI | 894 | EDVRLIHRD | 833 | RLIHR | 823 | −2.07 | −1.10 | |
| P04626-V8421 | 838 | EGLLIHRDR | 895 | DVRLIHRDL | 834 | LIHRD | 824 | −1.99 | −0.79 | |
| P04626-V8421 | 838 | DSALIHRDY | 896 | DVRLIHRDL | 834 | LIHRD | 824 | −2.02 | −0.79 | A3001 |
| P04626-V8421 | 838 | RSRLIHRDL | 897 | DVRLIHRDL | 834 | LIHRD | 824 | −2.01 | −1.69 | |
| P04626-V8421 | 838 | TIRLIHRDS | 898 | DVRLIHRDL | 834 | LIHRD | 824 | −2.06 | −1.69 | B0702 |
| P04626-V8421 | 835 | RVHDVRLIL | 899 | YLEDVRLIH | 831 | DVRLI | 821 | −1.98 | 0.06 | |
| P04626-V8421 | 835 | VPVDVRLIP | 900 | YLEDVRLIH | 831 | DVRLI | 821 | −2.00 | 0.06 | |
| P04626-V8421 | 838 | PPRLIHRDE | 901 | DVRLIHRDL | 834 | LIHRD | 824 | −2.03 | −1.78 | |
| P04626-V8421 | 838 | VPKLIHRDW | 902 | DVRLIHRDL | 834 | LIHRD | 824 | −1.98 | −1.78 | B0801 |
| P04626-V8421 | 835 | WGRDVRLIL | 903 | YLEDVRLIH | 831 | DVRLI | 821 | −2.05 | −0.10 | |
| P04626-V8421 | 835 | HPRDVRLIL | 904 | YLEDVRLIH | 831 | DVRLI | 821 | −2.00 | −0.10 | |
| P04626-V8421 | 838 | VLRLIHRDI | 905 | DVRLIHRDL | 834 | LIHRD | 824 | −2.06 | −1.07 | |
| P04626-V8421 | 838 | VLRLIHRDV | 906 | DVRLIHRDL | 834 | LIHRD | 824 | −2.04 | −1.07 | B1501 |
| P04626-V8421 | 835 | ADPDVRLIY | 907 | YLEDVRLIH | 831 | DVRLI | 821 | −2.00 | −0.77 | |
| P04626-V8421 | 835 | RLPDVRLIM | 908 | YLEDVRLIH | 831 | DVRLI | 821 | −2.06 | −0.77 | |
| P04626-V8421 | 838 | IVPLIHRDY | 909 | DVRLIHRDL | 834 | LIHRD | 824 | −2.03 | −0.64 | |
| P04626-V8421 | 838 | GSVLIHRDF | 910 | DVRLIHRDL | 834 | LIHRD | 824 | −2.08 | −0.64 | B5701 |
| P04626-V8421 | 835 | FSGDVRLIM | 911 | YLEDVRLIH | 831 | DVRLI | 821 | −2.05 | −0.43 | |
| P04626-V8421 | 835 | ATVDVRLIY | 912 | YLEDVRLIH | 831 | DVRLI | 821 | −2.00 | −0.43 | |
| P04626-V8421 | 836 | RGTVRLIHI | 913 | LEDVRLIHR | 832 | VRLIH | 822 | −2.00 | −0.25 | |
| P04626-V8421 | 836 | KGKVRLIHL | 914 | LEDVRLIHR | 832 | VRLIH | 822 | −2.04 | −0.25 | |
| P04626-V8421 | 837 | TAKRLIHRI | 915 | EDVRLIHRD | 833 | RLIHR | 823 | −2.06 | −0.26 | |
| P04626-V8421 | 837 | ATQRLIHRY | 916 | EDVRLIHRD | 833 | RLIHR | 823 | −2.08 | −0.26 | |
| P04626-V8421 | 838 | MAELIHRDL | 917 | DVRLIHRDL | 834 | IHRD | 824 | −1.99 | −1.24 | |
| P04626-V8421 | 838 | KGILIHRDP | 918 | DVRLIHRDL | 834 | LIHRD | 824 | −2.06 | −1.24 | |

TABLE 14
Bespoke peptides designed for common mutations of ERBB2 for MHC II alleles

| | | | | | | | | | Pre- | |
|---|---|---|---|---|---|---|---|---|---|---|
| | | | | | | | | Pre- | dicted | |
| | | | | | | | | dicted | binding | |
| | | | | | | | | binding | affin- | |
| | | | | | | | | affin- | ity | |
| | | | SEQ | origi- | SEQ | | SEQ | ity | origi- | |
| | | proposed | ID | nating | ID | TCEM | ID | proposed | nating | |
| gi | pos | peptide | NO.: | peptide | NO.: | core | NO.: | peptide | peptide | Allele |
| P04626-R678Q | 668 | DLFLGILIKRQPLPN | 943 | GVVFGILIKRQQQKI | 931 | GI~I~RQ | 919 | -2.08 | -0.30 | DRB1_0701 |
| P04626-R678Q | 668 | MLQYGILIKRQIWRL | 944 | GVVFGILIKRQQQKI | 931 | GI~I~RQ | 919 | −2.03 | −0.30 | |
| P04626-R678Q | 668 | GPLLGILIKRQPLAS | 945 | GVVFGILIKRQQQKI | 931 | GI~I~RQ | 919 | −2.06 | −0.46 | DRB1_0401 |
| P04626-R678Q | 668 | FPLLGILIKRQPLKQ | 946 | GVVFGILIKRQQQKI | 931 | GI~I~RQ | 919 | −2.01 | −0.46 | |
| P04626-R678Q | 669 | ERLLILIKRQQMAAY | 947 | VVFGILIKRQQQKIR | 932 | IL~K~QQ | 920 | −2.02 | −0.35 | |
| P04626-R678Q | 669 | SDWLILIKRQQLLQT | 948 | VVFGILIKRQQQKIR | 932 | IL~K~QQ | 920 | −2.01 | −0.35 | |
| P04626-R678Q | 671 | LIGIIKRQQQKFLPV | 949 | FGILIKRQQQKIRKY | 933 | IK~Q~QK | 921 | −2.05 | −0.97 | |
| P04626-R678Q | 671 | LFLVIKRQQQKLAKF | 950 | FGILIKRQQQKIRKY | 933 | IK~Q~QK | 921 | −2.01 | −0.97 | |
| P04626-R678Q | 668 | FNRFGILIKRQYNQA | 951 | GVVFGILIKRQQQKI | 931 | GI~I~RQ | 919 | −2.01 | −2.83 | DRB1_1101 |
| P04626-R678Q | 668 | TFRLGILIKRQLHTP | 952 | GVVFGILIKRQQQKI | 931 | GI~I~RQ | 919 | −2.01 | −2.83 | |
| P04626-R678Q | 669 | GLQFILIKRQQNFAW | 953 | VVFGILIKRQQQKIR | 932 | IL~K~QQ | 920 | −2.02 | −2.25 | |
| P04626-R678Q | 669 | VYAIILIKRQQENGS | 954 | VVFGILIKRQQQKIR | 932 | IL~K~QQ | 920 | −2.05 | −2.25 | |
| P04626-R678Q | 671 | FLIMIKRQQQKHGTL | 955 | FGILIKRQQQKIRKY | 933 | IK~Q~QK | 921 | −2.02 | −2.76 | |
| P04626-R678Q | 671 | LLLLIKRQQQKLLPS | 956 | FGILIKRQQQKIRKY | 933 | IK~Q~QK | 921 | −2.05 | −2.76 | |
| P04626-R678Q | 668 | LGICGILIKRQLVQE | 957 | GVVFGILIKRQQQKI | 931 | GI~I~RQ | 919 | −2.00 | −2.15 | DRB1_1501 |
| P04626-R678Q | 668 | DGSYGILIKRQMALR | 958 | GVVFGILIKRQQQKI | 931 | GI~I~RQ | 919 | −2.09 | −2.15 | |
| P04626-R678Q | 669 | EALFILIKRQQDGFL | 959 | VVFGILIKRQQQKIR | 932 | IL~K~QQ | 920 | −2.05 | −0.82 | |
| P04626-R678Q | 669 | VPVIILIKRQQYMGE | 960 | VVFGILIKRQQQKIR | 932 | IL~K~QQ | 920 | −2.09 | −0.82 | |
| P04626-R678Q | 671 | PLFLIKRQQQKTFLP | 961 | FGILIKRQQQKIRKY | 933 | IK~Q~QK | 921 | −2.08 | −1.30 | |
| P04626-R678Q | 671 | PLFHIKRQQQKLQPA | 962 | FGILIKRQQQKIRKY | 933 | IK~Q~QK | 921 | −1.99 | −1.30 | |
| P04626-S310F | 300 | KFQFLSTDVGFIWPH | 963 | PYNYLSTDVGFCTLV | 934 | LS~D~GF | 922 | −2.02 | −1.43 | DRB1_0701 |
| P04626-S310F | 300 | NNLYLSTDVGFIFEC | 964 | PYNYLSTDVGFCTLV | 934 | LS~D~GF | 922 | −2.04 | −1.43 | |
| P04626-S310F | 301 | RPPWSTDVGFCLICI | 965 | YNYLSTDVGFCTLVC | 935 | ST~V~FC | 923 | −2.08 | −1.77 | |
| P04626-S310F | 301 | KLIFSTDVGFCPPQI | 966 | YNYLSTDVGFCTLVC | 935 | ST~V~FC | 923 | −2.05 | −1.77 | |
| P04626-S310F | 305 | DDLFGFCTLVCGKMC | 967 | STDVGFCTLVCPLHN | 936 | GF~T~VC | 924 | −2.05 | −0.72 | |
| P04626-S310F | 306 | REFKFCTLVCPLTLY | 968 | TDVGFCTLVCPLHNQ | 937 | FC~L~CP | 925 | −2.02 | −1.14 | |
| P04626-S310F | 306 | TELRFCTLVCPPTYL | 969 | TDVGFCTLVCPLHNQ | 937 | FC~L~CP | 925 | −2.07 | −1.14 | |
| P04626-S310F | 300 | RPAILSTDVGFTTEF | 970 | PYNYLSTDVGFCTLV | 934 | LS~D~GF | 922 | −2.01 | −0.66 | DRB1_0401 |
| P04626-S310F | 300 | PNQWLSTDVGFKPTE | 971 | PYNYLSTDVGFCTLV | 934 | LS~D~GF | 922 | −2.05 | −0.66 | |
| P04626-S310F | 301 | SPFISTDVGFCPPVY | 972 | YNYLSTDVGFCTLVC | 935 | ST~V~FC | 923 | −2.00 | −1.39 | |
| P04626-S310F | 306 | YTVHFCTLVCPRTMR | 973 | TDVGFCTLVCPLHNQ | 937 | FC~L~CP | 925 | −2.02 | −0.77 | |
| P04626-S310F | 306 | STPWFCTLVCPATWE | 974 | TDVGFCTLVCPLHNQ | 937 | FC~L~CP | 925 | −2.01 | −0.77 | |
| P04626-S310F | 300 | TYACLSTDVGFWIRI | 975 | PYNYLSTDVGFCTLV | 934 | LS~D~GF | 922 | −2.09 | −0.40 | DRB1_1501 |
| P04626-S310F | 300 | DQWWLSTDVGFLFPA | 976 | PYNYLSTDVGFCTLV | 934 | LS~D~GF | 922 | −2.05 | −0.40 | |
| P04626-S310F | 305 | KDFFGFCTLVCLGPD | 977 | STDVGFCTLVCPLHN | 936 | GF~T~VC | 924 | −1.99 | −0.66 | |
| P04626-S310F | 306 | SDHCFCTLVCPNFYA | 978 | TDVGFCTLVCPLHNQ | 937 | FC~L~CP | 925 | −2.06 | −0.64 | |
| P04626-S310F | 306 | REIFFCTLVCPFRWR | 979 | TDVGFCTLVCPLHNQ | 937 | FC~L~CP | 925 | −2.00 | −0.64 | |
| P04626-V8421 | 832 | PDTPLEDVRLIVVWP | 980 | GMSYLEDVRLIHRDL | 938 | LE~V~LI | 926 | −2.03 | −0.25 | DRB1_0701 |
| P04626-V8421 | 832 | AYELLEDVRLIPLEN | 981 | GMSYLEDVRLIHRDL | 938 | LE~V~LI | 926 | −2.07 | −0.25 | |
| P04626-V8421 | 833 | PTPWEDVRLIHLLLG | 982 | MSYLEDVRLIHRDLA | 939 | ED~R~IH | 927 | −2.06 | −0.06 | |
| P04626-V8421 | 833 | QFSYEDVRLIHLWIM | 983 | MSYLEDVRLIHRDLA | 939 | ED~R~IH | 927 | −2.06 | −0.06 | |
| P04626-V8421 | 837 | SMPHLIHRDLALPTF | 984 | EDVRLIHRDLAARNV | 940 | LI~R~LA | 928 | −2.00 | −0.91 | |
| P04626-V8421 | 837 | LPIELIHRDLAHYQV | 985 | EDVRLIHRDLAARNV | 940 | LI~R~LA | 928 | −2.01 | −0.91 | |
| P04626-V8421 | 838 | ESWRIHRDLAAAYVC | 986 | DVRLIHRDLAARNVL | 941 | IH~D~AA | 929 | −2.02 | −1.06 | |
| P04626-V8421 | 838 | PWTRIHRDLAALSLG | 987 | DVRLIHRDLAARNVL | 941 | IH~D~AA | 929 | −2.00 | −1.06 | |
| P04626-V8421 | 832 | PWFELEDVRLITAMG | 988 | GMSYLEDVRLIHRDL | 938 | LE~V~LI | 926 | −2.04 | −0.63 | DRB1_0401 |
| P04626-V8421 | 832 | LTDFLEDVRLIGPIQ | 989 | GMSYLEDVRLIHRDL | 938 | LE~V~LI | 926 | −2.04 | −0.63 | |
| P04626-V8421 | 835 | IKLLVRLIHRDGWSV | 990 | YLEDVRLIHRDLAAR | 942 | VR~I~RD | 930 | −2.03 | −0.96 | |
| P04626-V8421 | 835 | RTLLVRLIHRDYSYV | 991 | YLEDVRLIHRDLAAR | 942 | VR~I~RD | 930 | −2.04 | −0.96 | |
| P04626-V8421 | 837 | LPFILIHRDLAGKNC | 992 | EDVRLIHRDLAARNV | 940 | LI~R~LA | 928 | −2.03 | −0.67 | |
| P04626-V8421 | 837 | QAYFLIHRDLAVEDQ | 993 | EDVRLIHRDLAARNV | 940 | LI~R~LA | 928 | −2.03 | −0.67 | |
| P04626-V8421 | 838 | ELFQIHRDLAASRLT | 994 | DVRLIHRDLAARNVL | 941 | IH~D~AA | 929 | −2.07 | −1.83 | |
| P04626-V8421 | 838 | PERWIHRDLAADGCF | 995 | DVRLIHRDLAARNVL | 941 | IH~D~AA | 929 | −2.00 | −1.83 | |
| P04626-V8421 | 835 | IGLFVRLIHRDLSAT | 996 | YLEDVRLIHRDLAAR | 942 | VR~I~RD | 930 | −2.03 | −1.54 | DRB1_1101 |
| P04626-V8421 | 835 | AMFIVRLIHRDGPWS | 997 | YLEDVRLIHRDLAAR | 942 | VR~I~RD | 930 | −2.01 | −1.54 | |
| P04626-V8421 | 837 | QIYRLIHRDLAPALA | 998 | EDVRLIHRDLAARNV | 940 | LI~R~LA | 928 | −2.04 | −2.10 | |
| P04626-V8421 | 837 | DHSWLIHRDLANRLR | 999 | EDVRLIHRDLAARNV | 940 | LI~R~LA | 928 | −2.06 | −2.10 | |
| P04626-V8421 | 838 | NLILIHRDLAAQKPP | 1000 | DVRLIHRDLAARNVL | 941 | IH~D~AA | 929 | −2.02 | −1.32 | |
| P04626-V8421 | 838 | EYILIHRDLAAPDEH | 1001 | DVRLIHRDLAARNVL | 941 | IH~D~AA | 929 | −2.02 | −1.32 | |
| P04626-V8421 | 833 | KPRFEDVRLIHIPPT | 1002 | MSYLEDVRLIHRDLA | 939 | ED~R~IH | 927 | −2.10 | −0.61 | DRB1_1501 |
| P04626-V8421 | 833 | TVVFEDVRLIHYWQL | 1003 | MSYLEDVRLIHRDLA | 939 | ED~R~IH | 927 | −1.99 | −0.61 | |
| P04626-V8421 | 835 | MDLCVRLIHRDLALY | 1004 | YLEDVRLIHRDLAAR | 942 | VR~I~RD | 930 | −2.00 | −0.52 | |
| P04626-V8421 | 835 | NSPIVRLIHRDNQFE | 1005 | YLEDVRLIHRDLAAR | 942 | VR~I~RD | 930 | −2.04 | −0.52 | |
| P04626-V8421 | 837 | RQIVLIHRDLAEISH | 1006 | EDVRLIHRDLAARNV | 940 | LI~R~LA | 928 | −2.10 | −1.48 | |
| P04626-V8421 | 837 | DPHYLIHRDLARVPT | 1007 | EDVRLIHRDLAARNV | 940 | LI~R~LA | 928 | −2.00 | −1.48 | |
| P04626-V8421 | 838 | TFLWIHRDLAAFRQS | 1008 | DVRLIHRDLAARNVL | 941 | IH~D~AA | 929 | −1.98 | −1.11 | |
| P04626-V8421 | 838 | NPGFIHRDLAAPHYI | 1009 | DVRLIHRDLAARNVL | 941 | IH~D~AA | 929 | −2.07 | −1.11 | |

### Example 12: Bespoke Peptides Targeting Common Mutations in PIK3CA

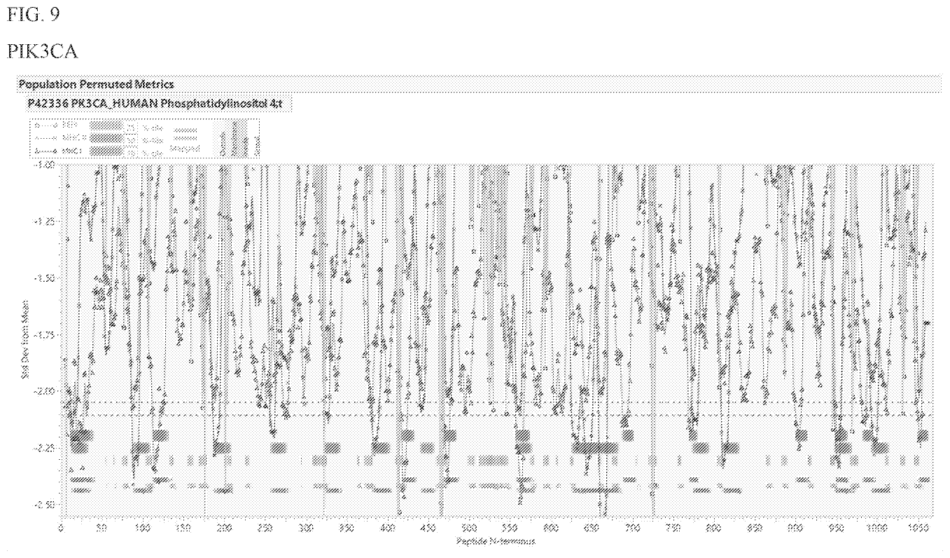

PIK3CA has long been recognized as a critical oncogene [76]. Phosphoinositide-3-kinase (PI3K) activates signaling cascades involved in cell growth, survival, proliferation, motility and morphology. PI3K has two subunits, catalytic and inhibitory. PIK3CA, the gene that encodes the catalytic subunit is a highly mutated protein in cancer. Of various types. Alterations in the PIK3CA gene are associated with poor prognosis of solid tumors. Most of the cancer-associated mutations are missense mutations. The most common are E542K; E545K and H1047R. Mutated isoforms participate in cellular transformation and tumorigenesis induced by oncogenic receptor tyrosine kinases (RTKs) and HRAS/KRAS. The amino acid sequence P42336 is the canonical sequence for PIK3CA, although other potential isoforms are recognized. FIG. 9 provides an overview of the immunome features. The common mutations noted above were mapped and bespoke peptides designed for a variety of example alleles. As noted in the above examples, the same approach could be applied to other mutations of this gene and to design peptides with a desired binding affinity for other alleles therefore the examples are considered non-limiting.

TABLE 15
Bespoke peptides designed for common mutations of PIK3CA for MHC I alleles

| | | | | | | | | | pre- | |
|---|---|---|---|---|---|---|---|---|---|---|
| | | | | | | | | pre- | dicted | |
| | | | | | | | | dicted | affinity | |
| | | | SEQ | origi- | SEQ | | SEQ | affinity | origi- | |
| | | proposed | ID | nating | ID | TCEM | ID | proposed | nating | |
| gi | pos | peptide | NO.: | peptide | NO.: | core | NO.: | peptide | peptide | Allele |
| P42336-E542K | 535 | INVDPLSKV | 1030 | STRDPLSKI | 1020 | DPLSK | 1010 | -2.03 | -0.18 | A0201 |
| P42336-E542K | 535 | IFSDPLSKL | 1031 | STRDPLSKI | 1020 | DPLSK | 1010 | −2.08 | −0.18 | |
| P42336-E542K | 535 | NLQDPLSKM | 1032 | STRDPLSKI | 1020 | DPLSK | 1010 | −2.01 | −1.76 | A2601 |
| P42336-E542K | 535 | EHLDPLSKY | 1033 | STRDPLSKI | 1020 | DPLSK | 1010 | −2.06 | −1.76 | |
| P42336-E542K | 536 | WSEPLSKIY | 1034 | TRDPLSKIT | 1021 | PLSKI | 1011 | −2.03 | 0.05 | |
| P42336-E542K | 536 | MVRPLSKIP | 1035 | TRDPLSKIT | 1021 | PLSKI | 1011 | −2.04 | 0.05 | |
| P42336-E542K | 537 | ENFLSKITM | 1036 | RDPLSKITE | 1022 | LSKIT | 1012 | −2.01 | −0.36 | |
| P42336-E542K | 537 | DNILSKITW | 1037 | RDPLSKITE | 1022 | LSKIT | 1012 | −2.02 | −0.36 | |
| P42336-E542K | 538 | LIPSKITEF | 1038 | DPLSKITEQ | 1023 | SKITE | 1013 | −2.00 | −0.94 | |
| P42336-E542K | 538 | MTKSKITEI | 1039 | DPLSKITEQ | 1023 | SKITE | 1013 | −2.02 | −0.94 | |
| P42336-E542K | 535 | LAKDPLSKK | 1040 | STRDPLSKI | 1020 | DPLSK | 1010 | −2.02 | −1.11 | A3001 |
| P42336-E542K | 535 | KGYDPLSKQ | 1041 | STRDPLSKI | 1020 | DPLSK | 1010 | −2.03 | −1.11 | |
| P42336-E542K | 537 | PYRLSKITE | 1042 | RDPLSKITE | 1022 | LSKIT | 1012 | −2.04 | −0.81 | |
| P42336-E542K | 537 | PNHLSKITA | 1043 | RDPLSKITE | 1022 | LSKIT | 1012 | −2.04 | −0.81 | |
| P42336-E542K | 535 | LLRDPLSKL | 1044 | STRDPLSKI | 1020 | DPLSK | 1010 | −2.01 | −1.22 | B0702 |
| P42336-E542K | 535 | LARDPLSKF | 1045 | STRDPLSKI | 1020 | DPLSK | 1010 | −2.05 | −1.22 | |
| P42336-E542K | 537 | PPNLSKITH | 1046 | RDPLSKITE | 1022 | LSKIT | 1012 | −2.04 | −0.39 | |
| P42336-E542K | 537 | DGPLSKITL | 1047 | RDPLSKITE | 1022 | LSKIT | 1012 | −2.02 | −0.39 | |
| P42336-E542K | 535 | PLKDPLSKL | 1048 | STRDPLSKI | 1020 | DPLSK | 1010 | −2.04 | −1.65 | B0801 |
| P42336-E542K | 535 | GLKDPLSKL | 1049 | STRDPLSKI | 1020 | DPLSK | 1010 | −2.06 | −1.65 | |
| P42336-E542K | 535 | EDQDPLSKF | 1050 | STRDPLSKI | 1020 | DPLSK | 1010 | −2.02 | −0.17 | B1501 |
| P42336-E542K | 535 | GDGDPLSKF | 1051 | STRDPLSKI | 1020 | DPLSK | 1010 | −2.04 | −0.17 | |
| P42336-E542K | 535 | VSQDPLSKY | 1052 | STRDPLSKI | 1020 | DPLSK | 1010 | −2.02 | −0.56 | |
| P42336-E542K | 535 | HLTDPLSKW | 1053 | STRDPLSKI | 1020 | DPLSK | 1010 | −2.00 | −0.56 | B5701 |
| P42336-E542K | 538 | FGRSKITER | 1054 | DPLSKITEQ | 1023 | SKITE | 1013 | −2.01 | 0.08 | |
| P42336-E542K | 538 | ETRSKITEF | 1055 | DPLSKITEQ | 1023 | SKITE | 1013 | −2.03 | 0.08 | |
| P42336-E545K | 538 | EITSEITKS | 1056 | DPLSEITKQ | 1024 | SEITK | 1014 | −2.00 | −1.37 | A2601 |
| P42336-E545K | 538 | LTGSEITKL | 1057 | DPLSEITKQ | 1024 | SEITK | 1014 | −2.06 | −1.37 | |
| P42336-E545K | 540 | KVPITKQEA | 1058 | LSEITKQEK | 1025 | ITKQE | 1015 | −2.07 | −0.32 | A3001 |
| P42336-E545K | 540 | DFKITKQEA | 1059 | LSEITKQEK | 1025 | ITKQE | 1015 | −2.05 | −0.32 | |
| P42336-E545K | 540 | IAFITKQEI | 1060 | LSEITKQEK | 1025 | ITKQE | 1015 | −2.01 | 0.08 | B5701 |
| P42336-E545K | 540 | AGYITKQEI | 1061 | LSEITKQEK | 1025 | ITKQE | 1015 | −2.07 | 0.08 | |
| P42336-H1047R | 1040 | ALPMNDARL | 1062 | MKQMNDARH | 1026 | MNDAR | 1016 | −2.09 | −0.09 | A0201 |
| P42336-H1047R | 1040 | FGLMNDARV | 1063 | MKQMNDARH | 1026 | MNDAR | 1016 | −2.06 | −0.09 | |
| P42336-H1047R | 1041 | GLPNDARHL | 1064 | KQMNDARHG | 1027 | NDARH | 1017 | −2.02 | −0.45 | |
| P42336-H1047R | 1041 | TIWNDARHL | 1065 | KQMNDARHG | 1027 | NDARH | 1017 | −2.04 | −0.45 | |
| P42336-H1047R | 1042 | YLTDARHGF | 1066 | QMNDARHGG | 1028 | DARHG | 1018 | −2.08 | −0.36 | |
| P42336-H1047R | 1042 | IIMDARHGL | 1067 | QMNDARHGG | 1028 | DARHG | 1018 | −2.01 | −0.36 | |
| P42336-H1047R | 1043 | SLLARHGGA | 1068 | MNDARHGGW | 1029 | ARHGG | 1019 | −2.10 | −0.08 | |
| P42336-H1047R | 1043 | LIGARHGGV | 1069 | MNDARHGGW | 1029 | ARHGG | 1019 | −2.09 | −0.08 | |
| P42336-H1047R | 1043 | RTSARHGGI | 1070 | MNDARHGGW | 1029 | ARHGG | 1019 | −2.00 | −0.57 | A2601 |
| P42336-H1047R | 1043 | KVMARHGGL | 1071 | MNDARHGGW | 1029 | ARHGG | 1019 | −2.02 | −0.57 | |
| P42336-H1047R | 1040 | TGPMNDARS | 1072 | MKQMNDARH | 1026 | MNDAR | 1016 | −2.06 | 0.26 | A3001 |
| P42336-H1047R | 1040 | ALKMNDARS | 1073 | MKQMNDARH | 1026 | MNDAR | 1016 | −2.04 | 0.26 | |
| P42336-H1047R | 1041 | YLRNDARHT | 1074 | KQMNDARHG | 1027 | NDARH | 1017 | −1.99 | −0.76 | |
| P42336-H1047R | 1041 | PARNDARHL | 1075 | KQMNDARHG | 1027 | NDARH | 1017 | −2.01 | −0.76 | |
| P42336-H1047R | 1042 | KLWDARHGK | 1076 | QMNDARHGG | 1028 | DARHG | 1018 | −2.04 | −0.42 | |
| P42336-H1047R | 1042 | DVWDARHGA | 1077 | QMNDARHGG | 1028 | DARHG | 1018 | −2.00 | −0.42 | |
| P42336-H1047R | 1042 | ETADARHGL | 1078 | QMNDARHGG | 1028 | DARHG | 1018 | −2.04 | −1.34 | B0702 |
| P42336-H1047R | 1042 | TSFDARHGL | 1079 | QMNDARHGG | 1028 | DARHG | 1018 | −2.02 | −1.34 | |
| P42336-H1047R | 1043 | KAPARHGGL | 1080 | MNDARHGGW | 1029 | ARHGG | 1019 | −2.05 | −0.11 | |
| P42336-H1047R | 1043 | LAHARHGGG | 1081 | MNDARHGGW | 1029 | ARHGG | 1019 | −2.07 | −0.11 | |
| P42336-H1047R | 1041 | YLRNDARHL | 1082 | KQMNDARHG | 1027 | NDARH | 1017 | −2.01 | −0.19 | B0801 |
| P42336-H1047R | 1042 | FRDARHGL | 1083 | QMNDARHGG | 1028 | DARHG | 1018 | −2.06 | −0.18 | |
| P42336-H1047R | 1042 | WFKDARHGI | 1084 | QMNDARHGG | 1028 | DARHG | 1018 | −2.04 | −0.18 | |
| P42336-H1047R | 1043 | GPQARHGGF | 1085 | MNDARHGGW | 1029 | ARHGG | 1019 | −2.02 | −1.34 | |
| P42336-H1047R | 1043 | RAPARHGGF | 1086 | MNDARHGGW | 1029 | ARHGG | 1019 | −2.05 | −1.34 | |
| P42336-H1047R | 1040 | LLNMNDARY | 1087 | MKQMNDARH | 1026 | MNDAR | 1016 | −2.03 | −0.0 | B1501 |
| P42336-H1047R | 1040 | HVPMNDARF | 1088 | MKQMNDARH | 1026 | MNDAR | 1016 | −2.01 | −0.01 | |
| P42336-H1047R | 1041 | TVLNDARHF | 1089 | KQMNDARHG | 1027 | NDARH | 1017 | −2.02 | −0.70 | |
| P42336-H1047R | 1041 | YARNDARHF | 1090 | KQMNDARHG | 1027 | NDARH | 1017 | −2.02 | −0.70 | |
| P42336-H1047R | 1042 | QFIDARHGF | 1091 | QMNDARHGG | 1028 | DARHG | 1018 | −2.06 | −0.54 | |
| P42336-H1047R | 1042 | NEVDARHGF | 1092 | QMNDARHGG | 1028 | DARHG | 1018 | −2.02 | −0.54 | |
| P42336-H1047R | 1043 | RTGARHGGY | 1093 | MNDARHGGW | 1029 | ARHGG | 1019 | −2.06 | −1.14 | |
| P42336-H1047R | 1043 | VATARHGGY | 1094 | MNDARHGGW | 1029 | ARHGG | 1019 | −2.04 | −1.14 | |
| P42336-H1047R | 1043 | EILARHGGW | 1095 | MNDARHGGW | 1029 | ARHGG | 1019 | −2.01 | −2.41 | B5701 |
| P42336-H1047R | 1043 | HGFARHGGR | 1096 | MNDARHGGW | 1029 | ARHGG | 1019 | −2.06 | −2.41 | |

TABLE 16
Bespoke peptides designed for common mutations of PIK3CA for MHC II alleles

| | | | | | | | | Pre- | Pre- | |
|---|---|---|---|---|---|---|---|---|---|---|
| | | | | | | | | dicted | affin- | |
| | | | | | | | | affin- | ity | |
| | | | SEQ | origi- | SEQ | | SEQ | ity | origi- | |
| | | proposed | ID | nating | ID | TCEM | ID | proposed | nating | |
| gi | pos | peptide | NO.: | peptide | NO.: | core | NO.: | peptide | peptide | Allele |
| P42336-E542K | 537 | DLLWSKITEQERSFC | 1121 | RDPLSKITEQEKDFL | 1109 | SK~T~QE | 1097 | -2.06 | 0.21 | DRB1_0401 |
| P42336-E542K | 537 | YFYLSKITEQEIQQC | 1122 | RDPLSKITEQEKDFL | 1109 | SK~T~QE | 1097 | −2.03 | 0.21 | |
| P42336-E542K | 538 | RLRFKITEQEKQLTL | 1123 | DPLSKITEQEKDFLW | 1110 | KI~E~EK | 1098 | −2.02 | 0.38 | |
| :P42336-E542K | 538 | SLFIKITEQEKLLMA | 1124 | DPLSKITEQEKDFLW | 1110 | KI~E~EK | 1098 | −2.07 | 0.38 | |
| P42336-E542K | 537 | SLFISKITEQEIVYI | 1125 | RDPLSKITEQEKDFL | 1109 | SK~T~QE | 1097 | −2.05 | 0.23 | DRB1_0701 |
| P42336-E542K | 537 | WFLESKITEQEIIIN | 1126 | RDPLSKITEQEKDFL | 1109 | SK~T~QE | 1097 | −2.04 | 0.23 | |
| P42336-E542K | 538 | YLLAKITEQEKLWFL | 1127 | DPLSKITEQEKDFLW | 1110 | KI~E~EK | 1098 | −2.02 | 0.23 | |
| P42336-E542K | 538 | WIWRKITEQEKLNIV | 1128 | DPLSKITEQEKDFLW | 1110 | KI~E~EK | 1098 | −2.05 | 0.23 | |
| P42336-E542K | 532 | IWEFTRDPLSKFCHS | 1129 | KAISTRDPLSKITEQ | 1111 | TR~P~SK | 1099 | −2.05 | 0.39 | DRB1_1101 |
| P42336-E542K | 532 | ESLWTRDPLSKCHIY | 1130 | KAISTRDPLSKITEQ | 1111 | TR~P~SK | 1099 | −2.05 | 0.39 | |
| P42336-E542K | 537 | FWFLSKITEQENMPP | 1131 | RDPLSKITEQEKDFL | 1109 | SK~T~QE | 1097 | −2.05 | −0.21 | |
| P42336-E542K | 537 | YLWLSKITEQEIKHP | 1132 | RDPLSKITEQEKDFL | 1109 | SK~T~QE | 1097 | −2.06 | −0.21 | |
| P42336-E542K | 532 | PLILTRDPLSKRISI | 1133 | KAISTRDPLSKITEQ | 1111 | TR~P~SK | 1099 | −2.00 | 0.62 | DRB1_1501 |
| P42336-E542K | 532 | GYLITRDPLSKFPIT | 1134 | KAISTRDPLSKITEQ | 1111 | TR~P~SK | 1099 | −2.05 | 0.62 | |
| P42336-E545K | 535 | NPIQPLSEITKRSII | 1135 | STRDPLSEITKQEKD | 1112 | PL~E~TK | 1100 | −2.00 | 0.50 | DRB1_0401 |
| P42336-E545K | 535 | KTFIPLSEITKELHT | 1136 | STRDPLSEITKQEKD | 1112 | PL~E~TK | 1100 | −2.04 | 0.50 | |
| P42336-E545K | 536 | DVLFLSEITKQPSSD | 1137 | TRDPLSEITKQEKDF | 1113 | LS~1~KQ | 1101 | −2.05 | −0.01 | |
| P42336-E545K | 536 | ANIFLSEITKQAILF | 1138 | TRDPLSEITKQEKDF | 1113 | LS~I~KQ | 1101 | −2.02 | −0.01 | |
| P42336-E545K | 538 | IQWFEITKQEKLLFY | 1139 | DPLSEITKQEKDFLW | 1114 | EI~K~EK | 1102 | −2.04 | 0.50 | DRB1_0701 |
| P42336-E545K | 538 | LVELEITKQEKLIFI | 1140 | DPLSEITKQEKDFLW | 1114 | EI~K~EK | 1102 | −2.04 | 0.50 | |
| P42336-E545K | 540 | FPNVTKQEKDFIFIF | 1141 | LSEITKQEKDFLWSH | 1115 | TK~E~DF | 1103 | −2.03 | 0.63 | |
| P42336-E545K | 540 | FFIITKQEKDFQILY | 1142 | LSEITKQEKDFLWSH | 1115 | TK~E~DF | 1103 | −2.04 | 0.63 | |
| P42336-E545K | 535 | FNWIPLSEITKEMKG | 1143 | STRDPLSEITKQEKD | 1112 | PL~E~TK | 1100 | −2.04 | 0.28 | DRB1_1101 |
| P42336-E545K | 535 | LLKWPLSEITKLCEN | 1144 | STRDPLSEITKQEKD | 1112 | PL~E~TK | 1100 | −2.07 | 0.28 | |
| P42336-E545K | 536 | YLPFLSEITKQLDVS | 1145 | TRDPLSEITKQEKDF | 1113 | LS~1~KQ | 1101 | −2.05 | 0.37 | |
| P42336-E545K | 536 | KRFLLSEITKQGLGI | 1146 | TRDPLSEITKQEKDF | 1113 | LS~I~KQ | 1101 | −2.03 | 0.37 | |
| P42336-E545K | 538 | IGLLEITKQEKLPLS | 1147 | DPLSEITKQEKDFLW | 1114 | EI~K~EK | 1102 | −2.05 | 0.08 | |
| P42336-E545K | 538 | IPTFEITKQEKISKS | 1148 | DPLSEITKQEKDFLW | 1114 | EI~K~EK | 1102 | −2.05 | 0.08 | |
| P42336-E545K | 538 | LRIFEITKQEKWLWF | 1149 | DPLSEITKQEKDFLW | 1114 | EI~K~EK | 1102 | −2.06 | 0.82 | DRB1_1501 |
| P42336-E545K | 538 | KLWVEITKQEKLFKI | 1150 | DPLSEITKQEKDFLW | 1114 | EI~K~EK | 1102 | −2.06 | 0.82 | |
| P42336-E545K | 540 | QALITKQEKDFLQFL | 1151 | LSEITKQEKDFLWSH | 1115 | TK~E~DF | 1103 | −2.01 | 0.32 | |
| P42336-E545K | 540 | SDIFTKQEKDFLIYI | 1152 | LSEITKQEKDFLWSH | 1115 | TK~E~DF | 1103 | −2.00 | 0.32 | |
| P42336-E545K | 541 | NEIVKQEKDFLLMMG | 1153 | SEITKQEKDFLWSHR | 1116 | KQ~K~FL | 1104 | −2.01 | 0.25 | |
| P42336-E545K | 541 | ELFWKQEKDFLPLRR | 1154 | SEITKQEKDFLWSHR | 1116 | KQ~K~FL | 1104 | −2.04 | 0.25 | |
| P42336-H1047R | 1037 | EFLFKQMNDARAQIK | 1155 | EYFMKQMNDARHGGW | 1117 | KQ~N~AR | 1105 | −2.02 | −0.77 | DRB1_0401 |
| P42336-H1047R | 1037 | LILFKQMNDARKIFD | 1156 | EYFMKQMNDARHGGW | 1117 | KQ~N~AR | 1105 | −2.05 | −0.77 | |
| P42336-H1047R | 1038 | IFLFQMNDARHAPFI | 1157 | YFMKQMNDARHGGWT | 1118 | QM~D~RH | 1106 | −2.06 | −0.74 | |
| P42336-H1047R | 1038 | YAWFQMNDARHLSEM | 1158 | YFMKQMNDARHGGWT | 1118 | QM~D~RH | 1106 | −2.01 | −0.74 | |
| P42336-H1047R | 1037 | WLIMKQMNDARLVFV | 1159 | EYFMKQMNDARHGGW | 1117 | KQ~N~AR | 1105 | −2.01 | −0.79 | DRB1_1101 |
| P42336-H1047R | 1037 | LLYLKQMNDARYKSR | 1160 | EYFMKQMNDARHGGW | 1117 | KQ~N~AR | 1105 | −2.02 | −0.79 | |
| P42336-H1047R | 1038 | ILFWQMNDARHENMD | 1161 | YFMKQMNDARHGGWT | 1118 | QM~D~RH | 1106 | −2.01 | −0.07 | |
| P42336-H1047R | 1038 | ILRYQMNDARHAFPL | 1162 | YFMKQMNDARHGGWT | 1118 | QM~D~RH | 1106 | −2.03 | −0.07 | |
| P42336-H1047R | 1040 | YFIWNDARHGGLDLQ | 1163 | MKQMNDARHGGWTTK | 1119 | ND~R~GG | 1107 | −2.04 | 0.36 | |
| P42336-H1047R | 1040 | LLWLNDARHGGLLRA | 1164 | MKQMNDARHGGWTTK | 1119 | ND~R~GG | 1107 | −2.02 | 0.36 | |
| P42336-H1047R | 1043 | LQIFRHGGWTTMKKQ | 1165 | MNDARHGGWTTKMDW | 1120 | RH~G~TT | 1108 | −2.03 | 0.32 | |
| P42336-H1047R | 1043 | KSYIRHGGWTTCERL | 1166 | MNDARHGGWTTKMDW | 1120 | RH~G~TT | 1108 | −2.05 | 0.32 | |
| P42336-H1047R | 1038 | ALQIQMNDARHVIYI | 1167 | YFMKQMNDARHGGWT | 1118 | QM~D~RH | 1106 | −2.02 | 0.70 | DRB1_1501 |
| P42336-H1047R | 1038 | SYLYQMNDARHLLIL | 1168 | YFMKQMNDARHGGWT | 1118 | QM~D~RH | 1106 | −2.02 | 0.70 | |

### Example 13: Bespoke Peptides Targeting Common Mutations in KRAS

Ras proteins (rat sarcoma family), including KRAS, bind GDP/GTP and possess intrinsic GTPase activity they therefore have an important role in the regulation of cell proliferation. KRAS is the most commonly mutated oncogene in cancer, functioning by silencing of tumor suppressor genes [77]. Approximately 90% of KRAS mutations are at position 12, although the distribution of mutations at this position varies between cancer types. G12C and G12V are very common in non-small cell lung cancer (smoking induced); whereas G12D is common in Colon cancer. The G12 and 13 positions are in the P loop of KRAS where they stabilize nucleotides, but differ in their effect on nucleotide exchange. In contrast; the Q61 position participates in conformational changes during the interconversion between structural states [78]. Hence the exact mutation has a strong effect on oncogenic function and prognosis. As yet there are no drugs which directly target KRAS [79].

The canonical sequence of human KRAS is P01116 (uniprot.org). FIG. 9 provides an overview of the immunome features. The common mutations noted above, G12V, G12D, G12C, G13D and Q61H, were mapped and bespoke peptides designed for a variety of example alleles. As noted in the above examples, the same approach could be applied to other mutations of this gene and to design peptides with a desired binding affinity for other alleles therefore the examples are considered non-limiting. It will be noted that in a few positions and for a few alleles the predicted binding in the bespoke is not significantly different for that in the originating peptide; in such situations the natural originating peptide would be selected for inclusion in the personalized vaccine.

TABLE 17
Bespoke peptides designed for common mutations of KRAS for MHC I alleles

| | | | | | | | | | Pre- | |
|---|---|---|---|---|---|---|---|---|---|---|
| | | | | | | | | Pre- | dicted | |
| | | | | | | | | dicted | binding | |
| | | | | | | | | binding | affin- | |
| | | | | | | | | affin- | ity | |
| | | | SEQ | origi- | SEQ | | SEQ | ity | origi- | |
| | | proposed | ID | nating | ID | TCEM | ID | proposed | nating | |
| gi | pos | peptide | NO.: | peptide | NO.: | core | NO.: | peptide | peptide | Allele |
| P01116_G12V | 5 | NMAVVGAVA | 2309 | KLVVVGAVG | 2289 | VVGAV | 2269 | -2.06 | -1.96 | A0201 |
| P01116_G12V | 5 | TLQVVGAVA | 2310 | KLVVVGAVG | 2289 | VVGAV | 2269 | −2.00 | −1.96 | |
| P01116_G12V | 6 | RITVGAVGV | 2311 | LVVVGAVGV | 2290 | VGAVG | 2270 | −2.05 | −2.51 | |
| P01116_G12V | 6 | DLAVGAVGV | 2312 | LVVVGAVGV | 2290 | VGAVG | 2270 | −2.05 | −2.51 | |
| P01116_G12V | 7 | ELPGAVGVV | 2313 | VVVGAVGVG | 2291 | GAVGV | 2271 | −2.01 | −1.30 | |
| P01116_G12V | 7 | AQIGAVGVV | 2314 | VVVGAVGVG | 2291 | GAVGV | 2271 | −2.04 | −1.30 | |
| P01116_G12V | 6 | KGTVGAVGP | 2315 | LVVVGAVGV | 2290 | VGAVG | 2270 | −2.00 | −0.13 | A1101 |
| P01116_G12V | 6 | ATSVGAVGY | 2316 | LVVVGAVGV | 2290 | VGAVG | 2270 | −2.06 | −0.13 | |
| P01116_G12V | 8 | RVGAVGVGP | 2317 | VVGAVGVGK | 2292 | AVGVG | 2272 | −2.01 | −2.54 | |
| P01116_G12V | 8 | QGYAVGVGP | 2318 | VVGAVGVGK | 2292 | AVGVG | 2272 | −2.05 | −2.54 | |
| P01116_G12V | 6 | ITTVGAVGY | 2319 | LVVVGAVGV | 2290 | VGAVG | 2270 | −2.08 | −1.49 | A2601 |
| P01116_G12V | 6 | QTPVGAVGY | 2320 | LVVVGAVGV | 2290 | VGAVG | 2270 | −2.03 | −1.49 | |
| P01116_G12V | 7 | YDVGAVGVM | 2321 | VVVGAVGVG | 2291 | GAVGV | 2271 | −2.07 | −0.97 | |
| P01116_G12V | 7 | RIYGAVGVM | 2322 | VVVGAVGVG | 2291 | GAVGV | 2271 | −2.07 | −0.97 | |
| P01116_G12V | 8 | RVGAVGVGF | 2323 | VVGAVGVGK | 2292 | AVGVG | 2272 | −2.03 | −0.86 | |
| P01116_G12V | 8 | EKGAVGVGY | 2324 | VVGAVGVGK | 2292 | AVGVG | 2272 | −2.03 | −0.86 | |
| P01116_G12V | 5 | YAKVVGAVQ | 2325 | KLVVVGAVG | 2289 | VVGAV | 2269 | −2.03 | −0.83 | A3001 |
| P01116_G12V | 5 | NAHVVGAVG | 2326 | KLVVVGAVG | 2289 | VVGAV | 2269 | −2.06 | −0.83 | |
| P01116_G12V | 7 | QTRGAVGVV | 2327 | VVVGAVGVG | 2291 | GAVGV | 2271 | −2.04 | 0.09 | |
| P01116_G12V | 7 | PTKGAVGVK | 2328 | VVVGAVGVG | 2291 | GAVGV | 2271 | −2.05 | 0.09 | |
| P01116_G12V | 6 | RALVGAVGF | 2329 | LVVVGAVGV | 2290 | VGAVG | 2270 | −2.08 | −1.32 | B0702 |
| P01116_G12V | 6 | HIQVGAVGL | 2330 | LVVVGAVGV | 2290 | VGAVG | 2270 | −2.08 | 1.32 | |
| P01116_G12V | 7 | RGQGAVGVV | 2331 | VVVGAVGVG | 2291 | GAVGV | 2271 | −2.07 | −0.46 | |
| P01116_G12V | 7 | HVDGAVGVL | 2332 | VVVGAVGVG | 2291 | GAVGV | 2271 | −2.05 | −0.46 | |
| P01116_G12V | 5 | EVRVVGAVV | 2333 | KLVVVGAVG | 2289 | VVGAV | 2269 | −2.02 | 0.17 | B0801 |
| P01116_G12V | 5 | RVRVVGAVL | 2334 | KLVVVGAVG | 2289 | VVGAV | 2269 | −2.00 | 0.17 | |
| P01116_G12V | 6 | KIKVGAVGI | 2335 | LVVVGAVGV | 2290 | VGAVG | 2270 | −2.03 | −0.40 | |
| P01116_G12V | 6 | VGRVGAVGL | 2336 | LVVVGAVGV | 2290 | VGAVG | 2270 | −2.03 | −0.40 | |
| P01116_G12V | 7 | FGRGAVGVV | 2337 | VVVGAVGVG | 2291 | GAVGV | 2271 | −2.02 | 0.29 | |
| P01116_G12V | 5 | QIQVVGAVY | 2338 | KLVVVGAVG | 2289 | VVGAV | 2269 | −2.07 | −0.55 | B1501 |
| P01116_G12V | 5 | EDKVVGAVF | 2339 | KLVVVGAVG | 2289 | VVGAV | 2269 | −2.06 | −0.55 | |
| P01116_G12V | 6 | TGLVGAVGY | 2340 | LVVVGAVGV | 2290 | VGAVG | 2270 | −2.01 | −1.58 | |
| P01116_G12V | 6 | EEFVGAVGF | 2341 | LVVVGAVGV | 2290 | VGAVG | 2270 | −2.04 | −1.58 | |
| P01116_G12V | 7 | TGQGAVGVY | 2342 | VVVGAVGVG | 2291 | GAVGV | 2271 | −2.02 | −0.80 | |
| P01116_G12V | 7 | TFPGAVGVY | 2343 | VVVGAVGVG | 2291 | GAVGV | 2271 | −2.05 | −0.80 | |
| P01116_G12V | 6 | LGEVGAVGM | 2344 | LVVVGAVGV | 2290 | VGAVG | 2270 | −2.03 | −0.27 | B5701 |
| P01116_G12V | 6 | RSYVGAVGL | 2345 | LVVVGAVGV | 2290 | VGAVG | 2270 | −2.02 | −0.27 | |
| P01116-G12C | 5 | GLEVVGACV | 2346 | KLVVVGACG | 2293 | VVGAC | 2273 | −2.05 | −1.70 | A0201 |
| P01116-G120 | 5 | KLRVVGACL | 2347 | KLVVVGACG | 2293 | VVGAC | 2273 | −2.06 | −1.70 | |
| P01116-G12C | 6 | MMYVGACGT | 2348 | LVVVGACGV | 2294 | VGACG | 2274 | −2.01 | −2.50 | |
| P01116-G12C | 6 | TMRVGACGL | 2349 | LVVVGACGV | 2294 | VGACG | 2274 | −2.05 | −2.50 | |
| P01116-G12C | 7 | SMKGACGVI | 2350 | VVVGACGVG | 2295 | GACGV | 2275 | −2.05 | −1.00 | |
| P01116-G12C | 7 | RLFGACGVS | 2351 | VVVGACGVG | 2295 | GACGV | 2275 | −2.03 | −1.00 | |
| P01116-G12C | 6 | YSKVGACGK | 2352 | LVVVGACGV | 2294 | VGACG | 2274 | −2.01 | −0.32 | A1101 |
| P01116-G12C | 6 | EASVGACGK | 2353 | LVVVGACGV | 2294 | VGACG | 2274 | −2.01 | −0.32 | |
| P01116-G12C | 8 | RQAACGVGK | 2354 | VVGACGVGK | 2296 | ACGVG | 2276 | −2.01 | −2.36 | |
| P01116-G12C | 8 | STNACGVGR | 2355 | VVGACGVGK | 2296 | ACGVG | 2276 | −2.04 | −2.36 | |
| P01116-G12C | 6 | DPIVGACGY | 2356 | LVVVGACGV | 2294 | VGACG | 2274 | −2.03 | −0.87 | A2601 |
| P01116-G12C | 6 | HLAVGACGY | 2357 | LVVVGACGV | 2294 | VGACG | 2274 | −2.10 | −0.87 | |
| P01116-G12C | 7 | ELPGACGVR | 2358 | VVVGACGVG | 2295 | GACGV | 2275 | −2.08 | −0.46 | |
| P01116-G12C | 7 | TGQGACGVY | 2359 | VVVGACGVG | 2295 | GACGV | 2275 | −2.07 | −0.46 | |
| P01116-G12C | 8 | QIRACGVGF | 2360 | VVGACGVGK | 2296 | ACGVG | 2276 | −2.04 | −1.53 | |
| P01116-G12C | 8 | SSIACGVGM | 2361 | VVGACGVGK | 2296 | ACGVG | 2276 | −2.09 | −1.53 | |
| P01116-G12C | 9 | HILCGVGKK | 2362 | VGACGVGKS | 2297 | CGVGK | 2277 | −2.02 | −0.84 | |
| P01116-G12C | 9 | HIFCGVGKK | 2363 | VGACGVGKS | 2297 | CGVGK | 2277 | −2.02 | −0.84 | |
| P01116-G12C | 5 | KQKVVGACS | 2364 | KLVVVGACG | 2293 | VVGAC | 2273 | −2.02 | −0.53 | A3001 |
| P01116-G12C | 5 | RAKVVGACD | 2365 | KLVVVGACG | 2293 | VVGAC | 2273 | −2.03 | −0.53 | |
| P01116-G12C | 6 | VARVGACGK | 2366 | LVVVGACGV | 2294 | VGACG | 2274 | −2.02 | 0.11 | |
| P01116-G12C | 6 | KGQVGACGS | 2367 | LVVVGACGV | 2294 | VGACG | 2274 | −2.01 | 0.11 | |
| P01116-G12C | 7 | YARGACGVK | 2368 | VVVGACGVG | 2295 | GACGV | 2275 | −2.01 | −0.19 | |
| P01116-G12C | 7 | YGRGACGVA | 2369 | VVVGACGVG | 2295 | GACGV | 2275 | −2.04 | −0.19 | |
| P01116-G12C | 9 | KTQCGVGKP | 2370 | VGACGVGKS | 2297 | CGVGK | 2277 | −2.02 | −0.33 | |
| P01116-G12C | 9 | GAKCGVGKT | 2371 | VGACGVGKS | 2297 | CGVGK | 2277 | −2.01 | −0.33 | |
| P01116-G12C | 6 | FIRVGACGY | 2372 | LVVVGACGV | 2294 | VGACG | 2274 | −2.02 | −1.42 | B0702 |
| P01116-G12C | 6 | KGHVGACGI | 2373 | LVVVGACGV | 2294 | VGACG | 2274 | −2.07 | −1.42 | |
| P01116-G12C | 7 | FPTGACGVG | 2374 | VVVGACGVG | 2295 | GACGV | 2275 | −2.07 | −0.62 | |
| P01116-G12C | 7 | SSRGACGVL | 2375 | VVVGACGVG | 2295 | GACGV | 2275 | −2.07 | −0.62 | |
| P01116-G12C | 8 | TAQACGVGI | 2376 | VVGACGVGK | 2296 | ACGVG | 2276 | −2.04 | −0.06 | |
| P01116-G12C | 8 | NIRACGVGT | 2377 | VVGACGVGK | 2296 | ACGVG | 2276 | −2.06 | −0.06 | |
| P01116-G12C | 9 | FVRCGVGKV | 2378 | VGACGVGKS | 2297 | CGVGK | 2277 | −2.05 | −0.19 | |
| P01116-G12C | 9 | KIRCGVGKI | 2379 | VGACGVGKS | 2297 | CGVGK | 2277 | −2.00 | −0.19 | |
| P01116-G12C | 6 | MLKVGACGM | 2380 | LVVVGACGV | 2294 | VGACG | 2274 | −2.02 | −0.13 | B0801 |
| P01116-G12C | 6 | YVKVGACGL | 2381 | LVVVGACGV | 2294 | VGACG | 2274 | −2.03 | −0.13 | |
| P01116-G12C | 6 | DSEVGACGF | 2382 | LVVVGACGV | 2294 | VGACG | 2274 | −2.04 | −1.56 | B1501 |
| P01116-G12C | 6 | ENGVGACGF | 2383 | LVVVGACGV | 2294 | VGACG | 2274 | −2.04 | 1.56 | |
| P01116-G12C | 7 | STRGACGVY | 2384 | VVVGACGVG | 2295 | GACGV | 2275 | −2.04 | −1.01 | |
| P01116-G12C | 7 | TVGGACGVY | 2385 | VVVGACGVG | 2295 | GACGV | 2275 | −2.06 | −1.01 | |
| P01116-G12C | 5 | PAGVVGACY | 2386 | KLVVVGACG | 2293 | VVGAC | 2273 | −2.03 | −0.12 | B5701 |
| P01116-G12C | 5 | NGTVVGACY | 2387 | KLVVVGACG | 2293 | VVGAC | 2273 | −2.07 | −0.12 | |
| P01116-G12C | 6 | DNYVGACGF | 2388 | LVVVGACGV | 2294 | VGACG | 2274 | −2.00 | −0.29 | |
| P01116-G12C | 6 | QSIVGACGL | 2389 | LVVVGACGV | 2294 | VGACG | 2274 | −2.07 | −0.29 | |
| P01116-G12C | 7 | EGSGACGVY | 2390 | VVVGACGVG | 2295 | GACGV | 2275 | −2.04 | −0.12 | |
| P01116-G12C | 7 | SADGACGVY | 2391 | VVVGACGVG | 2295 | GACGV | 2275 | −2.03 | −0.12 | |
| P01116-G12D | 5 | TTIVVGADV | 2392 | KLVVVGADG | 2298 | VVGAD | 2278 | −2.05 | −2.00 | A0201 |
| P01116-G12D | 5 | AQLVVGADV | 2393 | KLVVVGADG | 2298 | VVGAD | 2278 | −2.04 | −2.00 | |
| P01116-G12D | 6 | SIMVGADGV | 2394 | LVVVGADGV | 2299 | VGADG | 2279 | −2.01 | −2.05 | |
| P01116-G12D | 6 | HLIVGADGL | 2395 | LVVVGADGV | 2299 | VGADG | 2279 | −2.02 | −2.05 | |
| P01116-G12D | 8 | RVTADGVGR | 2396 | VVGADGVGK | 2200 | ADGVG | 2280 | −2.01 | −2.30 | A1101 |
| P01116-G12D | 8 | SMLADGVGR | 2397 | VVGADGVGK | 2200 | ADGVG | 2280 | −2.07 | −2.30 | |
| P01116-G12D | 6 | NVLVGADGI | 2398 | LVVVGADGV | 2299 | VGADG | 2279 | −2.11 | −1.88 | A2601 |
| P01116-G12D | 6 | ATIVGADGY | 2399 | LVVVGADGV | 2299 | VGADG | 2279 | −2.09 | −1.88 | |
| P01116-G12D | 8 | LTMADGVGF | 2300 | VVGADGVGK | 2200 | ADGVG | 2280 | −2.04 | −0.64 | |
| P01116-G12D | 8 | EVAADGVGV | 2301 | VVGADGVGK | 2200 | ADGVG | 2280 | −2.04 | −0.64 | |
| P01116-G12D | 5 | KIYVVGADA | 2302 | KLVVVGADG | 2298 | VVGAD | 2278 | −2.03 | −0.59 | A3001 |
| P01116-G12D | 5 | SQRVVGADK | 2303 | KLVVVGADG | 2298 | VVGAD | 2278 | −2.01 | −0.59 | |
| P01116-G12D | 6 | YVKVGADGV | 2304 | LVVVGADGV | 2299 | VGADG | 2279 | −2.07 | −1.29 | B0702 |
| P01116-G12D | 6 | LPVVGADGN | 2305 | LVVVGADGV | 2299 | VGADG | 2279 | −2.03 | −1.29 | |
| P01116-G12D | 7 | LPDGADGVS | 2306 | VVVGADGVG | 2201 | GADGV | 2281 | −2.04 | −1.39 | |
| P01116-G12D | 7 | QYRGADGVL | 2307 | VVVGADGVG | 2201 | GADGV | 2281 | −2.08 | −1.39 | |
| P01116-G12D | 8 | KLQADGVGF | 2308 | VVGADGVGK | 2200 | ADGVG | 2280 | −2.03 | −0.21 | |
| P01116-G12D | 8 | YKHADGVGI | 2309 | VVGADGVGK | 2200 | ADGVG | 2280 | −2.05 | −0.21 | |
| P01116-G12D | 5 | IGRVVGADV | 2310 | KLVVVGADG | 2298 | VVGAD | 2278 | −2.06 | 0.03 | B0801 |
| P01116-G12D | 5 | IIKVVGADV | 2311 | KLVVVGADG | 2298 | VVGAD | 2278 | −2.00 | 0.03 | |
| P01116-G12D | 6 | LYKVGADGI | 2312 | LVVVGADGV | 2299 | VGADG | 2279 | −2.01 | −0.46 | |
| P01116-G12D | 6 | KFKVGADGI | 2313 | LVVVGADGV | 2299 | VGADG | 2279 | −2.00 | −0.46 | |
| P01116-G12D | 5 | YIDVVGADY | 2314 | KLVVVGADG | 2298 | VVGAD | 2278 | −2.00 | −0.50 | B1501 |
| P01116-G12D | 5 | TLQVVGADY | 2315 | KLVVVGADG | 2298 | VVGAD | 2278 | −2.08 | −0.50 | |
| P01116-G12D | 6 | IVGVGADGY | 2316 | LVVVGADGV | 2299 | VGADG | 2279 | −2.00 | −1.29 | |
| P01116-G12D | 6 | IEQVGADGF | 2317 | LVVVGADGV | 2299 | VGADG | 2279 | −2.01 | −1.29 | |
| P01116-G13D | 6 | KMYVGAGDG | 2318 | LVVVGAGDV | 2300 | VGAGD | 2282 | −2.05 | −2.19 | A0201 |
| P01116-G13D | 6 | LMEVGAGDI | 2319 | LVVVGAGDV | 2300 | VGAGD | 2282 | −2.00 | −2.19 | |
| P01116-G13D | 7 | MLAGAGDVG | 2320 | VVVGAGDVG | 2301 | GAGDV | 2283 | −2.03 | −0.53 | |
| P01116-G13D | 7 | KQFGAGDVV | 2321 | VVVGAGDVG | 2301 | GAGDV | 2283 | −2.05 | −0.53 | |
| P01116-G13D | 8 | ELHAGDVGK | 2322 | VVGAGDVGK | 2302 | AGDVG | 2284 | −2.05 | −2.32 | A1101 |
| P01116-G13D | 8 | YMDAGDVGK | 2323 | VVGAGDVGK | 2302 | AGDVG | 2284 | −2.01 | −2.32 | |
| P01116-G13D | 6 | DLGVGAGDY | 2324 | LVVVGAGDV | 2300 | VGAGD | 2282 | −2.05 | −1.66 | A2601 |
| P01116-G13D | 6 | EYIVGAGDY | 2325 | LVVVGAGDV | 2300 | VGAGD | 2282 | −2.09 | −1.66 | |
| P01116-G13D | 9 | GARGDVGKT | 2326 | VGAGDVGKS | 2303 | GDVGK | 2285 | −2.04 | −0.02 | A3001 |
| P01116-G13D | 9 | YGKGDVGKP | 2327 | VGAGDVGKS | 2303 | GDVGK | 2285 | −2.01 | −0.02 | |
| P01116-G13D | 6 | IVKVGAGDL | 2328 | LVVVGAGDV | 2300 | VGAGD | 2282 | −2.01 | −0.81 | B0702 |
| P01116-G13D | 6 | LPYVGAGDS | 2329 | LVVVGAGDV | 2300 | VGAGD | 2282 | −2.01 | −0.81 | |
| P01116-G13D | 7 | RSFGAGDVL | 2330 | VVVGAGDVG | 2301 | GAGDV | 2283 | −2.09 | −0.62 | |
| P01116-G13D | 7 | RGIGAGDVL | 2331 | VVVGAGDVG | 2301 | GAGDV | 2283 | −2.08 | −0.62 | |
| P01116-G13D | 8 | QIVAGDVGL | 2332 | VVGAGDVGK | 2302 | AGDVG | 2284 | −2.02 | −0.43 | |
| P01116-G13D | 8 | EIQAGDVGL | 2333 | VVGAGDVGK | 2302 | AGDVG | 2284 | −2.09 | −0.43 | |
| P01116-G13D | 7 | MLKGAGDVL | 2334 | VVVGAGDVG | 2301 | GAGDV | 2283 | −2.01 | 0.25 | B0801 |
| P01116-G13D | 7 | FLRGAGDVI | 2335 | VVVGAGDVG | 2301 | GAGDV | 2283 | −1.99 | 0.25 | |
| P01116-G13D | 9 | GSEGDVGKV | 2336 | VGAGDVGKS | 2303 | GDVGK | 2285 | −2.04 | 0.47 | |
| P01116-G13D | 9 | IPTGDVGKL | 2337 | VGAGDVGKS | 2303 | GDVGK | 2285 | −2.00 | 0.47 | |
| P01116-G13D | 6 | RQTVGAGDY | 2338 | LVVVGAGDV | 2300 | VGAGD | 2282 | −2.07 | −1.38 | B1501 |
| P01116-G13D | 6 | NSYVGAGDY | 2339 | LVVVGAGDV | 2300 | VGAGD | 2282 | −2.01 | −1.38 | |
| P01116-G13D | 8 | TGKAGDVGI | 2340 | VVGAGDVGK | 2302 | AGDVG | 2284 | −2.06 | −0.23 | B5701 |
| P01116-G13D | 8 | PDYAGDVGY | 2341 | VVGAGDVGK | 2302 | AGDVG | 2284 | −2.03 | −0.23 | |

TABLE 18
Bespoke peptides designed for common mutations of KRAS for MHC II alleles

| | | | | | | | | | Pre- | |
|---|---|---|---|---|---|---|---|---|---|---|
| | | | | | | | | Pre- | dicted | |
| | | | | | | | | dicted | binding | |
| | | | | | | | | binding | affin- | |
| | | | | | | | | affin- | ity | |
| | | | SEQ | origi- | SEQ | | SEQ | ity | origi- | |
| | | proposed | ID | nating | ID | TCEM | ID | proposed | nating | |
| gi | pos | peptide | NO.: | peptide | NO.: | core | NO.: | peptide | peptide | Allele |
| P01116_G12V | 2 | TYKVLVVVGAVDFKS | 2374 | TEYKLVVVGAVGVGK | 2358 | LV~V~AV | 2342 | -2.01 | -0.33 | DRB1_0401 |
| P01116_G12V | 2 | KRLTLVVVGAVYTKR | 2375 | TEYKLVVVGAVGVGK | 2358 | LV~V~AV | 2342 | −2.07 | −0.33 | |
| P01116_G12V | 3 | SLQEVVVGAVGTYGQ | 2376 | EYKLVVVGAVGVGKS | 2359 | VV~G~VG | 2343 | −2.00 | 0.84 | |
| P01116_G12V | 3 | FTVIVVVGAVGKKEN | 2377 | EYKLVVVGAVGVGKS | 2359 | VV~G~VG | 2343 | −2.04 | −0.84 | |
| P01116_G12V | 5 | RVRLVGAVGVGKITT | 2378 | KLVVVGAVGVGKSAL | 2360 | VG~V~VG | 2344 | −2.02 | −2.36 | |
| P01116_G12V | 5 | YARIVGAVGVGRSGI | 2379 | KLVVVGAVGVGKSAL | 2360 | VG~V~VG | 2344 | −2.08 | −2.36 | |
| P01116_G12V | 7 | PRSFAVGVGKSYLIT | 2380 | VVVGAVGVGKSALTI | 2361 | AV~V~KS | 2345 | −2.04 | −0.14 | |
| P01116_G12V | 7 | EFRYAVGVGKSQALT | 2381 | VVVGAVGVGKSALTI | 2361 | AV~V~KS | 2345 | −2.06 | −0.14 | |
| P01116_G12V | 2 | HTDLLVVVGAVYYIK | 2382 | TEYKLVVVGAVGVGK | 2358 | LV~V~AV | 2342 | −2.00 | −1.06 | DRB1_0701 |
| P01116_G12V | 2 | ALQRLVVVGAVYYPD | 2383 | TEYKLVVVGAVGVGK | 2358 | LV~V~AV | 2342 | −2.01 | −1.06 | |
| P01116_G12V | 3 | PKQIVVVGAVGSFTV | 2384 | EYKLVVVGAVGVGKS | 2359 | VV~G~VG | 2343 | −2.01 | −1.35 | |
| P01116_G12V | 3 | TRNIVVVGAVGNTMI | 2385 | EYKLVVVGAVGVGKS | 2359 | VV~G~VG | 2343 | −1.99 | −1.35 | |
| P01116_G12V | 5 | LQRLVGAVGVGMRLV | 2386 | KLVVVGAVGVGKSAL | 2360 | VG~V~VG | 2344 | −2.01 | −1.42 | |
| P01116_G12V | 5 | RQKIVGAVGVGFMGI | 2387 | KLVVVGAVGVGKSAL | 2360 | VG~V~VG | 2344 | −2.00 | 1.42 | |
| P01116_G12V | 7 | SLYLAVGVGKSAYCV | 2388 | VVVGAVGVGKSALTI | 2361 | AV~V~KS | 2345 | −2.02 | −1.20 | |
| P01116_G12V | 7 | TFVYAVGVGKSGYLI | 2389 | VVVGAVGVGKSALTI | 2361 | AV~V~KS | 2345 | −2.05 | −1.20 | |
| P01116_G12V | 2 | KRLILVVVGAVFKAR | 2390 | TEYKLVVVGAVGVGK | 2358 | LV~V~AV | 2342 | −2.05 | 0.07 | DRB1_1101 |
| P01116_G12V | 2 | DTILLVVVGAVRKIR | 2391 | TEYKLVVVGAVGVGK | 2358 | LV~V~AV | 2342 | −2.01 | 0.07 | |
| P01116_G12V | 3 | RLYRVVVGAVGDKTQ | 2392 | EYKLVVVGAVGVGKS | 2359 | VV~G~VG | 2343 | −2.00 | 0.02 | |
| P01116_G12V | 3 | VIVVVVVGAVGRRTD | 2393 | EYKLVVVGAVGVGKS | 2359 | VV~G~VG | 2343 | −2.06 | 0.02 | |
| P01116_G12V | 5 | RVFMVGAVGVGVRKC | 2394 | KLVVVGAVGVGKSAL | 2360 | VG~V~VG | 2344 | −2.04 | 0.57 | |
| P01116_G12V | 2 | EDEILVVVGAVIDTE | 2395 | TEYKLVVVGAVGVGK | 2358 | LV~V~AV | 2342 | −2.01 | −0.18 | DRB1_1501 |
| P01116_G12V | 2 | KFDRLVVVGAVVFDD | 2396 | TEYKLVVVGAVGVGK | 2358 | LV~V~AV | 2342 | −2.01 | −0.18 | |
| P01116_G12V | 3 | YSHVVVVGAVGTLNG | 2397 | EYKLVVVGAVGVGKS | 2359 | VV~G~VG | 2343 | −2.07 | −0.16 | |
| P01116_G12V | 3 | RVRYVVVGAVGLDPE | 2398 | EYKLVVVGAVGVGKS | 2359 | VV~G~VG | 2343 | −2.02 | −0.16 | |
| P01116_G12V | 5 | ELKIVGAVGVGLYTD | 2399 | KLVVVGAVGVGKSAL | 2360 | VG~V~VG | 2344 | −2.06 | −0.38 | |
| P01116_G12V | 5 | FREIVGAVGVGQLQM | 2400 | KLVVVGAVGVGKSAL | 2360 | VG~V~VG | 2344 | −2.01 | −0.38 | |
| P01116_G12V | 7 | PLLVAVGVGKSIKKC | 2401 | VVVGAVGVGKSALTI | 2361 | AV~V~KS | 2345 | −2.02 | 0.38 | |
| P01116_G12V | 7 | KIILAVGVGKSALQC | 2402 | VVVGAVGVGKSALTI | 2361 | AV~V~KS | 2345 | −2.01 | 0.38 | |
| P01116-G12C | 2 | QSKVLVVVGACPGEI | 2403 | TEYKLVVVGACGVGK | 2362 | LV~V~AC | 2346 | −2.07 | −1.05 | DRB1_0401 |
| P01116-G12C | 2 | NYYSLVVVGACKKVQ | 2404 | TEYKLVVVGACGVGK | 2362 | LV~V~AC | 2346 | −2.06 | −1.05 | |
| P01116-G12C | 3 | SSFHVVVGACGTAIN | 2405 | EYKLVVVGACGVGKS | 2363 | VV~G~CG | 2347 | −2.06 | −0.81 | |
| P01116-G12C | 3 | TYRLVVVGACGDKIT | 2406 | EYKLVVVGACGVGKS | 2363 | VV~G~CG | 2347 | −2.04 | −0.81 | |
| P01116-G12C | 5 | RQIIVGACGVGFESA | 2407 | KLVVVGACGVGKSAL | 2364 | VG~C~VG | 2348 | −2.07 | −2.20 | |
| P01116-G12C | 5 | DYIFVGACGVGKSQA | 2408 | KLVVVGACGVGKSAL | 2364 | VG~C~VG | 2348 | −2.09 | −2.20 | |
| P01116-G12C | 2 | DKRVLVVVGACKVYL | 2409 | TEYKLVVVGACGVGK | 2362 | LV~V~AC | 2346 | −2.04 | −1.56 | DRB1_0701 |
| P01116-G12C | 2 | TNFKLVVVGACEVIG | 2410 | TEYKLVVVGACGVGK | 2362 | LV~V~AC | 2346 | −2.01 | 1.56 | |
| PO1116-G12C | 5 | QVDLVGACGVGFCHF | 2411 | KLVVVGACGVGKSAL | 2364 | VG~C~VG | 2348 | −2.00 | −0.98 | |
| P01116-G12C | 5 | KYLDVGACGVGKVLI | 2412 | KLVVVGACGVGKSAL | 2364 | VG~C~VG | 2348 | −1.99 | −0.98 | |
| P01116-G12C | 7 | YYDFACGVGKSIMFI | 2413 | VVVGACGVGKSALTI | 2365 | AC~V~KS | 2349 | −2.03 | −0.78 | |
| P01116-G12C | 7 | YFQYACGVGKSVLLM | 2414 | VVVGACGVGKSALTI | 2365 | AC~V~KS | 2349 | −2.06 | −0.78 | |
| P01116-G12C | 2 | IRDFLVVVGACAEPP | 2415 | TEYKLVVVGACGVGK | 2362 | LV~V~AC | 2346 | −2.03 | 0.26 | DRB1_1101 |
| P01116-G12C | 2 | QLEFLVVVGACKYDY | 2416 | TEYKLVVVGACGVGK | 2362 | LV~V~AC | 2346 | −2.04 | 0.26 | |
| P01116-G12C | 3 | YRYIVVVGACGNYSN | 2417 | EYKLVVVGACGVGKS | 2363 | VV~G~CG | 2347 | −2.07 | 0.26 | |
| P01116-G12C | 2 | YTEHLVVVGACGYKL | 2418 | TEYKLVVVGACGVGK | 2363 | LV~V~AC | 2346 | −2.01 | 0.11 | DRB1_1501 |
| P01116-G12C | 2 | RQYELVVVGACVVKV | 2419 | TEYKLVVVGACGVGK | 2362 | LV~V~AC | 2346 | −2.01 | 0.11 | |
| P01116-G12C | 3 | DYILVVVGACGKTYR | 2420 | EYKLVVVGACGVGKS | 2363 | VV~G~CG | 2347 | −2.05 | 0.37 | |
| P01116-G12C | 3 | DILYVVVGACGRYRR | 2421 | EYKLVVVGACGVGKS | 2363 | VV~G~CG | 2347 | −2.01 | 0.37 | |
| P01116-G12C | 5 | RARVVGACGVGYIVM | 2422 | KLVVVGACGVGKSAL | 2364 | VG~C~VG | 2348 | −2.04 | −0.14 | |
| P01116-G12C | 5 | EIKFVGACGVGILPN | 2423 | KLVVVGACGVGKSAL | 2364 | VG~C~VG | 2348 | −2.02 | −0.14 | |
| P01116-G12D | 2 | EYVDLVVVGADGRGL | 2424 | TEYKLVVVGADGVGK | 2366 | LV~V~AD | 2350 | −2.07 | −1.22 | DRB1_0401 |
| P01116-G12D | 2 | KRYLLVVVGADRTKT | 2425 | TEYKLVVVGADGVGK | 2366 | LV~V~AD | 2350 | −2.08 | −1.22 | |
| P01116-G12D | 3 | KIAYVVVGADGGGEF | 2426 | EYKLVVVGADGVGKS | 2367 | VV~G~DG | 2351 | −2.07 | 0.79 | |
| P01116-G12D | 3 | STTIVVVGADGIRGK | 2427 | EYKLVVVGADGVGKS | 2367 | VV~G~DG | 2351 | −2.04 | −0.79 | |
| P01116-G12D | 5 | LYILVGADGVGKQGV | 2428 | KLVVVGADGVGKSAL | 2368 | VG~D~VG | 2352 | −2.07 | −1.28 | |
| P01116-G12D | 2 | KFRELVVVGADLVPV | 2429 | TEYKLVVVGADGVGK | 2366 | LV~V~AD | 2350 | −2.03 | −1.00 | DRB1_0701 |
| P01116-G12D | 2 | LVQFLVVVGADKVDM | 2430 | TEYKLVVVGADGVGK | 2366 | LV~V~AD | 2350 | −2.05 | −1.00 | |
| P01116-G12D | 3 | PIDLVVVGADGDIVI | 2431 | EYKLVVVGADGVGKS | 2367 | VV~G~DG | 2351 | −2.03 | −0.24 | |
| P01116-G12D | 3 | RYTFVVVGADGGIEI | 2432 | EYKLVVVGADGVGKS | 2367 | VV~G~DG | 2351 | −2.01 | −0.24 | |
| P01116-G12D | 5 | KYLVVGADGVGFEFI | 2433 | KLVVVGADGVGKSAL | 2368 | VG~D~VG | 2352 | −2.00 | −0.54 | |
| P01116-G12D | 5 | KVSMVGADGVGTMFF | 2434 | KLVVVGADGVGKSAL | 2368 | VG~D~VG | 2352 | −2.05 | −0.54 | |
| P01116-G12D | 2 | QIKLLVVVGADPGKK | 2435 | TEYKLVVVGADGVGK | 2366 | LV~V~AD | 2350 | −2.07 | 0.17 | DRB1_1101 |
| P01116-G12D | 2 | DREVLVVVGADRETC | 2436 | TEYKLVVVGADGVGK | 2366 | LV~V~AD | 2350 | −2.03 | 0.17 | |
| P01116-G12D | 3 | RQIRVVVGADGTTQP | 2437 | EYKLVVVGADGVGKS | 2367 | VV~G~DG | 2351 | −2.06 | −0.15 | |
| P01116-G12D | 3 | DALFVVVGADGQTQT | 2438 | EYKLVVVGADGVGKS | 2367 | VV~G~DG | 2351 | −2.04 | −0.15 | |
| P01116-G12D | 5 | KHIFVGADGVGKTDD | 2439 | KLVVVGADGVGKSAL | 2368 | VG~D~VG | 2352 | −2.02 | 0.46 | |
| P01116-G12D | 5 | LYFIVGADGVGDRYK | 2440 | KLVVVGADGVGKSAL | 2368 | VG~D~VG | 2352 | −2.05 | 0.46 | |
| P01116-G12D | 2 | GRVILVVVGADMDYE | 2441 | TEYKLVVVGADGVGK | 2366 | LV~V~AD | 2350 | −2.05 | 0.07 | DRB1_1501 |
| P01116-G12D | 2 | RVDVLVVVGADAGLK | 2442 | TEYKLVVVGADGVGK | 2366 | LV~V~AD | 2350 | −2.04 | 0.07 | |
| P01116-G12D | 3 | EILLVVVGADGYQYT | 2443 | EYKLVVVGADGVGKS | 2367 | VV~G~DG | 2351 | −2.07 | −0.08 | |
| P01116-G12D | 3 | SVPFVVVGADGINRF | 2444 | EYKLVVVGADGVGKS | 2367 | VV~G~DG | 2351 | −2.03 | −0.08 | |
| P01116-G12D | 5 | YKVMVGADGVGLLKA | 2445 | KLVVVGADGVGKSAL | 2368 | VG~D~VG | 2352 | −2.01 | 0.16 | |
| P01116-G12D | 5 | KRYVVGADGVGIYPV | 2446 | KLVVVGADGVGKSAL | 2368 | VG~D~VG | 2352 | −2.03 | 0.16 | |
| P01116-G13D | 3 | LDRKVVVGAGDPPLA | 2447 | EYKLVVVGAGDVGKS | 2369 | VV~G~GD | 2353 | −2.01 | −0.89 | DRB1_0401 |
| P01116-G13D | 3 | IIEYVVVGAGDRSVK | 2448 | EYKLVVVGAGDVGKS | 2369 | VV~G~GD | 2353 | −2.04 | −0.89 | |
| P01116-G13D | 4 | GRRFVVGAGDVISGS | 2449 | YKLVVVGAGDVGKSA | 2370 | VV~A~DV | 2354 | −2.00 | −0.70 | |
| P01116-G13D | 4 | SPVFVVGAGDVQKTY | 2450 | YKLVVVGAGDVGKSA | 2370 | VV~A~DV | 2354 | −2.01 | −0.70 | |
| P01116-G13D | 3 | KAQFVVVGAGDCQIF | 2451 | EYKLVVVGAGDVGKS | 2369 | VV~G~GD | 2353 | −2.04 | −0.97 | DRB1_0701 |
| P01116-G13D | 3 | FYTIVVVGAGDFYKC | 2452 | EYKLVVVGAGDVGKS | 2369 | VV~G~GD | 2353 | −2.00 | −0.97 | |
| P01116-G13D | 4 | YTRYVVGAGDVFIKM | 2453 | YKLVVVGAGDVGKSA | 2370 | VV~A~DV | 2354 | −2.00 | −0.56 | |
| P01116-G13D | 4 | FYKIVVGAGDVKFHM | 2454 | YKLVVVGAGDVGKSA | 2370 | VV~A~DV | 2354 | −2.04 | −0.56 | |
| P01116-G13D | 6 | EFIFGAGDVGKVVFV | 2455 | LVVVGAGDVGKSALT | 2371 | GA~D~GK | 2355 | −2.06 | −0.17 | |
| P01116-G13D | 3 | EKIIVVVGAGDIVKV | 2456 | EYKLVVVGAGDVGKS | 2369 | VV~G~GD | 2353 | −2.07 | 0.11 | DRB1_1101 |
| P01116-G13D | 3 | IQRYVVVGAGDVHPI | 2457 | EYKLVVVGAGDVGKS | 2369 | VV~G~GD | 2353 | −2.06 | 0.11 | |
| P01116-G13D | 4 | FFLMVVGAGDVEERP | 2458 | YKLVVVGAGDVGKSA | 2370 | VV~A~DV | 2354 | −2.06 | 0.16 | |
| P01116-G13D | 4 | LPVYVVGAGDVPGKT | 2459 | YKLVVVGAGDVGKSA | 2370 | VV~A~DV | 2354 | −2.02 | 0.16 | |
| P01116-G13D | 3 | KVYYVVVGAGDDIFE | 2460 | EYKLVVVGAGDVGKS | 2369 | VV~G~GD | 2353 | −2.01 | 0.13 | DRB1_1501 |
| P01116-G13D | 3 | RYIIVVVGAGDGTRF | 2461 | EYKLVVVGAGDVGKS | 2369 | VV~G~GD | 2353 | −2.02 | 0.13 | |
| P01116-G13D | 4 | RTRYVVGAGDVPFKV | 2462 | YKLVVVGAGDVGKSA | 2370 | VV~A~DV | 2354 | −2.06 | 0.12 | |
| P01116-G13D | 4 | RTILVVGAGDVIVDC | 2463 | YKLVVVGAGDVGKSA | 2370 | VV~A~DV | 2354 | −2.01 | 0.12 | |
| P01116-Q61H | 52 | TSFFLDTAGHELSST | 2464 | LLDILDTAGHEEYSA | 2372 | LD~A~HE | 2356 | −2.06 | −0.88 | DRB1_0401 |
| P01116-Q61H | 52 | YLYYLDTAGHETPKD | 2465 | LLDILDTAGHEEYSA | 2372 | LD~A~HE | 2356 | −2.10 | −0.88 | |
| P01116-Q61H | 51 | DDGVILDTAGHQYIM | 2466 | CLLDILDTAGHEEYS | 2373 | IL~T~GH | 2357 | −2.01 | −1.08 | DRB1_0701 |
| P01116-Q61H | 51 | KTYLILDTAGHYNVS | 2467 | CLLDILDTAGHEEYS | 2373 | IL~T~GH | 2357 | −2.01 | −1.08 | |
| P01116-Q61H | 52 | LIIILDTAGHEECPI | 2468 | LLDILDTAGHEEYSA | 2372 | LD~A~HE | 2356 | −2.02 | −0.12 | |
| P01116-Q61H | 52 | PIYYLDTAGHEIFAT | 2469 | LLDILDTAGHEEYSA | 2372 | LD~A~HE | 2356 | −2.03 | −0.12 | |
| P01116-Q61H | 51 | LRRVILDTAGHVVSR | 2470 | CLLDILDTAGHEEYS | 2373 | IL~T~GH | 2357 | −2.01 | 0.24 | DRB1_1101 |
| P01116-Q61H | 51 | GKYYILDTAGHQVTK | 2471 | CLLDILDTAGHEEYS | 2373 | IL~T~GH | 2357 | −2.06 | 0.24 | |
| P01116-Q61H | 52 | DLFILDTAGHELKQG | 2472 | LLDILDTAGHEEYSA | 2372 | LD~A~HE | 2356 | −2.05 | −0.20 | |
| P01116-Q61H | 52 | VIYYLDTAGHEKKYQ | 2473 | LLDILDTAGHEEYSA | 2372 | LD~A~HE | 2356 | −2.06 | −0.20 | |
| P01116-Q61H | 51 | QQEGILDTAGHGYFM | 2474 | CLLDILDTAGHEEYS | 2373 | IL~T~GH | 2357 | −2.04 | 0.28 | DRB1_1501 |
| P01116-Q61H | 51 | QFFTILDTAGHIDDV | 2475 | CLLDILDTAGHEEYS | 2373 | IL~T~GH | 2357 | −2.00 | 0.28 | |
| P01116-Q61H | 52 | RYRMLDTAGHEIIDV | 2476 | LLDILDTAGHEEYSA | 2372 | LD~A~HE | 2356 | −2.06 | −0.31 | |
| P01116-Q61H | 52 | LVIFLDTAGHEPDYV | 2477 | LLDILDTAGHEEYSA | 2372 | LD~A~HE | 2356 | −2.06 | −0.31 | |

### Example 14 Bespoke Peptides Targeting KIAA1549-BRAF Fusions

Two mutations are highly characteristic of pediatric low-grade gliomas: KIAA1549-BRAF and BRAFV600E. In one study these together accounted for 68% of pediatric low-grade gliomas [80]. At present only two forms (long and short) of fusion of KIAA1549 to BRAF are described, providing unique neoepitopes at the fusion junction. The in-frame fusion maintains the kinase activity of BRAF while also truncating the N terminal through which the kinase activity of BRAF is regulated [81]. KIAA1549 has 4 recorded isoforms. Two “short forms” lack the initial 1216 amino acids and these may participate in fusions which occur most commonly at 1749 (exon 16). A second isoform of the long canonical form has a deletion of aa 1867-1882 (region absent in the fusion). The most common fusion site is KIAA1549 ex16: BRAF ex9 (approximately 80% cases), exemplified by Genbank gi 211920461, but fusions of KIAA1549ex16: BRAF ex11 (e.g., gi 211920463) and KIAA1549ex15:BRAFex9 (gi 211920465) are also recorded. In TABLE 19 and 20 we show the novel T cell exposed motifs that characterize these 3 fusion junctions and provide bespoke peptides that will target them.

TABLE 19
Bespoke peptides designed for unique motifs at junction site
of KIAA1549-BRAF fusions for MHC I alleles

| | | | | | | | | | | predicted | |
|---|---|---|---|---|---|---|---|---|---|---|---|
| | | | | | | | | | predicted | affin- | |
| | | | | | | | | | affin- | ity | |
| | | | | SEQ | origi- | SEQ | | SEQ | ity | origi- | |
| | | | proposed | ID | nating | ID | TCEM | ID | proposed | nating | |
| gi | pos | Fusion site | peptide | NO.: | peptide | NO.: | core | NO.: | peptide | peptide | Allele |
| 211920461 | 1743 | KIAA1549_BRAF 16_9 | GLPNPCSDL | 1400 | TANNPCSDL | 1389 | NPCSD | 1378 | -2.03 | -0.12 | A0201 |
| 211920461 | 1743 | KIAA1549_BRAF 16_9 | FAVNPCSDV | 1401 | TANNPCSDL | 1389 | NPCSD | 1378 | −2.02 | −0.12 | |
| 211920461 | 1744 | KIAA1549_BRAF 16_9 | NIYPCSDLV | 1402 | ANNPCSDLI | 1390 | PCSDL | 1379 | −2.02 | −0.08 | |
| 211920461 | 1744 | KIAA1549_BRAF 16_9 | VMQPCSDLL | 1403 | ANNPCSDLI | 1390 | PCSDL | 1379 | −2.02 | −0.08 | |
| 211920461 | 1745 | KIAA1549_BRAF 16_9 | IDTCSDLIK | 1404 | NNPCSDLIR | 1391 | CSDLI | 1380 | −2.01 | −0.74 | A0301 |
| 211920461 | 1745 | KIAA1549_BRAF 16_9 | EVRCSDLIP | 1405 | NNPCSDLIR | 1391 | CSDLI | 1380 | −2.02 | −0.74 | |
| 211920461 | 1745 | KIAA1549_BRAF 16_9 | RAECSDLIP | 1406 | NNPCSDLIR | 1391 | CSDLI | 1380 | −2.07 | −1.68 | A1101 |
| 211920461 | 1745 | KIAA1549_BRAF 16_9 | GSNCSDLIP | 1407 | NNPCSDLIR | 1391 | CSDLI | 1380 | −2.03 | −1.68 | |
| 211920461 | 1746 | KIAA1549_BRAF 16_9 | IVASDLIRY | 1408 | NPCSDLIRD | 1392 | SDLIR | 1381 | −2.08 | −0.79 | |
| 211920461 | 1746 | KIAA1549_BRAF 16_9 | SIQSDLIRP | 1409 | NPCSDLIRD | 1392 | SDLIR | 1381 | −2.01 | −0.79 | |
| 211920461 | 1745 | KIAA1549_BRAF 16_9 | EPRCSDLIR | 1410 | NNPCSDLIR | 1391 | CSDLI | 1380 | −2.07 | −1.68 | A6801 |
| 211920461 | 1745 | KIAA1549_BRAF 16_9 | RAVCSDLIK | 1411 | NNPCSDLIR | 1391 | CSDLI | 1380 | −2.06 | −1.68 | |
| 211920461 | 1746 | KIAA1549_BRAF 16_9 | VGTSDLIRK | 1412 | NPCSDLIRD | 1392 | SDLIR | 1381 | −2.07 | −0.58 | |
| 211920461 | 1746 | KIAA1549_BRAF 16_9 | TVLSDLIRK | 1413 | NPCSDLIRD | 1392 | SDLIR | 1381 | −2.07 | −0.58 | |
| 211920461 | 1743 | KIAA1549_BRAF 16_9 | APPNPCSDW | 1414 | TANNPCSDL | 1389 | NPCSD | 1378 | −2.04 | −0.34 | B0702 |
| 211920461 | 1743 | KIAA1549_BRAF 16_9 | NPLNPCSDA | 1415 | TANNPCSDL | 1389 | NPCSD | 1378 | −2.04 | −0.34 | |
| 211920461 | 1744 | KIAA1549_BRAF 16_9 | PGRPCSDLT | 1416 | ANNPCSDLI | 1390 | PCSDL | 1379 | −2.03 | −0.29 | |
| 211920461 | 1744 | KIAA1549_BRAF 16_9 | KGEPCSDLL | 1417 | ANNPCSDLI | 1390 | PCSDL | 1379 | −2.07 | −0.29 | |
| 211920461 | 1746 | KIAA1549_BRAF 16_9 | KPMSDLIRG | 1418 | NPCSDLIRD | 1392 | SDLIR | 1381 | −2.02 | −0.73 | |
| 211920461 | 1746 | KIAA1549_BRAF 16_9 | KPESDLIRA | 1419 | NPCSDLIRD | 1392 | SDLIR | 1381 | −2.02 | −0.73 | |
| 211920461 | 1743 | KIAA1549_BRAF 16_9 | GARNPCSDV | 1420 | TANNPCSDL | 1389 | NPCSD | 1378 | −2.02 | 0.22 | B0801 |
| 211920461 | 1743 | KIAA1549_BRAF 16_9 | AARNPCSDV | 1421 | TANNPCSDL | 1389 | NPCSD | 1378 | −2.03 | 0.22 | |
| 211920461 | 1744 | KIAA1549_BRAF 16_9 | YSRPCSDLV | 1422 | ANNPCSDLI | 1390 | PCSDL | 1379 | −2.04 | 0.10 | |
| 211920461 | 1744 | KIAA1549_BRAF 16_9 | RTRPCSDLV | 1423 | ANNPCSDLI | 1390 | PCSDL | 1379 | −2.04 | 0.10 | |
| 211920461 | 1745 | KIAA1549_BRAF 16_9 | YTFCSDLIT | 1424 | NNPCSDLIR | 1391 | CSDLI | 1380 | −2.01 | −1.89 | B2705 |
| 211920461 | 1745 | KIAA1549_BRAF 16_9 | RNECSDLIR | 1425 | NNPCSDLIR | 1391 | CSDLI | 1380 | −2.00 | −1.89 | |
| 211920461 | 1746 | KIAA1549_BRAF 16_9 | LGKSDLIRF | 1426 | NPCSDLIRD | 1392 | SDLIR | 1381 | −2.03 | −0.70 | B3501 |
| 211920461 | 1746 | KIAA1549_BRAF 16_9 | RVVSDLIRF | 1427 | NPCSDLIRD | 1392 | SDLIR | 1381 | −2.05 | −0.70 | |
| 211920463 | 1746 | KIAA1549_BRAF 16_11 | LVWSKTLGD | 1428 | NPCSKTLGR | 1393 | SKTLG | 1382 | −2.04 | −1.22 | A0301 |
| 211920463 | 1746 | KIAA1549_BRAF 16_11 | IPLSKTLGR | 1429 | NPCSKTLGR | 1393 | SKTLG | 1382 | −2.01 | −1.22 | |
| 211920463 | 1746 | KIAA1549_BRAF 16_11 | DAFSKTLGR | 1430 | NPCSKTLGR | 1393 | SKTLG | 1382 | −2.04 | −1.58 | A1101 |
| 211920463 | 1746 | KIAA1549_BRAF 16_11 | ASGSKTLGR | 1431 | NPCSKTLGR | 1393 | SKTLG | 1382 | −2.04 | −1.58 | |
| 211920463 | 1746 | KIAA1549_BRAF 16_11 | FSVSKTLGK | 1432 | NPCSKTLGR | 1393 | SKTLG | 1382 | −2.07 | −1.77 | A6801 |
| 211920463 | 1746 | KIAA1549_BRAF 16_11 | VAPSKTLGR | 1433 | NPCSKTLGR | 1393 | SKTLG | 1382 | −2.06 | −1.77 | |
| 211920463 | 1744 | KIAA1549_BRAF 16_11 | FMRPCSKTI | 1434 | ANNPCSKTL | 1394 | PCSKT | 1383 | −2.05 | −0.24 | B0702 |
| 211920463 | 1744 | KIAA1549_BRAF 16_11 | LPIPCSKTP | 1435 | ANNPCSKTL | 1394 | PCSKT | 1383 | −2.06 | −0.24 | |
| 211920463 | 1746 | KIAA1549_BRAF 16_11 | LASSKTLGL | 1436 | NPCSKTLGR | 1393 | SKTLG | 1382 | −2.06 | −1.05 | |
| 211920463 | 1746 | KIAA1549_BRAF 16_11 | AGMSKTLGL | 1437 | NPCSKTLGR | 1393 | SKTLG | 1382 | −2.06 | −1.05 | |
| 211920463 | 1744 | KIAA1549_BRAF 16_11 | VSKPCSKTV | 1438 | ANNPCSKTL | 1394 | PCSKT | 1383 | −2.05 | 0.12 | B0801 |
| 211920463 | 1744 | KIAA1549_BRAF 16_11 | KLRPCSKTL | 1439 | ANNPCSKTL | 1394 | PCSKT | 1383 | −2.06 | 0.12 | |
| 211920463 | 1745 | KIAA1549_BRAF 16_11 | ARSCSKTLS | 1440 | NNPCSKTLG | 1395 | CSKTL | 1384 | −2.02 | −0.61 | B2705 |
| 211920463 | 1745 | KIAA1549_BRAF 16_11 | PQLCSKTLL | 1441 | NNPCSKTLG | 1395 | CSKTL | 1384 | −2.03 | −0.61 | |
| 211920463 | 1746 | KIAA1549_BRAF 16_11 | LKDSKTLGS | 1442 | NPCSKTLGR | 1393 | SKTLG | 1382 | −2.05 | −0.22 | |
| 211920463 | 1746 | KIAA1549_BRAF 16_11 | HNFSKTLGR | 1443 | NPCSKTLGR | 1393 | SKTLG | 1382 | −2.01 | −0.22 | |
| 211920463 | 1746 | KIAA1549_BRAF 16_11 | VITSKTLGY | 1444 | NPCSKTLGR | 1393 | SKTLG | 1382 | −2.03 | −1.86 | B3501 |
| 211920463 | 1746 | KIAA1549_BRAF 16_11 | FKFSKTLGY | 1445 | NPCSKTLGR | 1393 | SKTLG | 1382 | −2.06 | −1.86 | |
| 211920465 | 1637 | KIAA1549_BRAF 15_9 | HMAIGCPDL | 1446 | SAYIGCPDL | 1396 | IGCPD | 1385 | −2.00 | −0.32 | A0201 |
| 211920465 | 1637 | KIAA1549_BRAF 15_9 | KIAIGCPDV | 1447 | SAYIGCPDL | 1396 | IGCPD | 1385 | −2.05 | −0.32 | |
| 211920465 | 1638 | KIAA1549_BRAF 15_9 | RVEGCPDLV | 1448 | AYIGCPDLI | 1397 | GCPDL | 1386 | −2.00 | −1.34 | |
| 211920465 | 1638 | KIAA1549_BRAF 15_9 | YLEGCPDLS | 1449 | AYIGCPDLI | 1397 | GCPDL | 1386 | −2.05 | −1.34 | |
| 211920465 | 1639 | KIAA1549_BRAF 15_9 | TSLCPDLIP | 1450 | YIGCPDLIR | 1398 | CPDLI | 1387 | −2.01 | −1.68 | A0301 |
| 211920465 | 1639 | KIAA1549_BRAF 15_9 | QAQCPDLIP | 1451 | YIGCPDLIR | 1398 | CPDLI | 1387 | −2.06 | −1.68 | |
| 211920465 | 1640 | KIAA1549_BRAF 15_9 | KTSPDLIRY | 1452 | IGCPDLIRD | 1399 | PDLIR | 1388 | −2.01 | −1.07 | |
| 211920465 | 1640 | KIAA1549_BRAF 15_9 | VSAPDLIRP | 1453 | IGCPDLIRD | 1399 | PDLIR | 1388 | −2.03 | −1.07 | |
| 211920465 | 1637 | KIAA1549_BRAF 15_9 | AVVIGCPDP | 1454 | SAYIGCPDL | 1396 | IGCPD | 1385 | −2.06 | −0.35 | A1101 |
| 211920465 | 1637 | KIAA1549_BRAF 15_9 | QNHIGCPDK | 1455 | SAYIGCPDL | 1396 | IGCPD | 1385 | −2.04 | −0.35 | |
| 211920465 | 1639 | KIAA1549_BRAF 15_9 | PEQCPDLIK | 1456 | YIGCPDLIR | 1398 | CPDLI | 1387 | −2.05 | −1.79 | |
| 211920465 | 1639 | KIAA1549_BRAF 15_9 | WSACPDLIP | 1457 | YIGCPDLIR | 1398 | CPDLI | 1387 | −2.02 | −1.79 | |
| 211920465 | 1640 | KIAA1549_BRAF 15_9 | AQSPDLIRR | 1458 | IGCPDLIRD | 1399 | PDLIR | 1388 | −2.01 | −1.22 | |
| 211920465 | 1640 | KIAA1549_BRAF 15_9 | SEYPDLIRR | 1459 | IGCPDLIRD | 1399 | PDLIR | 1388 | −2.07 | −1.22 | |
| 211920465 | 1639 | KIAA1549_BRAF 15_9 | KAVCPDLIR | 1460 | YIGCPDLIR | 1398 | CPDLI | 1387 | −2.05 | −1.83 | A6801 |
| 211920465 | 1639 | KIAA1549_BRAF 15_9 | QPTCPDLIR | 1461 | YIGCPDLIR | 1398 | CPDLI | 1387 | −2.02 | −1.83 | |
| 211920465 | 1640 | KIAA1549_BRAF 15_9 | RPPPDLIRR | 1462 | IGCPDLIRD | 1399 | PDLIR | 1388 | −2.07 | −1.20 | |
| 211920465 | 1640 | KIAA1549_BRAF 15_9 | SSLPDLIRK | 1463 | IGCPDLIRD | 1399 | PDLIR | 1388 | −2.03 | −1.20 | |
| 211920465 | 1637 | KIAA1549_BRAF 15_9 | SPLIGCPDY | 1464 | SAYIGCPDL | 1396 | IGCPD | 1385 | −2.04 | −0.93 | B0702 |
| 211920465 | 1637 | KIAA1549_BRAF 15_9 | RGRIGCPDF | 1465 | SAYIGCPDL | 1396 | IGCPD | 1385 | −2.06 | −0.93 | |
| 211920465 | 1638 | KIAA1549_BRAF 15_9 | HLTGCPDLV | 1466 | AYIGCPDLI | 1397 | GCPDL | 1386 | −2.04 | −0.50 | B0801 |
| 211920465 | 1638 | KIAA1549_BRAF 15_9 | EAKGCPDLV | 1467 | AYIGCPDLI | 1397 | GCPDL | 1386 | −2.04 | −0.50 | |
| 211920465 | 1638 | KIAA1549_BRAF 15_9 | TRRGCPDLL | 1468 | AYIGCPDLI | 1397 | GCPDL | 1386 | −2.01 | −0.43 | B2705 |
| 211920465 | 1638 | KIAA1549_BRAF 15_9 | YKSGCPDLP | 1469 | AYIGCPDLI | 1397 | GCPDL | 1386 | −2.01 | −0.43 | |
| 211920465 | 1637 | KIAA1549_BRAF 15_9 | YVPIGCPDW | 1470 | SAYIGCPDL | 1396 | IGCPD | 1385 | −2.06 | −0.61 | B3501 |
| 211920465 | 1637 | KIAA1549_BRAF 15_9 | QATIGCPDW | 1471 | SAYIGCPDL | 1396 | IGCPD | 1385 | −2.03 | −0.61 | |
| 211920465 | 1638 | KIAA1549_BRAF 15_9 | NAKGCPDLW | 1472 | AYIGCPDLI | 1397 | GCPDL | 1386 | −2.02 | −0.10 | |
| 211920465 | 1638 | KIAA1549_BRAF 15_9 | RLVGCPDLF | 1473 | AYIGCPDLI | 1397 | GCPDL | 1386 | −2.02 | −0.10 | |
| 211920465 | 1639 | KIAA1549_BRAF 15_9 | VPVCPDLIR | 1474 | YIGCPDLIR | 1398 | CPDLI | 1387 | −2.04 | −1.58 | |
| 211920465 | 1639 | KIAA1549_BRAF 15_9 | KLHCPDLIF | 1475 | YIGCPDLIR | 1398 | CPDLI | 1387 | −2.04 | −1.58 | |
| 211920465 | 1640 | KIAA1549_BRAF 15_9 | LPQPDLIRF | 1476 | IGCPDLIRD | 1399 | PDLIR | 1388 | −2.04 | −0.80 | |
| 211920465 | 1640 | KIAA1549_BRAF 15_9 | VASPDLIRF | 1477 | IGCPDLIRD | 1399 | PDLIR | 1388 | −2.03 | −0.80 | |

TABLE 20
Bespoke peptides designed for unique motifs at junction site of KIAA1549-BRAF fusions for MHC II alleles

| | | | | | | | | | predicted | predicted | |
|---|---|---|---|---|---|---|---|---|---|---|---|
| | | | | | | | | | binding | binding | |
| | | | | | | | | | affinity | affinity | |
| | | | proposed | SEQ ID | originating | SEQ ID | TCEM | SEQ ID | proposed | originating | |
| gi | pos | curation | peptide | NO.: | peptide | NO.: | core | NO.: | peptide | peptide | Allele |
| 211920461 | 1743 | KIAA1549_BRAF | IAFTPCSDLIRRTFS | 1490 | TANNPCSDLIRDQGF | 1484 | PC~D~IR | 1478 | −2.01 | −0.20 | DRB1_0301 |
| | | 16_9 | | | | | | | | | |
| 211920461 | 1743 | KIAA1549_BRAF | FGRLPCSDLIRVFDK | 1491 | TANNPCSDLIRDQGF | 1484 | PC~D~IR | 1478 | −2.02 | −0.20 | DRB1_0401 |
| | | 16_9 | | | | | | | | | |
| 211920461 | 1743 | KIAA1549_BRAF | RPRLPCSDLIRVPHP | 1492 | TANNPCSDLIRDQGF | 1484 | PC~D~IR | 1478 | −2.02 | 0.38 | |
| | | 16_9 | | | | | | | | | |
| 211920461 | 1743 | KIAA1549_BRAF | PSILPCSDLIRSTKL | 1493 | TANNPCSDLIRDQGF | 1484 | PC~D~IR | 1478 | −2.02 | 0.38 | |
| | | 16_9 | | | | | | | | | |
| 211920461 | 1745 | KIAA1549_BRAF | RSIISDLIRDQGFVA | 1494 | NNPCSDLIRDQGFRG | 1485 | SD~I~DQ | 1479 | −2.00 | 0.03 | DRB1_1501 |
| | | 16_9 | | | | | | | | | |
| 211920461 | 1745 | KIAA1549_BRAF | GSFLSDLIRDQMPPE | 1495 | NNPCSDLIRDQGFRG | 1485 | SD~I~DQ | 1479 | −2.04 | 0.03 | |
| | | 16_9 | | | | | | | | | |
| 211920463 | 1746 | KIAA1549_BRAF | TVLVKTLGRRDLWQL | 1496 | NPCSKTLGRRDSSDD | 1486 | KT~G~RD | 1480 | −2.03 | −0.17 | DRB1_0301 |
| | | 16_11 | | | | | | | | | |
| 211920463 | 1746 | KIAA1549_BRAF | IAAFKTLGRRDKNRE | 1497 | NPCSKTLGRRDSSDD | 1486 | KT~G~RD | 1480 | −2.06 | −0.17 | |
| | | 16_11 | | | | | | | | | |
| 211920465 | 1637 | KIAA1549_BRAF | ISYFGCPDLIRASLM | 1498 | SAYIGCPDLIRDQGF | 1487 | GC~D~IR | 1481 | −2.01 | 0.27 | DRB1_0101 |
| | | 15_9 | | | | | | | | | |
| 211920465 | 1637 | KIAA1549_BRAF | TRFLGCPDLIRGSLM | 1499 | SAYIGCPDLIRDQGF | 1487 | GC~D~IR | 1481 | −2.01 | 0.27 | |
| | | 15_9 | | | | | | | | | |
| 211920465 | 1638 | KIAA1549_BRAF | HLFICPDLIRDSLLH | 1500 | AYIGCPDLIRDQGFR | 1488 | CP~L~RD | 1482 | −2.00 | 0.27 | |
| | | 15_9 | | | | | | | | | |
| 211920465 | 1638 | KIAA1549_BRAF | AFSWCPDLIRDVSYH | 1501 | AYIGCPDLIRDQGFR | 1488 | CP~L~RD | 1482 | −2.06 | 0.27 | |
| | | 15_9 | | | | | | | | | |
| 211920465 | 1639 | KIAA1549_BRAF | DIYLPDLIRDQVGTS | 1502 | YIGCPDLIRDQGFRG | 1489 | PD~I~DQ | 1483 | −2.04 | 0.08 | |
| | | 15_9 | | | | | | | | | |
| 211920465 | 1639 | KIAA1549_BRAF | IPVLPDLIRDQFSLS | 1503 | YIGCPDLIRDQGFRG | 1489 | PD~I~DQ | 1483 | −2.05 | 0.08 | |
| | | 15_9 | | | | | | | | | |
| 211920465 | 1637 | KIAA1549_BRAF | LDMVGCPDLIRRVGV | 1504 | SAYIGCPDLIRDQGF | 1487 | GC~D~IR | 1481 | −2.01 | −0.49 | DRB1_0301 |
| | | 15_9 | | | | | | | | | |
| 211920465 | 1637 | KIAA1549_BRAF | VHEQGCPDLIRWFLH | 1505 | SAYIGCPDLIRDQGF | 1487 | GC~D~IR | 1481 | −2.05 | −0.49 | |
| | | 15_9 | | | | | | | | | |
| 211920465 | 1638 | KIAA1549_BRAF | SDLICPDLIRDPQQL | 1506 | AYIGCPDLIRDQGFR | 1488 | CP~L~RD | 1482 | −2.04 | −0.37 | |
| | | 15_9 | | | | | | | | | |
| 211920465 | 1638 | KIAA1549_BRAF | VELLCPDLIRDLNNQ | 1507 | AYIGCPDLIRDQGFR | 1488 | CP~L~RD | 1482 | −2.04 | −0.37 | |
| | | 15_9 | | | | | | | | | |
| 211920465 | 1639 | KIAA1549_BRAF | YLILPDLIRDQEQTW | 1508 | YIGCPDLIRDQGFRG | 1489 | PD~I~DQ | 1483 | −2.04 | −0.79 | |
| | | 15_9 | | | | | | | | | |
| 211920465 | 1639 | KIAA1549_BRAF | VYLRPDLIRDQQKFA | 1509 | YIGCPDLIRDQGFRG | 1489 | PD~I~DQ | 1483 | −2.05 | −0.79 | |
| | | 15_9 | | | | | | | | | |
| 211920465 | 1637 | KIAA1549_BRAF | YTWRGCPDLIRPTIF | 1510 | SAYIGCPDLIRDQGF | 1487 | GC~D~IR | 1481 | −2.05 | −0.31 | DRB1_0401 |
| | | 15_9 | | | | | | | | | |
| 211920465 | 1637 | KIAA1549_BRAF | YVVYGCPDLIRPQIV | 1511 | SAYIGCPDLIRDQGF | 1487 | GC~D~IR | 1481 | −2.05 | −0.31 | |
| | | 15_9 | | | | | | | | | |
| 211920465 | 1639 | KIAA1549_BRAF | TTVLPDLIRDQEAAR | 1512 | YIGCPDLIRDQGFRG | 1489 | PD~I~DQ | 1483 | −2.05 | −0.22 | |
| | | 15_9 | | | | | | | | | |
| 211920465 | 1639 | KIAA1549_BRAF | ARLEPDLIRDQLLSS | 1513 | YIGCPDLIRDQGFRG | 1489 | PD~I~DQ | 1483 | −2.03 | −0.22 | |
| | | 15_9 | | | | | | | | | |
| 211920465 | 1637 | KIAA1549_BRAF | QIQVGCPDLIRVLPL | 1514 | SAYIGCPDLIRDQGF | 1487 | GC~D~IR | 1481 | −2.09 | 0.18 | DRB1_1501 |
| | | 15_9 | | | | | | | | | |
| 211920465 | 1637 | KIAA1549_BRAF | TVLFGCPDLIRWNRT | 1515 | SAYIGCPDLIRDQGF | 1487 | GC~D~IR | 1481 | −2.05 | 0.18 | |
| | | 15_9 | | | | | | | | | |
| 211920465 | 1638 | KIAA1549_BRAF | FLKLCPDLIRDPAYP | 1516 | AYIGCPDLIRDQGFR | 1488 | CP~L~RD | 1482 | −2.01 | 0.00 | |
| | | 15_9 | | | | | | | | | |
| 211920465 | 1638 | KIAA1549_BRAF | HPVPCPDLIRDPLWT | 1517 | AYIGCPDLIRDQGFR | 1488 | CP~L~RD | 1482 | −2.05 | 0.00 | |
| | | 15_9 | | | | | | | | | |
| 211920465 | 1639 | KIAA1549_BRAF | RLLHPDLIRDQIVVG | 1518 | YIGCPDLIRDQGFRG | 1489 | PD~I~DQ | 1483 | −2.02 | −1.13 | |
| | | 15_9 | | | | | | | | | |
| 211920465 | 1639 | KIAA1549_BRAF | APEFPDLIRDQYYLA | 1519 | YIGCPDLIRDQGFRG | 1489 | PD~I~DQ | 1483 | −2.02 | −1.13 | |
| | | 15_9 | | | | | | | | | |

### Example 15: Bespoke Peptides Targeting EML4-ALK Fusions

Fusions of the echinoderm microtubule-associated protein-like 4 (EML4) gene with the intracellular signaling portion of the receptor tyrosine kinase of anaplastic lymphoma kinase (ALK) gene have been reported in up to 20% of non-small cell lung cancer (NSCLC) cases. EMLA-ALK fusions are also found on other cancers, including ovarian, thyroid, lymphomas and neural tissue tumors. These result in gain of function of the kinase. Several different EML4-ALK fusions are described which have variable clinical outcomes [82, 83]. The fusions have variable lengths of the EML4 component [84, 85]. However, they share a small number of unique T cell exposed motifs at the fusion junction which provide neoepitope targets. Most emphasis in addressing EMLA-ALK cancers has been placed on small molecule drugs [86], with considerable success. However, resistance emerges to the most broadly used drug, crizotinib, showing that alternative approaches including personalized neoepitope vaccines are needed. In the Examples below we provide embodiments of epitopes and bespoke peptides which can direct T cells to the unique junction motifs of EML4 and ALK.

TABLE 21
Bespoke peptides designed for unique motifs at junction site of EML4-ALK fusions for MHC I alleles

| | | | | | | | | | Predicted | Predicted | |
|---|---|---|---|---|---|---|---|---|---|---|---|
| | | | | | | | | | affinity | affinity | |
| | | | proposed | SEQ ID | originating | SEQ ID | TCEM | SEQ ID | proposed | originating | |
| gi | pos | curation | peptide | NO.: | peptide | NO.: | core | NO.: | peptide | peptide | Allele |
| 152002653 | 492 | EML4-ALK var 1 | LLPPKVYRF | 1581 | GKGPKVYRR | 1551 | PKVYR | 1520 | −2.10 | −0.41 | A0201 |
| 152002653 | 491 | EML4-ALK var 1 | KGWGPKVYN | 1582 | PGKGPKVYR | 1552 | GPKVY | 1521 | −2.07 | −1.86 | |
| 152002653 | 493 | EML4-ALK var 1 | MVKKVYRRP | 1583 | KGPKVYRRK | 1553 | KVYRR | 1522 | −1.98 | −1.98 | |
| 152002653 | 490 | EML4-ALK var 1 | HAPKGPKVK | 1584 | TPGKGPKVY | 1554 | KGPKV | 1523 | −2.05 | −0.15 | A1101 |
| 152002653 | 491 | EML4-ALK var 1 | AHEGPKVYK | 1585 | PGKGPKVYR | 1552 | GPKVY | 1521 | −2.02 | −0.51 | |
| 152002653 | 492 | EML4-ALK var 1 | WAIPKVYRP | 1586 | GKGPKVYRR | 1551 | PKVYR | 1520 | −2.02 | −1.22 | |
| 152002653 | 493 | EML4-ALK var 1 | YTMKVYRRR | 1587 | KGPKVYRRK | 1553 | KVYRR | 1522 | −2.05 | −2.39 | |
| 152002653 | 490 | EML4-ALK var 1 | TSPKGPKVR | 1588 | TPGKGPKVY | 1554 | KGPKV | 1523 | −2.01 | −0.19 | A6801 |
| 152002653 | 491 | EML4-ALK var 1 | VTTGPKVYK | 1589 | PGKGPKVYR | 1552 | GPKVY | 1521 | −2.02 | −2.25 | |
| 152002653 | 492 | EML4-ALK var 1 | ESSPKVYRK | 1590 | GKGPKVYRR | 1551 | PKVYR | 1520 | −1.99 | −0.53 | |
| 152002653 | 493 | EML4-ALK var 1 | NPYKVYRRR | 1591 | KGPKVYRRK | 1553 | KVYRR | 1522 | −2.00 | −1.74 | |
| 152002653 | 490 | EML4-ALK var 1 | PVNKGPKVL | 1592 | TPGKGPKVY | 1554 | KGPKV | 1523 | −2.10 | −1.42 | B0702 |
| 152002653 | 490 | EML4-ALK var 1 | NRQKGPKVR | 1593 | TPGKGPKVY | 1554 | KGPKV | 1523 | −2.01 | −0.36 | B2705 |
| 152002653 | 492 | EML4-ALK var 1 | TRGPKVYRL | 1594 | GKGPKVYRR | 1551 | PKVYR | 1520 | −2.00 | −0.92 | |
| 152002653 | 490 | EML4-ALK var 1 | DPSKGPKVF | 1595 | TPGKGPKVY | 1554 | KGPKV | 1523 | −2.04 | −1.97 | B3501 |
| 152002653 | 491 | EML4-ALK var 1 | CTSGPKVYW | 1596 | PGKGPKVYR | 1552 | GPKVY | 1521 | −2.05 | −0.30 | |
| 152002655 | 741 | EML4-ALK var 2 | THVEILYLG | 1597 | GDYEILYLY | 1555 | EILYL | 1524 | −1.98 | −0.30 | A0201 |
| 152002655 | 743 | EML4-ALK var 2 | LGILYLYRA | 1598 | YEILYLYRR | 1556 | LYLYR | 1525 | −1.97 | −0.04 | |
| 152002655 | 745 | EML4-ALK var 2 | KILLYRRKI | 1599 | ILYLYRRKH | 1556 | LYRRK | 1525 | −2.03 | −0.10 | |
| 152002655 | 741 | EML4-ALK var 2 | IEPEILYLR | 1600 | GDYEILYLY | 1555 | EILYL | 1524 | −1.99 | −0.06 | A0301 |
| 152002655 | 742 | EML4-ALK var 2 | TIAILYLYQ | 1601 | DYEILYLYR | 1556 | ILYLY | 1525 | −2.03 | −0.66 | |
| 152002655 | 744 | EML4-ALK var 2 | AASYLYRRK | 1602 | EILYLYRRK | 1557 | YLYRR | 1526 | −2.05 | −0.57 | |
| 152002655 | 741 | EML4-ALK var 2 | TVLEILYLN | 1603 | GDYEILYLY | 1555 | EILYL | 1524 | −2.09 | −1.89 | A1101 |
| 152002655 | 742 | EML4-ALK var 2 | VIFILYLYQ | 1604 | DYEILYLYR | 1556 | ILYLY | 1525 | −2.05 | −1.26 | |
| 152002655 | 743 | EML4-ALK var 2 | SLDLYLYRR | 1605 | YEILYLYRR | 1556 | LYLYR | 1525 | −2.02 | −2.21 | |
| 152002655 | 744 | EML4-ALK var 2 | STCYLYRRP | 1606 | EILYLYRRK | 1557 | YLYRR | 1526 | −1.99 | −2.40 | |
| 152002655 | 745 | EML4-ALK var 2 | PTELYRRKK | 1607 | ILYLYRRKH | 1556 | LYRRK | 1525 | −2.03 | −0.23 | |
| 152002655 | 741 | EML4-ALK var 2 | LFLEILYLR | 1608 | GDYEILYLY | 1555 | EILYL | 1524 | −2.03 | −0.73 | A6801 |
| 152002655 | 742 | EML4-ALK var 2 | IYAILYLYR | 1609 | DYEILYLYR | 1556 | ILYLY | 1525 | −2.06 | −1.79 | |
| 152002655 | 743 | EML4-ALK var 2 | YVGLYLYRK | 1610 | YEILYLYRR | 1556 | LYLYR | 1525 | −1.98 | −1.72 | |
| 152002655 | 744 | EML4-ALK var 2 | GIMYLYRRR | 1611 | EILYLYRRK | 1557 | YLYRR | 1526 | −2.02 | −2.49 | |
| 152002655 | 745 | EML4-ALK var 2 | STYLYRRKR | 1612 | ILYLYRRKH | 1556 | LYRRK | 1525 | −1.99 | −0.68 | |
| 152002655 | 741 | EML4-ALK var 2 | KPGEILYLY | 1613 | GDYEILYLY | 1555 | EILYL | 1524 | −2.00 | −0.07 | B0702 |
| 152002655 | 741 | EML4-ALK var 2 | DTPEILYLF | 1614 | GDYEILYLY | 1555 | EILYL | 1524 | −2.10 | −1.84 | B1501 |
| 152002655 | 745 | EML4-ALK var 2 | ILPLYRRKF | 1615 | ILYLYRRKH | 1556 | LYRRK | 1525 | −2.06 | −0.62 | |
| 152002655 | 741 | EML4-ALK var 2 | PYLEILYLA | 1616 | GDYEILYLY | 1555 | EILYL | 1524 | −2.07 | −1.63 | B2705 |
| 152002655 | 742 | EML4-ALK var 2 | NKEILYLYP | 1617 | DYEILYLYR | 1556 | ILYLY | 1525 | −2.08 | −0.16 | |
| 152002655 | 743 | EML4-ALK var 2 | ERSLYLYRP | 1618 | YEILYLYRR | 1556 | LYLYR | 1525 | −2.08 | −1.68 | |
| 152002655 | 745 | EML4-ALK var 2 | GRELYRRKK | 1619 | ILYLYRRKH | 1556 | LYRRK | 1525 | −2.08 | −0.22 | |
| 152002655 | 741 | EML4-ALK var 2 | LAFEILYLV | 1620 | GDYEILYLY | 1555 | EILYL | 1524 | −2.00 | −0.63 | B3501 |
| 152002655 | 742 | EML4-ALK var 2 | KGVILYLYM | 1621 | DYEILYLYR | 1556 | ILYLY | 1525 | −2.04 | −0.80 | |
| 152002655 | 743 | EML4-ALK var 2 | LPILYLYRL | 1622 | YEILYLYRR | 1556 | LYLYR | 1525 | −2.01 | −0.79 | |
| 152002655 | 745 | EML4-ALK var 2 | YPLLYRRKM | 1623 | ILYLYRRKH | 1556 | LYRRK | 1525 | −2.08 | −0.79 | |
| 194072593 | 214 | EML4-ALK var 3 | SFDDVIINL | 1624 | KHKDVIINQ | 1558 | DVIIN | 1527 | −1.98 | −0.08 | A0201 |
| | | isoform a | | | | | | | | | |
| 194072593 | 215 | EML4-ALK var 3 | PLIVIINQP | 1625 | HKDVIINQV | 1559 | VIINQ | 1528 | −1.96 | −0.79 | |
| | | isoform a | | | | | | | | | |
| 194072593 | 216 | EML4-ALK var 3 | PVIIINQVV | 1626 | KDVIINQVY | 1560 | IINQV | 1529 | −2.00 | −0.01 | |
| | | isoform a | | | | | | | | | |
| 194072593 | 214 | EML4-ALK var 3 | ASGDVIINP | 1627 | KHKDVIINQ | 1558 | DVIIN | 1527 | −2.09 | −1.18 | A0301 |
| | | isoform a | | | | | | | | | |
| 194072593 | 217 | EML4-ALK var 3 | YFNINQVYK | 1628 | DVIINQVYR | 1561 | INQVY | 1530 | −2.08 | −0.55 | |
| | | isoform a | | | | | | | | | |
| 194072593 | 216 | EML4-ALK var 3 | RAYIINQVP | 1629 | KDVIINQVY | 1560 | INQV | 1529 | −2.01 | −0.67 | A1101 |
| | | isoform a | | | | | | | | | |
| 194072593 | 217 | EML4-ALK var 3 | KIDINQVYR | 1630 | DVIINQVYR | 1561 | INQVY | 1530 | −2.01 | −2.19 | |
| | | isoform a | | | | | | | | | |
| 194072593 | 218 | EML4-ALK var 3 | SKVNQVYRK | 1631 | VIINQVYRR | 1562 | NQVYR | 1531 | −2.01 | −2.47 | |
| | | isoform a | | | | | | | | | |
| 194072593 | 214 | EML4-ALK var 3 | WKGDVIINR | 1632 | KHKDVIINQ | 1558 | DVIIN | 1527 | −1.98 | −0.27 | A6801 |
| | | isoform a | | | | | | | | | |
| 194072593 | 216 | EML4-ALK var 3 | RINIINQVK | 1633 | KDVIINQVY | 1560 | IINQV | 1529 | −1.99 | −1.08 | |
| | | isoform a | | | | | | | | | |
| 194072593 | 217 | EML4-ALK var 3 | QLVINQVYK | 1634 | DVIINQVYR | 1561 | INQVY | 1530 | −2.00 | −2.85 | |
| | | isoform a | | | | | | | | | |
| 194072593 | 218 | EML4-ALK var 3 | DSMNQVYRK | 1635 | VIINQVYRR | 1562 | NQVYR | 1531 | −2.00 | −2.22 | |
| | | isoform a | | | | | | | | | |
| 194072593 | 216 | EML4-ALK var 3 | RVRIINQVS | 1636 | KDVIINQVY | 1560 | IINQV | 1529 | −2.04 | −0.13 | B0702 |
| | | isoform a | | | | | | | | | |
| 194072593 | 216 | EML4-ALK var 3 | VSAIINQVY | 1637 | KDVIINQVY | 1560 | IINQV | 1529 | −2.04 | −2.40 | B1501 |
| | | isoform a | | | | | | | | | |
| 194072593 | 214 | EML4-ALK var 3 | LKDDVIINS | 1638 | KHKDVIINQ | 1558 | DVIIN | 1527 | −2.00 | −0.56 | B2705 |
| | | isoform a | | | | | | | | | |
| 194072593 | 215 | EML4-ALK var 3 | RKNVIINQG | 1639 | HKDVIINQV | 1559 | VIINQ | 1528 | −2.01 | −0.53 | |
| | | isoform a | | | | | | | | | |
| 194072593 | 216 | EML4-ALK var 3 | LKDIINQVH | 1640 | KDVIINQVY | 1560 | IINQV | 1529 | −2.06 | −1.93 | |
| | | isoform a | | | | | | | | | |
| 194072593 | 217 | EML4-ALK var 3 | FKTINQVYY | 1641 | DVIINQVYR | 1561 | INQVY | 1530 | −2.09 | −0.28 | |
| | | isoform a | | | | | | | | | |
| 194072593 | 216 | EML4-ALK var 3 | QPKIINQVY | 1642 | KDVIINQVY | 1560 | IINQV | 1529 | −2.00 | −1.33 | B3501 |
| | | isoform a | | | | | | | | | |
| 194072593 | 217 | EML4-ALK var 3 | FAMINQVYA | 1643 | DVIINQVYR | 1561 | INQVY | 1530 | −2.04 | −0.34 | |
| | | isoform a | | | | | | | | | |
| 194072593 | 218 | EML4-ALK var 3 | WLANQVYRI | 1644 | VIINQVYRR | 1562 | NQVYR | 1531 | −2.02 | −0.40 | |
| | | isoform a | | | | | | | | | |
| 194072595 | 218 | EML4-ALK var 3 | SLCNQAKML | 1645 | VIINQAKMS | 1563 | NQAKM | 1532 | −1.99 | −0.53 | A0201 |
| | | isoform b | | | | | | | | | |
| 194072595 | 219 | EML4-ALK var 3 | TIHQAKMSV | 1646 | IINQAKMST | 1564 | QAKMS | 1533 | −2.04 | −0.24 | |
| | | isoform b | | | | | | | | | |
| 194072595 | 220 | EML4-ALK var 3 | ELFAKMSTL | 1647 | INQAKMSTR | 1565 | AKMST | 1534 | −1.97 | −0.03 | |
| | | isoform b | | | | | | | | | |
| 194072595 | 222 | EML4-ALK var 3 | TLIMSTREA | 1648 | QAKMSTREK | 1566 | MSTRE | 1535 | −2.12 | −0.02 | |
| | | isoform b | | | | | | | | | |
| 194072595 | 224 | EML4-ALK var 3 | KLVTREKNL | 1649 | KMSTREKNS | 1567 | TREKN | 1536 | −2.08 | −0.44 | |
| | | isoform b | | | | | | | | | |
| 194072595 | 226 | EML4-ALK var 3 | RLQEKNSQV | 1650 | STREKNSQV | 1568 | EKNSQ | 1537 | −2.10 | −0.32 | |
| | | isoform b | | | | | | | | | |
| 194072595 | 228 | EML4-ALK var 3 | AAFNSQVYV | 1651 | REKNSQVYR | 1569 | NSQVY | 1538 | −2.11 | −0.16 | |
| | | isoform b | | | | | | | | | |
| 194072595 | 229 | EML4-ALK var 3 | VLESQVYRF | 1652 | EKNSQVYRR | 1570 | SQVYR | 1539 | −1.97 | −0.52 | |
| | | isoform b | | | | | | | | | |
| 194072595 | 216 | EML4-ALK var 3 | PDFIINQAK | 1653 | KDVIINQAK | 1571 | IINQA | 1540 | −2.02 | −0.33 | A0301 |
| | | isoform b | | | | | | | | | |
| 194072595 | 222 | EML4-ALK var 3 | NPFMSTREK | 1654 | QAKMSTREK | 1566 | MSTRE | 1535 | −2.02 | −2.02 | |
| | | isoform b | | | | | | | | | |
| 194072595 | 224 | EML4-ALK var 3 | EIITREKNK | 1655 | KMSTREKNS | 1567 | TREKN | 1536 | −2.04 | −0.15 | |
| | | isoform b | | | | | | | | | |
| 194072595 | 226 | EML4-ALK var 3 | RFSEKNSQK | 1656 | STREKNSQV | 1568 | EKNSQ | 1537 | −2.04 | −0.76 | |
| | | isoform b | | | | | | | | | |
| 194072595 | 228 | EML4-ALK var 3 | RNLNSQVYR | 1657 | REKNSQVYR | 1569 | NSQVY | 1538 | −2.03 | −1.48 | |
| | | isoform b | | | | | | | | | |
| 194072595 | 230 | EML4-ALK var 3 | GMEQVYRRK | 1658 | KNSQVYRRK | 1572 | QVYRR | 1541 | −2.00 | −0.94 | |
| | | isoform b | | | | | | | | | |
| 194072595 | 230 | EML4-ALK var 3 | GTPQVYRRR | 1659 | IINQVYRRK | 1572 | QVYRR | 1541 | −2.10 | −1.81 | A1101 |
| | | isoform b | | | | | | | | | |
| 194072595 | 216 | EML4-ALK var 3 | RMDIINQAK | 1660 | KDVIINQAK | 1571 | IINQA | 1540 | −1.98 | −1.90 | |
| | | isoform b | | | | | | | | | |
| 194072595 | 218 | EML4-ALK var 3 | LPFNQAKMK | 1661 | VIINQAKMS | 1563 | NQAKM | 1532 | −2.05 | −0.13 | |
| | | isoform b | | | | | | | | | |
| 194072595 | 219 | EML4-ALK var 3 | SSVQAKMSP | 1662 | IINQAKMST | 1564 | QAKMS | 1533 | −2.04 | −0.32 | |
| | | isoform b | | | | | | | | | |
| 194072595 | 220 | EML4-ALK var 3 | HTNAKMSTK | 1663 | INQAKMSTR | 1565 | AKMST | 1534 | −2.05 | −1.12 | |
| | | isoform b | | | | | | | | | |
| 194072595 | 222 | EML4-ALK var 3 | PSHMSTREK | 1664 | QAKMSTREK | 1566 | MSTRE | 1535 | −2.00 | −1.93 | |
| | | isoform b | | | | | | | | | |
| 194072595 | 225 | EML4-ALK var 3 | LAAREKNSK | 1665 | MSTREKNSQ | 1573 | REKNS | 1542 | −1.98 | −0.37 | |
| | | isoform b | | | | | | | | | |
| 194072595 | 228 | EML4-ALK var 3 | AEPNSQVYK | 1666 | REKNSQVYR | 1569 | NSQVY | 1538 | −1.98 | −0.82 | |
| | | isoform b | | | | | | | | | |
| 194072595 | 216 | EML4-ALK var 3 | SGNIINQAK | 1667 | KDVIINQAK | 1571 | IINQA | 1540 | −1.99 | −1.50 | A6801 |
| | | isoform b | | | | | | | | | |
| 194072595 | 217 | EML4-ALK var 3 | CPAINQAKK | 1668 | DVIINQAKM | 1574 | INQAK | 1543 | −1.99 | −0.46 | B0702 |
| | | isoform b | | | | | | | | | |
| 194072595 | 219 | EML4-ALK var 3 | RPSQAKMSR | 1669 | IINQAKMST | 1564 | QAKMS | 1533 | −2.01 | −0.03 | |
| | | isoform b | | | | | | | | | |
| 194072595 | 220 | EML4-ALK var 3 | ILIAKMSTR | 1670 | INQAKMSTR | 1565 | AKMST | 1534 | −1.98 | −1.49 | |
| | | isoform b | | | | | | | | | |
| 194072595 | 221 | EML4-ALK var 3 | QGRKMSTRK | 1671 | NQAKMSTRE | 1575 | KMSTR | 1544 | −2.06 | −0.46 | |
| | | isoform b | | | | | | | | | |
| 194072595 | 222 | EML4-ALK var 3 | ESSMSTREK | 1672 | QAKMSTREK | 1566 | MSTRE | 1535 | −2.01 | −1.81 | |
| | | isoform b | | | | | | | | | |
| 194072595 | 223 | EML4-ALK var 3 | HGSSTREKW | 1673 | AKMSTREKN | 1576 | STREK | 1545 | −2.02 | −0.45 | |
| | | isoform b | | | | | | | | | |
| 194072595 | 225 | EML4-ALK var 3 | SSIREKNSR | 1674 | MSTREKNSQ | 1573 | REKNS | 1542 | −2.07 | −1.26 | |
| | | isoform b | | | | | | | | | |
| 194072595 | 228 | EML4-ALK var 3 | IEWNSQVYR | 1675 | REKNSQVYR | 1569 | NSQVY | 1538 | −2.01 | −1.70 | |
| | | isoform b | | | | | | | | | |
| 194072595 | 229 | EML4-ALK var 3 | ETISQVYRK | 1676 | EKNSQVYRR | 1570 | SQVYR | 1539 | −2.06 | −1.51 | |
| | | isoform b | | | | | | | | | |
| 194072595 | 230 | EML4-ALK var 3 | KGMQVYRRK | 1677 | KNSQVYRRK | 1572 | QVYRR | 1541 | −2.06 | −1.28 | |
| | | isoform b | | | | | | | | | |
| 194072595 | 217 | EML4-ALK var 3 | SGRINQAKF | 1678 | DVIINQAKM | 1574 | INQAK | 1543 | −1.99 | −0.79 | |
| | | isoform b | | | | | | | | | |
| 194072595 | 219 | EML4-ALK var 3 | GPVQAKMSH | 1679 | IINQAKMST | 1564 | QAKMS | 1533 | −2.07 | −0.75 | |
| | | isoform b | | | | | | | | | |
| 194072595 | 224 | EML4-ALK var 3 | FPGTREKNN | 1680 | KMSTREKNS | 1567 | TREKN | 1536 | −2.05 | −0.03 | |
| | | isoform b | | | | | | | | | |
| 194072595 | 225 | EML4-ALK var 3 | CSTREKNSL | 1681 | MSTREKNSQ | 1573 | REKNS | 1542 | −2.12 | −0.44 | |
| | | isoform b | | | | | | | | | |
| 194072595 | 226 | EML4-ALK var 3 | FAMEKNSQL | 1682 | STREKNSQV | 1568 | EKNSQ | 1537 | −2.03 | −1.17 | |
| | | isoform b | | | | | | | | | |
| 194072595 | 228 | EML4-ALK var 3 | VPQNSQVYG | 1683 | REKNSQVYR | 1569 | NSQVY | 1538 | −1.97 | −0.14 | |
| | | isoform b | | | | | | | | | |
| 194072595 | 216 | EML4-ALK var 3 | LETIINQAY | 1684 | KDVIINQAK | 1571 | IINQA | 1540 | −2.09 | −0.20 | B1501 |
| | | isoform b | | | | | | | | | |
| 194072595 | 217 | EML4-ALK var 3 | YMLINQAKI | 1685 | DVIINQAKM | 1574 | INQAK | 1543 | −2.09 | −2.19 | |
| | | isoform b | | | | | | | | | |
| 194072595 | 220 | EML4-ALK var 3 | KSFAKMSTL | 1686 | INQAKMSTR | 1565 | AKMST | 1534 | −2.03 | −0.72 | |
| | | isoform b | | | | | | | | | |
| 194072595 | 221 | EML4-ALK var 3 | NVSKMSTRF | 1687 | NQAKMSTRE | 1575 | KMSTR | 1544 | −1.99 | −0.01 | |
| | | isoform b | | | | | | | | | |
| 194072595 | 224 | EML4-ALK var 3 | GFMTREKNF | 1688 | KMSTREKNS | 1567 | TREKN | 1536 | −1.99 | −1.02 | |
| | | isoform b | | | | | | | | | |
| 194072595 | 227 | EML4-ALK var 3 | SVAKNSQVY | 1689 | TREKNSQVY | 1577 | KNSQV | 1546 | −2.03 | −0.28 | |
| | | isoform b | | | | | | | | | |
| 194072595 | 216 | EML4-ALK var 3 | GKRIINQAR | 1690 | KDVIINQAK | 1571 | IINQA | 1540 | −2.01 | −2.60 | B2705 |
| | | isoform b | | | | | | | | | |
| 194072595 | 217 | EML4-ALK var 3 | SRDINQAKQ | 1691 | DVIINQAKM | 1574 | INQAK | 1543 | −2.08 | −0.31 | |
| | | isoform b | | | | | | | | | |
| 194072595 | 221 | EML4-ALK var 3 | SRTKMSTRM | 1692 | NQAKMSTRE | 1575 | KMSTR | 1544 | −1.98 | −0.49 | |
| | | isoform b | | | | | | | | | |
| 194072595 | 223 | EML4-ALK var 3 | RRGSTREKA | 1693 | AKMSTREKN | 1576 | STREK | 1545 | −2.02 | −1.25 | |
| | | isoform b | | | | | | | | | |
| 194072595 | 227 | EML4-ALK var 3 | KRQKNSQVI | 1694 | TREKNSQVY | 1577 | KNSQV | 1546 | −2.09 | −0.92 | |
| | | isoform b | | | | | | | | | |
| 194072595 | 228 | EML4-ALK var 3 | GRVNSQVYS | 1695 | REKNSQVYR | 1569 | NSQVY | 1538 | −2.06 | −0.89 | |
| | | isoform b | | | | | | | | | |
| 194072595 | 229 | EML4-ALK var 3 | PKGSQVYRE | 1696 | EKNSQVYRR | 1570 | SQVYR | 1539 | −2.07 | −0.74 | |
| | | isoform b | | | | | | | | | |
| 194072595 | 230 | EML4-ALK var 3 | YKAQVYRRK | 1697 | KNSQVYRRK | 1572 | QVYRR | 1541 | −1.98 | −0.96 | |
| | | isoform b | | | | | | | | | |
| 194072595 | 217 | EML4-ALK var 3 | WAQINQAKR | 1698 | DVIINQAKM | 1574 | INQAK | 1543 | −2.02 | −1.14 | B3501 |
| | | isoform b | | | | | | | | | |
| 194072595 | 219 | EML4-ALK var 3 | FPDQAKMSG | 1699 | IINQAKMST | 1564 | QAKMS | 1533 | −2.00 | −0.15 | |
| | | isoform b | | | | | | | | | |
| 194072595 | 220 | EML4-ALK var 3 | RSCAKMSTL | 1700 | INQAKMSTR | 1565 | AKMST | 1534 | −2.01 | −0.55 | |
| | | isoform b | | | | | | | | | |
| 194072595 | 225 | EML4-ALK var 3 | IYEREKNSF | 1701 | MSTREKNSQ | 1573 | REKNS | 1542 | −2.05 | −0.77 | |
| | | isoform b | | | | | | | | | |
| 194072595 | 227 | EML4-ALK var 3 | FPLKNSQVA | 1702 | TREKNSQVY | 1577 | KNSQV | 1546 | −2.05 | −0.40 | |
| | | isoform b | | | | | | | | | |
| 194072595 | 229 | EML4-ALK var 3 | YGISQVYRL | 1703 | EKNSQVYRR | 1570 | SQVYR | 1539 | −2.07 | −0.32 | |
| | | isoform b | | | | | | | | | |
| 227452649 | 513 | EML4-ALK var 6 | ALSHPPAVY | 1704 | AADHPPAVY | 1578 | HPPAV | 1547 | −2.06 | −0.14 | A0301 |
| 227452649 | 516 | EML4-ALK var 6 | VAAAVYRRK | 1705 | HPPAVYRRK | 1579 | AVYRR | 1548 | −2.01 | −0.78 | |
| 227452649 | 513 | EML4-ALK var 6 | KGGHPPAVP | 1706 | AADHPPAVY | 1578 | HPPAV | 1547 | −1.98 | −1.01 | A1101 |
| 227452649 | 514 | EML4-ALK var 6 | IKPPPAVYK | 1707 | ADHPPAVYR | 1580 | PPAVY | 1549 | −2.02 | −2.17 | |
| 227452649 | 515 | EML4-ALK var 6 | IEPPAVYRK | 1708 | DHPPAVYRR | 1581 | PAVYR | 1550 | −2.04 | −0.35 | |
| 227452649 | 516 | EML4-ALK var 6 | LSLAVYRRR | 1709 | HPPAVYRRK | 1579 | AVYRR | 1548 | −2.03 | −1.69 | |
| 227452649 | 513 | EML4-ALK var 6 | IAGHPPAVK | 1710 | AADHPPAVY | 1578 | HPPAV | 1547 | −2.05 | −0.60 | A6801 |
| 227452649 | 514 | EML4-ALK var 6 | LIIPPAVYK | 1711 | ADHPPAVYR | 1580 | PPAVY | 1549 | −2.05 | −1.52 | |
| 227452649 | 515 | EML4-ALK var 6 | TLLPAVYRR | 1712 | DHPPAVYRR | 1581 | PAVYR | 1550 | −2.04 | −1.32 | |
| 227452649 | 516 | EML4-ALK var 6 | DSRAVYRRK | 1713 | HPPAVYRRK | 1579 | AVYRR | 1548 | −1.99 | −2.12 | |
| 227452649 | 513 | EML4-ALK var 6 | LSGHPPAVL | 1714 | AADHPPAVY | 1578 | HPPAV | 1547 | −2.06 | −0.40 | B0702 |
| 227452649 | 513 | EML4-ALK var 6 | LPLHPPAVY | 1715 | AADHPPAVY | 1578 | HPPAV | 1547 | −2.02 | −1.99 | B1501 |
| 227452649 | 514 | EML4-ALK var 6 | ETSPPAVYM | 1716 | ADHPPAVYR | 1580 | PPAVY | 1549 | −2.07 | −0.19 | |
| 227452649 | 515 | EML4-ALK var 6 | DRGPAVYRE | 1717 | DHPPAVYRR | 1581 | PAVYR | 1550 | −2.04 | −0.79 | B2705 |
| 227452649 | 513 | EML4-ALK var 6 | KGVHPPAVM | 1718 | AADHPPAVY | 1578 | HPPAV | 1547 | −2.06 | −1.47 | B3501 |
| 227452649 | 516 | EML4-ALK var 6 | HPCAVYRRL | 1719 | HPPAVYRRK | 1579 | AVYRR | 1548 | −2.07 | −0.46 | |

TABLE 22
Bespoke peptides designed for unique motifs at junction site of EML4-ALK fusions for MHC II alleles

| | | | | | | | | | affinity | affinity | |
|---|---|---|---|---|---|---|---|---|---|---|---|
| | | | | SEQ | | SEQ | | SEQ | proposed | originating | |
| | | | | ID | originating | ID | TCEM | ID | peptide | peptide | |
| gi | pos | curation | proposed peptide | NO.: | peptide | NO.: | core | NO.: | Predicted | Predicted | Allele |
| 152002653 | 490 | EML4-ALK var 1 | CTLLGPKVYRRLYHK | 1772 | TPGKGPKVYRRKHQE | 1746 | GP~V~RR | 1720 | −1.97 | −0.19 | DRB1_0301 |
| 152002653 | 491 | EML4-ALK var 1 | VMIFPKVYRRKYKLS | 1773 | PGKGPKVYRRKHQEL | 1747 | PK~Y~RK | 1721 | −1.97 | −0.16 | |
| 152002653 | 492 | EML4-ALK var 1 | VQLYKVYRRKHRQDS | 1774 | GKGPKVYRRKHQELQ | 1748 | KV~R~KH | 1722 | −2.05 | −1.08 | |
| 152002655 | 741 | EML4-ALK var 2 | SMEIILYLYRRAFPL | 1775 | GDYEILYLYRRKHQE | 1749 | IL~L~RR | 1723 | −2.05 | −0.01 | DRB1_0101 |
| 152002655 | 742 | EML4-ALK var 2 | KHPYLYLYRRKTISY | 1776 | DYEILYLYRRKHQEL | 1750 | LY~Y~RK | 1724 | −2.07 | −0.57 | |
| 152002655 | 743 | EML4-ALK var 2 | LRVLYLYRRKHVPVE | 1777 | YEILYLYRRKHQELQ | 1751 | YL~R~KH | 1725 | −1.99 | −0.63 | |
| 152002655 | 744 | EML4-ALK var 2 | TFIHLYRRKHQLLTT | 1778 | EILYLYRRKHQELQA | 1752 | LY~R~HQ | 1726 | −2.01 | −0.65 | |
| 152002655 | 745 | EML4-ALK var 2 | IWLYYRRKHQELSPG | 1779 | ILYLYRRKHQELQAM | 1753 | YR~K~QE | 1727 | −2.04 | −0.45 | |
| 152002655 | 741 | EML4-ALK var 2 | LDPIILYLYRRNHPV | 1780 | GDYEILYLYRRKHQE | 1749 | IL~L~RR | 1723 | −2.08 | −1.64 | DRB1_0301 |
| 152002655 | 742 | EML4-ALK var 2 | DSNILYLYRRKFIED | 1781 | DYEILYLYRRKHQEL | 1750 | LY~Y~RK | 1724 | −1.98 | −1.66 | |
| 152002655 | 743 | EML4-ALK var 2 | PLPPYLYRRKHMPFE | 1782 | YEILYLYRRKHQELQ | 1751 | YL~R~KH | 1725 | −2.10 | −2.72 | |
| 152002655 | 744 | EML4-ALK var 2 | IEFTLYRRKHQKLRY | 1783 | EILYLYRRKHQELQA | 1752 | LY~R~HQ | 1726 | −2.04 | −2.03 | |
| 152002655 | 745 | EML4-ALK var 2 | LHARYRRKHQEIYWE | 1784 | ILYLYRRKHQELQAM | 1753 | YR~K~QE | 1727 | −1.98 | −1.46 | |
| 152002655 | 741 | EML4-ALK var 2 | KPHIILYLYRRFCGT | 1785 | GDYEILYLYRRKHQE | 1749 | IL~L~RR | 1723 | −2.09 | −0.41 | DRB1_0401 |
| 152002655 | 742 | EML4-ALK var 2 | KLSYLYLYRRKGNGI | 1786 | DYEILYLYRRKHQEL | 1750 | LY~Y~RK | 1724 | −2.04 | −1.53 | |
| 152002655 | 743 | EML4-ALK var 2 | DVYPYLYRRKHPSRF | 1787 | YEILYLYRRKHQELQ | 1751 | YL~R~KH | 1725 | −2.02 | −1.00 | |
| 152002655 | 744 | EML4-ALK var 2 | LGLYLYRRKHQSPKP | 1788 | EILYLYRRKHQELQA | 1752 | LY~R~HQ | 1726 | −2.07 | −1.40 | |
| 152002655 | 745 | EML4-ALK var 2 | IGWVYRRKHQEFSLI | 1789 | ILYLYRRKHQELQAM | 1753 | YR~K~QE | 1727 | −2.01 | −1.76 | |
| 152002655 | 741 | EML4-ALK var 2 | LSEYILYLYRRIPCP | 1790 | GDYEILYLYRRKHQE | 1749 | IL~L~RR | 1723 | −1.99 | −1.42 | DRB1_1501 |
| 152002655 | 742 | EML4-ALK var 2 | LIENLYLYRRKLLAR | 1791 | DYEILYLYRRKHQEL | 1750 | LY~Y~RK | 1724 | −2.07 | −1.95 | |
| 152002655 | 743 | EML4-ALK var 2 | KYINYLYRRKHALHE | 1792 | YEILYLYRRKHQELQ | 1751 | YL~R~KH | 1725 | −2.05 | −1.64 | |
| 152002655 | 744 | EML4-ALK var 2 | EKFLLYRRKHQVNFE | 1793 | EILYLYRRKHQELQA | 1752 | LY~R~HQ | 1726 | −2.05 | −2.05 | |
| 194072593 | 214 | EML4-ALK var 3 | VTVVVIINQVYIVLE | 1794 | KHKDVIINQVYRRKH | 1754 | VI~N~VY | 1728 | −2.07 | −1.10 | DRB1_0301 |
| | | isoform a | | | | | | | | | |
| 194072593 | 215 | EML4-ALK var 3 | IGWLIINQVYRRQGY | 1795 | HKDVIINQVYRRKHQ | 1755 | II~Q~YR | 1729 | −2.09 | −2.40 | |
| | | isoform a | | | | | | | | | |
| 194072593 | 216 | EML4-ALK var 3 | YTFVINQVYRRHKES | 1796 | KDVIINQVYRRKHQE | 1755 | IN~V~RR | 1729 | −1.98 | −3.26 | |
| | | isoform a | | | | | | | | | |
| 194072593 | 217 | EML4-ALK var 3 | QPLFNQVYRRKEIAI | 1797 | DVIINQVYRRKHQEL | 1756 | NQ~Y~RK | 1730 | −2.05 | −2.38 | |
| | | isoform a | | | | | | | | | |
| 194072593 | 216 | EML4-ALK var 3 | RVGYINQVYRRAYGP | 1798 | KDVIINQVYRRKHQE | 1755 | IN~V~RR | 1729 | −1.98 | −0.01 | DRB1_0401 |
| | | isoform a | | | | | | | | | |
| 194072593 | 217 | EML4-ALK var 3 | DRFLNQVYRRKYPMH | 1799 | DVIINQVYRRKHQEL | 1756 | NQ~Y~RK | 1730 | −2.07 | −0.11 | |
| | | isoform a | | | | | | | | | |
| 194072593 | 214 | EML4-ALK var 3 | DNFTVIINQVYPLYG | 1800 | KHKDVIINQVYRRKH | 1754 | VI~N~VY | 1728 | −1.99 | −0.87 | DRB1_1501 |
| | | isoform a | | | | | | | | | |
| 194072593 | 215 | EML4-ALK var 3 | TCMFIINQVYRLPGH | 1801 | HKDVIINQVYRRKHQ | 1755 | II~Q~YR | 1729 | −1.98 | −0.57 | |
| | | isoform a | | | | | | | | | |
| 194072593 | 216 | EML4-ALK var 3 | DLMFINQVYRRLSKT | 1802 | KDVIINQVYRRKHQE | 1755 | IN~V~RR | 1729 | −2.08 | −1.06 | |
| | | isoform a | | | | | | | | | |
| 194072593 | 217 | EML4-ALK var 3 | KIHVNQVYRRKILFF | 1803 | DVIINQVYRRKHQEL | 1756 | NQ~Y~RK | 1730 | −1.98 | −0.57 | |
| | | isoform a | | | | | | | | | |
| 194072595 | 216 | EML4-ALK var 3 | STMLINQAKMSWIMP | 1804 | KDVIINQAKMSTREK | 1757 | IN~A~MS | 1731 | −1.96 | −2.22 | DRB1_0301 |
| | | isoform b | | | | | | | | | |
| 194072595 | 218 | EML4-ALK var 3 | SPMFQAKMSTRNQQT | 1805 | VIINQAKMSTREKNS | 1758 | QA~M~TR | 1732 | −1.99 | −0.40 | |
| | | isoform b | | | | | | | | | |
| 194072595 | 220 | EML4-ALK var 3 | NENLKMSTREKRIHL | 1806 | INQAKMSTREKNSQV | 1759 | KM~T~EK | 1733 | −2.04 | −0.34 | |
| | | isoform b | | | | | | | | | |
| 194072595 | 221 | EML4-ALK var 3 | VIILMSTREKNDPEF | 1807 | NQAKMSTREKNSQVY | 1760 | MS~R~KN | 1734 | −1.98 | −0.31 | |
| | | isoform b | | | | | | | | | |
| 194072595 | 222 | EML4-ALK var 3 | DQFLSTREKNSFDDL | 1808 | QAKMSTREKNSQVYR | 1761 | ST~E~NS | 1735 | −2.10 | −0.13 | |
| | | isoform b | | | | | | | | | |
| 194072595 | 223 | EML4-ALK var 3 | MRILTREKNSQIERI | 1809 | AKMSTREKNSQVYRR | 1762 | TR~K~SQ | 1736 | −2.02 | −0.65 | |
| | | isoform b | | | | | | | | | |
| 194072595 | 224 | EML4-ALK var 3 | FMMIREKNSQVTIWT | 1810 | KMSTREKNSQVYRRK | 1763 | RE~N~QV | 1737 | −2.06 | −0.73 | |
| | | isoform b | | | | | | | | | |
| 194072595 | 225 | EML4-ALK var 3 | FSIFEKNSQVYFRPP | 1811 | MSTREKNSQVYRRKH | 1764 | EK~S~VY | 1738 | −1.96 | −1.06 | |
| | | isoform b | | | | | | | | | |
| 194072595 | 227 | EML4-ALK var 3 | NFVINSQVYRRQWLV | 1812 | TREKNSQVYRRKHQE | 1765 | NS~V~RR | 1739 | −1.96 | −1.34 | |
| | | isoform b | | | | | | | | | |
| 194072595 | 229 | EML4-ALK var 3 | SFLMQVYRRKHILKL | 1813 | EKNSQVYRRKHQELQ | 1766 | QV~R~KH | 1740 | −2.00 | −0.89 | |
| | | isoform b | | | | | | | | | |
| 194072595 | 216 | EML4-ALK var 3 | GYWSINQAKMSGTTT | 1814 | KDVIINQAKMSTREK | 1757 | IN~A~MS | 1731 | −2.05 | −0.93 | |
| | | isoform b | | | | | | | | | |
| 194072595 | | isoform b | PWFWNQAKMSTRNQY | 1815 | DVIINQAKMSTREKN | 1767 | NQ~K~ST | 1741 | −2.02 | −0.50 | |
| | 217 | EML4-ALK var 3 | | | | | | | | | |
| 194072595 | 216 | EML4-ALK var 3 | KIWFINQAKMSDVIT | 1816 | KDVIINQAKMSTREK | 1757 | IN~A~MS | 1731 | −1.98 | −0.51 | |
| | | isoform b | | | | | | | | | |
| 227452649 | 513 | EML4-ALK var 6 | IIRMPPAVYRRPWHA | 1817 | AADHPPAVYRRKHQE | 1768 | PP~V~RR | 1742 | −1.98 | −1.17 | |
| 227452649 | 514 | EML4-ALK var 6 | DSPLPAVYRRKDFLC | 1818 | ADHPPAVYRRKHQEL | 1769 | PA~Y~RK | 1743 | −1.97 | −0.70 | |
| 227452649 | 515 | EML4-ALK var 6 | HYFVAVYRRKHPHAE | 1819 | DHPPAVYRRKHQELQ | 1770 | AV~R~KH | 1744 | −2.10 | −0.88 | |
| 227452649 | 516 | EML4-ALK var 6 | YILWVYRRKHQTDRR | 1820 | HPPAVYRRKHQELQA | 1771 | VY~R~HQ | 1745 | −2.08 | −1.35 | |
| 194072595 | 230 | EML4-ALK var 6 | LLHTVYRRKHQILIA | 1821 | KNSQVYRRKHQELQA | 1771 | VY~R~HQ | 1745 | −2.00 | −0.08 | DRB1_1501 |

### Example 16: A Bespoke Peptide Library

As the above examples show a bespoke peptide can be designed to provide a peptide that is bound and provides optimal T cell stimulation for any particular combination of MHC alleles and T cell exposed motif. An individual subject may carry 6-12 different MHC alleles (being homozygous or heterozygous at each of MHC I A, B and C and MHC II DP DQ and multiple DR loci. DP and DQ alleles present a different challenge in that they comprise an A and B allele which, in a heterozygous individual, may assemble in four possible combinations. Hence, unless the subject is homozygous for these loci, a bespoke peptide will not be optimally bound in all possible combinations. The 70 MHC I alleles and 24 DRB alleles for which predictions are currently made in our methods provide coverage for approximately 85% humanity. A bespoke peptide can engage and stimulate a cognate T cell clone which can then respond when the same T cell exposed motif is presented naturally. A limitation is that not all T cell exposed motifs are competitively presented in their natural protein context, but with 6-12 alleles, depending on the degree of heterozygosity, and five positions for every T cell exposed motif there is a high probability that some of the positions will be to some degree. Excluding DP and DQ alleles, that represents up to 40-50 potential T cell exposed motif: allele combinations for each amino acid change.

Vogelstein et al describe the most common oncogenes and suppressors, comprising some 121 proteins, several of which are represented in the prior examples. Since that writing, 7 years ago, additional candidates have been added to the list of probable oncogenes and suppressors. It follows that using the methods described herein, and extending the examples described above to other proteins, a database can be assembled of suitable bespoke peptides, not only for each common mutation, but for every potential missense amino acid change in all cancer critical proteins and for each MHC allele. If we assume 121 proteins of average length 400 amino acids, 19 (20-1) alternate amino acids and 70 MHC I alleles (for which we currently predict) such a database when complete is just over 67 million peptides. Furthermore, given that stochastic mutations occur in all proteins, the concept can be extended to embody a database of bespoke peptides to stimulate T cell response to a mutated T cell exposed motif in any protein in the human proteome. This concept therefore applies to missense mutations but not to insertions and deletions or fusions.

### Example 17: Example of a Bespoke Neoepitope Array for an Individual Patient

In this example we provide in Table 23 an array of proteins that were determined to be mutated in the biopsy of a glioblastoma compared to normal tissue from the same subject and show a set of selected peptides proposed for use in a vaccine regimen for that subject. The upper tier of the Table provides the proposed MHC I binding peptides and the lower tier the proposed MHC II binding peptides. It will be noted that in one protein a naturally occurring peptide was deemed to have an appropriate binding affinity to serve as a CD4+ helper (marked as “native”). As always the predicted binding affinity is shown in standard deviation units below the mean. As a reference point a shift from a natural originating peptide binding at −0.66 SD units to a proposed peptide binding at 2.0 SD units is an increase in predicted binding of approximately 100 fold.

TABLE 23
Example of an array of mutated tumor proteins sequenced in a biopsy of a glioblastoma patient and corresponding T cell
exposed motifs and originating and proposed peptides for MHC I and MHC II alleles

| | | | | | | | | | predicted | predicted | |
|---|---|---|---|---|---|---|---|---|---|---|---|
| | Amino | | | | | SEQ | | SEQ | affinity of | affinity of | |
| gene | acid | | proposed | SEQ ID | Originating | ID | TCEM | ID | proposed | originating | |
| symbol | change | pos | peptide | NO.: | peptide | NO.: | core | NO.: | peptide | peptide | Allele |
| ATP1B1 | F15V | 8 | HALSWKKVL | 1984 | EEGSWKKVI | 1955 | SWKKV | 1926 | −2.02 | −1.11 | A3201 |
| CCT8 | G122C | 116 | DYKLRICLK | 1985 | EELLRICLS | 1956 | LRICL | 1927 | −2.02 | −1.00 | C0702 |
| DCAF15 | V556M | 549 | WLLRKSCMK | 1986 | SSYRKSCMD | 1957 | RKSCM | 1928 | −2.08 | −0.71 | C1203 |
| EGFR | A244V | 240 | DGSGVTCVV | 1987 | YSFGVTCVK | 1958 | GVTCV | 1929 | −2.08 | −0.69 | B5101 |
| EGFR | A244V | 238 | RSFSFGVTF | 1988 | GKYSFGVTC | 1959 | SFGVT | 1930 | −2.03 | −1.53 | B5101 |
| FLOT2 | E11K | 7 | SDFNKALVF | 1989 | VGPNKALVV | 1960 | NKALV | 1931 | −2.09 | −0.58 | A3201 |
| IST1 | S181C | 175 | DDIEPDCVL | 1990 | VPYEPDCVV | 1961 | EPDCV | 1932 | −2.05 | −0.69 | A3201 |
| MASP1 | G575R | 572 | SYERPEECF | 1991 | SWGRPEECG | 1962 | RPEEC | 1933 | −2.01 | −1.51 | C0702 |
| NBPF14 | E355G | 349 | EGKKLAGQV | 1992 | KEEKLAGQL | 1963 | KLAGQ | 1934 | −2.04 | −0.54 | B5101 |
| NEK4 | R565Q | 559 | ELYSKDQPL | 1993 | MSSSKDQPL | 1964 | SKDQP | 1935 | −2.07 | −0.76 | A3201 |
| PARD3B | P974A | 967 | WFSRSPSAR | 1994 | NVFRSPSAP | 1965 | RSPSA | 1936 | −2.03 | −0.41 | A2902 |
| PRDM11 | T21M | 16 | KADMVMVVG | 1995 | VGDMVMVVK | 1966 | MVMVV | 1937 | −2.08 | −1.27 | C1203 |
| RPGRIP1L | S199T | 193 | LGYYGNTLM | 1996 | FTKYGNTLL | 1967 | YGNTL | 1938 | −2.10 | −1.63 | B5101 |
| TYW1 | G393A | 390 | SWGACYKHQ | 1997 | GRGACYKHT | 1968 | ACYKH | 1939 | −2.03 | −1.21 | C0702 |
| WARS2 | M1301 | 123 | KEEILSCIL | 1998 | LSWILSCIV | 1969 | ILSCI | 1940 | −2.06 | −1.04 | A3201 |
| ATP1B1 | F15V | 8 | SWDLWKKVIWNVEEV | 1999 | EEGSWKKVIWNSEKK | 1970 | WK~V~WN | 1941 | −2.03 | −0.22 | DRB1_1602 |
| CCT8 | G122C | 118 | RRWVCLSVSEVTSSY | 2000 | LLRICLSVSEVIEGY | 1971 | CL~V~EV | 1942 | −2.05 | −1.19 | DRB3_0202 |
| DCAF15 | V556M | 549 | RNELKSCMDMVQCLV | 2001 | SSYRKSCMDMVMKWL | 1972 | KS~M~MV | 1943 | −2.04 | −0.37 | DRB1_1602 |
| EGFR | A244V | 239 | QRWLGVTCVKKLRTL | 2002 | KYSFGVTCVKKCPRN | 1973 | GV~C~KK | 1944 | −2.05 | −0.98 | DRB1_0301 |
| FLOT2 | E11K | 2 | KWETTVGPNKATNFC | 2003 | GNCHTVGPNKALVVS | 1974 | TV~P~KA | 1945 | −2.03 | −0.68 | DRB1_1602 |
| IST1 | S181C | 174 | NVPYEPDCVVMAEAP | 2004 | | | EP~C~VM | 1946 | native | −2.03 | DRB1_1602 |
| MASP1 | G575R | 570 | FKVLGRPEECGLRHF | 2005 | LVSWGRPEECGSKQV | 1976 | GR~E~CG | 1947 | −2.08 | −0.63 | DRB1_0301 |
| NBPF14 | E355G | 350 | SPVKAGQLKQAGESS | 2006 | EEKLAGQLKQAEELR | 1977 | AG~L~QA | 1948 | −2.08 | −0.78 | DRB1_1602 |
| PARD3B | P974A | 970 | TYLLAPRAGPFEMVR | 2007 | RSPSAPRAGPFGYPR | 1978 | AP~A~PF | 1949 | −2.05 | −0.77 | DRB1_1602 |
| PRDM11 | T21M | 12 | KSEPVGDMVMVVYGV | 2008 | TNAAVGDMVMVVKTE | 1979 | VG~M~MV | 1950 | −2.04 | −0.67 | DRB1_1602 |
| RPGRIP1L | S199T | 190 | HPLVTKYGNTLEWYS | 2009 | HPMFTKYGNTLLEEA | 1980 | TK~G~TL | 1951 | −2.07 | −1.66 | DRB1_0301 |
| TYW1 | G393A | 388 | VEYVGACYKHTFYPG | 2010 | LRGRGACYKHTFYGI | 1981 | GA~Y~HT | 1952 | −2.01 | −0.57 | DRB1_0301 |
| WARS2 | M1301 | 123 | KNSSLSCIVRLASGV | 2011 | LSWILSCIVRLPRLQ | 1982 | LS~I~RL | 1953 | −2.05 | −1.14 | DRB1_1602 |
| NEK4 | R565Q | 556 | KPAKSSSKDQPVSLC | 2012 | SSEMSSSKDQPLSAR | 1983 | SS~K~QP | 1954 | −1.99 | −0.51 | DRB1_1602 |

Gene symbols are HUGO; protein descriptions are omitted in the interest of space

### Example 18: Identification of a Unique Fusion Bridge

FusionGDB is a database resource and reference for functional annotation of fusion genes in cancer. The database comprises over 48 thousand fusion genes found in many different types of cancer and has been assembled from three representative fusion gene resources: 1) the improved database of chimeric transcripts and RNA-seq data (ChiTaRS 3.1), 2) an integrative resource for cancer-associated transcript fusions (TumorFusions) and 3), The Cancer Genome Atlas (TCGA) fusions from Gao et al [24]. The database provides functional annotations including gene assessment across pan-cancer fusion genes, open reading frame (ORF) assignment, and protein domain retention across multiple break points. The database also provides the fusion transcript and amino acid sequences for each break point and gene isoforms that are available for downloading [24, 87, 88].

The gene alignment program BLAST was one of the first gene alignment programs and has been in use for many years. magicBLAST is a more recent derivative program and is an accurate RNA sequence aligner that aligns and enumerates unknown sequences against a BLAST database. The FusionGDB transcripts were retrieved and a BLAST database of 150 nucleotide sequences across the defined breakpoint was constructed using the bioinformatic tool makeblastdb. With the BLAST database of fusion junctions magicBLAST was used to detect and enumerate fusions of chimeric genes in the paired read fastq files of tumor RNA sequences. Short reads of 76 nucleotides provided a basis of tabulating bridge junctions in the paired reads that matched any of the 48 thousand junctions that have previously been detected in various types of cancers.

Where there is an apparent fusion bridge, and where this is not a typical fusion found in all/most tumors, the sequence is verified to determine that there is an in-frame bridge. Possible open reading frames of the purported bridge are identified and matched to the sequence of each fusion partner. Predicted MHC binding and topologic features of each partner protein and of the fusion is determined (see e.g., U.S. Pat. No. 10,706,955 US Publ. 2017/0039314, each of which is incorporated herein by reference). These are compared to determine any indication of functional impact (e.g. acquired signal peptide, membrane insertion sited). The T cell exposed motifs which are unique only to the fusion are identified, and their predicted MHC binding determined for the HLA alleles of the particular subject. If the peptides comprising the unique TCEM motifs are found to bind naturally within a desired range they are incorporated into the selected peptide array in their natural form. If the binding is lower than desired but would still result in some natural presentation, an array of alternative peptides is generated according to the methods described above in Example 2.

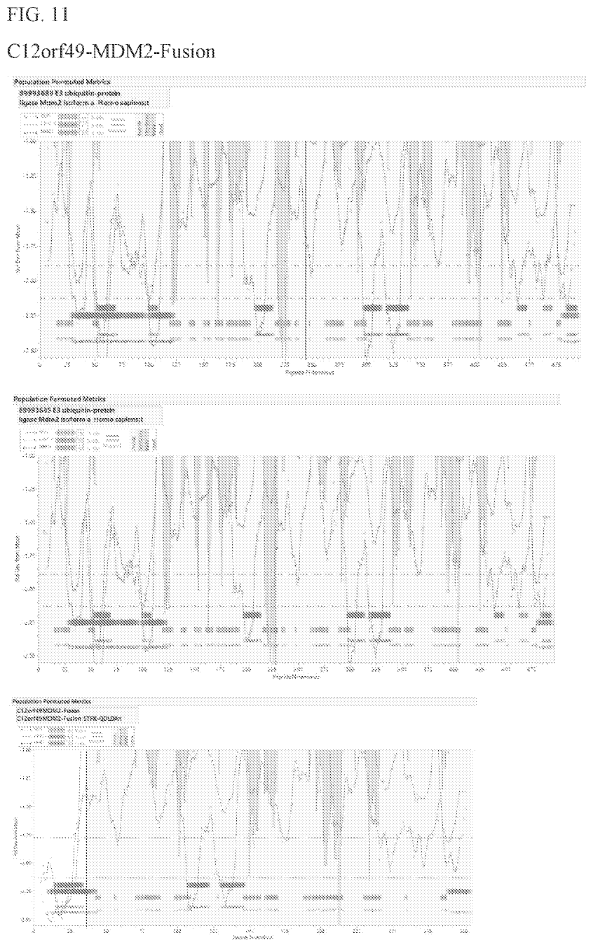

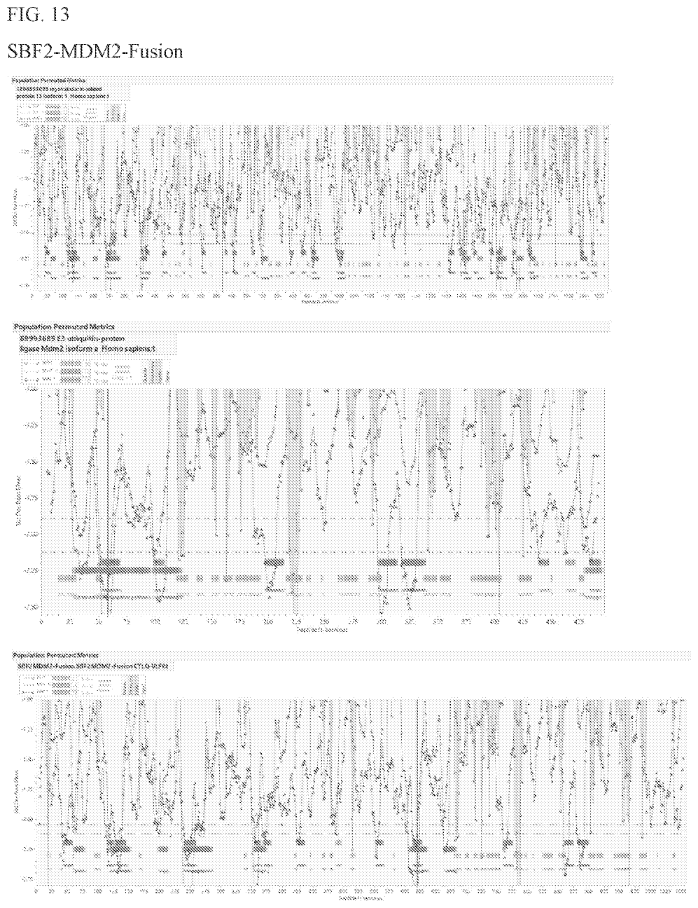

FIGS. 11-13 show one example of identification of in frame fusion bridged in one subject with an increased copy number of MDM2 and fusions comprising partial sequences of MDM2. Table 24 shows the fusion sequences and the protein sequences of the fusion partners. Table 25 show the fusion pairs and fusion sequences in this particular example.

TABLE 24
Parent and fusion protein sequence examples.
Amino acids are underlined 4 on each side
of fusion junction.

| | SEQ | |
|---|---|---|
| | ID | |
| Identification | NO.: | Sequence |
| NP_002383.2 | 2140 | MVRSRQMCNTNMSVPTDGAVTTSQIPASEQ |
| E3 ubiquitin- | | ETLVRPKPLLLKLLKSVGAQKDTYTMKEVL |
| protein ligase | | FYLGQYIMTKRLYDEKQQHIVYCSNDLLGD |
| Mdm2 isoform a | | LFGVPSFSVKEHRKIYTMIYRNLVVVNQQE |
| | | SSDSGTSVSENRCHLEGGSDQKDLVQELQE |
| | | EKPSSSHLVSRPSTSSRRRAISETEENSDE |
| | | LSGERQRKRHKSDSISLSFDESLALCVIRE |
| | | ICCERSSSSESTGTPSNPDLDAGVSEHSGD |
| | | WLDQDSVSDQFSVEFEVESLDSEDYSLSEE |
| | | GQELSDEDDEVYQVTVYQAGESDTDSFEED |
| | | PEISLADYWKCTSCNEMNPPLPSHCNRCWA |
| | | LRENWLPEDKGKDKGEISEKAKLENSTQAE |
| | | EGFDVPDCKKTIVNDSRESCVEENDDKITQ |
| | | ASQSQESEDYSQPSTSSSIIYSSQEDVKEF |
| | | EREETQDKEESVESSLPLNAIEPCVICQGR |
| | | PKNGCIVHGKTGHLMACFTCAKKLKKRNKP |
| | | CPVCRQPIQMIVLTYFP |
| NP_001373268.11 | 2141 | MARLADYFIVVGYDHEKPGSGEGLGKIIQR |
| myotubularin- | | FPQKDWDDTPFPQGIELFCQPGGWQLSRER |
| related protein | | KQPTFFVVVLTDIDSDRHYCSCLTFYEAEI |
| 13 isoform 1 | | NLQGTKKEEIEGEAKVSGLIQPAEVFAPKS |
| | | LVLVSRLYYPEIFRACLGLIYTVYVDSLNV |
| | | SLESLIANLCACLVPAAGGSQKLESLGAGD |
| | | RQLIQTPLHDSLPITGTSVALLFQQLGIQN |
| | | VLSLFCAVLTENKVLFHSASFQRLSDACRA |
| | | LESLMFPLKYSYPYIPILPAQLLEVLSSPT |
| | | PFIIGVHSVFKTDVHELLDVIIADLDGGTI |
| | | KIPECIHLSSLPEPLLHQTQSALSLILHPD |
| | | LEVADHAFPPPRTALSHSKMLDKEVRAVFL |
| | | RLFAQLFQGYRSCLQLIRIHAEPVIHFHKT |
| | | AFLGQRGLVENDFLTKVLSGMAFAGFVSER |
| | | GPPYRSCDLFDELVAFEVERIKVEENNPVK |
| | | MIKHVRELAEQLFKNENPNPHMAFQKVPRP |
| | | TEGSHLRVHILPFPEINEARVQELIQENVA |
| | | KNQNAPPATRIEKKCVVPAGPPVVSIMDKV |
| | | TTVFNSAQRLEVVRNCISFIFENKILETEK |
| | | TLPAALRALKGKAARQCLTDELGLHVQQNR |
| | | AILDHQQFDYIIRMMNCTLQDCSSLEEYNI |
| | | AAALLPLTSAFYRKLAPGVSQFAYTCVQDH |
| | | PIWTNQQFWETTFYNAVQEQVRSLYLSAKE |
| | | DNHAPHLKQKDKLPDDHYQEKTAMDLAAEQ |
| | | LRLWPTLSKSTQQELVQHEESTVFSQAIHF |
| | | ANLMVNLLVPLDTSKNKLLRTSAPGDWESG |
| | | SNSIVTNSIAGSVAESYDTESGFEDSENTD |
| | | IANSVVRFITRFIDKVCTESGVTQDHIKSL |
| | | HCMIPGIVAMHIETLEAVHRESRRLPPIQK |
| | | PKILRPALLPGEEIVCEGLRVLLDPDGREE |
| | | ATGGLLGGPQLLPAEGALFLTTYRILFRGT |
| | | PHDQLVGEQTVVRSFPIASITKEKKITMQN |
| | | QLQQNMQEGLQITSASFQLIKVAFDEEVSP |
| | | EVVEIFKKQLMKFRYPQSIFSTFAFAAGQT |
| | | TPQIILPKQKEKNTSFRTFSKTIVKGAKRA |
| | | GKMTIGRQYLLKKKTGTIVEERVNRPGWNE |
| | | DDDVSVSDESELPTSTTLKASEKSTMEQLV |
| | | EKACFRDYQRLGLGTISGSSSRSRPEYFRI |
| | | TASNRMYSLCRSYPGLLVVPQAVQDSSLPR |
| | | VARCYRHNRLPVVCWKNSRSGTLLLRSGGF |
| | | HGKGVVGLFKSQNSPQAAPTSSLESSSSIE |
| | | QEKYLQALLNAVSVHQKLRGNSTLTVRPAF |
| | | ALSPGTERRTSRMSTVLKQVVPGHLDVNPS |
| | | NSFAQGGVWASLRSSTRLISSPTSFIDVGA |
| | | RLAGKDHSASFSNSSYLQNQLLKRQAALYI |
| | | FGEKSQLRNFKVEFALNCEFVPVEFHEIRQ |
| | | VKASFKKLMRACIPSTIPTDSEVTFLKALG |
| | | DSEWFPQLHRIMQLAVVVSEVLENGSSVLV |
| | | CLEEGWDITAQVTSLVQLLSDPFYRTLEGF |
| | | QMLVEKEWLSFGHKFSQRSSLTLNCQGSGF |
| | | APVFLQFLDCVHQVHNQYPTEFEFNLYYLK |
| | | FLAFHYVSNRFKTFLLDSDYERLEHGTLFD |
| | | DKGEKHAKKGVCIWECIDRMHKRSPIFFNY |
| | | LYSPLEIEALKPNVNVSSLKKWDYYIEETL |
| | | STGPSYDWMMLTPKHFPSEDSDLAGEAGPR |
| | | SQRRTVWPCYDDVSCTQPDALTSLFSEIEK |
| | | LEHKLNQAPEKWQQLWERVTVDLKEEPRTD |
| | | RSQRHLSRSPGIVSTNLPSYQKRSLLHLPD |
| | | SSMGEEQNSSISPSNGVERRAATLYSQYTS |
| | | KNDENRSFEGTLYKRGALLKGWKPRWFVLD |
| | | VTKHQLRYYDSGEDTSCKGHIDLAEVEMVI |
| | | PAGPSMGAPKHTSDKAFFDLKTSKRVYNFC |
| | | AQDGQSAQQWMDKIQSCISDA |
| Q9H741 C12orf49 | 2142 | MVNLAAMVWRRLLRKRWVLALVFGLSLVYF |
| SPRNG_HUMAN | | LSSTFKQEERAVRDRNLLQVHDHNQPIPWK |
| SREBP | | VQFNLGNSSRPSNQCRNSIQGKHLITDELG |
| regulating | | YVCERKDLLVNGCCNVNVPSTKQYCCDGCW |
| gene protein | | PNGCCSAYEYCVSCCLQPNKQLLLERFLNR |
| | | AAVAFQNLFMAVEDHFELCLAKCRTSSQSV |
| | | QHENTYRDPIAKYCYGESPPELFPA |
| P14625 | 2143 | MRALWVLGLCCVLLTFGSVRADDEVDVDGT |
| ENPL_HUMAN | | VEEDLGKSREGSRTDDEVVQREEEAIQLDG |
| Endoplasmin | | LNASQIRELREKSEKFAFQAEVNRMMKLII |
| HSP90B1 | | NSLYKNKEIFLRELISNASDALDKIRLISL |
| | | TDENALSGNEELTVKIKCDKEKNLLHVTDT |
| | | GVGMTREELVKNLGTIAKSGTSEFLNKMTE |
| | | AQEDGQSTSELIGQFGVGFYSAFLVADKVI |
| | | VTSKHNNDTQHIWESDSNEFSVIADPRGNT |
| | | LGRGTTITLVLKEEASDYLELDTIKNLVKK |
| | | YSQFINFPIYVWSSKTETVEEPMEEEEAAK |
| | | EEKEESDDEAAVEEEEEEKKPKTKKVEKTV |
| | | WDWELMNDIKPIWQRPSKEVEEDEYKAFYK |
| | | SFSKESDDPMAYIHFTAEGEVTFKSILFVP |
| | | TSAPRGLFDEYGSKKSDYIKLYVRRVFITD |
| | | DFHDMMPKYLNFVKGVVDSDDLPLNVSRET |
| | | LQQHKLLKVIRKKLVRKTLDMIKKIADDKY |
| | | NDTFWKEFGTNIKLGVIEDHSNRTRLAKLL |
| | | RFQSSHHPTDITSLDQYVERMKEKQDKIYF |
| | | MAGSSRKEAESSPFVERLLKKGYEVIYLTE |
| | | PVDEYCIQALPEFDGKRFQNVAKEGVKFDE |
| | | SEKTKESREAVEKEFEPLLNWMKDKALKDK |
| | | IEKAVVSQRLTESPCALVASQYGWSGNMER |
| | | IMKAQAYQTGKDISTNYYASQKKTFEINPR |
| | | HPLIRDMLRRIKEDEDDKTVLDLAVVLFET |
| | | ATLRSGYLLPDTKAYGDRIERMLRLSLNID |
| | | PDAKVEEEPEEEPEETAEDTTEDTEQDEDE |
| | | EMDVGTDEEEETAKESTAEKDEL |
| SBF2-MDM2- | 2144 | MARLADYFIVVGYDHEKPGSGEGLGKIIQR |
| Fusion CTLQ- | | FPQKDWDDTPFPQGIELFCQPGGWQLSRER |
| VLFY | | KQPTFFVVVLTDIDSDRHYCSCLTFYEAEI |
| | | NLQGTKKEEIEGEAKVSGLIQPAEVFAPKS |
| | | LVLVSRLYYPEIFRACLGLIYTVYVDSLNV |
| | | SLESLIANLCACLVPAAGGSQKLFSLGAGD |
| | | RQLIQTPLHDSLPITGTSVALLFQQLGIQN |
| | | VLSLFCAVLTENKVLFHSASFQRLSDACRA |
| | | LESLMFPLKYSYPYIPILPAQLLEVLSSPT |
| | | PFIIGVHSVFKTDVHELLDVIIADLDGGTI |
| | | KIPECIHLSSLPEPLLHQTQSALSLILHPD |
| | | LEVADHAFPPPRTALSHSKMLDKEVRAVFL |
| | | RLFAQLFQGYRSCLQLIRIHAEPVIHFHKT |
| | | AFLGQRGLVENDFLTKVLSGMAFAGFVSER |
| | | GPPYRSCDLFDELVAFEVERIKVEENNPVK |
| | | MIKHVRELAEQLFKNENPNPHMAFQKVPRP |
| | | TEGSHLRVHILPFPEINEARVQELIQENVA |
| | | KNQNAPPATRIEKKCVVPAGPPVVSIMDKV |
| | | TTVFNSAQRLEVVRNCISFIFENKILETEK |
| | | TLPAALRALKGKAARQCLTDELGLHVQQNR |
| | | AILDHQQFDYIIRMMNCTLQVLFYLGQYIM |
| | | TKRLYDEKQQHIVYCSNDLLGDLFGVPSFS |
| | | VKEHRKIYTMIYRNLVVVNQQESSDSGTSV |
| | | SENRCHLEGGSDQKDLVQELQEEKPSSSHL |
| | | VSRPSTSSRRRAISETEENSDELSGERQRK |
| | | RHKSDSISLSFDESLALCVIREICCERSSS |
| | | SESTGTPSNPDLDAGVSEHSGDWLDQDSVS |
| | | DQFSVEFEVESLDSEDYSLSEEGQELSDED |
| | | DEVYQVTVYQAGESDTDSFEEDPEISLADY |
| | | WKCTSCNEMNPPLPSHCNRCWALRENWLPE |
| | | DKGKDKGEISEKAKLENSTQAEEGFDVPDC |
| | | KKTIVNDSRESCVEENDDKITQASQSQESE |
| | | DYSQPSTSSSIIYSSQEDVKEFEREETQDK |
| | | EESVESSLPLNAIEPCVICQGRPKNGCIVH |
| | | GKTGHLMACFTCAKKLKKRNKPCPVCRQPI |
| | | QMIVLTYFP |
| C12orf49- | 2145 | MVNLAAMVWRRLLRKRWVLALVFGLSLVYF |
| MDM2-Fusion | | LSSTFKQDLDAGVSEHSGDWLDQDSVSDQF |
| STFK-QDLDA | | SVEFEVESLDSEDYSLSEEGQELSDEDDEV |
| | | YQVTVYQAGESDTDSFEEDPEISLADYWKC |
| | | TSCNEMNPPLPSHCNRCWALRENWLPEDKG |
| | | KDKGEISEKAKLENSTQAEEGFDVPDCKKT |
| | | IVNDSRESCVEENDDKITQASQSQESEDYS |
| | | QPSTSSSIIYSSQEDVKEFEREETQDKEES |
| | | VESSLPLNAIEPCVICQGRPKNGCIVHGKT |
| | | GHLMACFTCAKKLKKRNKPCPVCRQPIQMI |
| | | VLTYFP |
| HSP90B1- | 2146 | MRALWVLGLCCVLLTFGSVRADDEVDVDGT |
| MDM2-fusion | | VEEDLGKSREGSRTDDEVVQREEEAIQLDG |
| ETAK-DLDA | | LNASQIRELREKSEKFAFQAEVNRMMKLII |
| | | NSLYKNKEIFLRELISNASDALDKIRLISL |
| | | TDENALSGNEELTVKIKCDKEKNLLHVTDT |
| | | GVGMTREELVKNLGTIAKSGTSEFLNKMTE |
| | | AQEDGQSTSELIGQFGVGFYSAFLVADKVI |
| | | VTSKHNNDTQHIWESDSNEFSVIADPRGNT |
| | | LGRGTTITLVLKEEASDYLELDTIKNLVKK |
| | | YSQFINFPIYVWSSKTETVEEPMEEEEAAK |
| | | EEKEESDDEAAVEEEEEEKKPKTKKVEKTV |
| | | WDWELMNDIKPIWQRPSKEVEEDEYKAFYK |
| | | SFSKESDDPMAYIHFTAEGEVTFKSILFVP |
| | | TSAPRGLFDEYGSKKSDYIKLYVRRVFITD |
| | | DFHDMMPKYLNFVKGVVDSDDLPLNVSRET |
| | | LQQHKLLKVIRKKLVRKTLDMIKKIADDKY |
| | | NDTFWKEFGTNIKLGVIEDHSNRTRLAKLL |
| | | RFQSSHHPTDITSLDQYVERMKEKQDKIYF |
| | | MAGSSRKEAESSPFVERLLKKGYEVIYLTE |
| | | PVDEYCIQALPEFDGKRFQNVAKEGVKFDE |
| | | SEKTKESREAVEKEFEPLLNWMKDKALKDK |
| | | IEKAVVSQRLTESPCALVASQYGWSGNMER |
| | | IMKAQAYQTGKDISTNYYASQKKTFEINPR |
| | | HPLIRDMLRRIKEDEDDKTVLDLAVVLFET |
| | | ATLRSGYLLPDTKAYGDRIERMLRLSLNID |
| | | PDAKVEEEPEEEPEETAEDTTEDTEQDEDE |
| | | EMDVGTDEEEETAKDLDAGVSEHSGDWLDQ |
| | | DSVSDQFSVEFEVESLDSEDYSLSEEGQEL |
| | | SDEDDEVYQVTVYQAGESDTDSFEEDPEIS |
| | | LADYWKCTSCNEMNPPLPSHCNRCWALREN |
| | | WLPEDKGKDKGEISEKAKLENSTQAEEGFD |
| | | VPDCKKTIVNDSRESCVEENDDKITQASQS |
| | | QESEDYSQPSTSSSIIYSSQEDVKEFEREE |
| | | TQDKEESVESSLPLNAIEPCVICQGRPKNG |
| | | CIVHGKTGHLMACFTCAKKLKKRNKPCPVC |
| | | RQPIQMIVLTYFP |

TABLE 25

| | | | | | | | | Predicted | |
|---|---|---|---|---|---|---|---|---|---|
| | | SEQ | | SEQ | | SEQ | | binding | Original |
| | Original | ID | Modified | ID | TCEM | ID | | modified | predicted |
| description | peptide | NO.: | peptide | NO.: | core | NO.: | Allele | peptide | binding |
| C12orf49MDM2-Fusion | LSSTFKQDL | 2147 | ENLTFKQDM | 2160 | ~~~TFKQD~ | 2174 | B_5701 | −2.03 | −1.74 |
| STFK-QDLDA | | | | | | | | | |
| C12orf49MDM2-Fusion | LSSTFKQDL | 2148 | MSNTFKQDM | 2161 | ~~~TFKQD~ | 2175 | C_0501 | −2.02 | −1.05 |
| STFK-QDLDA | | | | | | | | | |
| C12orf49MDM2-Fusion | STFKQDLDA | 2149 | SMLKQDLDS | 2162 | ~~~KQDLD~ | 2176 | A_0201 | −2.08 | −1.17 |
| STFK-QDLDA | | | | | | | | | |
| HSP90B1MDM2-fusion | EEEETAKDL | 2150 | MSSETAKDM | 2163 | ~~~ETAKD~ | 2177 | C_0501 | −1.98 | −1.27 |
| ETAK-DLDA ## | | | | | | | | | |
| HSP90B1MDM2-fusion | EEETAKDLD | 2151 | MEMTAKDLE | 2164 | ~~~TAKDL~ | 2178 | B_1801 | −2.06 | −1.20 |
| ETAK-DLDA | | | | | | | | | |
| HSP90B1MDM2-fusion | EEETAKDLD | 2152 | NSNTAKDLM | 2165 | ~~~TAKDL~ | 2179 | C_0501 | −2.05 | −0.61 |
| ETAK-DLDA | | | | | | | | | |
| SBF2MDM2-Fusion | MNCTLQVLF | 2153 | NTNTLQVLM | 2166 | ~~~TLQVL~ | 2180 | B_5701 | −2.08 | −1.94 |
| CTLQ-VLFY | | | | | | | | | |
| SBF2MDM2-Fusion | NCTLQVLFY | 2154 | ETELQVLFT | 2167 | ~~~LQVLF~ | 2181 | A_0101 | −2.03 | −1.55 |
| CTLQ-VLFY | | | | | | | | | |
| SBF2MDM2-Fusion | CTLQVLFYL | 2155 | LEEQVLFYE | 2168 | ~~~QVLFY~ | 2182 | B_1801 | −2.05 | −1.18 |
| CTLQ-VLFY | | | | | | | | | |
| | | | | | | | | Predicted | |
| | | SEQ | | SEQ | | SEQ | | binding | Original |
| | | ID | Modified | ID | TCEM | ID | | modified | predicted |
| description | | NO.: | peptide | NO.: | core | NO.: | Allele | peptide | binding |
| C12orf49MDM2-Fusion | VYFLSSTFK | 2156 | SEIFSSTF | 2169 | SS~F~QD | 2183 | DRB1_0301 | −1.99 | −1.00 |
| STFK-QDLDA | QDLDAG | | KQDREMV | | | | | | |
| SBF2MDM2-Fusion | RMMNCTLQV | 2157 | EELICTLQ | 2170 | CT~Q~LF | 2184 | DRB3_0202 | −1.99 | −1.74 |
| CTLQ-VLFY | LFYLGQ | | VLFMRDR | | | | | | |
| SBF2MDM2-Fusion | NCTLQVLFY | 2158 | RFTRQVLF | 2171 | QV~F~LG | 2185 | DRB1_0701 | −1.98 | −1.33 |
| CTLQ-VLFY | LGQYIM | | YLGDRTV | | | | | | |
| SBF2MDM2-Fusion | IIRMMNCTL | 2159 | IIRMMNCT | 2172 | MN~T~QV | 2186 | DRB1_0701 | −1.83 | −1.83 |
| CTLQ-VLFY ** | QVLFYL | | LQVLFYL | | | | | | |

** Note that the natural peptide was used in this case as binding was in desired range
## No corresponding MHC II binding peptide could be designed for this fusion bridge as the predicted binding of the original peptide was too low to permit presentation in the natural context.

### Example 19: Method to Determine the RNA Fraction

Bulk RNA transcript enumeration is carried out using a bioinformatic process that has been designed to tally transcription of different genes. The resulting data is expressed as the FPKM (fragments per kilobase per million total reads) that normalizes the metric for both the length of the transcribed coding region and the number of total reads in the bulk sample detected by the sequencing machine. The bioinformatic software used for transcript enumeration (Magic-BLAST from NCBI) has been designed to assess gene expression and as such is not directly capable of measuring the frequency of potentially mutated codons within the transcripts. In order to compute the mutant frequency in the mRNA transcripts it is necessary to separately enumerate the normal and mutant transcripts. This is achieved by creating a version of the SAM (sequence alignment map) file of the RNA sequences with a bioinformatic software that modifies the cigar (compact idiosyncratic gapped alignment report) strings that map the alignments of the (missing) intronic sequences in the mRNA. Once this modified SAM file is created it can be processed with the standard mutation detection tool, such as mutect 2 that provides the differential mutant and normal read tallies. The ratios of these read tallies are thus the mutant and normal frequency of the allele in the mRNA transcripts. If both parental chromosomes are being expressed equally then the frequency of the mutant and normal allele in the RNA will correlate with the frequency in the DNA. Allele specific differences in expression will give rise to poor correlations. In the extreme, where there is highly differential expression of the parental chromosomes, the mutant may be the only one expressed or may not be expressed at all compared to the normal.

Therefore, in preferred embodiments, the RNA fraction comprising the mutant amino acid is compared to the tumor DNA tumor fraction encoding the gene mutation. In some embodiments tumor specific mutations which can be targeted by T cells are selected from those in which the RNA/DNA ratio exceeds 10%. In most preferred embodiments the targetable mutations are selected from those in which the RNA/DNA ratio exceeds 20%.

### Example 20: Down Selection of Target Peptides Based on RNA Transcription

Patient ISW was diagnosed with a glioblastoma. Biopsy sequencing for DNA and RNA was performed and DNA sequencing of a normal PBMC sample. A listing of mutants was established as described in Example 1 and the HLA were determined as described in Example 3. Of 203 missense mutations identified, 51 were present at greater than 10% of the tumor DNA (tumor fraction). These were ranked based on the ratio of tumor fraction and RNA reads as shown in Table 26.

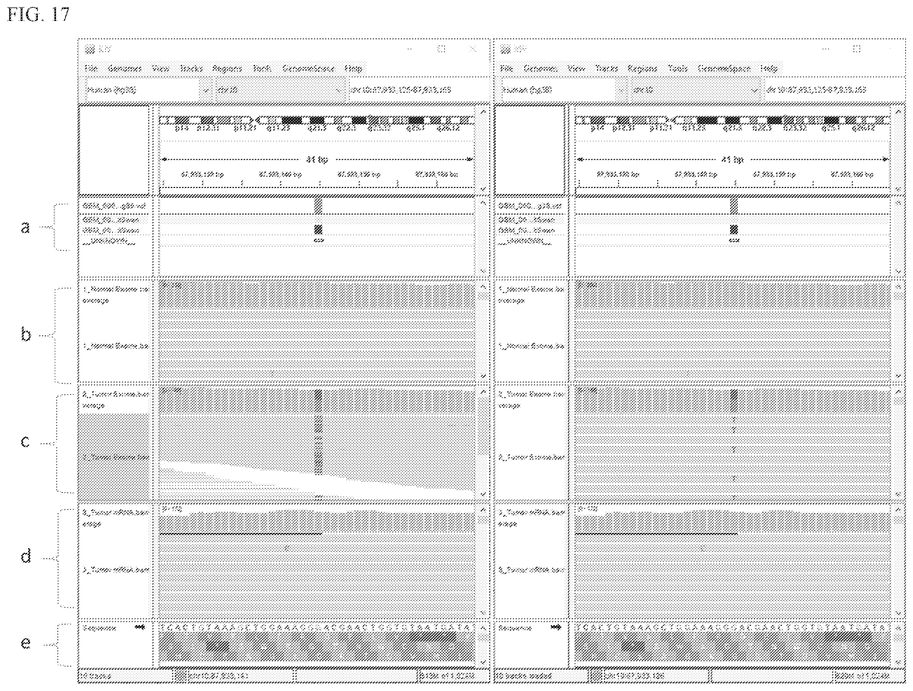

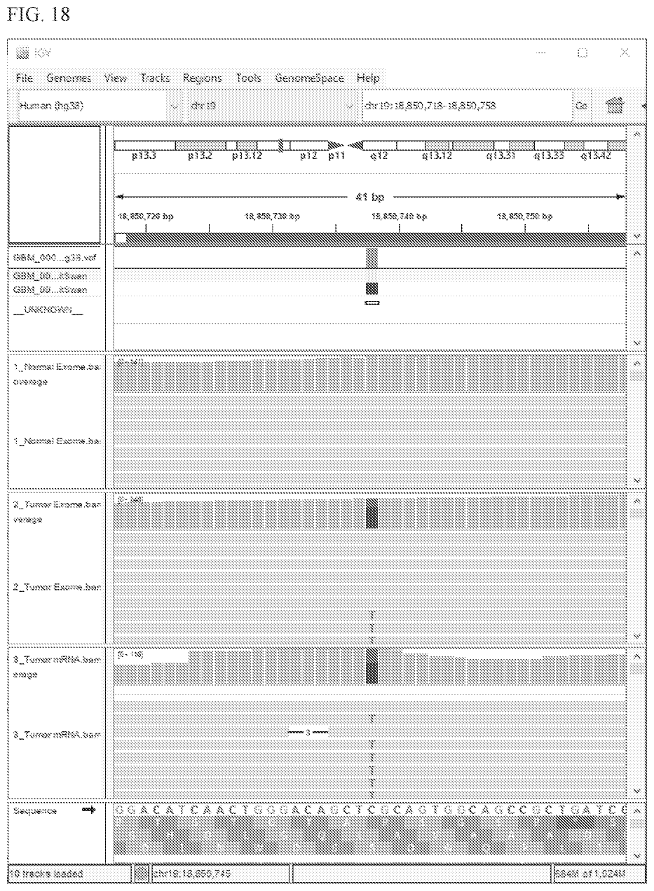

Examination of the sequences of reads immediately surrounding the genomic region containing the mutations was viewed in the Integrated Genome Browser (IGV Broad Institute). IGV enables visual comparison of the aligned DNA of the exome sequences of the tumor and the normal blood sample with those of the aligned expressed mRNA in the same genomic region. The general expectation is that both chromosome arms are being transcribed and translated and thus the fraction of the RNA transcripts containing the mutant would be similar to that in the exome DNA. For many genes this is precisely the situation and the relative proportion of mRNA sequences with the mutation is very similar to the proportion in the exomic DNA. Unexpectedly, however, there are a number of cases where this is not the case. In this representative patient example there are a number of genes that were determined to be mutated in the exomic DNA but for which mutations in the mRNA are not detected. This is not only in poorly expressed genes where the level of detection might be an issue, but is also seen in many genes being expressed at relatively high levels. This implies epigenetic control resulting in allele specific expression where only one chromosome arm (the one without the mutant) is being expressed. This was particularly apparent and interesting for the PTEN G129V mutation as shown in FIGS. 17-18. PTEN is a recognized oncogene. If the mutation is being expressed, it would be a targetable mutation of interest. Consequently, because it is not being expressed it was excluded from consideration as a neoepitope target. For a series of other proteins in this subject's tumor both the native and mutant alleles are being expressed. For highly expressed genes where the mutant fractions can be more accurately determined, the frequency in the mRNA and exome is similar as shown as the first three on the list in Table 26.

TABLE 26
Example of differential RNA expression.

| | | | Tumor | RNA seq | RNA seq | Tumor |
|---|---|---|---|---|---|---|
| | Protein | FPKM | Fraction | reads | reads | Fraction |
| Gene Symbol | Change | (standardized) | (Exomes) | native | mutant | (RNA Seq) |
| EGFR | A244D | 2.41 | 21% | 836 | 272 | 0.249 |
| UPF1 | S227L | 0.68 | 32% | 60 | 46 | 0.48 |
| CD3EAP | A92V | 0.38 | 18% | 45 | 11 | 0.132 |
| USP24 | S1035T | 0.53 | 36% | 33 | 16 | 0.284 |
| KCTD3 | P104A | 1.31 | 31% | 32 | 44 | 0.529 |
| WSCD1 | I549T | 0.53 | 12% | 32 | 26 | 0.472 |
| FAM234A | L159F | 0.72 | 34% | 25 | 4 | 0.183 |
| LIPE | V784I | −0.48 | 24% | 12 | 4 | 0.312 |
| SMPD4 | F484V | 0.14 | 13% | 9 | 5 | 0.375 |
| ARHGEF28 | R555H | −0.82 | 24% | 1 | 12 | 0.857 |
| TTN | P27465L | −0.80 | 27% | 0 | 4 | 0.833 |
| PABPC3 | R424Q | −0.93 | 12% | 0 | 13 | 0.933 |
| PTEN | G129V | 0.84 | 47% | 0 | 0 | 0 |
| CALHM5 | F96C | −1.30 | 44% | 0 | 0 | 0 |
| CDON | S455L | −0.32 | 38% | 0 | 0 | 0 |
| DNAH12 | P180S | −2.10 | 37% | 0 | 0 | 0 |
| SCN7A | L57I | −0.81 | 36% | 0 | 0 | 0 |
| SDC1 | R277H | −0.47 | 36% | 0 | 0 | 0 |
| CDH17 | I530M | −2.03 | 35% | 0 | 0 | 0 |
| TENM3 | R143H | −0.55 | 34% | 0 | 0 | 0 |
| USP6 | R410H | −1.40 | 34% | 0 | 0 | 0 |
| SIGLEC1 | R1446H | −0.71 | 34% | 0 | 0 | 0 |
| CSMD1 | V2823I | −1.45 | 33% | 0 | 0 | 0 |
| CASK | I755V | 0.81 | 33% | 0 | 0 | 0 |
| WNT11 | R82W | −1.83 | 33% | 0 | 0 | 0 |
| TENM2 | R1261H | −1.53 | 33% | 0 | 0 | 0 |
| SYT5 | R366W | −1.02 | 32% | 0 | 0 | 0 |
| IDO1 | A127V | −0.86 | 31% | 0 | 0 | 0 |
| GABRQ | E430D | −1.34 | 31% | 0 | 0 | 0 |
| PLA2R1 | A96T | −1.33 | 30% | 0 | 0 | 0 |
| ARX | R384C | −0.91 | 30% | 0 | 0 | 0 |
| CD244 | R355C | −1.68 | 30% | 0 | 0 | 0 |
| OR5AS1 | K303M | −1.43 | 29% | 0 | 0 | 0 |
| ZBTB8B | R286H | −1.14 | 29% | 0 | 0 | 0 |
| TMC1 | A460T | −1.99 | 28% | 0 | 0 | 0 |
| ANO1 | A22V | −1.15 | 27% | 0 | 0 | 0 |
| FBN2 | M523T | −1.46 | 27% | 0 | 0 | 0 |
| ERCC6L | Q598K | −0.75 | 26% | 0 | 0 | 0 |
| ARHGAP5 | E489K | 1.17 | 26% | 0 | 0 | 0 |
| DNAH12 | V2405M | −2.10 | 26% | 0 | 0 | 0 |
| ST6GALNAC1 | T163M | −1.52 | 25% | 0 | 0 | 0 |
| PTPRH | R433Q | −1.11 | 24% | 0 | 0 | 0 |
| MATK | A109T | −1.42 | 21% | 0 | 0 | 0 |
| HTR3E | R260C | −1.90 | 19% | 0 | 0 | 0 |
| SLC4A10 | V414F | −0.60 | 16% | 0 | 0 | 0 |
| FRG2C | G83R | −2.11 | 15% | 0 | 0 | 0 |
| NDUFV2 | V134A | 1.63 | 13% | 0 | 0 | 0 |
| PABPC3 | K444M | −0.93 | 12% | 0 | 0 | 0 |
| DNAH17 | P1229T | −1.22 | 12% | 0 | 0 | 0 |
| PABPC3 | S446G | −0.93 | 12% | 0 | 0 | 0 |
| CNTNAP3 | G1195R | −0.88 | 12% | 0 | 0 | 0 |

FPKM = fragments per kilobase per million total reads. “standardized” = standard deviation units. Transformed to a zero mean and unit standard deviation after logarithmic transformation using a SHASH distribution algorithm.

### Example 21: Polarity and Log P of Peptides

The method for prediction of MHC binding applied has been described in detail elsewhere (See, e.g., U.S. Pat. No. 10,706,955 incorporated herein by reference in its entirety). Briefly, each amino acid is described by multiple principal components (PC) derived by eigen decomposition and principal component analysis of the correlation matrices between 31 amino acid physical properties derived from experimental studies. PC1 is strongly influenced by the polarity of the amino acid [90, 91]. Thus, to arrive at an index of the polarity of each peptide the average of the PC1 of the constituent amino acids is used. The PC of each amino acid are shown in Table 27.

TABLE 27
Parameters of amino acids

| | Amino | AA | | | Log | | | |
|---|---|---|---|---|---|---|---|---|
| AA name & code | acid | MW | Log P | pl | D7.4 | PC1 | PC2 | PC3 |
| Phenylalanine (Phe) | F | 165.19 | −1.63 | 5.48 | 1.16 | 7.19 | −1.53 | 0.05 |
| Isoleucine (Ile) | I | 131.18 | −1.72 | 6.20 | 0.69 | 6.65 | 0.29 | 0.04 |
| Leucine (Leu) | L | 131.18 | −1.61 | 5.98 | 0.80 | 6.59 | −0.20 | 1.17 |
| Tryptophan (Trp) | W | 204.23 | −1.75 | 5.89 | 1.46 | 5.68 | −3.50 | 0.16 |
| Valine (Val) | V | 117.15 | −2.08 | 5.96 | 0.32 | 4.79 | 1.98 | −0.35 |
| Methionine (Met) | M | 149.21 | −1.84 | 5.74 | 0.51 | 4.14 | −0.43 | −1.46 |
| Tyrosine (Tyr) | Y | 181.19 | −2.42 | 5.66 | 0.55 | 2.58 | −2.06 | 0.37 |
| Cysteine (Cys) | C | 121.16 | −2.49 | 5.07 | 0.82 | 2.11 | 2.74 | −3.84 |
| Alanine (Ala) | A | 89.09 | −2.89 | 6.00 | −0.27 | 0.72 | 2.48 | 1.42 |
| Proline (Pro) | P | 115.13 | −2.50 | 6.30 | 0.15 | −0.03 | −0.36 | 1.87 |
| Glycine (Gly) | G | 75.07 | −3.25 | 5.97 | −0.22 | −0.76 | 3.08 | 1.21 |
| Threonine (Thr) | T | 119.12 | −2.92 | 5.60 | −0.26 | −1.43 | 0.80 | 0.94 |
| Histidine (His) | H | 155.16 | −3.56 | 7.59 | −0.44 | −2.55 | −1.00 | −1.94 |
| Serine (Ser) | S | 105.09 | −3.30 | 5.68 | −0.45 | −2.65 | 1.84 | 1.30 |
| Glutamine (Gln) | Q | 146.15 | −3.24 | 5.65 | −1.00 | −3.97 | −0.47 | 0.15 |
| Asparagine (Asn) | N | 132.12 | −3.41 | 5.41 | −0.98 | −4.35 | 0.22 | 0.30 |
| Glutamic Acid (Glu) | E | 147.13 | −2.94 | 3.22 | −2.19 | −5.70 | 0.34 | −1.46 |
| Asparic Acid (Asp) | D | 133.10 | −3.38 | 2.77 | −2.06 | −6.04 | 0.03 | −0.18 |
| Arginine (Arg) | R | 174.20 | −4.20 | 10.76 | −1.65 | −6.30 | −2.93 | −0.91 |
| Lysine (Lys) | K | 146.19 | −4.44 | 9.74 | −2.27 | −6.68 | −1.32 | 1.16 |

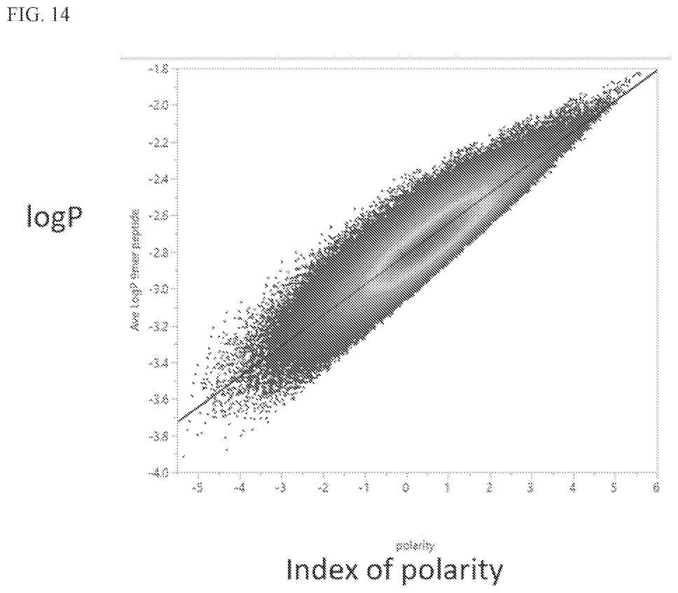

Table 27 also lists the log P for the octanol:water partition coefficient. The peptide log P is determined for each individual amino acid log Ps divided by the number of amino acids in the peptide. Overall, the average log P of a 9mer peptide (as shown in Table 27) has a value of −2.78, which is equivalent to <0.1% distribution in octanol and 99.9% in water. Peptides with a log P in excess of −2 is equivalent to approximately 1% in octanol and 99% in water. FIG. 14 provides an example of the distribution of index of polarity and log P calculated for MHC I allele A2902 across a group of alterative peptides derived from proteins mutated in a subject's tumor, A subset of these are shown in Table 28, from which selected peptides are drawn for a vaccine.

Table 28 shows, using 5 example mutated proteins, how different alternative peptides selected for each constant T cell exposed motif pentamer have an array of different polarities and partition coefficients. Hence a selection can be made of those alternative peptides which most favor solubility.

This figure and table are provided for illustration and so considered non limiting examples as the same approach can be applied to other mutated proteins and for other alleles, including both MHC I and MHC II alleles.

TABLE 28
Variation in logP and index of polarity among selected alternative peptides

| | | | | | | | | | A_2902 | | | |
|---|---|---|---|---|---|---|---|---|---|---|---|---|
| | | | | | | | | A_2902 | pre- | | | |
| | | | | | | | | pre- | dicted | | Ave | |
| | | | | | | | | dicted | binding | Index | LogP | |
| | | originat- | SEQ | | SEQ | | SEQ | binding | originat- | of | 9mer | molwt |
| gene | aa | ing | ID | TCEM | ID | proposed | ID | proposed | ing | polar- | pep- | 9mer |
| id | mutation | peptide | NO.: | core | NO.: | peptide | NO.: | peptide | peptide | ity | tide | peptide |
| NEK4 | | :EMSSSKDQP | 2101 | SSKDQ | 2109 | MMQSSKDQR | 2117 | −1.99 | −0.21 | −2.66 | −3.20 | 1110.39 |
| | | | | | | LSWSSKDQW | 2118 | −2.01 | −0.21 | −0.74 | −2.90 | 1136.35 |
| | | | | | | FVESSKDQY | 2119 | −1.98 | −0.21 | −1.46 | −2.97 | 1102.28 |
| | | SKDQPLSAR | 2102 | QPLSA | 2110 | EFDQPLSAY | 2120 | −2.04 | −1.05 | −0.15 | −2.66 | 1069.25 |
| | | | | | | LHSQPLSAY | 2121 | −2.00 | −1.05 | 0.52 | −2.71 | 1015.26 |
| | | | | | | TGAQPLSAY | 2122 | −2.06 | −1.05 | 0.20 | −2.78 | 907.11 |
| IST1 | S181C | PYEPDCVVM | 2103 | PDCVV | 2111 | MMEPDCVVY | 2123 | −2.08 | −0.56 | 1.20 | −2.40 | 1086.43 |
| | | | | | | NMPPDCVVY | 2124 | −2.01 | −0.56 | 0.89 | −2.52 | 1037.34 |
| | | | | | | YFNPDCVVY | 2125 | −2.03 | −0.56 | 1.51 | −2.49 | 1119.38 |
| FLOT2 | E11K | CHTVGPNKA | 2104 | VGPNK | 2112 | VTWVGPNKY | 2126 | −1.99 | −0.42 | 0.51 | −2.76 | 1063.35 |
| | | | | | | NVLVGPNKW | 2127 | −2.07 | −0.42 | 0.63 | −2.73 | 1026.34 |
| | | | | | | MESVGPNKW | 2128 | −2.08 | −0.42 | −0.62 | −2.83 | 1047.32 |
| | | VGPNKALVV | 2105 | NKALV | 2113 | RYVNKALVF | 2129 | −2.01 | −0.61 | 1.04 | −2.75 | 1109.46 |
| | | | | | | PWSNKALVR | 2130 | −2.03 | −0.61 | −0.25 | −2.91 | 1070.38 |
| | | | | | | WLYNKALVR | 2131 | −2.08 | −0.61 | 1.07 | −2.71 | 1162.53 |
| EGFR | A244V | YSFGVTCVK | 2106 | GVTCV | 2114 | VWEGVTCVR | 2132 | −2.02 | −0.44 | 0.89 | −2.64 | 1048.36 |
| | | | | | | SGVGVTCVY | 2133 | −2.01 | −0.44 | 1.50 | −2.65 | 884.15 |
| | | | | | | KAVGVTCVY | 2134 | −2.04 | −0.44 | 1.21 | −2.74 | 939.27 |
| | | SFGVTCVKK | 2107 | VTCVK | 2115 | EFEVTCVKR | 2135 | −2.02 | −1.11 | −0.77 | −2.86 | 1110.42 |
| | | | | | | GAEVTCVKY | 2136 | −2.07 | −1.11 | 0.05 | −2.83 | 969.25 |
| | | | | | | GYRVTCVKY | 2137 | −2.04 | −1.11 | 0.19 | −2.92 | 1088.42 |
| DCAF15 | V556M | SYRKSCMDM | 2108 | KSCMD | 2116 | IHSKSCMDY | 2138 | −2.08 | −0.60 | −0.56 | −2.94 | 1083.37 |

### Example 22: Example of Down Selection of Alternative Peptides Based on Criteria of Binding and Desirable Formulation Properties for 15 Mutated Tumor Proteins

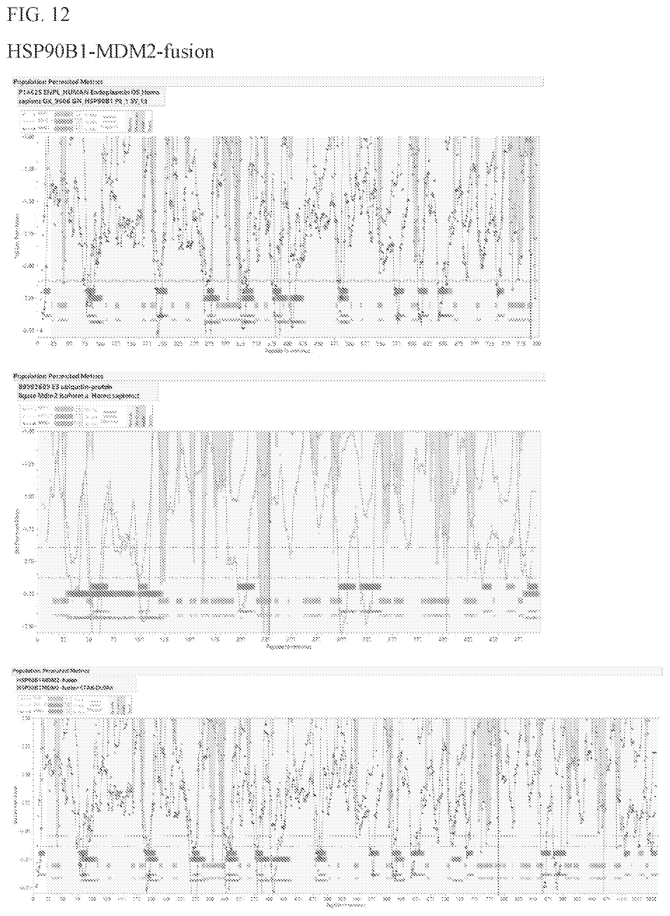

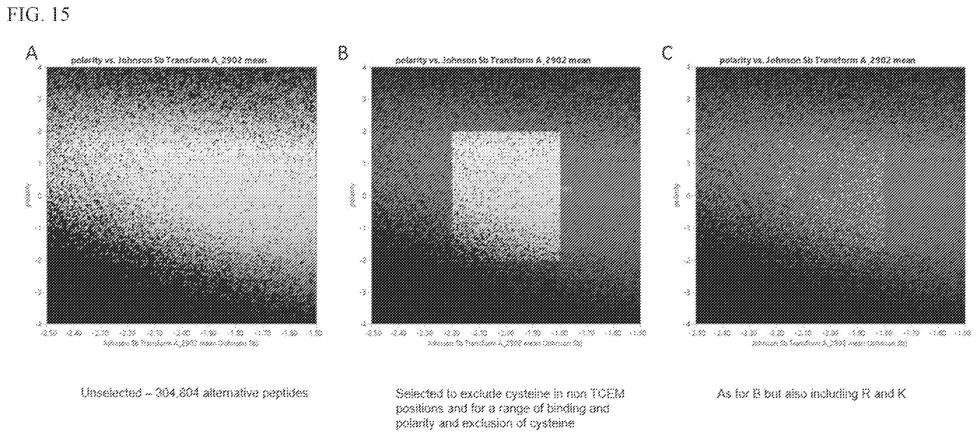

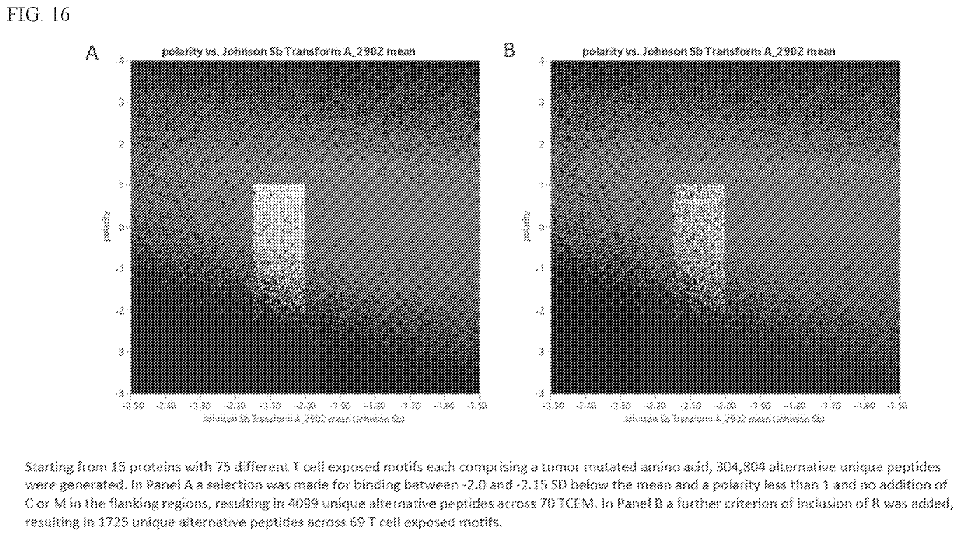

A cancer patient presented with 15 identified mutated proteins. Each mutated amino acid appears in 5 different T cell exposed motifs for potential targeting by CD8+ cells. Following the methods of Examples 1 and 2 peptides an array of possible alternative peptides is generated for each allele. FIGS. 15 and 16 document the results for A2902. The process was repeated for the other identified alleles the subject carried: A3201, B5101, C0702 and C1202 and C0704. As shown in FIGS. 15 and 16, for A2902 304,804 alternative unique peptides were generated with above average binding for A2902. FIG. 12 shows how this may number may be down selected based on polarity (represented by the index of polarity), binding affinity and inclusion or exclusion of specific amino acids which may impact aggregation (exclude cysteine), oxidation (exclude cysteine and methionine) or solubility (include arginine or lysine). In FIG. 16 we show how by applying selected criteria the number of candidate peptides can be reduced to 1725 peptides which can be used to target 69 of the T cell exposed motifs presented by this allele. From this set a further reduction to 2 peptides per T cell exposed motif is made resulting in 136 peptides. These are combined with a similar down selected set for the other alleles and a final selection made for inclusion in a vaccine designed to target a variety of T cell exposed motifs representing all 15 proteins and using different alleles

### Example 23: Example of Down Selection of Alternative Peptides Based on Criteria of Binding and Desirable Formulation Properties

Many allergens can cause life-threatening disease. Examples of these include, but are not limited to, the peanut allergens [92-94] and allergens to parasites of fish, in particular Anisakis spp. [95-97]. Others are not life threatening but cause persistent discomfort, for example cat allergy.

T cell epitopes linked to the induction of allergic reactions have been identified (iedb.org), and in particular MHC II peptides which likely serve as T cell CD4+ helpers. By increasing binding affinity to selected MHC II alleles it would be possible to induce T cell exhaustion or anergy following gradual increase of exposure. In Table 29 Example 24 we show how peptides may be designed with extremely high binding affinity to the relevant T cell epitopes. The table shows examples for three selected DRB alleles and the three selected allergens noted above. It will be noted that the peptides have also been selected based on their polarity index, calculated as noted above. The array of peptides from which the peptides shown were selected, i.e., those having a predicted binding affinity of <−2.51 standard deviation units below the mean, ranged in polarity index from −3.12 to 4.16 for the 3 alleles examined. indicating a wide range of probable solubility. These examples are for illustrative purposes and are considered non-limiting as the same approach can be applied to other alleles and other allergen epitopes.

### Example 24 Bespoke Peptides for Downregulation of Immune Responses

Many allergens can cause life-threatening disease. Examples of these include, but are not limited to, the peanut allergens [92-94] and allergens to parasites of fish, in particular Anisakis spp. [95-97]. Others are not life threatening but cause persistent discomfort, for example cat allergy.

T cell epitopes linked to the induction of allergic reactions have been identified (iedb.org), and in particular MHC II peptides which likely serve as T cell CD4+ helpers. By increasing binding affinity to selected MHC II alleles it would be possible to induce T cell exhaustion or anergy following gradual increase of exposure. In the following Table 29 we show how peptides may be designed with extremely high binding affinity to the relevant T cell epitopes. The table shows examples for 3 selected DRB alleles and the three selected allergens noted above; these are for illustrative purposes and are considered non-limiting as the same approach can be applied to other alleles and other allergen epitopes.

TABLE 29
Examples of bespoke peptides designed to provide very high binding affinity to induce CD4+ helper anergy in 3 example
allergens

| | | | | | | | | | Predicted | Predicted | | |
|---|---|---|---|---|---|---|---|---|---|---|---|---|
| | | | | | | | | | binding | binding | | |
| | | | | SEQ | | SEQ | | SEQ | affinity | affinity | | |
| | | | | ID | originating | ID | TCEM | ID | proposed | originating | | |
| gi | pos | curation | proposed peptide | NO.: | peptide | NO.: | core | NO.: | peptide | peptide | polarity | Allele |
| 26245447 | 39 | Ara h 2_02 | PLCLRPCEQHLRRRM | 1862 | RANLRPCEQHLMQKI | 1842 | RP~E~HL | 1822 | −2.98 | −1.60 | −0.62 | DRB1_0301 |
| 26245447 | 39 | Ara h 2_02 | RLMERPCEQHLLFCE | 1863 | RANLRPCEQHLMQKI | 1842 | RP~E~HL | 1822 | −3.04 | −1.60 | −0.06 | |
| 26245447 | 40 | Ara h 2_02 | LCAFPCEQHLMKLNC | 1864 | ANLRPCEQHLMQKIQ | 1843 | PC~Q~LM | 1823 | −2.83 | −1.82 | 0.99 | |
| 26245447 | 40 | Ara h 2_02 | QGQCPCEQHLMYQKM | 1865 | ANLRPCEQHLMQKIQ | 1843 | PC~Q~LM | 1823 | −2.74 | −1.82 | −0.66 | |
| 26245447 | 41 | Ara h 2_02 | HMLMCEQHLMQQLFC | 1866 | NLRPCEQHLMQKIQR | 1844 | CE~H~MQ | 1824 | −2.72 | −1.03 | 1.39 | |
| 26245447 | 41 | Ara h 2_02 | HQLICEQHLMQQERP | 1867 | NLRPCEQHLMQKIQR | 1844 | CE~H~MQ | 1824 | −2.77 | −1.03 | −0.84 | |
| 26245447 | 107 | Ara h 2_02 | IRGLFENNQRCMFEGL | 1868 | LNEFENNQRCMCEAL | 1845 | EN~Q~CM | 1825 | −2.71 | −0.54 | −0.29 | |
| 26245447 | 107 | Ara h 2_02 | LELIENNQRCMLLYE | 1869 | LNEFENNQRCMCEAL | 1845 | EN~Q~CM | 1825 | −2.59 | −0.54 | 0.39 | |
| 26245447 | 111 | Ara h 2_02 | LRYLRCMCEALDKLN | 1870 | ENNQRCMCEALQQIM | 1846 | RC~C~AL | 1826 | −2.89 | −1.06 | 0.18 | |
| 26245447 | 111 | Ara h 2_02 | AYLLRCMCEALHPGY | 1871 | ENNQRCMCEALQQIM | 1846 | RC~C~AL | 1826 | −2.74 | −1.06 | 1.29 | |
| 26245447 | 112 | Ara h 2_02 | ESMLCMCEALQQRPR | 1872 | NNQRCMCEALQQIME | 1847 | CM~E~LQ | 1827 | −2.82 | −0.91 | −0.55 | |
| 26245447 | 112 | Ara h 2_02 | APRFCMCEALQQYKR | 1873 | NNQRCMCEALQQIME | 1847 | CM~E~LQ | 1827 | −2.94 | −0.91 | −0.45 | |
| 26245447 | 112 | Ara h 2_02 | VLFYCMCEALQPGPR | 1874 | NNQRCMCEALQQIME | 1847 | CM~E~LQ | 1827 | −2.52 | −0.90 | 1.34 | DRB1_0401 |
| 26245447 | 112 | Ara h 2_02 | ALMFCMCEALQPTDN | 1875 | NNQRCMCEALQQIME | 1847 | CM~E~LQ | 1827 | −2.53 | −0.90 | 0.85 | |
| 26245447 | 113 | Ara h 2_02 | RTLFMCEALQQVQYG | 1876 | NQRCMCEALQQIMEN | 1848 | MC~A~QQ | 1828 | −2.55 | −0.82 | 0.57 | DRB1_0101 |
| 26245447 | 113 | Ara h 2_02 | KPWWMCEALQQLRTL | 1877 | NQRCMCEALQQIMEN | 1848 | MC~A~QQ | 1828 | −2.60 | −0.82 | 0.67 | |
| 26245447 | 113 | Ara h 2_02 | NMLVMCEALQQRRFA | 1878 | NQRCMCEALQQIMEN | 1848 | MC~A~QQ | 1828 | −2.54 | −1.78 | 0.43 | DRB1_0301 |
| 26245447 | 113 | Ara h 2_02 | LSILMCEALQQKMWR | 1879 | NQRCMCEALQQIMEN | 1848 | MC~A~QQ | 1828 | −2.69 | −1.78 | 0.93 | |
| 26245447 | 113 | Ara h 2_02 | PWIMMCEALQQGWPT | 1880 | NQRCMCEALQQIMEN | 1848 | MC~A~QQ | 1828 | −3.00 | −0.64 | 1.32 | DRB1_0401 |
| 26245447 | 113 | Ara h 2_02 | PAFWMCEALQQLRNL | 1881 | NQRCMCEALQQIMEN | 1848 | MC~A~QQ | 1828 | −2.52 | −0.64 | 1.07 | |
| 47605452 | 44 | Ani s 1 | RQIIVAWWHDDSPGV | 1882 | VKPSVAWWHDDKSGI | 1849 | VA~W~DD | 1829 | −2.54 | −0.73 | 0.44 | DRB1_0401 |
| 47605452 | 44 | Ani s 1 | KHFYVAWWHDDTGLM | 1883 | VKPSVAWWHDDKSGI | 1849 | VA~W~DD | 1829 | −2.66 | −0.73 | 0.75 | |
| 47605452 | 45 | Ani s 1 | YWLYAWWHDDKRLTQ | 1884 | KPSVAWWHDDKSGIC | 1850 | AW~H~DK | 1830 | −2.67 | −0.64 | 0.21 | DRB1_0101 |
| 47605452 | 45 | Ani s 1 | AWYWAWWHDDKIQPT | 1885 | KPSVAWWHDDKSGIC | 1850 | AW~H~DK | 1830 | −2.83 | −0.64 | 0.44 | |
| 47605452 | 45 | Ani s 1 | GHQFAWWHDDKAAFL | 1886 | KPSVAWWHDDKSGIC | 1850 | AW~H~DK | 1830 | −2.88 | −1.27 | 0.39 | DRB1_0401 |
| 47605452 | 45 | Ani s 1 | LLFIAWWHDDKQEYG | 1887 | KPSVAWWHDDKSGIC | 1850 | AW~H~DK | 1830 | −3.05 | −1.27 | 0.66 | |
| 47605452 | 46 | Ani s 1 | RDYLWWHDDKSQALI | 1888 | PSVAWWHDDKSGICL | 1851 | WW~D~KS | 1831 | −2.69 | −0.98 | −0.39 | DRB1_0101 |
| 47605452 | 46 | Ani s 1 | DYGLWWHDDKSFGFL | 1889 | PSVAWWHDDKSGICL | 1851 | WW~D~KS | 1831 | −2.56 | −0.98 | 0.66 | |
| 47605452 | 46 | Ani s 1 | EEHYWWHDDKSHDRE | 1890 | PSVAWWHDDKSGICL | 1851 | WW~D~KS | 1831 | −2.80 | −1.24 | −2.97 | DRB1_0301 |
| 47605452 | 46 | Ani s 1 | QRMFWWHDDKSNKSR | 1891 | PSVAWWHDDKSGICL | 1851 | WW~D~KS | 1831 | −3.02 | −1.24 | −2.10 | |
| 47605452 | 46 | Ani s 1 | TGALWWHDDKSFEAL | 1892 | PSVAWWHDDKSGICL | 1851 | WW~D~KS | 1831 | −2.64 | −2.50 | 0.09 | DRB1_0401 |
| 47605452 | 46 | Ani s 1 | KFELWWHDDKSMGDY | 1893 | PSVAWWHDDKSGICL | 1851 | WW~D~KS | 1831 | −2.52 | −2.50 | −0.75 | |
| 47605452 | 47 | Ani s 1 | LPWLWHDDKSGATTQ | 1894 | SVAWWHDDKSGICLS | 1852 | WH~D~SG | 1832 | −2.81 | −1.89 | −0.42 | DRB1_0101 |
| 47605452 | 47 | Ani s 1 | FFRFWHDDKSGLILH | 1895 | SVAWWHDDKSGICLS | 1852 | WH~D~SG | 1832 | −3.46 | −1.89 | 0.90 | |
| 47605452 | 47 | Ani s 1 | YVFKWHDDKSGEEWL | 1896 | SVAWWHDDKSGICLS | 1852 | WH~D~SG | 1832 | −2.53 | −1.95 | −0.69 | DRB1_0301 |
| 47605452 | 47 | Ani s 1 | YNFLWHDDKSGQQKH | 1897 | SVAWWHDDKSGICLS | 1852 | WH~D~SG | 1832 | −2.51 | −1.95 | −1.61 | |
| 47605452 | 47 | Ani s 1 | IAQFWHDDKSGPAAP | 1898 | SVAWWHDDKSGICLS | 1852 | WH~D~SG | 1832 | −2.65 | −1.98 | −0.47 | DRB1_0401 |
| 47605452 | 47 | Ani s 1 | QLFLWHDDKSGKTST | 1899 | SVAWWHDDKSGICLS | 1852 | WH~D~SG | 1832 | −2.91 | −1.98 | −0.99 | |
| 47605452 | 48 | Ani s 1 | QFFFHDDKSGIFLQR | 1900 | VAWWHDDKSGICLSF | 1853 | HD~K~GI | 1833 | −3.67 | −1.34 | 0.20 | DRB1_0101 |
| 47605452 | 48 | Ani s 1 | MMFIHDDKSGILSLA | 1901 | VAWWHDDKSGICLSF | 1853 | HD~K~GI | 1833 | −3.31 | −1.34 | 1.02 | |
| 47605452 | 48 | Ani s 1 | DLMWHDDKSGIKRYF | 1902 | VAWWHDDKSGICLSF | 1853 | HD~K~GI | 1833 | −2.69 | −0.75 | −0.73 | DRB1_0301 |
| 47605452 | 118 | Ani s 1 | KRMMQCKMMAFAMID | 1903 | PNGYQCKMMAFMGLC | 1854 | QC~M~AF | 1834 | −2.68 | −0.84 | 0.56 | |
| 47605452 | 118 | Ani s 1 | RPLLQCKMMAFYQWE | 1904 | PNGYQCKMMAFMGLC | 1854 | QC~M~AF | 1834 | −2.96 | −0.84 | 0.87 | |
| 47605452 | 118 | Ani s 1 | RKMFQCKMMAFLQLS | 1905 | PNGYQCKMMAFMGLC | 1854 | QC~M~AF | 1834 | −2.76 | −0.62 | 0.84 | DRB1_0401 |
| 47605452 | 119 | Ani s 1 | PTIWCKMMAFMGQLK | 1906 | NGYQCKMMAFMGLCC | 1855 | CK~M~FM | 1835 | −2.65 | −0.91 | 1.45 | DRB1_0101 |
| 47605452 | 119 | Ani s 1 | RLFWCKMMAFMTAGD | 1907 | NGYQCKMMAFMGLCC | 1855 | CK~M~FM | 1835 | −2.60 | −0.61 | 1.43 | DRB1_0401 |
| 47605452 | 120 | Ani s 1 | QRLWKMMAFMGVETG | 1908 | GYQCKMMAFMGLCCP | 1856 | KM~A~MG | 1836 | −2.88 | −1.14 | 0.79 | DRB1_0101 |
| 47605452 | 120 | Ani s 1 | KLILKMMAFMGGRQG | 1909 | GYQCKMMAFMGLCCP | 1856 | KM~A~MG | 1836 | −2.56 | −1.14 | 0.95 | |
| 47605452 | 120 | Ani s 1 | KLVLKMMAFMGQGKC | 1910 | GYQCKMMAFMGLCCP | 1856 | KM~A~MG | 1836 | −2.53 | −1.09 | 0.99 | DRB1_0401 |
| 47605452 | 120 | Ani s 1 | KQRLKMMAFMGGSAT | 1911 | GYQCKMMAFMGLCCP | 1856 | KM~A~MG | 1836 | −2.86 | −1.09 | −0.11 | |
| 47605452 | 121 | Ani s 1 | KLMTMMAFMGLKSKC | 1912 | YQCKMMAFMGLCCPT | 1857 | MM~F~GL | 1837 | −2.69 | −1.11 | 0.99 | DRB1_0101 |
| 47605452 | 121 | Ani s 1 | LRRLMMAFMGLSGRG | 1913 | YQCKMMAFMGLCCPT | 1857 | MM~F~GL | 1837 | −2.81 | −1.11 | 1.08 | |
| 47605452 | 121 | Ani s 1 | YEFLMMAFMGLKKMR | 1914 | YQCKMMAFMGLCCPT | 1857 | MM~F~GL | 1837 | −2.78 | −1.09 | 1.42 | DRB1_0301 |
| 47605452 | 121 | Ani s 1 | MKTLMMAFMGLKRMK | 1915 | YQCKMMAFMGLCCPT | 1857 | MM~F~GL | 1837 | −2.96 | −1.09 | 0.88 | |
| 47605452 | 122 | Ani s 1 | KIDIMAFMGLCANRG | 1916 | QCKMMAFMGLCCPTK | 1858 | MA~M~LC | 1838 | −2.75 | −1.57 | 0.93 | DRB1_0101 |
| 47605452 | 122 | Ani s 1 | KKIYMAFMGLCDTLA | 1917 | QCKMMAFMGLCCPTK | 1858 | MA~M~LC | 1838 | −4.00 | −1.57 | 1.32 | |
| 47605452 | 122 | Ani s 1 | RWLRMAFMGLCQAES | 1918 | QCKMMAFMGLCCPTK | 1858 | MA~M~LC | 1838 | −2.87 | −0.52 | 0.81 | DRB1_0401 |
| 47605452 | 122 | Ani s 1 | ERPWMAFMGLCPEFG | 1919 | QCKMMAFMGLCCPTK | 1858 | MA~M~LC | 1838 | −2.60 | −0.52 | 1.23 | |
| 47605452 | 123 | Ani s 1 | QMRFAFMGLCCNTIS | 1920 | CKMMAFMGLCCPTKE | 1859 | AF~G~CC | 1839 | −2.54 | −1.28 | 1.43 | DRB1_0101 |
| 47605452 | 123 | Ani s 1 | IDFWAFMGLCCPDTD | 1921 | CKMMAFMGLCCPTKE | 1859 | AF~G~CC | 1839 | −2.84 | −1.28 | 1.47 | |
| 47605452 | 123 | Ani s 1 | NAHWAFMGLCCAGKR | 1922 | CKMMAFMGLCCPTKE | 1859 | AF~G~CC | 1839 | −2.56 | −0.76 | 0.57 | DRB1_0401 |
| 163823 | 19 | major | RLMMTCPIFYDKSDE | 1923 | KMAETCPIFYDVFFA | 1860 | TC~I~YD | 1840 | −2.72 | −0.11 | −0.10 | DRB1_0401 |
| | | allergen I | | | | | | | | | | |
| | | Felis catus | | | | | | | | | | |
| 163823 | 19 | major | CFLFTCPIFYDDKDI | 1924 | KMAETCPIFYDVFFA | 1860 | TC~I~YD | 1840 | −2.66 | −0.11 | 1.47 | DRB1_0401 |
| | | allergen I | | | | | | | | | | |
| | | Felis catus | | | | | | | | | | |
| 163823 | 20 | major | KMRFCPIFYDVANYD | 1925 | MAETCPIFYDVFFAV | 1861 | CP~F~DV | 1841 | −2.54 | −0.28 | 0.57 | DRB1_0101 |
| | | allergen I | | | | | | | | | | |
| | | Felis catus | | | | | | | | | | |

1. Lefranc M P, Giudicelli V, Ginestoux C, Jabado-Michaloud J, Folch G, Bellahcene F, et al. IMGT, the international ImMunoGeneTics information system. Nucleic acids research. 2009; 37(Database issue): D1006-12. Epub 2008 Nov. 4. doi: 10.1093/nar/gkn838. PubMed PMID: 18978023; PubMed Central PMCID: PMC2686541.
2. Huang H, Ostroff G R, Lec C K, Wang J P, Specht C A, Levitz S M. Distinct patterns of dendritic cell cytokine release stimulated by fungal beta-glucans and toll-like receptor agonists. Infect Immun. 2009; 77(5): 1774-81. Epub 2009 Mar. 11. doi: 10.1128/IAI.00086-09. PubMed PMID: 19273561; PubMed Central PMCID: PMCPMC2681737.
3. Soto E R, Ostroff G R. Characterization of multilayered nanoparticles encapsulated in yeast cell wall particles for DNA delivery. Bioconjugate chemistry. 2008; 19(4):840-8. Epub 2008 Apr. 2. doi: 10.1021/bc700329p. PubMed PMID: 18376856.
4. Adusumilli P S, Cha E, Cornfeld M, Davis T, Diab A, Dubensky T W, Jr., et al. New Cancer Immunotherapy Agents in Development: a report from an associated program of the 31(st)Annual Meeting of the Society for Immunotherapy of Cancer, 2016. J Immunother Cancer. 2017; 5:50. Epub 2017 Jun. 27. doi: 10.1186/s40425-017-0253-2. PubMed PMID: 28649381; PubMed Central PMCID: PMCPMC5477277.
5. Ilyas S, Yang J C. Landscape of Tumor Antigens in T Cell Immunotherapy. J Immunol. 2015; 195(11):5117-22. Epub 2015 Nov. 22. doi: 10.4049/jimmunol.1501657. PubMed PMID: 26589749; PubMed Central PMCID: PMCPMC4656134.
6. Aldous A R, Dong J Z. Personalized neoantigen vaccines: A new approach to cancer immunotherapy. Bioorg Med Chem. 2018; 26(10):2842-9. Epub 2017 Nov. 8. doi: 10.1016/j.bmc.2017.10.021. PubMed PMID: 29111369.
7. Ophir E, Bobisse S, Coukos G, Harari A, Kandalaft L E. Personalized approaches to active immunotherapy in cancer. Biochim Biophys Acta. 2016; 1865(1):72-82. Epub 2015 Aug. 5. doi: 10.1016/j.bbcan.2015.07.004. PubMed PMID: 26241169.
8. Fennemann F L, de Vries I J M, Figdor C G, Verdoes M. Attacking Tumors From All Sides: Personalized Multiplex Vaccines to Tackle Intratumor Heterogeneity. Frontiers in immunology. 2019; 10:824. Epub 2019 May 2. doi: 10.3389/fimmu.2019.00824. PubMed PMID: 31040852; PubMed Central PMCID: PMCPMC6476980.
9. Ott P A, Hu Z, Keskin D B, Shukla S A, Sun J, Bozym D J, et al. An immunogenic personal neoantigen vaccine for patients with melanoma. Nature. 2017; 547(7662):217-21. Epub 2017 Jul. 6. doi: 10.1038/nature22991. PubMed PMID: 28678778; PubMed Central PMCID: PMCPMC5577644.
10. Sahin U, Derhovanessian E, Miller M, Kloke B P, Simon P, Lower M, et al. Personalized RNA mutanome vaccines mobilize poly-specific therapeutic immunity against cancer. Nature. 2017; 547(7662):222-6. Epub 2017 Jul. 6. doi: 10.1038/nature23003. PubMed PMID: 28678784.
11. Li F, Chen C, Ju T, Gao J, Yan J, Wang P, et al. Rapid tumor regression in an Asian lung cancer patient following personalized neo-epitope peptide vaccination. Oncoimmunology. 2016; 5(12):c1238539. Epub 2017 Jan. 27. doi: 10.1080/2162402X.2016.1238539. PubMed PMID: 28123873; PubMed Central PMCID: PMCPMC5214696.
12. Hilf N, Kuttruff-Coqui S, Frenzel K, Bukur V, Stevanovic S, Gouttefangeas C, et al. Actively personalized vaccination trial for newly diagnosed glioblastoma. Nature. 2019; 565(7738):240-5. Epub 2018 Dec. 21. doi: 10.1038/s41586-018-0810-y. PubMed PMID: 30568303.
13. Keskin D B, Anandappa A J, Sun J, Tirosh I, Mathewson N D, Li S, et al. Neoantigen vaccine generates intratumoral T cell responses in phase Ib glioblastoma trial. Nature. 2019; 565(7738):234-9. Epub 2018 Dec. 21. doi: 10.1038/s41586-018-0792-9. PubMed PMID: 30568305.
14. Rabizadeh S, Garner C, Sanborn J Z, Benz S C, Reddy S, Soon-Shiong P. Comprehensive genomic transcriptomic tumor-normal gene panel analysis for enhanced precision in patients with lung cancer. Oncotarget. 2018; 9(27): 19223-32. Epub 2018 May 4. doi: 10.18632/oncotarget.24973. PubMed PMID: 29721196; PubMed Central PMCID: PMCPMC5922390.
15. Yadav M, Jhunjhunwala S, Phung Q T, Lupardus P, Tanguay J, Bumbaca S, et al. Predicting immunogenic tumour mutations by combining mass spectrometry and exome sequencing. Nature. 2014; 515(7528):572-6. Epub 2014 Nov. 28. doi: 10.1038/nature 14001. PubMed PMID: 25428506.
16. Abelin J G, Keskin D B, Sarkizova S, Hartigan C R, Zhang W, Sidney J, et al. Mass Spectrometry Profiling of HLA-Associated Peptidomes in Mono-allelic Cells Enables More Accurate Epitope Prediction. Immunity. 2017; 46(2):315-26. Epub 2017 Feb. 24. doi: 10.1016/j.immuni.2017.02.007. PubMed PMID: 28228285; PubMed Central PMCID: PMCPMC5405381.
17. Hoof I, Peters B, Sidney J, Pedersen L E, Sette A, Lund O, et al. NetMHCpan, a method for MHC class I binding prediction beyond humans. Immunogenetics. 2009; 61(1):1-13. doi: 10.1007/s00251-008-0341-z [doi].
18. Hanahan D, Weinberg R A. Hallmarks of cancer: the next generation. Cell. 2011; 144(5):646-74. Epub 2011 Mar. 8. doi: 10.1016/j.cell.2011.02.013. PubMed PMID: 21376230.
19. Chen D S, Mellman I. Elements of cancer immunity and the cancer-immune set point. Nature. 2017; 541(7637):321-30. Epub 2017 Jan. 20. doi: 10.1038/nature21349. PubMed PMID: 28102259.
20. Cornella H, Alsinet C, Sayols S, Zhang Z, Hao K, Cabellos L, et al. Unique genomic profile of fibrolamellar hepatocellular carcinoma. Gastroenterology. 2015; 148(4):806-18 e10. Epub 2015 Jan. 6. doi: 10.1053/j.gastro.2014.12.028. PubMed PMID: 25557953; PubMed Central PMCID: PMCPMC4521774.
21. Biernacki M A, Bleakley M. Neoantigens in Hematologic Malignancies. Frontiers in immunology. 2020; 11:121. Epub 2020 Mar. 3. doi: 10.3389/fimmu.2020.00121. PubMed PMID: 32117272; PubMed Central PMCID: PMCPMC7033457.
22. Georgescu M M, Islam M Z, Li Y, Traylor J, Nanda A. Novel targetable FGFR2 and FGFR3 alterations in glioblastoma associate with aggressive phenotype and distinct gene expression programs. Acta Neuropathol Commun. 2021; 9(1):69. Epub 2021 Apr. 16. doi: 10.1186/s40478-021-01170-1. PubMed PMID: 33853673; PubMed Central PMCID: PMCPMC8048363.
23. De Luca A, Esposito Abate R, Rachiglio A M, Maiello M R, Esposito C, Schettino C, et al. FGFR Fusions in Cancer: From Diagnostic Approaches to Therapeutic Intervention. Int J Mol Sci. 2020; 21(18). Epub 2020 Sep. 24. doi: 10.3390/ijms21186856. PubMed PMID: 32962091; PubMed Central PMCID: PMCPMC7555921.
24. Gao Q, Liang W W, Foltz S M, Mutharasu G, Jayasinghe R G, Cao S, et al. Driver Fusions and Their Implications in the Development and Treatment of Human Cancers. Cell reports. 2018; 23(1):227-38 e3. Epub 2018 Apr. 5. doi: 10.1016/j.celrep.2018.03.050. PubMed PMID: 29617662; PubMed Central PMCID: PMCPMC5916809.
25. French C A. NUT Carcinoma: Clinicopathologic features, pathogenesis, and treatment. Pathol Int. 2018; 68(11):583-95. Epub 2018 Oct. 27. doi: 10.1111/pin.12727. PubMed PMID: 30362654.
26. Dyall R, Bowne W B, Weber L W, LeMaoult J, Szabo P, Moroi Y, et al. Heteroclitic immunization induces tumor immunity. J Exp Med. 1998; 188(9): 1553-61. Epub 1998 Nov. 6. doi: 10.1084/jem. 188.9.1553. PubMed PMID: 9802967; PubMed Central PMCID: PMCPMC2212523.
27. Zirlik K M, Zahrich D, Neuberg D, Gribben J G. Cytotoxic T cells generated against heteroclitic peptides kill primary tumor cells independent of the binding affinity of the native tumor antigen peptide. Blood. 2006; 108(12):3865-70. Epub 2006 Aug. 12. doi: 10.1182/blood-2006-04-014415. PubMed PMID: 16902144; PubMed Central PMCID: PMCPMC1895467.
28. Purcell A W, Mccluskey J, Rossjohn J. More than one reason to rethink the use of peptides in vaccine design. Nature reviews Drug discovery. 2007; 6(5):404-14. Epub 2007 May 3. doi: 10.1038/nrd2224. PubMed PMID: 17473845.
29. Cole D K, Miles K M, Madura F, Holland C J, Schauenburg A J, Godkin A J, et al. T-cell receptor (TCR)-peptide specificity overrides affinity-enhancing TCR-major histocompatibility complex interactions. J Biol Chem. 2014; 289(2):628-38. Epub 2013 Nov. 8. doi: 10.1074/jbc.M113.522110. PubMed PMID: 24196962; PubMed Central PMCID: PMC3887192.
30. Madura F, Rizkallah P J, Holland C J, Fuller A, Bulek A, Godkin A J, et al. Structural basis for ineffective T-cell responses to MHC anchor residue-improved “heteroclitic” peptides. European journal of immunology. 2015; 45(2):584-91. Epub 2014 Dec. 5. doi: 10.1002/cji.201445114. PubMed PMID: 25471691; PubMed Central PMCID: PMCPMC4357396.
31. Cavalluzzo B, Ragone C, Mauriello A, Petrizzo A, Manolio C, Caporale A, et al. Identification and characterization of heteroclitic peptides in TCR-binding positions with improved HLA-binding efficacy. Journal of translational medicine. 2021; 19(1):89. Epub 2021 Feb. 28. doi: 10.1186/s12967-021-02757-x. PubMed PMID: 33637105; PubMed Central PMCID: PMCPMC7913412.
32. Rosenberg S A, Yang J C, Schwartzentruber D J, Hwu P, Marincola F M, Topalian S L, et al. Immunologic and therapeutic evaluation of a synthetic peptide vaccine for the treatment of patients with metastatic melanoma. Nat Med. 1998; 4(3):321-7. Epub 1998 Mar. 21. doi: 10.1038/nm0398-321. PubMed PMID: 9500606; PubMed Central PMCID: PMCPMC2064864.
33. Valmori D, Fonteneau J F, Lizana C M, Gervois N, Lienard D, Rimoldi D, et al. Enhanced generation of specific tumor-reactive CTL in vitro by selected Melan-A/MART-1 immunodominant peptide analogues. J Immunol. 1998; 160(4):1750-8. Epub 1998 Feb. 20. PubMed PMID: 9469433.
34. Topalian S L, Gonzales M I, Parkhurst M, Li Y F, Southwood S, Sette A, et al. Melanoma-specific CD4+ T cells recognize nonmutated HLA-DR-restricted tyrosinase epitopes. J Exp Med. 1996; 183(5): 1965-71. Epub 1996 May 1. doi: 10.1084/jem. 183.5.1965. PubMed PMID: 8642306; PubMed Central PMCID: PMCPMC2192565.
35. Parkhurst M R, Salgaller M L, Southwood S, Robbins P F, Sette A, Rosenberg S A, et al. Improved induction of melanoma-reactive CTL with peptides from the melanoma antigen gp100 modified at HLA-A*0201-binding residues. J Immunol. 1996; 157(6):2539-48. Epub 1996 Sep. 15. PubMed PMID: 8805655.
36. Terasawa H, Tsang K Y, Gulley J, Arlen P, Schlom J. Identification and characterization of a human agonist cytotoxic T-lymphocyte epitope of human prostate-specific antigen. Clin Cancer Res. 2002; 8(1):41-53. Epub 2002 Jan. 22. PubMed PMID: 11801539.
37. Zauderer M G, Tsao A S, Dao T, Panageas K, Lai W V, Rimner A, et al. A Randomized Phase II Trial of Adjuvant Galinpepimut-S, WT-1 Analogue Peptide Vaccine, After Multimodality Therapy for Patients with Malignant Pleural Mesothelioma. Clin Cancer Res. 2017; 23(24):7483-9. Epub 2017 Oct. 4. doi: 10.1158/1078-0432.CCR-17-2169. PubMed PMID: 28972039; PubMed Central PMCID: PMCPMC5732877.
38. Tynan F E, Burrows S R, Buckle A M, Clements C S, Borg N A, Miles J J, et al. T cell receptor recognition of a ‘super-bulged’ major histocompatibility complex class I-bound peptide. Nat Immunol. 2005; 6(11):1114-22. Epub 2005 Sep. 28. doi: 10.1038/ni1257. PubMed PMID: 16186824.
39. Baumgartner C K, Ferrante A, Nagaoka M, Gorski J, Malherbe L P. Peptide-MHC class II complex stability governs CD4+ T cell clonal selection. J Immunol. 2010; 184(2):573-81. doi: jimmunol.0902107 [pii]; 10.4049/jimmunol.0902107 [doi].
40. Petrova G, Ferrante A, Gorski J. Cross-reactivity of T cells and its role in the immune system. Critical reviews in immunology. 2012; 32(4):349-72. Epub 2012 Dec. 15. PubMed PMID: 23237510; PubMed Central PMCID: PMC3595599.
41. Petrova G V, Gorski J. Cross-reactive responses to modified M1(5)(8)-(6)(6) peptides by CD8(+) T cells that use noncanonical BV genes can describe unknown repertoires. European journal of immunology. 2012; 42(11):3001-8. Epub 2012 Aug. 7. doi: 10.1002/cji.201242596. PubMed PMID: 22865108; PubMed Central PMCID: PMC3817827.
42. Havel J J, Chowell D, Chan T A. The evolving landscape of biomarkers for checkpoint inhibitor immunotherapy. Nature reviews Cancer. 2019; 19(3): 133-50. Epub 2019 Feb. 14. doi: 10.1038/s41568-019-0116-x. PubMed PMID: 30755690.
43. Mandal R, Samstein R M, Lee K W, Havel J J, Wang H, Krishna C, et al. Genetic diversity of tumors with mismatch repair deficiency influences anti-PD-1 immunotherapy response. Science. 2019; 364(6439):485-91. Epub 2019 May 3. doi: 10.1126/science.aau0447. PubMed PMID: 31048490.
44. Gibney G T, Weiner L M, Atkins M B. Predictive biomarkers for checkpoint inhibitor-based immunotherapy. The lancet oncology. 2016; 17(12):c542-e51. Epub 2016 Dec. 8. doi: 10.1016/S1470-2045(16)30406-5. PubMed PMID: 27924752; PubMed Central PMCID: PMCPMC5702534.
45. Bajwa R, Cheema A, Khan T, Amirpour A, Paul A, Chaughtai S, et al. Adverse Effects of Immune Checkpoint Inhibitors (Programmed Death-1 Inhibitors and Cytotoxic T-Lymphocyte-Associated Protein-4 Inhibitors): Results of a Retrospective Study. J Clin Med Res. 2019; 11(4):225-36. Epub 2019 Apr. 3. doi: 10.14740/jocmr3750. PubMed PMID: 30937112; PubMed Central PMCID: PMCPMC6436564.
46. Gubin M M, Zhang X, Schuster H, Caron E, Ward J P, Noguchi T, et al. Checkpoint blockade cancer immunotherapy targets tumour-specific mutant antigens. Nature. 2014; 515(7528):577-81. Epub 2014 Nov. 28. doi: 10.1038/nature13988. PubMed PMID: 25428507; PubMed Central PMCID: PMCPMC4279952.
47. Cohen C J, Gartner J J, Horovitz-Fried M, Shamalov K, Trebska-McGowan K, Bliskovsky V V, et al. Isolation of neoantigen-specific T cells from tumor and peripheral lymphocytes. J Clin Invest. 2015; 125(10):3981-91. Epub 2015 Sep. 22. doi: 10.1172/JCI82416. PubMed PMID: 26389673; PubMed Central PMCID: PMCPMC4607110.
48. Lauvau G, Soudja S M. Mechanisms of Memory T Cell Activation and Effective Immunity. Adv Exp Med Biol. 2015; 850:73-80. Epub 2015 Sep. 2. doi: 10.1007/978-3-319-15774-0_6. PubMed PMID: 26324347; PubMed Central PMCID: PMCPMC4836952.
49. Zehn D, Lee S Y, Bevan M J. Complete but curtailed T-cell response to very low-affinity antigen. Nature. 2009; 458(7235):211-4. Epub 2009 Feb. 3. doi: 10.1038/nature07657. PubMed PMID: 19182777; PubMed Central PMCID: PMCPMC2735344.
50. Soudja S M, Chandrabos C, Yakob E, Veenstra M, Palliser D, Lauvau G. Memory-T-cell-derived interferon-gamma instructs potent innate cell activation for protective immunity. Immunity. 2014; 40(6):974-88. Epub 2014 Jun. 17. doi: 10.1016/j.immuni.2014.05.005. PubMed PMID: 24931122; PubMed Central PMCID: PMCPMC4105986.
51. Jain D, Mahammad S S, Singh P P, Kodipyaka R. A review on parenteral delivery of peptides and proteins. Drug Dev Ind Pharm. 2019; 45(9): 1403-20. Epub 2019 Jun. 20. doi: 10.1080/03639045.2019.1628770. PubMed PMID: 31215293.
52. Badurdeen S, Valladares D B, Farrar J, Gozzer E, Kroeger A, Kuswara N, et al. Sharing experiences: towards an evidence based model of dengue surveillance and outbreak response in Latin America and Asia. BMC public health. 2013; 13:607. doi: 10.1186/1471-2458-13-607. PubMed PMID: 23800243; PubMed Central PMCID: PMC3697990.
53. Reed S G, Orr M T, Fox C B. Key roles of adjuvants in modern vaccines. Nat Med. 2013; 19(12): 1597-608. Epub 2013 Dec. 7. doi: 10.1038/nm.3409. PubMed PMID: 24309663.
54. Wosen J E, Mukhopadhyay D, Macaubas C, Mellins E D. Epithelial MHC Class II Expression and Its Role in Antigen Presentation in the Gastrointestinal and Respiratory Tracts. Frontiers in immunology. 2018; 9:2144. Epub 2018 Oct. 16. doi: 10.3389/fimmu.2018.02144. PubMed PMID: 30319613; PubMed Central PMCID: PMCPMC6167424.
55. Mowat A M, Agace W W. Regional specialization within the intestinal immune system. Nature reviews Immunology. 2014; 14(10):667-85. Epub 2014 Sep. 23. doi: 10.1038/nri3738. PubMed PMID: 25234148.
56. Shao L, Kamalu O, Mayer L. Non-classical MHC class I molecules on intestinal epithelial cells: mediators of mucosal crosstalk. Immunol Rev. 2005; 206:160-76. Epub 2005 Jul. 29. doi: 10.1111/j.0105-2896.2005.00295.x. PubMed PMID: 16048548.
57. Rani S, Rana R, Saraogi G K, Kumar V, Gupta U. Self-Emulsifying Oral Lipid Drug Delivery Systems: Advances and Challenges. AAPS PharmSciTech. 2019; 20(3): 129. Epub 2019 Mar. 1. doi: 10.1208/s12249-019-1335-x. PubMed PMID: 30815765.
58. Szolek A, Schubert B, Mohr C, Sturm M, Feldhahn M, Kohlbacher O. OptiType: precision HLA typing from next-generation sequencing data. Bioinformatics. 2014; 30(23):3310-6. Epub 2014 Aug. 22. doi: 10.1093/bioinformatics/btu548. PubMed PMID: 25143287; PubMed Central PMCID: PMCPMC4441069.
59. Boratyn G M, Thierry-Mieg J, Thierry-Mieg D, Busby B, Madden T L. Magic-BLAST, an accurate RNA-seq aligner for long and short reads. BMC Bioinformatics. 2019; 20(1):405. Epub 2019 Jul. 28. doi: 10.1186/s12859-019-2996-x. PubMed PMID: 31345161; PubMed Central PMCID: PMCPMC6659269.
60. Larjo A, Eveleigh R, Kilpelainen E, Kwan T, Pastinen T, Koskela S, et al. Accuracy of Programs for the Determination of Human Leukocyte Antigen Alleles from Next-Generation Sequencing Data. Frontiers in immunology. 2017; 8:1815. Epub 2018 Jan. 13. doi: 10.3389/fimmu.2017.01815. PubMed PMID: 29326702; PubMed Central PMCID: PMCPMC5733459.
61. An Z, Aksoy O, Zheng T, Fan Q W, Weiss W A. Epidermal growth factor receptor and EGFRvIII in glioblastoma: signaling pathways and targeted therapies. Oncogene. 2018; 37(12): 1561-75. Epub 2018 Jan. 13. doi: 10.1038/s41388-017-0045-7. PubMed PMID: 29321659; PubMed Central PMCID: PMCPMC5860944.
62. Daubon T, Hemadou, A., Romero-Garmendia, I., Salch, M. Glioblastoma Immune Landscape and the Potential of New Immunotherapies. Frontiers in immunology. 2020; 11:Article 585616. doi: doi: 10.3389/fimmu.2020.585616.
63. Mueller S, Taitt J M, Villanueva-Meyer J E, Bonner E R, Nejo T, Lulla R R, et al. Mass cytometry detects H3.3K27M-specific vaccine responses in diffuse midline glioma. J Clin Invest. 2020. Epub 2020 Aug. 21. doi: 10.1172/JCI140378. PubMed PMID: 32817593.
64. Ochs K, Ott M, Bunse T, Sahm F, Bunse L, Deumelandt K, et al. K27M-mutant histone-3 as a novel target for glioma immunotherapy. Oncoimmunology. 2017; 6(7):e1328340. Epub 2017 Aug. 16. doi: 10.1080/2162402X.2017.1328340. PubMed PMID: 28811969; PubMed Central PMCID: PMCPMC5543817.
65. Turcan S, Rohle D, Goenka A, Walsh L A, Fang F, Yilmaz E, et al. IDHI mutation is sufficient to establish the glioma hypermethylator phenotype. Nature. 2012; 483(7390):479-83. Epub 2012 Feb. 22. doi: 10.1038/nature 10866. PubMed PMID: 22343889; PubMed Central PMCID: PMCPMC3351699.
66. Schumacher T, Bunse L, Pusch S, Sahm F, Wiestler B, Quandt J, et al. A vaccine targeting mutant IDH1 induces antitumour immunity. Nature. 2014; 512(7514):324-7. Epub 2014 Jul. 22. doi: 10.1038/nature13387. PubMed PMID: 25043048.
67. Schumacher T, Bunse L, Wick W, Platten M. Mutant IDH1: An immunotherapeutic target in tumors. Oncoimmunology. 2014; 3(12):e974392. Epub 2015 May 13. doi: 10.4161/2162402X.2014.974392. PubMed PMID: 25964867; PubMed Central PMCID: PMCPMC4353168.
68. Vogelstein B, Papadopoulos N, Velculescu V E, Zhou S, Diaz L A, Jr., Kinzler K W. Cancer genome landscapes. Science. 2013; 339(6127): 1546-58. Epub 2013 Mar. 30. doi: 10.1126/science.1235122. PubMed PMID: 23539594; PubMed Central PMCID: PMCPMC3749880.
69. Kandoth C, McLellan M D, Vandin F, Ye K, Niu B, Lu C, et al. Mutational landscape and significance across 12 major cancer types. Nature. 2013; 502(7471):333-9. Epub 2013 Oct. 18. doi: 10.1038/nature 12634. PubMed PMID: 24132290; PubMed Central PMCID: PMCPMC3927368.
70. Zhu G, Pan C, Bei J X, Li B, Liang C, Xu Y, et al. Mutant p53 in Cancer Progression and Targeted Therapies. Frontiers in oncology. 2020; 10:595187. Epub 2020 Nov. 27. doi: 10.3389/fonc.2020.595187. PubMed PMID: 33240819; PubMed Central PMCID: PMCPMC7677253.
71. Smith S L, Pitt A R, Spickett C M. Approaches to Investigating the Protein Interactome of PTEN. Journal of proteome research. 2020. Epub 2020 Oct. 20. doi: 10.1021/acs.jproteome.0c00570. PubMed PMID: 33074689.
72. Crockett D K, Fillmore G C, Elenitoba-Johnson K S, Lim M S. Analysis of phosphatase and tensin homolog tumor suppressor interacting proteins by in vitro and in silico proteomics. Proteomics. 2005; 5(5):1250-62. Epub 2005 Feb. 18. doi: 10.1002/pmic.200401046. PubMed PMID: 15717329.
73. Wu H, Goel V, Haluska F G. PTEN signaling pathways in melanoma. Oncogene. 2003; 22(20):3113-22. Epub 2003 Jun. 6. doi: 10.1038/sj.onc. 1206451. PubMed PMID: 12789288.
74. Subramanian J, Katta A, Masood A, Vudem D R, Kancha R K. Emergence of ERBB2 Mutation as a Biomarker and an Actionable Target in Solid Cancers. Oncologist. 2019; 24(12):e1303-e14. Epub 2019 Jul. 12. doi: 10.1634/theoncologist.2018-0845. PubMed PMID: 31292270; PubMed Central PMCID: PMCPMC6975965.
75. Oh D Y, Bang Y J. HER2-targeted therapies—a role beyond breast cancer. Nat Rev Clin Oncol. 2020; 17(1):33-48. Epub 2019 Sep. 25. doi: 10.1038/s41571-019-0268-3. PubMed PMID: 31548601.
76. Arafeh R, Samuels Y. PIK3CA in cancer: The past 30 years. Semin Cancer Biol. 2019; 59:36-49. Epub 2019 Feb. 12. doi: 10.1016/j.semcancer.2019.02.002. PubMed PMID: 30742905.
77. Simanshu D K, Nissley D V, McCormick F. RAS Proteins and Their Regulators in Human Disease. Cell. 2017; 170(1): 17-33. Epub 2017 Jul. 1. doi: 10.1016/j.cell.2017.06.009. PubMed PMID: 28666118; PubMed Central PMCID: PMCPMC5555610.
78. Haigis K M. KRAS Alleles: The Devil Is in the Detail. Trends Cancer. 2017; 3(10):686-97. Epub 2017 Sep. 30. doi: 10.1016/j.trecan.2017.08.006. PubMed PMID: 28958387; PubMed Central PMCID: PMCPMC5824632.
79. McCormick F. KRAS as a Therapeutic Target. Clin Cancer Res. 2015; 21(8): 1797-801. Epub 2015 Apr. 17. doi: 10.1158/1078-0432.CCR-14-2662. PubMed PMID: 25878360; PubMed Central PMCID: PMCPMC4407814.
80. Ryall S, Zapotocky M, Fukuoka K, Nobre L, Guerreiro Stucklin A, Bennett J, et al. Integrated Molecular and Clinical Analysis of 1,000 Pediatric Low-Grade Gliomas. Cancer Cell. 2020; 37(4):569-83 e5. Epub 2020 Apr. 15. doi: 10.1016/j.ccell.2020.03.011. PubMed PMID: 32289278; PubMed Central PMCID: PMCPMC7169997.
81. Jones D T, Kocialkowski S, Liu L, Pearson D M, Backlund L M, Ichimura K, et al. Tandem duplication producing a novel oncogenic BRAF fusion gene defines the majority of pilocytic astrocytomas. Cancer Res. 2008; 68(21):8673-7. Epub 2008 Nov. 1. doi: 10.1158/0008-5472.CAN-08-2097. PubMed PMID: 18974108; PubMed Central PMCID: PMCPMC2577184.
82. Bayliss R, Choi J, Fennell D A, Fry A M, Richards M W. Molecular mechanisms that underpin EML4-ALK driven cancers and their response to targeted drugs. Cell Mol Life Sci. 2016; 73(6): 1209-24. Epub 2016 Jan. 13. doi: 10.1007/s00018-015-2117-6. PubMed PMID: 26755435; PubMed Central PMCID: PMCPMC4761370.
83. Sabir S R, Yeoh S, Jackson G, Bayliss R. EML4-ALK Variants: Biological and Molecular Properties, and the Implications for Patients. Cancers (Basel). 2017; 9(9). Epub 2017 Sep. 6. doi: 10.3390/cancers9090118. PubMed PMID: 28872581; PubMed Central PMCID: PMCPMC5615333.
84. Shaw A T, Yeap B Y, Mino-Kenudson M, Digumarthy S R, Costa D B, Heist R S, et al. Clinical features and outcome of patients with non-small-cell lung cancer who harbor EML4-ALK. Journal of clinical oncology: official journal of the American Society of Clinical Oncology. 2009; 27(26):4247-53. Epub 2009 Aug. 12. doi: 10.1200/JCO.2009.22.6993. PubMed PMID: 19667264; PubMed Central PMCID: PMCPMC2744268.
85. Horn L, Pao W. EML4-ALK: honing in on a new target in non-small-cell lung cancer. Journal of clinical oncology: official journal of the American Society of Clinical Oncology. 2009; 27(26):4232-5. Epub 2009 Aug. 12. doi: 10.1200/JCO.2009.23.6661. PubMed PMID: 19667260; PubMed Central PMCID: PMCPMC6955145.
86. Villaruz L C, Socinski M A. Personalized therapy for non-small cell lung cancer: which drug for which patient? Semin Thorac Cardiovasc Surg. 2011; 23(4):281-90. Epub 2011 Jan. 1. doi: 10.1053/j.semtcvs.2012.01.001. PubMed PMID: 22443647; PubMed Central PMCID: PMCPMC4836182.
87. Kim P, Tan H, Liu J, Lec H, Jung H, Kumar H, et al. FusionGDB 2.0: fusion gene annotation updates aided by deep learning. Nucleic acids research. 2021. Epub 2021 Nov. 11. doi: 10.1093/nar/gkab1056. PubMed PMID: 34755868.
88. Kim P, Zhou X. FusionGDB: fusion gene annotation DataBase. Nucleic acids research. 2019; 47(D1):D994-D1004. Epub 2018 Nov. 9. doi: 10.1093/nar/gky1067. PubMed PMID: 30407583; PubMed Central PMCID: PMCPMC6323909.
89. Benjamin D, Sato, T., Cibulskis, K., Getz, G., Stewart, C., Lichtenstein, L. Calling Somatic SNVs and Indels with Mutect2. bioRXiv. 2019. doi: doi.org/10.1101/861054.
90. Bremel R D, Homan E J. An integrated approach to epitope analysis II: A system for proteomic-scale prediction of immunological characteristics. ImmunomeRes. 2010; 6(1):8. doi: 1745-7580-6-8 [pii]; 10.1186/1745-7580-6-8 [doi].
91. Bremel R D, Homan E J. An integrated approach to epitope analysis I: Dimensional reduction, visualization and prediction of MHC binding using amino acid principal components and regression approaches. Immunome research. 2010; 6:7. Epub 2010 Nov. 4. doi: 10.1186/1745-7580-6-7. PubMed PMID: 21044289; PubMed Central PMCID: PMC2990731.
92. Croote D, Braslavsky I, Quake S R. Addressing Complex Matrix Interference Improves Multiplex Food Allergen Detection by Targeted LC-MS/MS. Anal Chem. 2019; 91(15):9760-9. Epub 2019 Jul. 25. doi: 10.1021/acs.analchem.9b01388. PubMed PMID: 31339301.
93. Asai Y, Eslami A, van Ginkel C D, Akhabir L, Wan M, Yin D, et al. A Canadian genome-wide association study and meta-analysis confirm HLA as a risk factor for peanut allergy independent of asthma. J Allergy Clin Immunol. 2018; 141(4):1513-6. Epub 2018 Jan. 13. doi: 10.1016/j.jaci.2017.10.047. PubMed PMID: 29325868.
94. Martino D J, Ashley S, Koplin J, Ellis J, Saffery R, Dharmage S C, et al. Genomewide association study of peanut allergy reproduces association with amino acid polymorphisms in HLA-DRB1. Clin Exp Allergy. 2017; 47(2):217-23. Epub 2016 Nov. 25. doi: 10.1111/cea.12863. PubMed PMID: 27883235.
95. Gonzalez-Fernandez J, Rivas L, Luque-Ortega J R, Nunez-Ramirez R, Campioli P, Garate T, et al. Recombinant vs native Anisakis haemoglobin (Ani s 13): Its appraisal as a new gold standard for the diagnosis of allergy. Exp Parasitol. 2017; 181:119-29. Epub 2017 Aug. 19. doi: 10.1016/j.exppara.2017.08.010. PubMed PMID: 28818650.
96. Morishima R, Motojima S, Tsuneishi D, Kimura T, Nakashita T, Fudouji J, et al. Anisakis is a major cause of anaphylaxis in seaside areas: An epidemiological study in Japan. Allergy. 2020; 75(2):441-4. Epub 2019 Jul. 18. doi: 10.1111/all.13987. PubMed PMID: 31315145.
97. Nieuwenhuizen N E. Anisakis—immunology of a foodborne parasitosis. Parasite Immunol. 2016; 38(9):548-57. Epub 2016 Jul. 19. doi: 10.1111/pim.12349. PubMed PMID: 27428817.

All publications and patents mentioned in the above specification are herein incorporated by reference as if expressly set forth herein. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in relevant fields are intended to be within the scope of the following claims.

1. A method for producing a personalized composition to treat a subject with cancer comprising designing a group of one or more tumor-specific T-cell stimulating peptides, or nucleic acids encoding T cell stimulating peptides, which have a desired predicted binding affinity for the MHC alleles of the subject, comprising the following steps:
obtaining a biopsy of the subject's tumor and a normal tissue sample;
obtaining DNA sequences from the tumor biopsy and normal tissue and RNA sequences from the tumor biopsy
obtaining sequences for proteins in the biopsy;
identifying proteins from the biopsy containing mutated amino acids and the peptide comprising each of the mutated amino acids;
determining T cell exposed motifs which comprise mutated amino acids in each of the proteins;
determining the predicted binding affinity to the subject's MHC alleles of peptides which comprises each of the T cell exposed motifs, or a subset thereof;
generating an array of alternative peptides not present in the tumor, wherein each peptide in the array comprises the amino acids of one of the T cell exposed motifs, and in which the amino acids not within the T cell exposed motif are substituted to change the predicted MHC binding affinity;
selecting a group of one or more selected peptides from the array of alternative peptides which have a desired predicted binding affinity for one or more of the subject's MHC alleles;
selecting from the array of alternative peptides those peptides in which those amino acids not located within the T cell exposed motif provide desired characteristics for formulation and delivery; and
synthesizing the group of one or more selected peptides, or nucleic acids encoding the selected peptides.

2. The method of claim 1 further comprising:
a. determining the fraction of the DNA in the tumor biopsy comprising genes that encode each of the proteins containing mutated amino acids, and the fraction of RNA transcribed from that gene locus and expressing the protein containing mutated amino acids;
b. selecting from the proteins containing mutated amino acids in the biopsy those which are present in at least 10% of the DNA in the biopsy and expressed in at least 10% of the RNA transcribed from that gene locus in the biopsy; and
c. generating the array of alternative peptides from these selected proteins.

3. The method of claim 1 further comprising:
a. determining the fraction of the DNA in the tumor biopsy comprising genes that encode each of the proteins containing mutated amino acids and the fraction of RNA transcribed from that gene locus and expressing the protein containing mutated amino acids;
b. selecting from the proteins containing mutated amino acids in the biopsy those which are present in at least 3% of the DNA in the biopsy and expressed in at least 10% of the RNA transcribed from that gene locus in the biopsy; and
c. generating the array of alternative peptides from these selected proteins.

4. The method of claim 1 further comprising
a. determining the fraction of the DNA in the tumor biopsy comprising genes that encode each of the proteins containing mutated amino acids and the fraction of RNA transcribed from that gene locus and expressing the protein containing mutated amino acids;
b. selecting from the proteins containing mutated amino acids in the biopsy those which are present in at least 10% of the DNA in the biopsy and expressed in at least 20% of the RNA transcribed from that gene locus in the biopsy; and
c. generating the array of alternative peptides from these selected proteins.

5. The method of claim 1, wherein the MHC alleles are MHC I alleles.

6. The method of claim 5, wherein the selected peptides are 8 to 10 amino acids in length.

7. The method of claim 1, wherein the MHC alleles are MHC II alleles.

8. The method of claim 7, wherein the selected peptides are from 11 to 22 amino acids in length.

9. The method of claim 1, wherein the T cell response is a cytotoxic T cell response.

10. The method of claim 1, wherein the T cell response is a T helper response.

11. The method of claim 1, wherein the group of selected peptides, or nucleic acids encoding the selected peptides, comprise peptides which bind an MHC I allele and peptides which bind an MHC II allele.

12. The method of claim 1, wherein the desired binding affinity to an MHC allele is less than 500 nM.

13. The method of claim 1, wherein the desired binding affinity to an MHC allele is less than 200 nM.

14. The method of claim 1, wherein the desired binding affinity to an MHC allele is less than 100 nM.

15. The method of claim 1, wherein the desired binding affinity to an MHC allele is less than 50 nM.

16. The method of claim 1, wherein each of the peptides identified in the biopsy as comprising a mutated amino acid in the step of identifying proteins from the biopsy containing mutated amino acids and the peptide comprising each of the mutated amino acids is bound to one or more of the subject's MHC alleles with an affinity that is higher than 500 nanomolar.

17. The method of claim 1, wherein the T cell exposed motif comprising the mutated amino acid is absent from the normal human proteome.

18. The method of claim 1, wherein the T cell exposed motif comprising the mutated amino acid occurs in less than 10 other protein sequence contexts in the human proteome.

19. The method of claim 1, wherein the T cell exposed motif comprising the mutated amino acid occurs in less than 10 other protein sequence contexts in the human proteome that have a predicted binding to the subject's MHC of <200 nM.

20. The method of claim 1, wherein the mutated amino acids comprise a substituted amino acid that is a product of a missense mutation.

21-199. (canceled)
